(19) United States
(12) Patent Application Publication
(10) Pub. No.: US 2024/0115595 A1
(43) Pub. Date: Apr. 11, 2024

(54) MATERIAL COMPOSITIONS AND METHODS OF USE IMPROVING RNA STABILITY

(71) Applicant: Team Medical, LLC, Boulder, CO (US)

(72) Inventors: Kyle P. HEIM, Boulder, CO (US); Warren P. HEIM, Boulder, CO (US)

(73) Assignee: Team Medical, LLC, Boulder, CO (US)

(21) Appl. No.: 18/464,129

(22) Filed: Sep. 8, 2023

Related U.S. Application Data
(60) Provisional application No. 63/374,928, filed on Sep. 8, 2022.

(51) Int. Cl. A61K 31/7105; A61K 47/18; A61K 47/22; A61K 47/24; A61K 47/26; A61K 47/36

(52) U.S. Cl. CPC A61K 31/7105; A61K 47/186; A61K 47/22; A61K 47/24; A61K 47/26; A61K 47/36

(57) ABSTRACT

Formulations of substances comprising at least one RNA stabilizing substance and at least one substance comprising extracellular RNA or based on RNA and methods of using the formulations to improve the storage and use stability of substances comprising extracellular RNA or based on RNA at temperatures of 4° C. and above in solution without lyophilization.

### CROSS-REFERENCE TO RELATED APPLICATIONS

This application claims priority to U.S. Provisional Application No. 63/374,928, entitled “RNA STABILIZATION”, filed Sep. 8, 2022, which is incorporated by reference herein in its entirety.

### FIELD OF THE INVENTION

The present disclosure relates to formulations and methods of using the formulations to improve the stability of various types of extracellular RNA and substances based on various types of extracellular RNA for storage and use in non-clinical applications and clinical applications including for therapeutic uses to diagnose the health or improve the health of living organisms including plants and animals including treating humans including diagnosis of diseases and treatment of diseases or other adverse health effects in animals including in humans.

### BACKGROUND OF THE INVENTION

Ribonucleic acid (RNA) is responsible for the transcription of the genetic information stored in deoxyribonucleic acid (DNA) in a form that can be used in cells to synthesize proteins. The use of therapeutic RNA to beneficially produce proteins, regulate gene expression, or induce immune responses to specific antigens and biomarkers has become an emerging part of the field of nucleic acid therapy. The potential applications and recognized advantages of RNA based nucleic acid therapies continue to increase. For example, the recent COVID-19 infections in humans have led to vaccines developed using messenger RNA (mRNA). RNA therapies, such as vaccines using mRNA have advantages compared to other therapies, such as traditional vaccines that use inactivated, attenuated, or genetically modified microorganisms, purified antigens, or viral vectors. These other therapies can lead to adverse reactions, side effects, allergic reactions, or can develop mutations, either during manufacturing or administration, that can alter the efficacy or lead to safety concerns. RNA encodes the genetic information for the target therapy to be produced endogenously within the host cell without the need to synthesize and purify individual antigens, thereby creating greater flexibility to specifically tailor different therapies for a variety of diseases and simplifying the manufacturing process by allowing the target cells to facilitate the production of the necessary proteins and reduce or eliminate the complications of traditional vaccines.

Using RNA for nucleic acid therapies has distinct advantages compared to DNA based therapies due to the relatively short half-life of RNA and the transient message encoded within the RNA that does not require entry into the nucleus for proper expression necessary to carry out the desired function. Furthermore, DNA can potentially integrate into the host cell genome and alter genomic DNA or also become inherited by progeny. However, the investigation of uses of RNA and the production of products using RNA is complicated by the limited stability of RNA. RNA is not as stable as DNA due to RNA's single stranded nature in many biological systems and the substitution of ribose within the sugar phosphate backbone (instead of deoxyribose in DNA), leading to the presence of a 2′-hydroxy group within the structure of the RNA backbone. The single stranded nature of RNA and the presence of the 2′-hydroxy makes RNA susceptible to hydrolysis, in which the RNA molecule is cleaved by breaking the phosphodiester bond in the sugar-phosphate backbone, leading to degradation of the RNA molecule. Storing RNA, including storing mRNA-based vaccines, requires conditions that slow or prevent RNA from degrading, as described below, and interfering with the desired effects that RNA induces in targeted living cells.

Storage at extreme low temperatures below the freezing point of water, such as at or below about −20° C. or even at or below about −80° C., is known to be useful or even required for durable storage of RNA, including, but not limited to, for example, vaccines based on mRNA.

Storing at extreme low temperatures is more complicated than storing at more easily achieved temperatures such as temperatures approximately at the freezing point of water or refrigerated temperatures or even room temperatures or other ambient temperatures. Among the complications associated with temperatures below ambient temperatures are that refrigeration means are needed. Such refrigeration means includes cooling using thermodynamic cycles such as mechanical refrigeration (including freezers and refrigerators), frozen carbon dioxide (dry ice), or frozen water (ice).

Storing at extreme low temperatures such as at or below about −70° C. or even at or below about −80° C. or at or below about −20° C. or at or below about 4° C. complicates and adds expense to storing substances containing or based on RNA, including the storage, transportation, and therapeutic access for administration of vaccines. The complexity of using RNA is reduced the closer to room temperatures or other ambient temperatures that RNA substances can be stable.

To reduce the complexity of storing RNA substances, including mRNA and substances containing or based on mRNA such as mRNA vaccines or other therapeutic products, materials and methods for improving the stability of RNA substances so that storage can be done at temperatures greater than extremely cold temperatures are needed.

Lyophilization or freeze-drying RNA substances is used to improve the storage stability of RNA and reduce the need for storing RNA at cold temperatures or even extreme cold temperatures. However, freeze-drying and lyophilization requires specialized equipment and adds significant time, expense, and complexity to the production and storage of RNA, including but not limited to mRNA and substances containing or based on mRNA such as mRNA vaccines or other therapeutic products.

Encapsulating or complexing RNA substances with nanoparticles or lipid nanoparticles is used to improve the delivery of an RNA substance to a cell or tissue. Nanoparticles may incorporate polyethylene glycol (PEG) modified lipids, PEG conjugated lipids, or similar modifications to improve nanoparticle stability. These modifications improve the stability of the nanoparticle by decreasing aggregation and agglomeration, as well as reducing protein binding and opsonization. However, improving nanoparticle stability relates to maintaining consistent nanoparticle size and distribution as well as improving circulation half-life and reducing systemic clearance of nanoparticles following administration of the encapsulated or complexed RNA and does not necessarily improve RNA stability by preventing or usefully reducing RNA degradation during storage or shipping or reducing the need for storing or shipping RNA at cold temperatures or even extreme cold temperatures.

### SUMMARY OF THE INVENTION

Accordingly, a primary objective of the present disclosure is to provide substances to the fields of therapeutics, diagnostics, and agriculture (including, without being limited, both human and non-human animals and plants) that increase the stability of RNA substances and reduce degradation of RNA substances in storage environments, including storage above temperatures that require freezing or refrigeration temperatures above about −80° C., above about −20° C., above about 0° C., above about 4° C., or above about 10° C.

Another primary objective of the present disclosure is to provide substances for storage environments for RNA substances and methods using substances in storage environments for RNA substances that reduces degradation of RNA substances so that RNA substances have improved stability when stored at temperatures at or above about −20° C. Another primary objective of the present disclosure is to provide substances for storage environments and methods using substances in storage environments for RNA substances that reduce degradation of RNA substances so that RNA substances have improved stability when stored at temperatures at or above about 4° C. Another primary objective of the present disclosure is to provide substances for storage environments and methods using substances in storage environments for RNA substances that reduce degradation of RNA substances so that RNA substances have improved stability when stored at temperatures at or above about 10° C.

Another primary objective of the present disclosure is to provide storage environments for RNA substances that reduce the needs for storing, transporting, distributing, or storing at a point of use such as a point for therapeutic administration (collectively “storage” or “storage and use” hereinafter) using thermodynamic cycle cooling such as vapor compression mechanical cooling or absorption cooling. Another primary objective of the present disclosure is to provide storage environments for RNA substances that reduce the needs for storage and use of RNA substances using dry ice. Another primary objective of the present disclosure is to provide storage environments for RNA substances that reduce the needs for storage and use of RNA substances using ice. Another primary objective of the present disclosure is to provide storage environments for RNA substances that reduce the needs for storage and use of RNA substances at temperatures of about −80° C. Another primary objective of the present disclosure is to provide storage environments for RNA substances that reduce the needs for storage and use of RNA substances at temperatures of about −20° C. Another primary objective of the present disclosure is to provide storage environments for RNA substances that reduce the needs for storage and use of RNA substances at temperatures of about 4° C.

The storage environment for RNA substances may contain at least one or more RNA stabilizing substance that is at least intimately associated with or at least partially contacting or at least partially surrounding at least one RNA substance such as by mixing, pipetting, blending, submerging, vortexing, shaking, lyophilizing, vaporizing, or sublimating. The storage environment includes the immediate environment of the RNA substance such as occurs when the RNA substance is mixed or is otherwise in close association or at least partially or substantially contacting one or more RNA stabilizing substances. As a non-limiting example, the storage environment for RNA substances may be at least some RNA stabilizing substance contacting at least part of one or more RNA substances at the molecular level such as may result from submerging, blending, or mixing one or more RNA substances with the one or more RNA stabilizing substance.

The inventors have discovered that mixtures comprising selected previously known substances that were not previously known to stabilize RNA or RNA substances and one or more RNA substance improves RNA stability. The inventors have also discovered that RNA stability may be improved with mixtures comprising one or more RNA substance and two, three, four, five, or more selected previously known substances not previously recognized as RNA stabilizing substances.

### Terminology

As used herein, the terms stabilize RNA or stabilizing RNA means reducing degradation of RNA substances. RNA degradation refers to cleavage of the phosphodiester backbone resulting in the reduction in molecular weight of one or more RNA molecules, such as by the loss of one or more nucleotides.

As used herein, an RNA stabilizing substance means a substance that stabilizes RNA of at least one or more RNA substance. RNA stabilizing substances provide a storage environment for the RNA substance that makes the RNA substance at least as stable at a higher temperature than the stability the RNA substance would have at a lower temperature.

As used herein a combination of one or more RNA substances with one or more RNA stabilizing substances comprises one or more RNA substances and one or more RNA stabilizing substances and the combination may include one or more additional other substances.

As used herein, cells means in vivo, in vitro, in situ, or ex vivo cells, including but not limited to eukaryotic cells, prokaryotic cells, plant cells, fungal cells, insect cells, bacterial cells, mycoplasma, protozoa, plasmodium, or mammalian cells, including but not limited to the cells of vertebrate animals and the cells of humans.

As used herein, nucleic acid means DNA, RNA, polymeric, single-stranded, double-stranded, or more highly aggregated hybridization motifs, and may include chemical modifications or analogs thereof. Modifications may include but are not limited to modifications comprising backbone modifications, sugar modifications, or base modifications. Modifications may also include, but are not limited to, those providing chemical groups that incorporate additional charge, polarizability, hydrogen bonding, electrostatic interaction, points of attachment or functionality to the nucleic acid bases or to the nucleic acid as a whole.

As used herein, the term RNA means ribonucleic acid, and may include chemical modifications or analogs thereof, with the exception of a chemical modification rendering the RNA into DNA. RNA may include RNA analogs, including nucleotide analogs. RNA may also include non-natural synthetic ribonucleotides. The RNA may be polymeric, single-stranded, or double stranded, or a more highly aggregated form. The RNA may be provided by one or more means known in the art, including but not limited to, in vitro transcription, purification from an organism, chemical synthesis, or a combination thereof. The RNA may be, but is not limited to, mRNA, rRNA, tRNA, microRNA, small interfering RNA (siRNA), self-amplifying RNA, small activating RNA, tmRNA, dsRNA, shRNA, snRNA, antisense RNA (asRNA), eRNA, RNA enzymes, CRISPR RNA, or total RNA. The RNA may be purified RNA (e.g., purified mRNA, purified rRNA, purified tRNA, purified microRNA, purified siRNA, purified self-amplifying RNA, purified small activating RNA, purified tmRNA, purified dsRNA, purified shRNA, purified snRNA, purified asRNA, purified eRNA, purified RNA enzymes, purified CRISPR RNA, or purified total RNA). Furthermore, RNA modifications may include sugar modifications or base modifications. RNA modifications may also include 5′ modifications or 3′ modifications, including but not limited to 5′-cap, 5′-cap structures, 5′-cap modifications, or 5′-cap analogs.

As used herein, the terms RNA substance or RNA substances means a substance or substances comprising at least one of extracellular RNA or purified extracellular RNA. RNA substance or RNA substances may include substances comprising one or more polymeric forms of RNA, including, but not limited to, single stranded or double stranded forms that may include, but are not limited to, coding or non-coding forms of RNA. RNA substance or RNA substances may also include, but are not limited to, mRNA and vaccines, therapeutics, diagnostics, or medicaments based on RNA, mRNA, or sections of RNA or other forms of ribonucleic acid that may be used for, including but not limited to, therapeutic, diagnostic, analysis, in vitro, in vivo, ex vivo, in situ, or other purposes. RNA substance or RNA substances may include, but are not limited to, mRNA, self-amplifying RNA, small activating RNA, rRNA, tRNA, microRNA, siRNA, tmRNA, dsRNA, shRNA, snRNA, asRNA, eRNA, RNA enzymes, CRISPR RNA, or total RNA. RNA substance or RNA substances may include, but are not limited to, purified RNA, including but not limited to, purified mRNA, purified rRNA, purified tRNA, purified microRNA, purified siRNA, purified self-amplifying RNA, purified small activating RNA, purified tmRNA, purified dsRNA, purified shRNA, purified snRNA, purified asRNA, purified eRNA, purified RNA enzymes, purified CRISPR RNA, or purified total RNA.

As used herein, RNA substances may include RNA modifications that may include, but are not limited to, those described in US Patent Application Pub. No. US 2020/0383922, incorporated herein by reference, as RNA modifications, chemical modifications, backbone modifications, sugar modifications, base modifications, nucleotide analogues/modifications, modified nucleosides, nucleoside modifications, lipid modifications, or 5′-CAP structures.

As used herein, RNA substances may include RNA modifications that may include, but are not limited to, those described in U.S. Pat. No. 10,702,600, incorporated herein by reference, as chemical modifications, modifications of polynucleotides, modified RNA polynucleotides, nucleoside or nucleotide modifications, modified nucleobases, or naturally occurring or non-naturally occurring modifications.

As used herein, RNA substances may include RNA modifications that may include, but are not limited to, those described in WO Patent Application Pub. No. WO 2021/156267 A1, incorporated herein by reference, as 5′-cap structures, cap analogues, RNA modifications, modified RNA, chemical modifications, backbone modifications, sugar modifications, base modifications, nucleotide analogues/modifications, or modified nucleotides.

As used herein, RNA substances may include RNA modifications that may include, but are not limited to, those described in US Patent Application Pub. No. US 2021/0260097, incorporated herein by reference, as modified mRNAs, mmRNAs, modified nucleobases, modified nucleosides, modified nucleotides, chemically modified mRNAs, or nucleoside modifications.

As used herein, RNA substances may include RNA modifications that may include, but are not limited to, those described in WO Patent Application Pub. No. WO 2021/213945 A1, incorporated herein by reference, as modified nucleosides, further modified nucleosides, modified nucleobases, modified nucleotides, 5′-cap, 5′cap-analog, or capping structure at the 5′-end of the RNA.

In some embodiments RNA substances may comprise an open reading frame. In some embodiments RNA substances may comprise a 5′-cap or 5′-cap structure. In some embodiments RNA substances may comprise a 5′ UTR. In some embodiments RNA substances may comprise a 3′ UTR. In some embodiments RNA substances may comprise a poly (A)-tail. In some embodiments RNA substances may be polymeric. In some embodiments RNA substances may be single stranded. In some embodiments RNA substances may be double stranded.

In some embodiments RNA substances may have one or more complimentary strands or partially complimentary strands, wherein a complimentary or partially complementary strand may include, but is not limited to, an RNA strand, DNA strand, peptide nucleic acid strand or other type of complementary or partially complementary strand.

In some embodiments RNA substances may comprise a coding RNA. As a non-limiting example, a coding RNA may include, but is not limited to, mRNA or self-amplifying RNA.

In some embodiments RNA substances may comprise one or more the following: mRNA or self-amplifying RNA.

In some embodiments RNA substances may comprise a non-coding RNA. As a non-limiting example, a non-coding RNA may include, but is not limited to, microRNA, siRNA, CRISPR RNA, antisense RNA, small activating RNA, or RNA enzymes.

In some embodiments RNA substances may comprise one or more of the following: microRNA, siRNA, CRISPR RNA, antisense RNA, small activating RNA, or RNA enzymes.

In some embodiments RNA substances may comprise at least 10 or more nucleotides, 50 or more nucleotides, 100 or more nucleotides, 200 or more nucleotides, 500 or more nucleotides, or 1000 or more nucleotides.

As used herein, the term storage environment means the substances in which one or more RNA substance is present other than substances when one or more RNA substance is present by being synthesized or produced or transcribed or deployed for immediate use.

Descriptions of substances herein may include one or more forms of the substance. These forms may include, but are not limited to, one more counterions, ionic forms, conjugate bases, conjugate acids, protonated or deprotonated forms, stereo isomers, chiral forms, salts, or combinations thereof.

As used herein, organic or organic substance means a substance comprising at least one or more carbon atom, wherein at least one or more carbon atom is covalently bonded to at least one or more hydrogen atom.

As used herein, an anion is an atom or group of atoms, with a negative charge at about pH 5-9.

As used herein, a cation is an atom or group of atoms, with a positive charge at about pH 5-9.

As used herein, a zwitterion is comprising both a positively charged cationic moiety and a negatively charged anionic moiety at about pH 5-9.

As used herein, an organosulfate comprises a sulfate group, wherein at least one oxygen atom is covalently bonded to at least one carbon atom.

As used herein, an organophosphate comprises a phosphate group, wherein at least one oxygen atom is covalently bonded to a carbon atom. Furthermore, an organophosphate may comprise one oxygen atom of a phosphate group covalently bonded to a carbon atom or two oxygen atoms of a phosphate group each covalently bonded to different carbon atoms.

As used herein, a heteroatom is a nitrogen, oxygen, or sulfur.

As used herein, alkyl refers to a branched or unbranched hydrocarbon group that lacks any double bonds; an alkyl may nevertheless be substituted to result in a substituted alkyl that may comprise (a) one or more atoms other than hydrogen and carbon and/or (b) one or more double bonds. When alkyl is methyl, which lacks any double bonds, for example, then alkyl may be substituted with oxo to result in formyl or alkyl may be substituted with phenyl to result in benzyl, both of which substituted alkyls comprise double bonds.

As used herein, alkenyl refers to a branched or unbranched hydrocarbon group that comprises at least one carbon-carbon double bond and that lacks any triple bonds.

Herein the term quaternary ammonium is used and the term quaternary amine is used and they are synonyms and mean the same thing.

As used herein, NDSB means non-detergent sulfobetaine.

### BRIEF DESCRIPTION OF THE DRAWINGS

For a more complete understanding of the present disclosure, and further advantages thereof, reference is now made to the following detailed description, taken in conjunction with the drawings, as described below.

FIG. 1 shows purified RNA following in vitro transcription analyzed by denaturing agarose gel electrophoresis.

FIG. 2 shows the results of denaturing agarose gel electrophoresis comparing stabilized RNA to a control in accordance with the present disclosure.

FIG. 3 shows the results of denaturing agarose gel electrophoresis comparing stabilized RNA to a control in accordance with the present disclosure.

FIG. 4 shows the results of denaturing agarose gel electrophoresis comparing stabilized RNA to a control in accordance with the present disclosure.

FIG. 5 shows the results of denaturing agarose gel electrophoresis comparing stabilized RNA to a control in accordance with the present disclosure.

FIG. 6 shows the results of denaturing agarose gel electrophoresis comparing stabilized RNA to a control in accordance with the present disclosure.

FIG. 7 shows the results of denaturing agarose gel electrophoresis comparing stabilized RNA to a control in accordance with the present disclosure.

FIG. 8 shows the results of denaturing agarose gel electrophoresis comparing stabilized RNA to a control in accordance with the present disclosure.

FIG. 9 shows the results of denaturing agarose gel electrophoresis comparing stabilized RNA to a control in accordance with the present disclosure.

FIG. 10 shows the results of denaturing agarose gel electrophoresis comparing stabilized RNA to a control in accordance with the present disclosure.

FIG. 11 shows the results of denaturing agarose gel electrophoresis comparing stabilized RNA to a control in accordance with the present disclosure.

FIG. 12 shows the results of denaturing agarose gel electrophoresis comparing stabilized RNA to a control in accordance with the present disclosure.

FIG. 13 shows the results of denaturing agarose gel electrophoresis comparing stabilized RNA to a control in accordance with the present disclosure.

FIG. 14 shows the results of denaturing agarose gel electrophoresis comparing stabilized RNA to a control in accordance with the present disclosure.

FIG. 15 shows the results of denaturing agarose gel electrophoresis comparing stabilized RNA to a control in accordance with the present disclosure.

FIG. 16 shows the results of denaturing agarose gel electrophoresis comparing stabilized RNA to a control in accordance with the present disclosure.

FIG. 17 shows the results of denaturing agarose gel electrophoresis comparing stabilized RNA to a control in accordance with the present disclosure.

FIG. 18 shows the results of denaturing agarose gel electrophoresis comparing stabilized RNA to a control in accordance with the present disclosure.

FIG. 19 shows the results of denaturing agarose gel electrophoresis comparing stabilized RNA to a control in accordance with the present disclosure.

FIG. 20 shows the results of denaturing agarose gel electrophoresis comparing stabilized RNA to a control in accordance with the present disclosure.

FIG. 21 shows the results of denaturing agarose gel electrophoresis comparing stabilized RNA to a control in accordance with the present disclosure.

FIG. 22 shows the results of denaturing agarose gel electrophoresis comparing stabilized RNA to a control in accordance with the present disclosure.

FIG. 23 shows the results of denaturing agarose gel electrophoresis comparing stabilized RNA to a control in accordance with the present disclosure.

FIG. 24 shows the results of denaturing agarose gel electrophoresis comparing stabilized RNA to a control in accordance with the present disclosure.

FIG. 25 shows the results of denaturing agarose gel electrophoresis comparing stabilized RNA to a control in accordance with the present disclosure.

FIG. 26 shows the results of denaturing agarose gel electrophoresis comparing stabilized RNA to a control in accordance with the present disclosure.

FIG. 27 shows the results of denaturing agarose gel electrophoresis comparing stabilized RNA to a control in accordance with the present disclosure.

FIG. 28 shows the results of denaturing agarose gel electrophoresis comparing stabilized RNA to a control in accordance with the present disclosure.

FIG. 29 shows the results of denaturing agarose gel electrophoresis comparing stabilized RNA to a control in accordance with the present disclosure.

FIG. 30 shows the results of denaturing agarose gel electrophoresis comparing stabilized RNA to a control in accordance with the present disclosure.

FIG. 31 shows the results of denaturing agarose gel electrophoresis comparing stabilized RNA to a control in accordance with the present disclosure.

FIG. 32 shows the results of denaturing agarose gel electrophoresis comparing stabilized RNA to a control in accordance with the present disclosure.

FIG. 33 shows the results of denaturing agarose gel electrophoresis comparing stabilized RNA to a control in accordance with the present disclosure.

FIG. 34 shows the results of denaturing agarose gel electrophoresis comparing stabilized RNA to a control in accordance with the present disclosure.

FIG. 35 shows the results of denaturing agarose gel electrophoresis comparing stabilized RNA to a control in accordance with the present disclosure.

FIG. 36 shows the results of denaturing agarose gel electrophoresis comparing stabilized RNA to a control in accordance with the present disclosure.

FIG. 37 shows the results of denaturing agarose gel electrophoresis comparing stabilized RNA to a control in accordance with the present disclosure.

FIG. 38 shows the results of denaturing agarose gel electrophoresis comparing stabilized RNA to a control in accordance with the present disclosure.

FIG. 39 shows the results of denaturing agarose gel electrophoresis comparing stabilized RNA to a control in accordance with the present disclosure.

FIG. 40 shows the results of denaturing agarose gel electrophoresis comparing stabilized RNA to a control in accordance with the present disclosure.

FIG. 41 shows the results of denaturing agarose gel electrophoresis comparing stabilized RNA to a control in accordance with the present disclosure.

FIG. 42 shows the results of denaturing agarose gel electrophoresis comparing stabilized RNA to a control in accordance with the present disclosure.

FIG. 43 shows the results of denaturing agarose gel electrophoresis comparing stabilized RNA to a control in accordance with the present disclosure.

FIG. 44 shows the results of denaturing agarose gel electrophoresis comparing stabilized RNA to a control in accordance with the present disclosure.

FIG. 45 shows the results of denaturing agarose gel electrophoresis comparing stabilized RNA to a control in accordance with the present disclosure.

FIG. 46 shows the results of denaturing agarose gel electrophoresis comparing stabilized RNA to a control in accordance with the present disclosure.

FIG. 47 shows the results of denaturing agarose gel electrophoresis comparing stabilized RNA to a control in accordance with the present disclosure.

FIG. 48 shows the results of denaturing agarose gel electrophoresis comparing stabilized RNA to a control in accordance with the present disclosure.

FIG. 49 shows the results of denaturing agarose gel electrophoresis comparing stabilized RNA to a control in accordance with the present disclosure.

FIG. 50 shows the results of denaturing agarose gel electrophoresis comparing stabilized RNA to a control in accordance with the present disclosure.

FIG. 51 shows the results of denaturing agarose gel electrophoresis comparing stabilized RNA to a control in accordance with the present disclosure.

FIG. 52 shows the results of denaturing agarose gel electrophoresis comparing stabilized RNA to a control in accordance with the present disclosure.

FIG. 53 shows the results of denaturing agarose gel electrophoresis comparing stabilized RNA to a control in accordance with the present disclosure.

FIG. 54 shows the results of denaturing agarose gel electrophoresis comparing stabilized RNA to a control in accordance with the present disclosure.

FIG. 55 shows the results of denaturing agarose gel electrophoresis comparing stabilized RNA to a control in accordance with the present disclosure.

FIG. 56 shows the results of denaturing agarose gel electrophoresis comparing stabilized RNA to a control in accordance with the present disclosure.

FIG. 57 shows a multi-compartment syringe loaded with components of an RNA composition in accordance with the present disclosure.

FIG. 58 shows an RNA composition kit in accordance with the present disclosure.

FIG. 59 is a flow chart illustrating a process for producing and using an RNA product in accordance with the present disclosure.

FIG. 60 is a flow chart illustrating a process for producing and using an RNA product in accordance with the present disclosure.

FIG. 61 shows a vial filled with components of an RNA composition in accordance with the present disclosure.

FIG. 62 shows a single-compartment syringe filled with components of an RNA composition in accordance with the present disclosure.

FIG. 63 shows an embedded complex with components of an RNA composition in accordance with the present disclosure.

FIG. 64 illustrates an embodiment of the present disclosure comprising an RNA substance and an RNA stabilizing substance.

FIG. 65 illustrates an embodiment of the present disclosure comprising a chamber and an RNA substance and an RNA stabilizing substance.

FIG. 66 illustrates an embodiment of the present disclosure comprising a kit.

FIG. 67 illustrates an embodiment of the present disclosure comprising an RNA substance and an RNA stabilizing substance.

### DETAILED DESCRIPTION OF THE INVENTION

Before explaining various embodiments of RNA stabilizing substances and storage environments of RNA substances in detail, it should be noted that the illustrative embodiments and examples are not limited in application or use to the details of construction and arrangement of parts illustrated in the accompanying drawings and description. The illustrative embodiments and examples may be implemented or incorporated in other embodiments, variations and modifications, and may be practiced or carried out in various ways. Further, unless otherwise indicated, the terms and expressions employed herein have been chosen for the purpose of describing the illustrative embodiments for the convenience of the reader and are not for the purpose of limitation thereof. Also, it will be appreciated that one or more of the following-described embodiments, expressions of embodiments and/or examples, may be combined with one or more of the other following-described embodiments, expressions of embodiments and/or examples.

As used herein the term “or” means and/or unless the context states otherwise.

As used herein the term “a” means one or more unless the context states otherwise.

As used herein the term “an” means one or more unless the context states otherwise.

The headings in this section are present for convenience and are not limiting descriptions of embodiments of the present disclosure.

It is of great interest to the field of therapeutics, diagnostics, and agriculture to increase the stability of RNA substances and reduce degradation of RNA substances. Described herein, are compositions (including pharmaceutical compositions and therapeutic compositions) and methods for the design, preparation, manufacture and/or formulation of storage environments to stabilize RNA substances. Also provided are systems, processes, and devices for selection, design, and/or utilization of the storage environments to stabilize RNA substances described herein.

This disclosure describes newly discovered compositions that are previously unknown RNA stabilizing substances, comprising previously known substances that were not previously known to stabilize RNA or RNA substances. The present inventors have discovered previously known substances that were previously unrecognized to stabilize RNA or RNA substances, surprisingly stabilize RNA and RNA substances.

The present inventors have discovered compositions of previously known substances that were previously unrecognized to stabilize RNA or RNA substances, that surprisingly stabilize RNA or RNA substances. The present inventors have discovered combinations of selected previously known materials, materials previously unrecognized to stabilize RNA or RNA substances, wherein the combinations surprisingly stabilize RNA or RNA substances.

The present inventors have discovered that selected previously known materials, previously unrecognized to stabilize RNA or RNA substances, may be used individually or in combinations as RNA stabilizing substances and that compositions comprising these previously unrecognized RNA stabilizing substances and RNA substances may increase RNA stability. The present inventors have discovered that compositions comprising at least one or more RNA stabilizing substance and at least one or more RNA substance may increase RNA stability compared to compositions containing at least one RNA substance without at least one or more RNA stabilizing substances.

The inventors have also discovered that the stability of RNA substances may be enhanced in compositions comprising an RNA substance and one or more RNA stabilizing substance. The inventors have also discovered that the stability of RNA substances may be enhanced in an RNA storage environment comprising one or more RNA stabilizing substance.

The inventors have discovered families of compounds comprising specific moieties may be used to improve the stability of RNA substances. The inventors have discovered families of compounds comprising quaternary ammonium, tertiary sulfonium, carboxylate, sulfonate, sulfate, and phosphate groups (collectively NSCSSP compounds or NSCSSP groups or NSCSSP moieties) may be RNA stabilizing substances. These specific moieties are shared across many different types of RNA stabilizing substances. The inventors have surprisingly discovered, that while RNA stabilizing substances may have various chemical formulas and structures, they often share specific elements that act as a common thread. As a non-limiting example, the inventors have surprisingly discovered that many RNA stabilizing substances often contain the following groups:

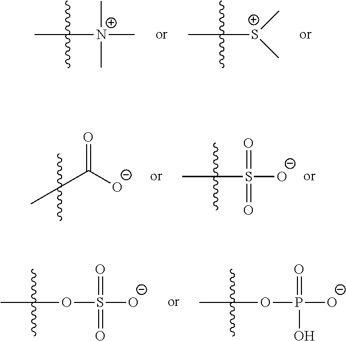

These groups are often shared among a variety of RNA stabilizing substances and help to classify RNA stabilizing substances into several families or categories.

The inventors have discovered families of compounds comprising NSCSSP moieties may stabilize RNA substances in chambers larger than nanoscale and in solution without lyophilization at refrigerated temperatures and above refrigerated temperatures for hours, days, or months.

The inventors have discovered categories of existing compounds that were previously unrecognized to stabilize RNA or RNA substances, surprisingly stabilize RNA or RNA substances. These existing compounds are members of families of RNA stabilizing substances described in this disclosure.

As described in this disclosure, the families of RNA stabilizing substances comprising NSCSSP moieties are classified into categories of chemical compounds with defined compositions.

The inventors have also discovered compounds that may not comprise NSCSSP moieties that stabilize RNA substances (as a non-limiting example, 3-O-ethyl-L-ascorbic acid) and those substances also have defined compositions described herein.

The inventors have also discovered that compositions comprising RNA stabilizing substances comprising multiple NSCSSP moieties and defined compositions of those RNA stabilizing substances are described herein (as a non-limiting example, dimethylsulfoniopropionate (DMSP)).

The inventors have also discovered that compositions comprising RNA stabilizing substances comprising modified carbohydrates comprising NSCSSP moieties and defined compositions of those RNA stabilizing substances are described herein (as a non-limiting example, phytate).

The inventors have also discovered that compositions comprising RNA stabilizing substances comprising polymers comprising NSCSSP moieties and defined compositions of those RNA stabilizing substances are described herein (as a non-limiting example, poly(2-(trimethylamino)ethyl methacrylate) (PTMAEMA)).

Non-limiting embodiments of the present disclosure may comprise one or more RNA stabilizing substances. As a non-limiting example, one or more RNA stabilizing substances may be prepared and packaged or otherwise stored for later use in a kit comprising a package with one or more RNA stabilizing substances and labeling that may, for example, describe the contents of the kit and its intended use to be mixed with at least one RNA substance to improve the stability of the at least one RNA substance. As another non-limiting example, the one or more RNA stabilizing substance may be a component in a mixture further comprising an RNA substance.

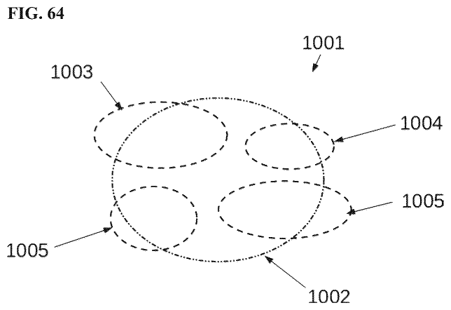

Non-limiting embodiments of the present disclosure may comprise one or more RNA substance and one or more RNA stabilizing substance. FIG. 64 schematically portrays a non-limiting example of the present disclosure comprising an RNA stabilizing substance and an RNA containing substance. Stabilized composition 1001 comprises at least one RNA substance 1002 and RNA stabilizing substance 1 1003. Stabilized composition 1001 may also comprise additional RNA stabilizing substances, as illustrated by RNA stabilizing substance 2 1004. Stabilizing composition 1001 may comprise property enhancing substance 1005 that enhances properties of stabilized composition 1001. As a non-limiting example, property enhancing substance 1005, may improve the biological performance of stabilized composition 1001, when stabilized composition 1001 is a medicant that will be administered to provide one or more therapies, such as property enhancing substance 1005 being a cellular uptake agent. In some embodiments of the present disclosure a composition comprising one or more RNA substance and one or more RNA stabilizing substance may be at least partially biocompatible.

Non-limiting embodiments of the present disclosure may include compositions comprising one or more RNA substance and one or more RNA stabilizing substance. Non-limiting embodiments of the present disclosure may include compositions of materials that comprise one or more RNA substance and at least one or more RNA stabilizing substance. Non-limiting embodiments of the present disclosure include compositions with improved RNA stability that may comprise one or more RNA substance and one or more RNA stabilizing substance. Non-limiting embodiments of the present disclosure may include compositions comprising one or more RNA stabilizing substance and one or more nucleic acid substance. Non-limiting embodiments of the present disclosure may include compositions comprising one or more RNA stabilizing substance and one or more DNA substance.

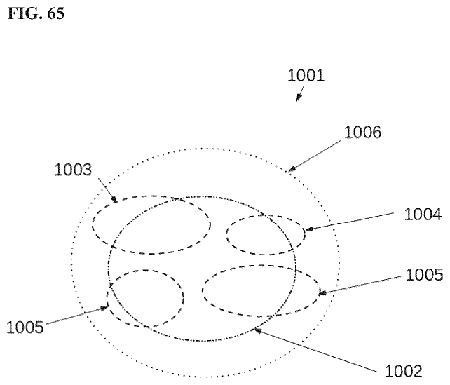

Non-limiting embodiments of the present disclosure may include compositions comprising one or more RNA substance and one or more RNA stabilizing substance in a chamber. FIG. 65 schematically portrays a non-limiting example of the present disclosure comprising an RNA stabilizing substance, an RNA containing substance, and a chamber. Stabilized composition 1001 comprises the components illustrated in FIG. 64 and those components are in chamber 1006. Stabilized composition chamber 1006 will be larger than nanoscale, as a non-limiting example, with at least one exterior dimension (e.g., length, width, thickness, diameter, perimeter) larger than about 10 micrometers. Stabilized composition chamber 1006 may be, as non-limiting examples, an ingestible capsule containing at least one RNA substance 1002 and at least one RNA stabilizing substance 1 1003 or an implantable chamber containing at least one RNA substance 1002 and at least one RNA stabilizing substance 1 1003. Chamber 1006 may be, as non-limiting examples, a hermetically sealed vial containing at least one RNA substance 1002 and at least on RNA stabilizing substance 1 1003 or a prefilled syringe containing at least one RNA substance 1002 and at least on RNA stabilizing substance 1 1003. Non-limiting embodiments of the present disclosure that improve the stability of RNA substances may comprise a storage environment comprising at least one or more vapor, liquid, powder, or solid RNA stabilizing substance.

In non-limiting example embodiments of the present disclosure, the storage environment may comprise an RNA stabilizing substance that is a liquid at the storage temperature. In non-limiting example embodiments of the present disclosure, the storage environment may comprise an RNA stabilizing substance that is a solid at the storage temperature. As a non-limiting example, some substances, such as DMSO, change from being at least approximately a solid to at least approximately a liquid at its melting point of about 19° C. at one atmosphere pressure. As a non-limiting example, the storage environment comprises a material that undergoes a phase change from solid liquid with the solid phase condition being desired for at least part of the storage duration and the liquid phase being desired for at least another part of the storage duration. As a non-limiting example, the solid phase may provide better RNA stability than the liquid phase and the liquid phase may provide better egress or dispensing from storage chambers. As a non-limiting example the transitions between liquid and solid or solid to liquid may help stabilize the temperature of the storage environment (and thus help maintain the temperature of RNA substances) by releasing energy when the phase changes from liquid to solid or by absorbing energy when the phase changes from solid to liquid.

In non-limiting example embodiments of the present disclosure, the storage environment may comprise an RNA stabilizing substance that changes to vapor phase at storage conditions. As a non-limiting example, the energy used to transition from liquid to vapor to solid to vapor may be thermal energy removed from the storage environment and help maintain a stable temperature, or energy may be released to the storage environment to help maintain a stable environment when the transition is from vapor to liquid.

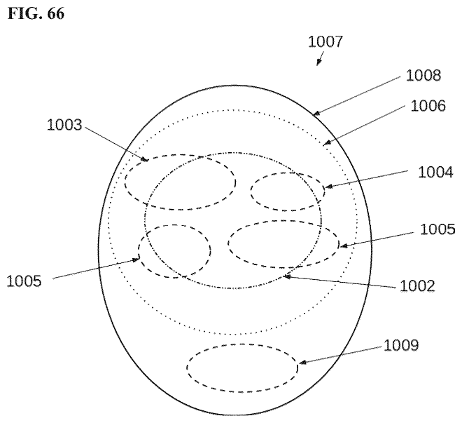

In non-limiting example embodiments of the present disclosure, components included in a composition comprising one or more RNA substance and one or more RNA stabilizing substance, may be stored separately, such as in a kit, or such as individual substances or as mixtures of one or more substance, and then combined later to produce a composition comprising one or more RNA substance and one or more RNA stabilizing substance. In non-limiting embodiments of the present disclosure components of a composition comprising one or more RNA substance and one or more RNA stabilizing substance, may be stored together and be part of a kit comprising instructions for use or other labeling. FIG. 66 schematically portrays a non-limiting example of the present disclosure comprising an RNA stabilizing composition kit 1007. Stabilized composition kit 1007 comprises the components illustrated in FIG. 65 and those components are in kit package 1008. As non-limiting examples, kit package 1008 may be an outer wrap such as a box or bag made from a suitable material such as hard or flexible plastic or paper or cardboard. Stabilized composition kit 1007 may also comprise labeling 1009 which, as a non-limiting example, may comprise directions for use or descriptions of RNA substance 1002. Such labeling may part of kit 1007 by, as non-limiting examples, being at least partially affixed to or inside of kit package 1008 or being part of chamber 1006 by, as non-limiting examples, being labeling affixed to or being part of chamber 1006.

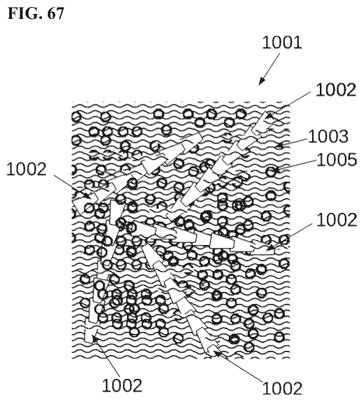

FIG. 67 illustrates a non-limiting embodiment of the present disclosure. Stabilized composition 1001 comprises at least one RNA substance 1002 that at least partly contacts RNA stabilizing substance 1003. As described in more detail later, substance 1005 may be another RNA stabilizing substance or may be a substance that enhances properties of stabilized composition 1001 such as improving the biological performance of stabilized composition 1001 when it is a medicant that will be administered to provide one or more therapies. RNA stabilizing substance 1003 may be, as a non-limiting example, a liquid at approximately 20° C. that is stabilizing RNA substance 1002 that is at substantially the same temperature as RNA stabilizing substance 1003. As a non-limiting example, stabilized composition 1001 may be part of a kit (not shown) in which the kit (not shown) comprises a chamber (not shown) containing stabilized composition 1001. In non-limiting embodiments of the present disclosure a composition comprising one or more RNA substance and one or more RNA stabilizing substance may be at least partially biocompatible.

Embodiments of the present disclosure may comprise one or more composition described herein comprising at least one RNA stabilizing substance and may also include one or more additional other substance, such as one or more cellular uptake agent, or one more additional RNA stabilizing substance, additive substances, or water as non-limiting examples. Such embodiments may be used as at least part of therapeutic substances, pharmaceutical compositions, medicaments, vaccines, or biostimulants, such as, as a non-limiting example, vaccines deploying mRNA to one or more living organisms (which may include humans or may include non-human animals) with at least one RNA stabilizing substance improving the stability of the therapeutic substance and, if present, at least one cellular uptake agent improving the efficacy of the therapeutic substance.

Embodiments of the present disclosure may comprise one or more RNA stabilizing substance and one or more cellular uptake agent. Such embodiments may be used as at least part of one or more therapeutic substances, pharmaceutical compositions, medicaments, vaccines, or biostimulants, such as, as a non-limiting example, vaccines deploying mRNA to one or more living organisms (which may include humans or may include non-human animals or may include plants) with at least one RNA stabilizing substance improving the stability of the therapeutic substance and at least one cellular uptake agent improving the efficacy of the therapeutic substance.

Non-limiting embodiments of the present disclosure may include a combination or mixture comprising one or more RNA substance and one or more RNA stabilizing substance. Non-limiting embodiments of the present disclosure that comprise one or more RNA substance and one or more RNA stabilizing substance may include combining, such as by mixing, one or more RNA substance with one or more substance that comprises at least one or more RNA stabilizing substance. In non-limiting embodiments of the present disclosure compositions with improved RNA stability may comprise a mixture of one or more RNA substance and one or more RNA stabilizing substance. Non-limiting embodiments of the present disclosure that comprise one or more RNA substance and one or more RNA stabilizing substance may include combining, such as by mixing, one or more RNA substance with one or more substance that comprises at least one or more RNA stabilizing substance. In non-limiting embodiments of the present disclosure a composition comprising one or more RNA substance and one or more RNA stabilizing substance produces a mixture with at least one or more RNA substance and at least one or more RNA stabilizing substance. In non-limiting embodiments of the present disclosure a combination comprising one or more RNA substance and one or more RNA stabilizing substance produces a mixture with at least one or more RNA substance and at least one or more RNA stabilizing substance. As non-limiting embodiments of the present disclosure, a composition combining at least one or more RNA substance and one or more RNA stabilizing substance may produce a mixture with at least one or more RNA substance and at least one or more RNA stabilizing substance.

### Mechanisms and Theory

RNA is naturally unstable, including in aqueous solutions. RNA is susceptible to degradation including, but not limited to the following types of degradation: enzymatic, autocatalytic, metal-catalyzed, autohydrolysis, hydrolysis, temperature induced, pH induced, chemically induced, oxidation induced, reduction induced, or radiation induced. Without being bound to any particular mechanism or mode of action, it is believed that the present disclosure provides a storage environment that increases the stability of RNA substances at warmer than extreme cold conditions by reducing the chemical reactions that degrade RNA such as by reducing exposure of RNA substances to substances within the storage environment that may induce RNA degradation.

The present inventors have discovered that RNA stabilizing substances can surprisingly increase the stability of RNA substances at temperatures above about −80° C. The present inventors have also discovered that RNA stabilizing substances can surprisingly increase the stability of RNA substances at temperatures above about −20° C. The present inventors have also discovered that RNA stabilizing substances can surprisingly increase the stability of RNA substances at temperatures above about 0° C. The present inventors have also discovered that RNA stabilizing substances can surprisingly increase the stability of RNA substances at temperatures above about 4° C. The present inventors have also discovered that RNA stabilizing substances can surprisingly increase the stability of RNA substances at temperatures above about 10° C. The present inventors have also discovered that RNA stabilizing substances can surprisingly increase the stability of RNA substances at temperatures above about 20° C.

The inventors have discovered that RNA stabilizing substances may improve the stability of RNA substances in the presence of water. Embodiments of the present disclosure may include compositions that may comprise at least one RNA stabilizing substance, at least one RNA substance, and water. These embodiments that may comprise water may be one or more composition as described herein that may also comprise water. Embodiments of the present disclosure may comprise one or more composition described herein and may include one or more additional other substances of which water may be one of the additional other substances.

Without being bound to any particular mechanism or mode of action, RNA hydrolysis can be initiated by a base removing a proton from the 2′-OH on the ribose sugar, leading to the subsequent nucleophilic attack of the 2′ oxygen on the adjacent phosphorus atom. Base catalyzed hydrolysis activates the 2′-OH by removing the proton and creating a negatively charged 2′ oxygen and promoting nucleophilic attack of the 2′ oxygen on the adjacent phosphorus atom of the phosphodiester backbone of RNA. Water's protic nature to both donate and accept protons allows water to act as both an acid and a base at about physiologic pH. Therefore, water is capable of acting as a proton acceptor and activating the 2′-OH to promote the nucleophilic attack of the 2′ oxygen on the adjacent phosphorus atom of the phosphodiester backbone of the RNA molecule to promote RNA hydrolysis.

Without being bound to any particular mechanism or mode of action, RNA stabilizing substances of the present disclosure may at least inhibit a base removing a proton from the 2′-OH on the ribose sugar or nucleophilic attack of the 2′ oxygen on the adjacent phosphorus atom. For chemical reactions to occur reactants or reagents need to be jointly accessible and enough energy needs to be present to overcome the activation energy required for a reaction to proceed. As a non-limiting example, increasing the activation energy required for a reaction to proceed or depriving access or availability to reactants or reagents may be one mechanism by which RNA stabilizing substances improve RNA stability. Without being bound to any particular mechanism or mode of action, one or more RNA stabilizing substance may at least reduce access to the RNA substance by materials that may promote RNA hydrolysis or degradation of the RNA substance. In a non-limiting example, one or more RNA stabilizing substance in the environment of the RNA substance may create an environment that excludes water from the RNA substance or reduces the concentration of water in the environment around the RNA substance or alters the water structure or hydrogen bonding network of water or the environment around the RNA substance. Therefore, if one or more RNA stabilizing substances substantially displace all of the water in the environment of the RNA substance then the RNA substance is substantially not exposed to water, ions, or other materials that may promote RNA hydrolysis. In another non-limiting example, one or more RNA stabilizing substance in the environment of the RNA substance may also create an environment that limits the molecular mobility of water, ions, or other materials and thereby limit and/or prevent the exposure of the RNA substance to water, ions, or other materials that may promote RNA hydrolysis Double stranded RNA substances are more stable than single stranded RNA substances.

Without being bound to any particular mechanism or mode of action, the increased stability of double stranded RNA is at least partially a result of the decreased flexibility of the double stranded RNA substance which reduces the movement of the RNA substance creating a lower probability that a 2′-OH will be in close enough proximity to an adjacent phosphorus atom to perform a nucleophilic attack and initiate RNA hydrolysis. In a non-limiting example, one or more RNA stabilizing substance that reduces the flexibility or molecular movement of a single stranded RNA substance reduces the likelihood that a 2′-OH will be in close enough proximity to an adjacent phosphorus atom to perform a nucleophilic attack and initiate RNA hydrolysis.

### RNA Stabilizing Substances

The inventors have discovered that RNA stabilizing substances may stabilize RNA substances.

The inventors have also discovered that the stability of RNA substances may be enhanced in an RNA storage environment comprising one or more RNA stabilizing substance.

The inventors have also discovered that the stability of RNA substances may be enhanced in compositions comprising an RNA substance and one or more RNA stabilizing substance.

Embodiments of the present disclosure may include a composition comprising one or more RNA substance and one or more RNA stabilizing substance.

Embodiments of the present disclosure may include a combination or mixture comprising one or more RNA substance and one or more RNA stabilizing substance.

Embodiments of the present disclosure that comprise one or more RNA substance and one or more RNA stabilizing substance may include combining, such as by mixing, one or more RNA substance with one or more substance that comprises at least one or more RNA stabilizing substance.

Embodiments of the present disclosure may include compositions of materials that comprise one or more RNA substance and at least one or more RNA stabilizing substance. The storage environment that improves the stability of RNA substances may comprise at least one or more vapor, liquid, powder, or solid RNA stabilizing substance.

Embodiments of the present disclosure may include combinations of materials that comprise one or more RNA substance and at least one or more RNA stabilizing substance.

In some embodiments of the present disclosure a composition comprising one or more RNA substance and one or more RNA stabilizing substance, produces a mixture with at least one or more RNA substance and at least one or more RNA stabilizing substance.

In some embodiments of the present disclosure a combination comprising one or more RNA substance and one or more RNA stabilizing substance, produces a mixture with at least one or more RNA substance and at least one or more RNA stabilizing substance.

In some embodiments of the present disclosure a composition with improved RNA stability may comprise one or more RNA substance and one or more RNA stabilizing substance.

In some embodiments of the present disclosure a composition with improved RNA stability may comprise a mixture of one or more RNA substance and one or more RNA stabilizing substance.

In some embodiments of the present disclosure, each component included in a composition comprising one or more RNA substance and one or more RNA stabilizing substance, may be stored separately, such as in a kit, or such as individual substances or as mixtures of one or more substance, and then combined later to produce a composition comprising one or more RNA substance and one or more RNA stabilizing substance.

In some embodiments of the present disclosure a composition comprising one or more RNA substance and one or more RNA stabilizing substance may be at least partially biocompatible.

In some embodiments of the present disclosure a combination comprising one or more RNA substance and one or more RNA stabilizing substance may be at least partially biocompatible.

The inventors have also discovered that the stability of RNA substances may be enhanced in an RNA storage environment comprising multiple RNA stabilizing substances, wherein a composition or combination may comprise an RNA substance and two more RNA stabilizing substances, or three or more RNA stabilizing substances, or four or more RNA stabilizing substances, or five or more RNA stabilizing substances, or greater.

The inventors have also discovered that the stability of RNA substances may be enhanced in compositions comprising an RNA substance and multiple RNA stabilizing substances, wherein a composition may comprise an RNA substance and two more RNA stabilizing substances, or three or more RNA stabilizing substances, or four or more RNA stabilizing substances, or five or more RNA stabilizing substances, or greater.

Embodiments of the present disclosure may include compositions comprising one or more RNA substance and one or more RNA stabilizing substance. Other embodiments of the present disclosure may include compositions comprising one or more RNA substance and multiple RNA stabilizing substances, wherein a composition may comprise an RNA substance and two more RNA stabilizing substances, or three or more RNA stabilizing substances, or four or more RNA stabilizing substances, or five or more RNA stabilizing substances, or greater.

Embodiments of the present disclosure may include a combination or mixture comprising one or more RNA substance and one or more RNA stabilizing substance. Other embodiments of the present disclosure may include a combination or mixture comprising one or more RNA substance and multiple RNA stabilizing substances, wherein a combination or mixture may comprise an RNA substance and two more RNA stabilizing substances, or three or more RNA stabilizing substances, or four or more RNA stabilizing substances, or five or more RNA stabilizing substances, or greater.

The inventors have discovered that compositions or mixtures comprising at least one or more RNA substance and at least one or more RNA stabilizing substance may improve RNA stability. The inventors have also discovered that RNA stability may be improved with compositions or mixtures comprising at least one or more RNA substance and multiple RNA stabilizing substances, wherein a composition or mixture may comprise an RNA substance and two more RNA stabilizing substances, or three or more RNA stabilizing substances, or four or more RNA stabilizing substances, or five or more RNA stabilizing substances, or greater.

Embodiments comprising one or more RNA substance and one or more RNA stabilizing substance, may also comprise multiple types of RNA substances, wherein the number of different types of RNA substances may be two or more, three or more, four or more, five or more, six or more, or greater. In some embodiments comprising multiple types of RNA substances, the RNA substances may be coding or non-coding RNA, or combinations thereof.

Embodiments comprising one or more RNA substance and multiple RNA stabilizing substances, may include multiple RNA stabilizing substances from the same category of RNA stabilizing substances or different categories of RNA stabilizing substances.

In some embodiments of the present disclosure is the method whereby one or more RNA stabilizing substance may be combined, such as by mixing, with at least one or more RNA substance to produce a mixture comprising at least one or more RNA stabilizing substance and at least one or more RNA substance. As a non-limiting example one or more RNA substance may be mixed with one or more RNA stabilizing substance or multiple RNA stabilizing substances. These same methods may be used to combine one or more additional substances, including, but not limited to, one or more cellular uptake agents, additive substances, or water, with one or more RNA substance and one or more RNA stabilizing substance to produce a mixture comprising one or more RNA substance, one or more RNA stabilizing substance, and one or more additional substances.

In some embodiments of the present disclosure is the method whereby one or more RNA stabilizing substance may be combined, such as by mixing, with at least one or more RNA substance to produce a composition comprising at least one or more RNA substance and at least one or more RNA stabilizing substance. As a non-limiting example one or more RNA substance may be combined with one or more RNA stabilizing substance or multiple RNA stabilizing substances to produce a composition comprising one or more RNA substance and one or more RNA stabilizing substance or multiple RNA stabilizing substances. These same methods may be used to combine one or more additional substances, including, but not limited to, one or more cellular uptake agents, additive substances, or water, with one or more RNA substance and one or more RNA stabilizing substance to produce a composition comprising one or more RNA substance, one or more RNA stabilizing substance, and one or more additional substances.

Embodiments of the present disclosure may include compositions that comprise one or more RNA stabilizing substance, at least one or more RNA substance, and water. These embodiments comprising water may include one or more composition described herein that may also comprise water.

Embodiments of the present disclosure may comprise one or more composition described herein and one or more substances that are not RNA stabilizing substances or RNA substances of which water may be one substance that is not an RNA stabilizing substance or an RNA substance.

Embodiments of the present disclosure may include compositions that comprise one or more RNA stabilizing substance, at least one or more RNA substance, and one or more cellular uptake agent. These embodiments comprising one or more cellular uptake agent may include one or more composition described herein that may also comprise one or more cellular uptake agent.

### Multiple RNA Stabilizing Substances

The inventors have discovered that combinations of RNA stabilizing substances comprising compounds from more than one RNA stabilizing substance category may be synergistic and provide better RNA stability than either individual compound (e.g., trimethylglycine (TMG) with DMSO). Non-limiting embodiments of the present disclosure include RNA stabilizing substances that may comprise one or more compounds that are members of stabilizing substance categories as described herein. Embodiments of the present disclosure may include a combination or mixture comprising one or more RNA substance and one or more RNA stabilizing substance.

The inventors have discovered that compositions comprising at least one or more RNA substance and at least one or more RNA stabilizing substance may improve RNA stability. The inventors have discovered that RNA stability may be improved in compositions comprising at least one or more RNA substance and multiple RNA stabilizing substances such as X or more RNA stabilizing substances where X may be 2, 3 4, 5, or more than 5. As a non-limiting example, RNA stability may be improved in compositions comprising at least one or more RNA substance and multiple RNA stabilizing substances where the number of multiple RNA stabilizing substances is five.

Other non-limiting example embodiments of the present disclosure may include compositions that may comprise at least one RNA substance and multiple RNA stabilizing substances, in which the composition may comprise X or more RNA stabilizing substances where X is 2, 3, 4, 5, or an integer greater than 5. As a non-limiting example, a composition may comprise at least one or more RNA substance and multiple RNA stabilizing substances where the number of multiple RNA stabilizing substances is five.

A non-limiting example embodiment of the present disclosure may include a composition that may comprise at least one or more RNA substance and at least one or more RNA stabilizing substance that improves RNA stability. The inventors have discovered that RNA stability may be improved with compositions comprising at least one or more RNA substance and multiple RNA stabilizing substances such as the composition comprising X or more RNA stabilizing substances where X may be between two and five, or greater than five. As non-limiting example embodiments, RNA stability may be improved with compositions comprising at least one or more RNA substance and multiple RNA stabilizing substances such as comprising X RNA stabilizing substances where X may be 2, 3, 4, 5, or more than 5

### Categories of RNA Stabilizing Substances

The inventors have discovered that RNA stabilizing substances may stabilize RNA substances. Furthermore, the inventors have discovered that RNA stabilizing substances may comprise defined categories of compounds. These categories of RNA stabilizing substances may comprise defined chemical structures. Therefore, the inventors have classified RNA stabilizing substances into sets of defined chemical compounds comprising defined chemical structures. As described herein, these categories of RNA stabilizing substances may be used either individually or combined to produce a composition comprising one or more RNA stabilizing substance and one or more RNA substance.

As described below, the inventors have discovered RNA stabilizing substances comprising defined compounds from one or more of the defined chemical structures and additional defined example compounds. Embodiments of the present disclosure comprise one or more of these defined compounds. Other embodiments of the present disclosure may comprise one or more RNA substance and one or more substance of the defined compounds of RNA stabilizing substances.

The descriptions herein of RNA stabilizing substances use chemical structures, formulas, and examples to clarify the invention. For conciseness and clarity, it is understood that a person of ordinary skill in the art will understand from the disclosures herein, including the descriptions and examples herein disclosed, that RNA stabilizing substances may be made and formulated and used alone or in combinations using the disclosed descriptions. The disclosed descriptions of RNA stabilizing substances and those compounds and formulations and uses are embodiments of the present disclosure. The chiral and stereoisomer forms of the compounds described herein are also embodiments of the present disclosure.

The inventors have surprisingly discovered that certain types of chemical structures may stabilize RNA substances. These specific types of chemical structures are shared across selected categories of RNA stabilizing substances. The inventors have surprisingly discovered, that selected categories of RNA stabilizing substances often contain the following groups:

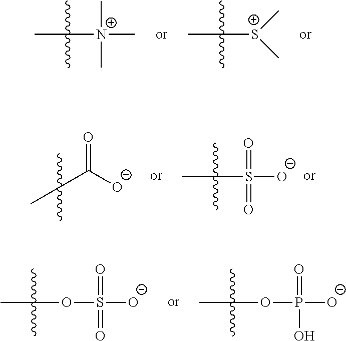

Where each group is bonded to a carbon atom of one or more defined compounds.

Without being bound to any particular mechanism or mode of action, the inventors believe that quaternary ammonium and tertiary sulfonium compounds interact with the negatively charged phosphodiester backbone of an RNA molecule and may offer rigidity or help stabilize regions of an RNA molecule that may be prone to hydrolysis. Furthermore, quaternary ammonium and tertiary sulfonium thereby help to shield the RNA backbone from the surrounding environment.

Without being bound to any particular mechanism or mode of action, the inventors believe that the electron resonance between oxygen atoms of carboxylate, sulfonate, sulfate, and phosphate groups may help to stabilize the water structure and hydrogen ion exchange in an aqueous solution to help prevent RNA hydrolysis. Furthermore, the negatively charged groups may help to repel the negatively charged RNA backbone and limit RNA flexibility in solution.

Without being bound to any particular mechanism or mode of action, the inventors have surprisingly discovered that RNA stabilizing substances comprising these quaternary ammonium, tertiary sulfonium, carboxylate, sulfonate, sulfate, and phosphate groups (e.g. NSCSSP groups) often display synergies in improving RNA stability. While RNA stabilizing substances may improve the stability of RNA, a composition containing multiple RNA stabilizing substances often increases the stability beyond any one RNA stabilizing substance. This synergy and greater RNA stability when more than one RNA stabilizing substance is present, may be a result of multiple NSCSSP groups stabilizing RNA through multiple interactions that may not necessarily be achieved by a single compound.

The following detailed descriptions of RNA stabilizing substances and structures of RNA stabilizing substances often follow a common theme of compounds containing at least one or more NSCSSP group. However, in some cases an RNA stabilizing substance may not contain an NSCSSP group, therefore the following descriptions disclose additional embodiments and are not intended as limitations.

One of ordinary skill in the art would appreciate that the functional groups described herein (such as carboxylate, sulfate, sulfonate, and phosphate groups, as non-limiting examples) may include one or more protonated or deprotonated form, such as a conjugate acid or conjugate base as non-limiting examples. These functional groups may also include one or more associated counterions to balance one or more charges, such as positively charged (e.g. Li, Na, K, Ca, Mg, or NH₄, or others) or negatively charged (e.g. Cl, Br, or SO₄, or others) counterions as non-limiting examples. Embodiments of the present disclosure include where one or more protonated or deprotonated forms of a compound may exist, these embodiments may also include these protonated or deprotonated forms and one or more associated counterions.

The following descriptions of structures and formulas are meant to clarify the invention and not the limitation thereof. The following formulas and structures are intended as a guide and follow standard bonding rules and may or may not include descriptions or depictions of hydrogens. In some cases hydrogens may or may not be shown, such as hydrogens attached to carbons or protons that may be part of one or more conjugate acid, conjugate base, or protonated or deprotonated forms.

As used herein, one or two of hydroxy or oxo can refer to substitutions with one hydroxy, two hydroxy, one hydroxy and one oxo, one oxo, or two oxo.

As used herein, one or two of hydroxy, oxo, or amino can refer to substitutions with one hydroxy, two hydroxy, one hydroxy and one oxo, one oxo, two oxo, one amino, two amino, one hydroxy and one amino, or one oxo and one amino.

As used herein, substituted with hydroxy means the replacement of a hydrogen atom with a hydroxy group.

As used herein, substituted with amino means the replacement of a hydrogen atom with an amino group

As used herein, substituted with oxo means the replacement of two hydrogen atoms of a single carbon with an oxygen atom to change the single carbon atom from an aliphatic carbon atom to a carbonyl carbon atom.

As used herein, substituted with acetoxy means the replacement of a hydrogen atom with an acetoxy group.

As used herein an acetoxy group is —O—(C═O)—CH₃.

As used herein, a heteroatom is oxygen (O), nitrogen (N), or sulfur (S).

Acyclic Quaternary Ammonium and Tertiary Sulfonium Section

In some embodiments an RNA stabilizing substance may comprise an acyclic quaternary ammonium substance. In some embodiments an RNA stabilizing substance may comprise an acyclic tertiary sulfonium substance. In some embodiments an RNA stabilizing substance may comprise an acyclic quaternary ammonium substance or acyclic tertiary sulfonium substance that has the formula [Formula 1-A]:

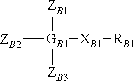

wherein:
G_{B1} is selected from nitrogen (N) or sulfur (S) and bonded to at least 3 carbon atoms;
Z_{B1}, Z_{B2}, and Z_{B3} are independent Z_{B} groups and a Z_{B} group is independently selected at each occurrence from a C₁₋₄ alkyl group, that is optionally substituted with one or two of hydroxy or oxo;
In some embodiments Z_{B3} may be absent;
If GB₁ is sulfur, then Z_{B3} is absent;
X_{B1} is an independent X_{B} group and an X_{B} group is independently selected from a C₁₋₆ alkyl or alkenyl group, that is optionally substituted with one or two of hydroxy, oxo, acetoxy or amino;
R_{B1} is an independent R_{B} group and an R_{B} group is selected from the following:

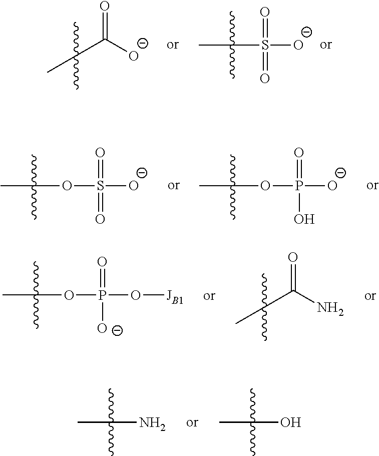

where (—COO⁻) is a carboxylate group, (—SO₃⁻) is sulfonate group, (—O—SO₃⁻) is a sulfate group, (—O—PO₃⁻H) is a phosphate group, (—O—(PO₂)—O—J_{B1}) is an organophosphate group, (—(C═O)—NH₂) is an amide group, (—NH₂) is an amino group, and (—OH) is a hydroxy group;
J_{B1} is an independent J_{B} group and a J_{B} group is independently selected from C₁₋₆ alkyl group, that is optionally substituted with two, three, four, or five hydroxys.

In some embodiments R_{B1} may be selected from carboxylate (—COO⁻), sulfonate (—SO₃⁻), sulfate (—O—SO₃⁻), phosphate (—O—PO₃⁻H), organophosphate (—O—(PO₂)—O-J_{B1}), or hydroxy (—OH). In some embodiments R_{B1} may be selected from carboxylate (—COO⁻), sulfonate (—SO₃⁻), phosphate (—O—PO₃⁻H), organophosphate (—O—(PO₂)—O-J_{B1}), or hydroxy (—OH).

In some embodiments Z_{B1}, Z_{B2}, and Z_{B3} may be the same. In some embodiments at least two of Z_{B1}, Z_{B2}, and Z_{B3} may be the same. In some embodiments a Z_{B} group may be a methyl, ethyl, propyl, butyl, or benzyl group, that is optionally substituted with one hydroxy. In some embodiments a Z_{B} group may be a C₁₋₄ alkyl group, that is optionally substituted with one hydroxy. In some embodiments a Z_{B} group may be a C₁₋₂ alkyl group, that is optionally substituted with one hydroxy.

In some embodiments X_{B1} may be a C₁₋₆ alkyl group, that is optionally substituted with one or two of hydroxy or amino. In some embodiments X_{B1} may be a C₁₋₆ alkyl group, that is optionally substituted with one hydroxy. In some embodiments X_{B1} may be a C₁₋₄ alkyl group, that is optionally substituted with one hydroxy.

In some embodiments J_{B1} may be a C₁₋₃ alkyl group, that is optionally substituted with two hydroxys.

In some embodiments a Z_{B} group may be an alcohol group, such as a methanol, ethanol, butanol, or propanol group as non-limiting examples. In some embodiments Z_{B3} may be absent.

In some embodiments a Z_{B} group may comprise 1-4, 1-3, or 1-2 carbons (e.g. 1, 2, 3, or 4 carbons). In some embodiments a Z_{B} group may be a straight chain or branched. In some embodiments a Z_{B} group may comprise 1-2 hydroxy or oxo groups (e.g. 1 or 2 groups).

In some embodiments a J_{B} group may comprise 1-6, 1-4, or 1-3, carbons (e.g. 1, 2, 3, 4, 5, or 6 carbons). In some embodiments a J_{B} group may be a straight chain or branched. In some embodiments a J_{B} group may comprise 2-5 or 2 hydroxy groups or (e.g. 2, 3, 4, or 5 groups).

In some embodiments an X_{B} group may comprise 1-6, 1-4, or 1-3, carbons (e.g. 1, 2, 3, 4, 5, or 6 carbons). In some embodiments an X_{B} group may be a straight chain or branched. In some embodiments an X_{B} group may comprise 1-2 hydroxy, amino, or oxo groups (e.g. 1 or 2 groups).

In some embodiments an X_{B} group may saturated, monounsaturated, or polyunsaturated.

In some embodiments a heteroatom is selected from N or O. In some embodiments a heteroatom is O.

Acyclic Quaternary Ammonium

In some embodiments an RNA stabilizing substance may comprise an acyclic quaternary ammonium substance that has the formula [Formula 1-B]:

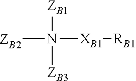

Where N is nitrogen;
Z_{B1}-Z_{B2} are independent Z_{B} groups described in this section;
Z_{B3} is absent or an independent Z_{B} group described in this section;
X_{B1} is an independent X_{B} group described in this section;
R_{B1} is an independent R_{B} group described in this section.

A non-limiting example of an acyclic quaternary ammonium substance of [Formula 1-B] is choline, wherein Z_{B1}, Z_{B2}, and Z_{B3} are CH₃, X_{B1} is (CH₂)₂ and R_{B1} is (—OH)

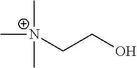

A non-limiting example of an acyclic quaternary ammonium substance of [Formula 1-B] is trimethylglycine (TMG) (also known as glycine betaine, or N,N,N-trimethylglycine), wherein Z_{B1}, Z_{B2}, and Z_{B3} are CH₃, X_{B1} is CH₂ and R_{B1} is (—COO⁻).

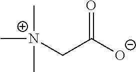

A non-limiting example of an acyclic quaternary ammonium substance of [Formula 1-B] is NDSB-195 (also known as dimethylethylammoniumpropane sulfonate or 3-[ethyl(dimethyl)ammonio]-1-propanesulfonate), wherein Z_{B1}, and Z_{B3} are CH₃, Z_{B2} is (CH₂)CH₃ and X_{B1} is (CH₂)₃ and R_{B1} is (—SO₃⁻).

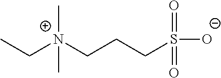

Other non-limiting examples of acyclic quaternary ammonium substances may include the following:

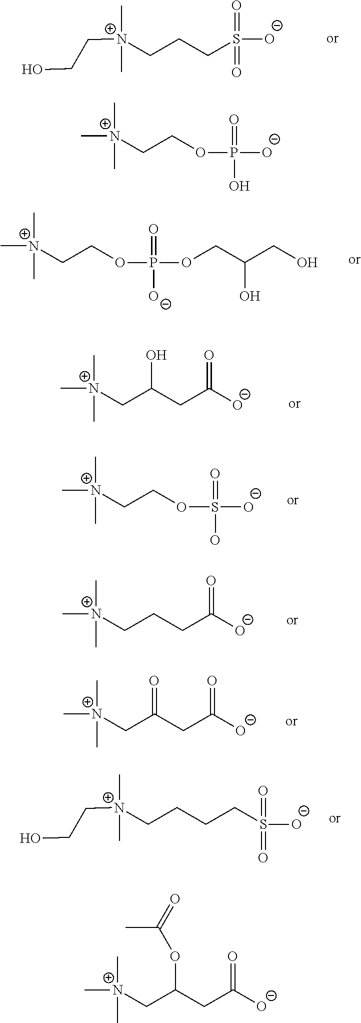

In some embodiments other acyclic quaternary ammonium substances may include: trimethylglycine, choline, carnitine, O-acetyl-carnitine, NDSB-195, 3-[trimethylammonio]-1-propanesulfonate, 3-[ethyl(dimethyl)ammonio]-1-butanesulfonate, 3-[diethyl(methyl)ammonio]-1-propanesulfonate, 3-[triethylammonio]-1-propanesulfonate, 3-[trimethylammonio]-1-butanesulfonate, 3-[dimethyl-(2-hydroxyethyl)ammonio]-1-propanesulfonate (NDSB-211), 3-[dimethyl-(2-hydroxyethyl)ammonio]-1-butanesulfonate, choline sulfate, phosphorylcholine, alpha-glycerophosphorylcholine (alpha-GPC), alanine betaine, beta-alanine betaine, N,N-dimethyl beta-alanine, N,N-dimethylglycine, gamma-butyrobetaine, valine betaine, N,N-dimethyl valine, Nε, Nε, Nε-trimethyllysine, and Nε, Nε-dimethyllysine.

Acyclic quaternary ammonium substances may include one or more stereoisomers (e.g. L-isomers or D-isomers).

In some embodiments an RNA stabilizing substance comprising an acyclic quaternary ammonium substance may be used in one or more composition described herein. In some embodiments an RNA stabilizing substance comprising an acyclic quaternary ammonium substance may be used in one or more composition described herein, where the concentration of an acyclic quaternary ammonium substance may be between about 50 mM-5M, or between about 50 mM-3M, or between about 100 mM-2M, or between about 100 mM-1M (e.g. about 50 mM, 100 mM, 200 mM, 400 mM, 500 mM, 600 mM, 800 mM, or 1M as non-limiting examples).

Acyclic Tertiary Sulfonium

In some embodiments an RNA stabilizing substance may comprise an acyclic tertiary sulfonium substance that has the formula [Formula 1-C]:

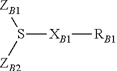

Where S is sulfur;
Z_{B1} and Z_{B2} are independent Z_{B} groups described in this section;
X_{B1} is an independent X_{B} group described in this section;
R_{B1} is an independent R_{B} group described in this section.

In some embodiments Z_{B1} and Z_{B2} may be the same.

A non-limiting example of an acyclic tertiary sulfonium substance of [Formula 1-C] is dimethylsulfoniopropionate (DMSP), wherein Z_{B1} and Z_{B2} are CH₃, X_{B1} is (CH₂)₂ and R_{B1} is (—COO⁻)

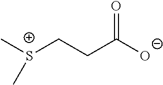

Other non-limiting examples of acyclic tertiary sulfonium substances may include the following:

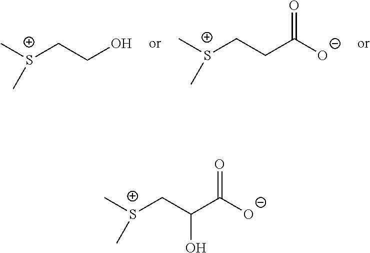

In some embodiments other acyclic tertiary sulfonium substances may include: dimethylsulfoniopropionate, S-methylmethionine, dimethylsulfonioacetate, diethylsulfoniopropionate, ethylmethylsulfoniopropionate, or methylpropylsulfoniopropionate.

In some embodiments an RNA stabilizing substance comprising an acyclic tertiary sulfonium substance may be used in one or more composition described herein. In some embodiments an RNA stabilizing substance comprising an acyclic tertiary sulfonium substance may be used in one or more composition described herein, where the concentration of an acyclic tertiary sulfonium substance may be between about 10 mM-3M, or between about 50 mM-2M, or between about 10 mM-1M, or between about 50 mM-1M (e.g. about 10 mM, 500 mM, 1000 mM, 200 mM, 400 mM, 500 mM, 600 mM, 800 mM, or 1M, as non-limiting examples).

Substituted Piperidine and Substituted Morpholine Section

In some embodiments an RNA stabilizing substance may comprise a substituted piperidine substance. In some embodiments an RNA stabilizing substance may comprise a substituted morpholine substance. In some embodiments an RNA stabilizing substance may comprise a substituted piperidine or substituted morpholine substance that has the formula [Formula 2-A]:

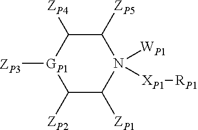

wherein:
N is a nitrogen bonded to at least 2 carbon atoms;
G_{P1} is selected from CH or oxygen (O);
Z_{P1}, Z_{P2}, Z_{P3}, Z_{P4}, and Z_{P5} are independent Z_{P} groups and a Z_{P} group is independently selected from hydrogen (H), hydroxy (—OH), oxo (═O), or carboxylate (—COO⁻);
W_{P1} is absent or selected from hydrogen (H) or a C₁₋₄ alkyl group, that is optionally substituted with one or two of hydroxy or oxo;
If G_{P1} is O, Z_{P3} is absent and W_{P1} is selected from a C₁₋₄ alkyl group, that is optionally substituted with one or two of hydroxy or oxo;
X_{P1} is absent or selected from a C₁₋₆ alkyl or alkenyl group, that is optionally substituted with one or two of hydroxy or oxo;
If X_{P1} is absent then R_{P1} is also absent;
If W_{P1} is hydrogen and X_{P1} is absent then at least one Z_{P} group is a carboxylate;
R_{P1} is absent or an independent R_{P} group and an R_{P} group selected from hydrogen (H) or the following:

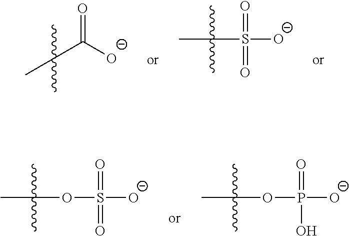

where (—COO⁻) is a carboxylate group, (—SO₃⁻) is sulfonate group, (—O—SO₃⁻) is a sulfate group, and (—O—PO₃⁻H) is a phosphate group.

In some embodiments a Z_{P} group may be independently selected from hydrogen (H), hydroxy (—OH), oxo (═O), or carboxylate (—COO⁻). In some embodiments at least one Z_{P} group is a carboxylate. In some embodiments at least one Z_{P} group is a hydroxy or oxo. In some embodiments up to 2 Z_{P} groups may be a carboxylate, hydroxy, or oxo. In some embodiments up to 3 Z_{P} groups may be a carboxylate, hydroxy, or oxo.

In some embodiments W_{P1} may be absent. In some embodiments R_{P1} may be absent. In some embodiments X_{P1} may be absent.

In some embodiments R_{P1} may be selected from carboxylate (—COO⁻), sulfonate (—SO₃⁻), or phosphate (—O—PO₃⁻H). In some embodiments R_{P1} may be selected from carboxylate (—COO⁻) or sulfonate (—SO₃⁻). In some embodiments R_{P1} may be sulfonate (—SO₃⁻).

In some embodiments W_{P1} may be a C₁₋₄ alkyl group, that is optionally substituted with one of hydroxy or oxo. In some embodiments W_{P1} may be a C₁₋₃ alkyl group, that is optionally substituted with one of hydroxy or oxo. In some embodiments W_{P1} may be a methyl, ethyl, propyl, or butyl group.

In some embodiments X_{P1} may be a C₁₋₆ alkyl group, that is optionally substituted with one hydroxy. In some embodiments X_{P1} may be a C₁₋₄ alkyl group, that is optionally substituted with one hydroxy.

In some embodiments at least one of Z_{P1}-Z_{P5} may be a carboxylate. In some embodiments at least one of Z_{P1}-Z_{P5} may be a hydroxy or oxo. In some embodiments Z_{P3} may be absent.

In some embodiments W_{P1} may be a methyl, ethyl, propyl, or butyl, optionally substituted with 1 heteroatom or 1 hydroxy group. In some embodiments W_{P1} may be an alcohol group, such as a methanol, ethanol, butanol, or propanol group as non-limiting examples.

In some embodiments W_{P1} may comprise 1-4, 1-3, or 1-2 carbons (e.g. 1, 2, 3, or 4 carbons). In some embodiments W_{P1} may be a straight chain or branched. In some embodiments W_{P1} may comprise 1-2 hydroxy groups or oxo groups (e.g. 1 or 2 groups).

In some embodiments X_{P1} may comprise 1-6, 1-4, or 1-3, carbons (e.g. 1, 2, 3, 4, 5, or 6 carbons). In some embodiments X_{P1} may be a straight chain or branched. In some embodiments X_{P1} may comprise 1-2 hydroxy or oxo groups (e.g. 1 or 2 groups). In some embodiments X_{P1} may saturated, monounsaturated, or polyunsaturated.

Substituted Piperidine

In some embodiments an RNA stabilizing substance may comprise a substituted piperidine substance that has the formula [Formula 2-B]:

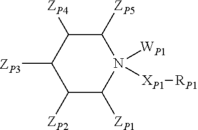

Where N is nitrogen;
Z_{P1}-Z_{P5} are independent Z_{P} groups described in this section;
W_{P1} is absent or selected from hydrogen (H) or a C₁₋₄ alkyl group, that is optionally substituted with one or two of hydroxy or oxo;
X_{P1} is absent or selected from a C₁₋₆ alkyl or alkenyl group, that is optionally substituted with one or two of hydroxy or oxo;
If X_{P1} is absent then R_{P1} is also absent;
If W_{P1} is hydrogen and X_{P1} is absent then at least one Z_{P} group is a carboxylate;
R_{P1} is absent or an independent R_{P} group described in this section;
A non-limiting example of a substituted piperidine substance of [Formula 2-B] is pipecolic acid betaine (also known as homostachydrine), wherein Z_{P1}-Z_{P4} are H, and Z_{P5} is a carboxylate, X_{P1} and W_{P1} are CH₃ and R_{P1} is absent.

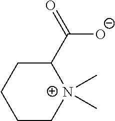

A non-limiting example of a substituted piperidine substance of [Formula 2-B] is mepiquat (also known as 1,1-dimethylpiperidinium or N,N-dimethylpiperidinium) wherein, Z_{P1}-Z_{P5} are H, X_{P1} and W_{P1} are CH₃ and R_{P1} is absent

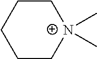

A non-limiting example of a substituted piperidine substance of [Formula 2-B] is 3-hydroxy-pipecolic acid (shown as 3-hydroxy-pipecolate; also known as 3-hydroxypiperidine-2-carboxylate) wherein, Z_{P1}-Z_{P3} are H, Z_{P4} is hydroxy and Z_{P5} is carboxylate, W_{P1} is H, and X_{P1} and R_{P1} are absent.

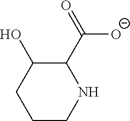

Other non-limiting examples of substituted piperidine substances may include the following:

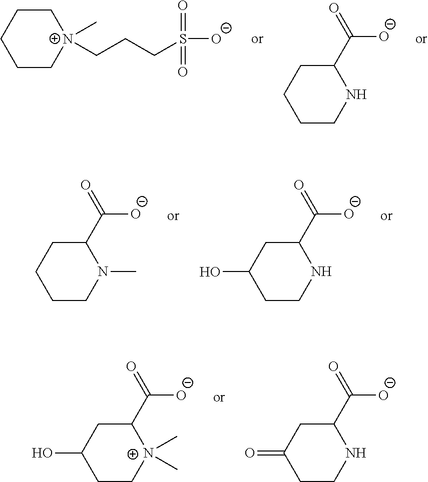

In some embodiments other substituted piperidine substances may include: pipecolic acid betaine, mepiquat, 1,1-diethylpiperidinium, 1-ethyl-1-methylpiperidinium, (3, 4, 5, or 6)-hydroxy-pipecolate, 3-(1-methylpiperidinio)-1-propanesulfonate (NDSB-221), pipecolate, (3, 4, 5, or 6)-oxo-pipecolate, (3, 4, 5, or 6)-hydroxy-pipecolic acid betaine, (3, 4, 5, or 6)-oxo-pipecolic acid betaine, or 3-(1-methylpiperidinio)-1-butanesulfonate.

Substituted piperidine substances may include one or more stereoisomers (e.g. L-isomers or D-isomers).

In some embodiments an RNA stabilizing substance comprising a substituted piperidine substance may be used in one or more composition described herein. In some embodiments an RNA stabilizing substance comprising a substituted piperidine substance may be used in one or more composition described herein, where the concentration of a substituted piperidine substance may be between about 10 mM-3M, or between about 10 mM-2M, or between about 50 mM-2M, or between about 50 mM-1M (e.g. about 50 mM, 100 mM, 200 mM, 400 mM, 500 mM, 600 mM, 800 mM, or 1M, as non-limiting examples).

Substituted Morpholine

In some embodiments an RNA stabilizing substance may comprise a substituted morpholine substance that has the formula [Formula 2-C]:

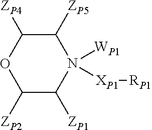

Where N is nitrogen and O is oxygen;
Z_{P1}, Z_{P2}, Z_{P4}, and Z_{P5} are independent Z_{P} groups described in this section;
W_{P1} is selected from hydrogen (H) or a C₁₋₄ alkyl group, that is optionally substituted with one or two of hydroxy or oxo;
X_{P1} is selected from a C₁₋₆ alkyl or alkenyl group, that is optionally substituted with one or two of hydroxy or oxo;
R_{P1} is absent or an independent R P group described in this section.

A non-limiting example of a substituted morpholine substance of [Formula 2-C] is NDSB-223 (also known as, N-methyl-N-(3-sulfopropyl)morpholinium), wherein Z_{P1}, Z_{P2}, Z_{P4}, and Z_{P5} are H, W_{P1} is CH₃, X_{P1} is (CH₂)₃ and R_{P1} is sulfonate.

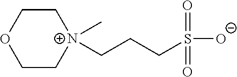

In some embodiments other substituted morpholine substances may include: NDSB-223, N-methyl-N-(3-sulfobutyl)morpholinium, N-ethyl-N-(3-sulfopropyl)morpholinium, or N-ethyl-N-(3-sulfobutyl)morpholinium.

In some embodiments an RNA stabilizing substance comprising a substituted morpholine substance may be used in one or more composition described herein. In some embodiments an RNA stabilizing substance comprising a substituted morpholine substance may be used in one or more composition described herein, where the concentration of a substituted morpholine substance may be between about 10 mM-3M, or between about 10 mM-2M, or between about 50 mM-2M, or between about 50 mM-1M (e.g. about 50 mM, 100 mM, 200 mM, 400 mM, 500 mM, 600 mM, 800 mM, or 1M, as non-limiting examples).

Substituted Pyrrolidine Section

In some embodiments an RNA stabilizing substance may comprise a substituted pyrrolidine substance. In some embodiments an RNA stabilizing substance may comprise a substituted pyrrolidine substance that has the formula [Formula 3]:

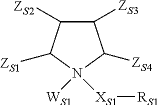

wherein:
N is a nitrogen bonded to at least 2 carbon atoms;
Z_{S1}, Z_{S2}, Z_{S3}, and Z_{S4} are independent Z_{S} groups and a Z_{S} group is independently selected from hydrogen (H), hydroxy (—OH), oxo (═O), or carboxylate (—COO⁻);
W_{S1} is absent or selected from hydrogen (H), or acetyl —(C═O)CH₃, or a C₁₋₄ alkyl group, that is optionally substituted with one or two of hydroxy or oxo;
X_{S1} is absent or selected from a C₁₋₆ alkyl or alkenyl group, that is optionally substituted with one or two of hydroxy or oxo;
If X_{S1} is absent then R_{S1} is absent;
R_{S1} is absent or selected from:

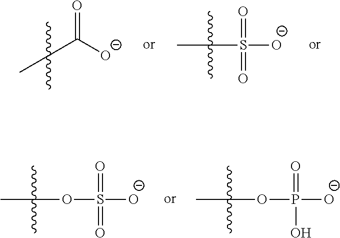

where (—COO⁻) is a carboxylate group, (—SO₃⁻) is sulfonate group, (—O—SO₃⁻) is a sulfate group, and (—O—PO₃⁻H) is a phosphate group.

In some embodiments W_{S1}, X_{S1} or R_{S1} may be absent.

In some embodiments a Z_{S} group may be independently selected from hydrogen (H), hydroxy (—OH), oxo (═O), or carboxylate (—COO⁻). In some embodiments at least one Z_{S} group is a carboxylate. In some embodiments at least one Z_{S} group is a hydroxy or oxo. In some embodiments up to 2 Z_{S} groups may be a carboxylate, hydroxy, or oxo. In some embodiments up to 3 Z_{S} groups may be a carboxylate, hydroxy, or oxo.

In some embodiments R_{S1} may be selected from carboxylate (—COO⁻), sulfonate (—SO₃⁻), or phosphate (—O—PO₃⁻H). In some embodiments R_{S1} may be selected from carboxylate (—COO⁻) or sulfonate (—SO₃⁻). In some embodiments R_{S1} may be sulfonate (—SO₃⁻).

In some embodiments W_{S1} may be a C₁₋₄ alkyl group, that is optionally substituted with one hydroxy or one oxo. In some embodiments W_{S1} may be a C₁₋₃ alkyl group, that is optionally substituted with one hydroxy or one oxo. In some embodiments W_{S1} may be a methyl, ethyl, propyl, or butyl group.

In some embodiments X_{S1} may be a C₁₋₆ alkyl group, that is optionally substituted with one hydroxy or one oxo. In some embodiments X_{S1} may be a C₁₋₄ alkyl group, that is optionally substituted with one hydroxy or one oxo. In some embodiments X_{S1} may be a methyl, ethyl, propyl, or butyl group.

In some embodiments at least one of Z_{S1}-Z_{S4} may be a carboxylate. In some embodiments at least one of Z_{S1}-Z_{S4} may be a hydroxy or oxo.

In some embodiments W_{S1} may be a methyl, ethyl, propyl, or butyl, that is optionally substituted with one hydroxy or one oxo. In some embodiments a W_{S1} may be an alcohol group, such as a methanol, ethanol, butanol, or propanol group, as non-limiting examples.

In some embodiments W_{S1} may comprise 1-4, 1-3, or 1-2 carbons (e.g. 1, 2, 3, or 4 carbons). In some embodiments W_{S1} may be a straight chain or branched. In some embodiments W_{S1} may comprise 1-2 hydroxy groups or oxo groups (e.g. 1 or 2 groups).

In some embodiments X_{S1} may comprise 1-6, 1-4, or 1-3, carbons (e.g. 1, 2, 3, 4, 5, or 6 carbons). In some embodiments X_{S1} may be a straight chain or branched. In some embodiments X_{S1} may comprise 1-2 hydroxy groups or oxo groups (e.g. 1 or 2 groups). In some embodiments X_{S1} may saturated, monounsaturated, or polyunsaturated.

A non-limiting example of a substituted pyrrolidine substance of [Formula 3] is stachydrine (also known as proline betaine) wherein Z_{S1}-Z_{S3} are H, Z_{S4} is carboxylate, W_{S1} and X_{S1} are both CH₃, and R_{S1} is absent.

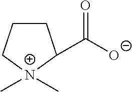

A non-limiting example of a substituted pyrrolidine substance of [Formula 3] is pyroglutamic acid (also known as 5-oxoproline) wherein Z_{S1} is an oxo group, Z_{S2} and Z_{S3} are H, Z_{S4} is carboxylate, W_{S1} is H, and X_{S1} and R_{S1} are both absent.

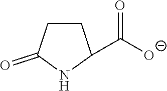

A non-limiting example of a substituted pyrrolidine substance of [Formula 3] is 1-butyl-1-methylpyrrolidinium wherein Z_{S1}-Z_{S4} are H, W_{S1} is CH₃, X_{S1} is a butyl group, and R_{S1} is absent.

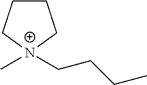

Other non-limiting examples of substituted pyrrolidine substances may include the following:

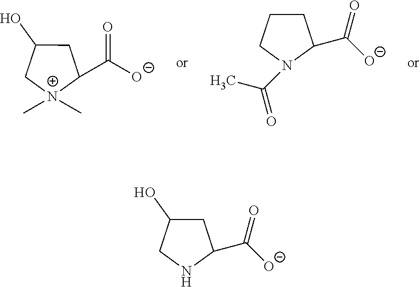

In some embodiments other substituted pyrrolidine substances may include: proline betaine, N-acetyl proline, (3 or 4)-hydroxyproline, 1-acetyl-3-hydroxyproline, 1-acetyl-4-hydroxyproline, 4-hydroxy-proline betaine, (4 or 5)-oxoproline, N-methyl proline, or N-methyl-4-hydroxy proline.

Substituted pyrrolidine substances may include one or more stereoisomers (e.g. L-isomers or D-isomers).

In some embodiments an RNA stabilizing substance comprising a substituted pyrrolidine substance may be used in one or more composition described herein. In some embodiments an RNA stabilizing substance comprising a substituted pyrrolidine substance may be used in one or more composition described herein, where the concentration of a substituted pyrrolidine substance may be between about 50 mM-3M, or between about 50 mM-2M, or between about 50 mM-1M (e.g. about 50 mM, 100 mM, 200 mM, 400 mM, 500 mM, 600 mM, 800 mM, or 1M, as non-limiting examples).

Substituted Imidazole Section

In some embodiments an RNA stabilizing substance may comprise a substituted imidazole substance. In some embodiments an RNA stabilizing substance may comprise a substituted imidazole substance that has the formula [Formula 4]:

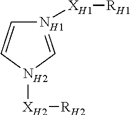

wherein:
NH₁ and NH₂ are each nitrogen (N) and each bonded to at least 2 carbon atoms;
X_{H1} is absent or an independent X_{H} group and an X_{H} group is independently selected from hydrogen (H) or a C₁₋₆ alkyl or alkenyl group or C₇₋₈ aralkyl group, that is optionally substituted with one or two of hydroxy or oxo;
X_{H2} is an independent X_{H} group and an X_{H} group is independently selected from hydrogen (H) or a C₁₋₆ alkyl or alkenyl group, that is optionally substituted with one or two of hydroxy or oxo;
If X_{H1} is absent, then R_{H1} is also absent and X_{H2} is selected from a C₁₋₆ alkyl or alkenyl group, that is optionally substituted with one or two of hydroxy or oxo;
R_{H1} is absent or an independent R_{H} group;
R_{H2} is an independent R_{H} group;
An R_{H} group is independently selected from:

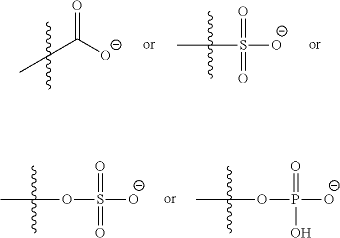

where (—COO⁻) is a carboxylate group, (—SO₃⁻) is sulfonate group, (—O—SO₃⁻) is a sulfate group, and (—O—PO₃⁻H) is a phosphate group;
In some embodiments X_{H1} or R_{H1} may be absent.

In some embodiments an R_{H} group may be selected from carboxylate (—COO⁻), sulfonate (—SO₃⁻), or phosphate (—O—PO₃⁻H). In some embodiments an R_{H} group may be selected from carboxylate (—COO⁻) or sulfonate (—SO₃⁻). In some embodiments an R_{H} group may be sulfonate (—SO₃⁻).

In some embodiments an X_{H} group may be a C₁₋₆ alkyl group, that is optionally substituted with one of hydroxy or oxo. In some embodiments an X_{H} group may be a C₁₋₄ alkyl group, that is optionally substituted with one of hydroxy or oxo. In some embodiments an X_{H} group may be a methyl, ethyl, propyl, butyl, or benzyl group, that is optionally substituted with one hydroxy

In some embodiments an X_{H} group may comprise 1-6, 1-4, or 1-3, carbons (e.g. 1, 2, 3, 4, 5, or 6 carbons). In some embodiments an X_{H} group may be a straight chain or branched. In some embodiments an X_{H} group may comprise 1-2 hydroxy or oxo groups (e.g. 1 or 2 groups). In some embodiments an X_{H} group may saturated, monounsaturated, or polyunsaturated. In some embodiments a heteroatom is selected from N or O. In some embodiments a heteroatom is O.

A non-limiting example of a substituted imidazole substance of [Formula 4] may be 1-benzyl-3-methylimidazolium wherein R_{H1} and R_{H2} are absent, X_{H1} is CH₃, and X_{H2} is a benzyl group.

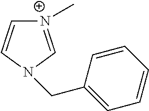

A non-limiting example of a substituted imidazole substance of [Formula 4] may be 1-butylsulfonate-3-methylimidazolium (also known as 1-methyl-3-(4-sulfobutyl)imidazolium) wherein R_{H1} is absent and R_{H2} is sulfonate, X_{H1} is CH₃, and X_{H2} is (CH₂)₄.

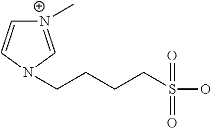

A non-limiting example of a substituted imidazole substance of [Formula 4] may be 1,3-bis(3-carboxypropyl)-1H-imidazole (also known as 1,3-bis(3-carboxypropyl)-1H-imidazolium) wherein R_{H1} and R_{H2} are both carboxylate and X_{H1} and X_{H2} are both (CH₂)₃

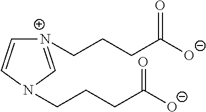 [SMILES: O=C([O-])CCCn1cc[n+](CCCC(=O)[O-])c1]

Other non-limiting examples of substituted imidazole substances may include the following:

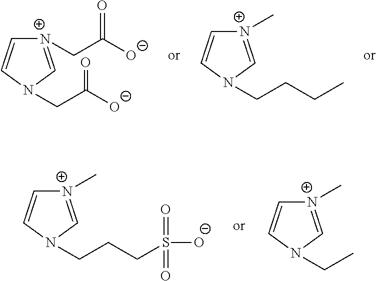

In some embodiments other substituted imidazole substances may include: 1-methyl-3-(4-sulfopropyl)imidazolium), 1-ethyl-3-(4-sulfobutyl)imidazolium), 1-ethyl-3-(4-sulfopropyl)imidazolium), 1-(2-hydroxyethyl)imidazole, 1,3-bis(3-carboxypropyl)-1H-imidazole, 1-butylsulfonate-3-methylimidazolium, 1-propylsulfonate-3-methylimidazolium, 1-benzyl-3-methylimidazolium, 1,3-Bis(carboxymethyl)-1H-imidazolium, 1,3-bis(carboxyethyl)-1H-imidazolium, 1-ethyl-3-methylimidazolium, 1-butyl-3-methylimidazolium, or 1-propyl-3-methylimidazolium.

In some embodiments an RNA stabilizing substance comprising a substituted imidazole substance may be used in one or more composition described herein. In some embodiments an RNA stabilizing substance comprising a substituted imidazole substance may be used in one or more composition described herein, where the concentration of a substituted imidazole substance may be between about 10 mM-2M, or between about 50 mM-2M, or between about 50 mM-1M (e.g. 50 mM, 100 mM, 200 mM, 400 mM, 500 mM, 600 mM, 800 mM, or 1M, as non-limiting examples).

### Substituted Benzene Section

In some embodiments an RNA stabilizing substance may comprise a substituted benzene substance. In some embodiments an RNA stabilizing substance may comprise a substituted benzene substance that has the formula [Formula 5]:

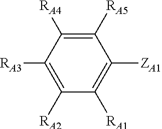

wherein:
R_{A1}-R_{A5} are each independent R_{A} groups and an R_{A} group is independently selected from hydrogen (H), hydroxy (—OH), carboxylate (—COO⁻), methoxy (—O—CH₃), ethoxy (—OCH₂CH₃), or acetoxy (—O(C═O)CH₃);
Z_{A1} is selected from:

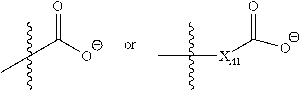

X_{A1} is selected from a C₁₋₂ alkyl or alkenyl group, that is optionally substituted with one hydroxy.

In some embodiments X_{A1} may be a C₁₋₂ alkyl, that is optionally substituted with one hydroxy. In some embodiments if an R_{A} group is a hydroxy, an R_{A} group may be etherified or esterified. In some embodiments if an R_{A} group is a hydroxy, an R_{A} group may be acetylated.

In some embodiments if Z_{A1} is a carboxylate, at least one R_{A} group is a carboxylate or hydroxy.

In some embodiments a Z_{A} group may be selected from carboxylate (—COO). In some embodiments an R_{A} group may be selected from hydrogen (H), hydroxy (—OH), methoxy (—O—CH₃), ethoxy (—OCH₂CH₃), or acetoxy (—O(C═O)CH₃). In some embodiments an R_{A} group may be selected from hydrogen (H), hydroxy (—OH), methoxy (—O—CH₃), or acetoxy (—O(C═O)CH₃). In some embodiments an R_{A} group may be selected from hydrogen (H), hydroxy (—OH), or carboxylate (—COO⁻). In some embodiments an R_{A} group may be selected from hydrogen (H), hydroxy (—OH), or acetoxy (—O(C═O)CH₃). In some embodiments an R_{A} group may be selected from hydrogen (H), or hydroxy (—OH).

In some embodiments up to 2 R_{A} groups (e.g. 1 or 2) may be a carboxylate. In some embodiments up to 2 or up to 3 R_{A} groups (e.g. 1, 2, or 3) may be a hydroxy. In some embodiments up to 2 R_{A} groups (e.g. 1 or 2) may be a methoxy or ethoxy. In some embodiments up to 2 R_{A} groups (e.g. 1 or 2) may be acetoxy. In some embodiments up to 5 R_{A} groups may be hydrogen (e.g. 1, 2, 3, 4, or 5).

In some embodiments a Z_{A} group or R_{A} group may comprise one or more conjugate acid or conjugate base or one or more protonated or deprotonated forms.

A non-limiting example of a substituted benzene substance of [Formula 5] is gallate (also known as 3,4,5-trihydroxybenzoate, or gallic acid (shown in the deprotonated form)) wherein Z_{A1} is carboxylate, R_{A2}-R_{A4} are hydroxy (OH) and R_{A1} and R_{A5} are hydrogen.

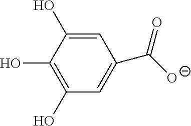 [SMILES: O=C([O-])c1cc(O)c(O)c(O)c1]

A non-limiting example of a substituted benzene substance of [Formula 5] is mandelate (also known as mandelic acid (shown in the deprotonated form), or 2-hydroxy-2-phenylacetate) wherein Z_{A1} is (X_{A1})—(COO⁻), X_{A1} is an C₁ alkyl that is substituted with one hydroxy group, and R_{A1}-R_{A5} are hydrogen.

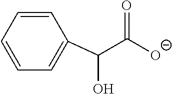 [SMILES: O=C([O-])C(O)c1ccccc1]

A non-limiting example of a substituted benzene substance of [Formula 5] is sinapinate (also known as sinapinic acid, shown in the deprotonated form) wherein Z_{A1} is (X_{A1})—(COO⁻), X_{A1} is a C₂ alkenyl, R_{A1} and R_{A5} are hydrogen, R_{A2} and R_{A4} are methoxy (—OCH₃) and R_{A3} is hydroxy.

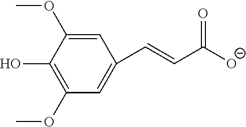 [SMILES: COc1cc(/C=C/C(=O)[O-])cc(OC)c1O]

Other non-limiting examples of substituted benzene substances may include the following:

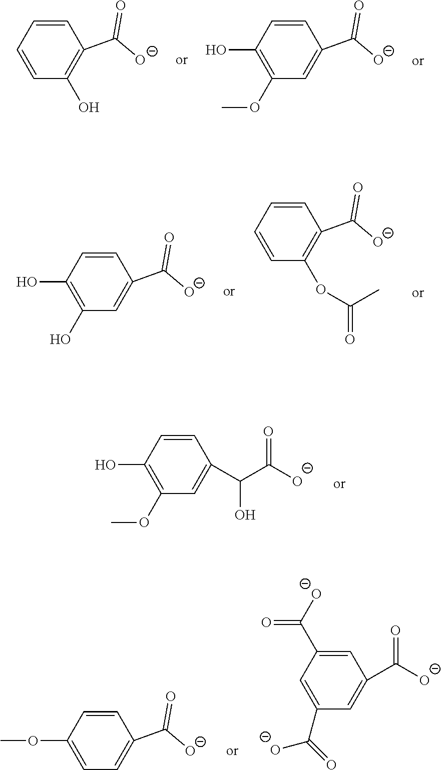

In some embodiments other substituted benzene substances may include: gallate, 3-hydroxybenzoate, 4-hydroxybenzoate, salicylate (also known as 2-hydroxybenzoate), 2-acetoxybenzoate, 1,2-benzenedicarboxylate, 1,3-benzenedicarboxylate, 1,4-benzenedicarboxylate, trimesate (e.g. deprotonated trimesic acid), benzene-1,3,5-tricarboxylate, cinnamate (e.g deprotonated cinnamic acid), combinations of (2-5)-trimethoxybenzoate (e.g 3,4,5-trimethoxybenzoate or 2,4,5-trimethoxybenzoate or combinations thereof), 4-hydroxy-3-methoxybenzoate, vanillate (e.g. deprotonated vanillic acid), 4-hydroxy-3-methoxybenzoate, 5-hydroxy-3-methoxybenzoate, 4-hydroxy-3,5-dimethoxybenzoate, or 2-hydroxy benzoate, 3-hydroxy benzoate, or 4 hydroxy benzoate, or combinations of (2-6)-dihydroxy benzoate (e.g. 2,4-dihydroxybenzoate, 3,4-dihydroxybenzoate, or 2,6-dihydroxybenzoic acid, as non-limiting examples).

In some embodiments an RNA stabilizing substance comprising a substituted benzene substance may be used in one or more composition described herein. In some embodiments an RNA stabilizing substance comprising a substituted benzene substance may be used in one or more composition described herein, where the concentration of a substituted benzene substance may be between about 5 mM-1M, or between about 20 mM-1M, or between about 20 mM-500 mM (e.g. about 20 mM, 50 mM, 100 mM, 150 mM, 200 mM, 300 mM, or 500 mM, as non-limiting examples).

Substituted Pyridine Section

In some embodiments an RNA stabilizing substance may comprise a substituted pyridine substance. In some embodiments an RNA stabilizing substance may comprise a substituted pyridine substance has the formula [Formula 6]:

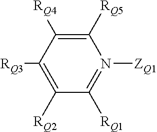

wherein:
R_{Q1}-R_{Q5} are each independent R_{Q} groups and an R_{Q} group is independently selected from hydrogen (H), hydroxy (—OH), carboxylate (—COO⁻), amide ((—C═O)—NH₂)), amino (—NH₂), methoxy (—O—CH₃), ethoxy (—OCH₂CH₃), or acetoxy (—O(C═O)CH₃);
Z_{Q1} is absent or selected from the following:

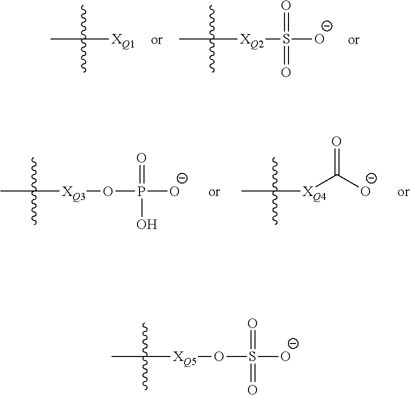

where X_{Q1}-X_{Q5} are independent X_{Q} groups and an X_{Q} group is independently selected from a C₁₋₆ alkyl or alkenyl group, that is optionally substituted with one or two of hydroxy or oxo;
If Z_{Q1} is absent then at least one R_{Q} group is a carboxylate (—COO⁻);
If an R_{Q} group is an amide, then a hydrogen on the amide nitrogen may be substituted with a carboxymethyl group (—CH₂)—(COO⁻).

In some embodiments Z_{Q1} is further selected from a methyl group (—CH₃) or an oxide (—O⁻).

In some embodiments if an R_{Q} group is a hydroxy, an R_{Q} group may be etherified or esterified. In some embodiments if an R_{Q} group is a hydroxy, an R_{Q} group may be acetylated

In some embodiments if an R_{Q} group is an amide, then a hydrogen on the amide nitrogen may be substituted with a carboxymethyl group (—CH₂)—(COO⁻) (e.g. an amide group becomes (—C═O)—(NH)—(CH₂)—(COO⁻)).

In some embodiments Z_{Q1} is further selected from:

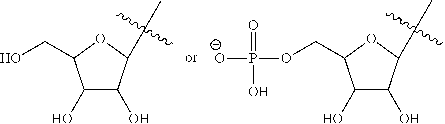 [SMILES: *C1OC(CO)C(O)C1O ; *C1OC(COP(=O)([O-])O)C(O)C1O]

In some embodiments Z_{Q1} may be selected from —X_{Q1}, (—X_{Q2})—(SO₃⁻), (—X_{Q3})—(O—PO₃⁻H), or (—X_{Q4})—(COO⁻). In some embodiments Z_{Q1} may be selected from —X_{Q1}, (—X_{Q2})—(SO₃⁻), or (—X_{Q4})—(COO⁻). In some embodiments Z_{Q1} may be selected from —X_{Q1} or (—X_{Q2})—(SO₃⁻).

In some embodiments an R_{Q} group may be selected from hydrogen (H), hydroxy (—OH), carboxylate (—COO⁻), amide ((—C═O)—NH₂)), amino (—NH₂), methoxy (—O—CH₃), ethoxy (—OCH₂CH₃), or acetoxy (—O(C═O)CH₃). In some embodiments an R_{Q} group may be selected from hydrogen (H), hydroxy (—OH), carboxylate (—COO⁻), amide ((—C═O)—NH₂)), methoxy (—O—CH₃), or acetoxy (—O(C═O)CH₃). In some embodiments an R_{Q} group may be selected from hydrogen (H), hydroxy (—OH), carboxylate (—COO⁻), methoxy (—O—CH₃), or acetoxy (—O(C═O)CH₃). In some embodiments an R_{Q} group may be selected from hydrogen (H), carboxylate (—COO⁻), or hydroxy (—OH). In some embodiments an R_{Q} group may be selected from hydrogen (H) or carboxylate (—COO⁻).

In some embodiments up to 2 R_{Q} groups (e.g. 1 or 2) may be methoxy (—O—CH₃) or ethoxy (—OCH₂CH₃). In some embodiments up to 2 R_{Q} groups (e.g. 1 or 2) may be acetoxy (—O(C═O)CH₃).

In some embodiments up to 2 R_{Q} groups or up to 3 R_{Q} groups (e.g. 1, 2, or 3) may be a carboxylate. In some embodiments up to 2 or up to 3 R_{Q} groups (e.g. 1, 2, or 3) may be a hydroxy.

In some embodiments up to 2 R_{Q} groups (e.g. 1 or 2) may be an amide. In some embodiments up to 2 R_{Q} groups (e.g. 1 or 2) may be an amide wherein a hydrogen on the amide nitrogen is substituted with a carboxymethyl group (—CH₂)—(COO⁻). In some embodiments up to 2 R_{Q} groups (e.g. 1 or 2) may be an amino. In some embodiments up to 5 R_{Q} groups may be hydrogen (e.g. 1, 2, 3, 4, or 5).

In some embodiments an X_{Q} group may be a C₁₋₆ alkyl group, that is optionally substituted with one of hydroxy or oxo. In some embodiments an X_{Q} group may be a C₁₋₄ alkyl group, that is optionally substituted with one of hydroxy or oxo.

In some embodiments an X_{Q} group may comprise 1-6, 1-4, or 1-3, carbons (e.g. 1, 2, 3, 4, 5, or 6 carbons). In some embodiments an X_{Q} group may be a straight chain or branched. In some embodiments an X_{Q} group may comprise 1-2 hydroxy or oxo groups (e.g. 1 or 2 groups). In some embodiments an X_{Q} group may saturated, monounsaturated, or polyunsaturated. In some embodiments a heteroatom is selected from N or O. In some embodiments a heteroatom is O.

In some embodiments a Z_{Q} group or R_{Q} group may comprise one or more conjugate acid or conjugate base or one or more protonated or deprotonated forms.

A non-limiting example of substituted pyridine substance of [Formula 6] is quinolinate (also known 2,3-pyridine dicarboxylate, or quinolinic acid, shown in the deprotonated form) wherein Z_{Q1} is absent, R_{Q1} and R_{Q2} are carboxylate, and R_{Q3}-R_{Q5} are hydrogen.

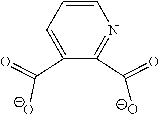 [SMILES: O=C([O-])c1cccnc1C(=O)[O-]]

A non-limiting example of substituted pyridine substance of [Formula 6] is 1-methylnicotinamide (MNA) wherein Z_{Q1} is CH₃, R_{Q4} is amide, and R_{Q1}-R_{Q3} are hydrogen and R_{Q5} is hydrogen.

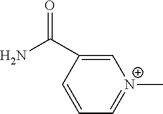

A non-limiting example of substituted pyridine substance of [Formula 6] is NDSB-201 (also known as 3-(1-pyridinio)-1-propanesulfonate) wherein Z_{Q1} is (X_{Q2})—(SO₃⁻), and X_{Q2} is (CH₂)₃, and R_{Q1}-R_{Q5} are hydrogen.

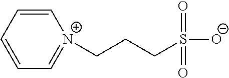 [SMILES: O=S(=O)([O-])CCC[n+]1ccccc1]

Other non-limiting examples of substituted pyridine substances may include the following:

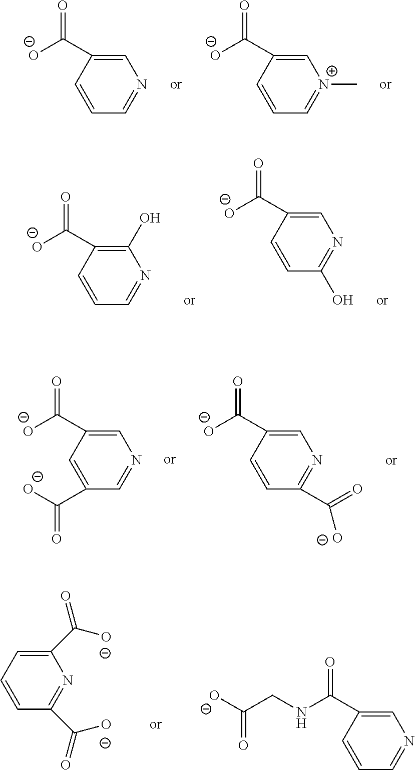

In some embodiments a substituted pyridine substance may be a pyridine dicarboxylate, wherein a pyridine dicarboxylate may include: pyridine-2,3-dicarboxylate, pyridine-2,4-dicarboxylate, pyridine-2,5-dicarboxylate, pyridine-2,6-dicarboxylate, pyridine-3,4-dicarboxylate, pyridine-3,5-dicarboxylate, or 4-hydroxy-pyridine-2,6-dicarboxylate.

In some embodiments a substituted pyridine substance may be a pyridine carboxylate, wherein a pyridine carboxylate may include: pyridine-3-carboxylate, pyridine-2-carboxylate, pyridine-4-carboxylate, 6-hydroxypyridine-2-carboxylate, 5-hydroxypyridine-2-carboxylate, 4-hydroxypyridine-2-carboxylate, 3-hydroxypyridine-2-carboxylate, 2-hydroxypyridine-3-carboxylate, 4-hydroxypyridine-3-carboxylate, 5-hydroxypyridine-3-carboxylate, 6-hydroxypyridine-3-carboxylate, 2-hydroxypyridine-4-carboxylate, or 3-hydroxypyridine-4-carboxylate.

In some embodiments other substituted pyridine substances may include: pyridine-2,3-dicarboxylate, pyridine-2,4-dicarboxylate, pyridine-2,5-dicarboxylate, pyridine-2,6-dicarboxylate, pyridine-3,4-dicarboxylate, pyridine-3,5-dicarboxylate, dinicotinate (also known as deprotonated dinicotinic acid), dipicolinate (also known as deprotonated dipicolinic acid), trigonelline (also known as 1-methylpyridinium-3-carboxylate), quinolinate, NDSB-201, 1-methylnicotinamide (MNA), nicotinamide N-oxide (NAO), nicotinurate (also known as deprotonated nicotinuric acid), nicotinate (also known as pyridine-3-carboxylate), pyridine-2-carboxylate, pyridine-4-carboxylate, nicotinamide (also known as pyridine-3-carboxamide), pyridine-4-carboxamide, pyridine-2-carboxamide, 6-hydroxypyridine-2-carboxylate, 5-hydroxypyridine-2-carboxylate, 4-hydroxypyridine-2-carboxylate, 3-hydroxypyridine-2-carboxylate, 2-hydroxypyridine-3-carboxylate, 4-hydroxypyridine-3-carboxylate, 5-hydroxypyridine-3-carboxylate, 6-hydroxypyridine-3-carboxylate, 2-hydroxypyridine-4-carboxylate, or 3-hydroxypyridine-4-carboxylate, 4-hydroxy-pyridine-2,6-dicarboxylate, nicotinamide mononucleotide (NMN) (also known as nicotinamide ribonucleoside 5′-phosphate, or 3-(aminocarbonyl)-1-(5-O-phosphono-beta-D-ribofuranosyl)-pyridinium), nicotinamide riboside (also known as 1-(beta-D-ribofuranosyl)nicotinamide), nicotinate mononucleotide (also known, nicotinate ribonucleoside 5′-phosphate, or 3-carboxy-1-(5-O-phosphono-beta-D-ribofuranosyl)-pyridinium), nicotinate riboside (also known as 1-(beta-D-ribofuranosyl)nicotinate), or 3-(1-pyridinio)-1-butylsulfonate).

In some embodiments an RNA stabilizing substance comprising a substituted pyridine substance may be used in one or more composition described herein. In some embodiments an RNA stabilizing substance comprising a substituted pyridine substance may be used in one or more composition described herein, where the concentration of a substituted pyridine substance may be between about 5 mM-2M, or between about 20 mM-2M, or between about 20 mM-1M (e.g. about 20 mM, 50 mM, 100 mM, 150 mM, 200 mM, 300 mM, 500 mM, or 1M).

Diazine Carboxylate Section

In some embodiments an RNA stabilizing substance may comprise a diazine carboxylate substance. In some embodiments an RNA stabilizing substance may comprise a diazine carboxylate substance that has the formula [Formula 7]:

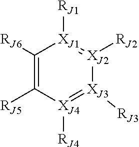

wherein
X_{J1}-X_{J4} are selected from carbon (C) or nitrogen (N);
Two X_{J} groups are selected from N and two X_{J} groups are selected from C such that there are two nitrogens and four carbons in the six membered ring. As a non-limiting example, if X_{J1} and X_{J3} are nitrogen, then X_{J2} and X_{J4} are carbon;
R_{J1}-R_{J6} are absent or an independent R_{J} group and an R_{J} group independently selected from hydrogen (H), hydroxy (—OH), carboxylate (—COO⁻), methoxy (—O—CH₃), ethoxy (—OCH₂CH₃), or acetoxy (—O(C═O)CH₃);
At least one of R_{J1}-R_{J6} is carboxylate;
If an R_{J} group is bonded to nitrogen then that R_{J} group is absent.

In some embodiments an R_{J} group may be selected from hydrogen (H), hydroxy (—OH), carboxylate (—COO⁻), methoxy (—O—CH₃), or acetoxy (—O(C═O)CH₃). In some embodiments an R_{J} group may be selected from hydrogen (H), hydroxy (—OH), or carboxylate (—COO⁻).

In some embodiments up to 2 R_{J} groups (e.g. 1 or 2) may be a carboxylate. In some embodiments up to 2 R_{J} groups (e.g. 1 or 2) may be a hydroxy. In some embodiments up to 2 R_{J} groups (e.g. 1 or 2) may be a methoxy group. In some embodiments up to 2 R_{J} groups (e.g. 1 or 2) may be a ethoxy group. In some embodiments up to 2 R_{J} groups (e.g. 1 or 2) may be an acetoxy group.

In some embodiments an R_{J} group may comprise one or more conjugate acid or conjugate base or one or more protonated or deprotonated forms.

A non-limiting example of a diazine carboxylate substance of [Formula 7] is pyrimidine-2-carboxylate, wherein X_{J1} and X_{J3} are nitrogen, X_{J2} and X_{J4} are carbon, R_{J1} and R_{J3} are absent, R_{J4}-R_{J6} are hydrogen, and R_{J2} is carboxylate.

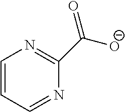

A non-limiting example of a diazine carboxylate substance of [Formula 7] is pyrazinecarboxylate, wherein X_{J1} and X_{J4} are nitrogen, X_{J2} and X_{J3} are carbon, R_{J1} and R_{J4} are absent, R_{J3}, R_{J5}, and R_{J6} are hydrogen, and R_{J2} is carboxylate.

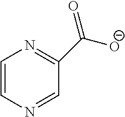

In some embodiments other diazine carboxylate substances may include: pyrazinecarboxylate, pyrimidine-2-carboxylate, pyrimidine-4-carboxylate, pyrimidine-5-carboxylate, pyrazine-2,3-dicarboxylate, or pyrimidine-4,6-dicarboxylate.

In some embodiments an RNA stabilizing substance comprising a diazine carboxylate substance may be used in one or more composition described herein. In some embodiments an RNA stabilizing substance comprising a diazine carboxylate substance may be used in one or more composition described herein, where the concentration of a diazine carboxylate substance may be between about 5 mM-1M, or between about 5 mM-500 mM, or between about 5 mM-300 mM (e.g. about 5 mM, 10 mM, 20 mM, 50 mM, 100 mM, 150 mM, 200 mM, or 300 mM, as non-limiting examples).

### Acyclic Carboxylate Section

In some embodiments an RNA stabilizing substance may comprise an acyclic carboxylate substance. In some embodiments an RNA stabilizing substance may comprise an acyclic carboxylate substance that has the formula [Formula 8]:

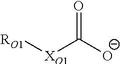

wherein:
O is oxygen;
X_{O1} is an independent X_{O} group and an X_{O} group is absent or an independent C₁₋₁₀ alkyl or alkenyl group, that is optionally substituted with one, two, three, four, or five hydroxys.

If X_{O1} is absent, then R_{O1} must be present and is bonded directly to the carboxylate (—COO⁻) in place of X_{O1};
R_{O1} is an independent R_{O} group and an R_{O} group is independently selected from hydrogen (—H), hydroxy (—OH), or carboxylate (—COO⁻);
If X_{O1} is absent, then R_{O1} is carboxylate (—COO⁻);
If X_{O1} is a C₁ alkyl (e.g. CH₂), then R_{O1} is selected from hydroxy (—OH) or carboxylate (—COO⁻).

If X_{O1} is a C₆₋₁₀ alkyl or alkenyl group, then R_{O1} is carboxylate.

In some embodiments if X_{O1} is a C₅₋₁₀ alkyl or alkenyl group, then R_{O1} is carboxylate. In some embodiments if X_{O1} has greater than 6 carbons, then R_{O1} is carboxylate. In some embodiments if X_{O1} has greater than 5 carbons, then R_{O1} is carboxylate.

In some embodiments an X_{O} group may be a C₁₋₁₀ alkyl or alkenyl group, that is optionally substituted with one, two, three, four, or five hydroxys. In some embodiments an X_{O} group may be a C₁₋₈ alkyl group, that is optionally substituted with one or two hydroxys. In some embodiments an X_{O} group may be a C₁₋₆ alkyl group, that is optionally substituted with one or two hydroxys. In some embodiments an X_{O} group may be a C₂₋₁₀ alkyl or alkenyl group, that is optionally substituted with one, two, three, four, or five hydroxys. In some embodiments an X_{O} group may be a C₂₋₈ alkyl group, that is optionally substituted with one or two hydroxys. In some embodiments an X_{O} group may be a C₂₋₆ alkyl group, that is optionally substituted with one, two, three, four, or five hydroxys. In some embodiments an X_{O} group may comprise 1 double bond, or up to 2, or up to 3 double bonds (e.g. 1, 2, or 3 double bonds).

In some embodiments an X_{O} group may comprise a carbon chain with 1-10, 1-8 1-6, or 1-4 carbons (e.g. 1, 2, 3, 4, 5, 6, 7, 8, 9 or 10 carbons). In some embodiments an X_{O} group may comprise a carbon chain with 2-10, 2-8, or 2-6 carbons (e.g. 2, 3, 4, 5, 6, 7, 8, 9, or 10 carbons).

In some embodiments an X_{O} group may be saturated, monounsaturated, or polyunsaturated. In some embodiments a heteroatom is selected from O or N. In some embodiments a heteroatom is O. In some embodiments an X_{O} group may be substituted with 1-5, or 1-4 or 1-2 hydroxy groups (e.g. 1, 2, 3, 4, or 5 hydroxy groups).

In some embodiments [Formula 8] or an R_{O} group may comprise one or more conjugate acid or conjugate base or one or more protonated or deprotonated forms.

A non-limiting example of an acyclic carboxylate substance of [Formula 8] is nonanedioate (also known as azelaic acid, shown in the deprotonated form) where X_{O1} is (CH₂)₇, and R_{O1} is carboxylate.

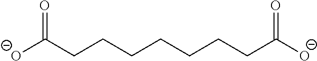 [SMILES: O=C([O-])CCCCCCCC(=O)[O-]]

A non-limiting example of an acyclic carboxylate substance of [Formula 8] is glycolate where X_{O1} is CH₂, and R_{O1} is hydroxy.

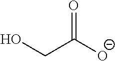

In some embodiments other acyclic carboxylate substances may include: glycolate, butanoate, propanoate, pentanoate, oxalate, 2-hydroxypropanoate, 3-hydroxypropanoate, 3-hydroxybutanoate, 4-hydroxybutanoate, 4-hydroxypentanoate, 5-hydroxypentanoate, 5-hydroxyhexanoate, 6-hydroxyhexanoate, propanedioate (also known as malonate), butanedioate (also known as succinate), pentanedioate (also known as glutarate), hexanedioate (also known as adipate), heptanedioate (also known as pimelate), octanedioate (also known as suberate), or nonanedioate (also known as deprotonated azelaic acid).

In some embodiments an RNA stabilizing substance comprising an acyclic carboxylate substance may be used in one or more composition described herein. In some embodiments an RNA stabilizing substance comprising an acyclic carboxylate substance may be used in one or more composition described herein, where the concentration of an acyclic carboxylate substance may be between about 5 mM-2M, or between about 5 mM-1M, or between about 10 mM-500 mM (e.g. about 10 mM, 20 mM, 50 mM, 100 mM, 150 mM, 200 mM, 300 mM, or 500 mM, as non-limiting examples).

### Ascorbic Acid Derivatives Section

In some embodiments an RNA stabilizing substance may comprise an ascorbic acid derivative substance. In some embodiments an RNA stabilizing substance may comprise an ascorbic acid derivative substance that has the formula [Formula 9-A]:

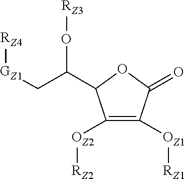

wherein
O_{Z1} and O_{Z2} are oxygen (O);
G_{Z1} is selected from oxygen (O) or NH;
R_{Z1} is absent or an independent R_{Z} group;
R_{Z2} is absent or an independent R_{Z} group;
R_{Z3} and R_{Z4} are independent R_{Z} groups;

An R_{Z} group is independently selected from hydrogen (H) or the following:

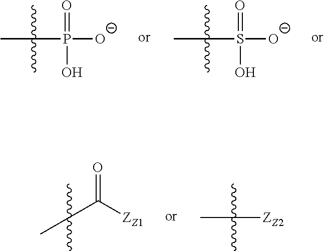

where Z_{Z1} and Z_{Z2} are independent Z_{Z} groups and a Z_{Z} group is independently selected from C₁₋₆ alkyl or alkenyl group, that is optionally substituted with one, two, three, four, or five hydroxys;

In some embodiments R_{Z1} may be further selected from the following glucopyranosyl group:

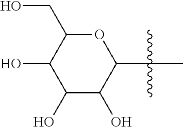 [SMILES: *C1OC(CO)C(O)C(O)C1O]

In some embodiments O_{Z1} and O_{Z2} may optionally be oxidized, wherein if O_{Z1} and O_{Z2} are oxidized then the corresponding R_{Z} groups (R_{Z1} and R_{Z2}) are both absent and the bond between the two adjacent carbon atoms bonded to O_{Z1} and O_{Z2} is a single bond as shown in the following non-limiting example formula [Formula 9-13]:

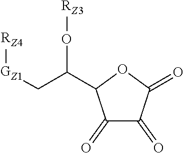

Where G_{Z1} is selected from oxygen (O) or NH;
R_{Z3} and R_{Z4} are independent R_{Z} groups as described herein.

In some embodiments one of R_{Z1} or R_{Z2} may be absent. In some embodiments both of R_{Z1} and R_{Z2} may be absent.

In some embodiments a Z_{Z} group may be a C₁₋₆ alkyl group, that is optionally substituted with one or two hydroxy. In some embodiments a Z_{Z} group may be a C₁₋₆ alkyl group, that is optionally substituted with one hydroxy. In some embodiments a Z_{Z} group may be a C₁₋₄ alkyl group, that is optionally substituted with one or two hydroxys. In some embodiments a Z_{Z} group may be a C₂₋₆ alkyl group, that is optionally substituted with one, two, three, four, or five hydroxys. In some embodiments a Z_{z} group may be a C₂₋₆ alkyl group, that is optionally substituted with one, or two hydroxys. In some embodiments a Z_{z} group may be a C₂₋₄ alkyl group, that is optionally substituted with one or two hydroxys. In some embodiments a Z_{Z} group may comprise 1 double bond, or up to 2, or up to 3 double bonds (e.g. 1, 2, or 3 double bonds).

In some embodiments a Z_{Z} group may comprise a carbon chain with 1-6 or 1-4 carbons (e.g. 1, 2, 3, 4, 5, or 6 carbons). In some embodiments a Z_{Z} group may comprise a carbon chain with 2-6 or 2-4 carbons (e.g. 2, 3, 4, 5, or 6 carbons).

In some embodiments [Formula 9-A] or an R_{Z} group may comprise one or more conjugate acid or conjugate base or one or more protonated or deprotonated forms.

A non-limiting example of an ascorbic acid derivative substance of [Formula 9-A] is 2-phospho-L-ascorbic acid where G_{Z1} is O, R_{Z2}-R_{Z4} are H and R_{Z1} is (—PO₃⁻H).

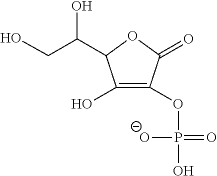 [SMILES: O=C1OC(C(O)CO)C(O)=C1OP(=O)([O-])O]

A non-limiting example of an ascorbic acid derivative substance of [Formula 9-A] is L-ascorbic acid 2,6 dibutyrate where G_{Z1} is O, R_{Z1} and R_{Z4} are both —(C═O)-Z_{Z1} and Z_{Z1} is a C₃ alkyl, and R_{Z2} and R_{Z3} are both H.

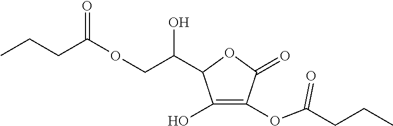 [SMILES: CCCC(=O)OCC(O)C1OC(=O)C(OC(=O)CCC)=C1O]

Other non-limiting examples of ascorbic acid derivative substances may include the following:

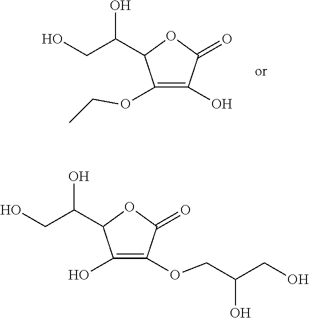 [SMILES: CCOC1=C(O)C(=O)OC1C(O)CO ; "or" ; O=C1OC(C(O)CO)C(O)=C1OCC(O)CO]

Ascorbic acid derivative substances include isoascorbic acid. Ascorbic acid derivative substances include conjugate bases of ascorbic acid (e.g ascorbate or isoascorbate).

In some embodiments other ascorbic acid derivative substances may include: 3-O-methyl-ascorbic acid, 3-O-ethyl-ascorbic acid, 3-O-propyl-ascorbic acid, 3-O-butyl-ascorbic acid, 2-O-methyl-ascorbic acid, 2-O-ethyl-ascorbic acid, 2-O-propyl-ascorbic acid, 2-O-butyl-ascorbic acid, 2-O-(2,3-dihydroxypropyl) ascorbic acid, 3-O-(2,3-dihydroxypropyl) ascorbic acid, 2-O-alpha-D-glucopyranosyl-ascorbic acid, 2-phospho-ascorbic acid, 3-phospho-ascorbic acid, ascorbyl 2,6-dibutyrate, or (+)-5,6-O-isopropylidene-L-ascorbic acid.

Ascorbic acid derivative substances may include one or more stereoisomers of ascorbic acid (e.g. isoascorbic acid, or L-isomers or D-isomers).

In some embodiments an RNA stabilizing substance comprising a ascorbic acid derivative substance may be used in one or more composition described herein. In some embodiments an RNA stabilizing substance comprising an ascorbic acid derivative substance may be used in one or more composition described herein, where the concentration of an ascorbic acid derivative substance may be between about 5 mM-2M, or between about 10 mM-1M, or between about 50 mM-1M, (e.g. about 50 mM, 100 mM, 200 mM, 300 mM, 500 mM, 800 mM, or 1M).

### Modified Amino Acids

In some embodiments an RNA stabilizing substance may comprise a modified amino acid substance. In some embodiments an RNA stabilizing substance may comprise a modified amino acid substance that has the formula [Formula 10-A]:

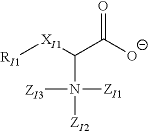

wherein:
Z_{I1} is selected from hydrogen (H), methyl (—CH₃), or acetyl (—(C═O)CH₃);
Z_{I2} is selected from hydrogen (H) or methyl (—CH₃);
Z_{I3} is absent or selected from methyl (—CH₃);
X_{I1} is an X₁ group and an X₁ group is independently selected from hydrogen (H) or a C₁₋₄ alkyl group, that is optionally substituted with one or two of hydroxy or oxo;
If X_{I1} is H, then R_{I1} is absent
R_{I1} is absent or selected from hydrogen (H), hydroxy (—OH), amino (—NH₂), carboxylate (—COO⁻), amide (—(C═O)—NH₂), phenyl, p-hydroxyphenyl, methylthio (—S—CH₃), or the following:

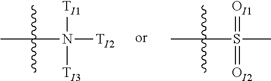

where
T_{I1} is selected from hydrogen (H), methyl (—CH₃), or acetyl (—(C═O)CH₃);
T_{I2} is selected from hydrogen (H) or methyl (—CH₃);
T_{I3} is absent or selected from methyl (—CH₃);
O_{I1} is absent or oxygen (O);
O_{I2} is absent or oxygen (O);
In some embodiments R_{I1} may be selected from hydrogen (H), hydroxy (—OH), amino (—NH₂), carboxylate (—COO⁻), p-hydroxyphenyl, or (—S—CH₃). In some embodiments Rut may be selected from amino (—NH₂), carboxylate (—COO⁻), p-hydroxyphenyl, (—S—CH₃), —(O_{I1}═S═O_{I2})—CH₃, or —(N-(T_{I1-3}).

In some embodiments when R_{I1} is hydroxy (—OH) or p-hydroxyphenyl, the hydroxy group or the hydroxy on the p-hydroxyphenyl may be substituted with phosphate (—OPO₃⁻H).

In some embodiments an X₁ group may be a C₁₋₄ alkyl group, that is optionally substituted with one of hydroxy or oxo. In some embodiments and X_{I} group may be a C₂₋₄ alkyl group, that is optionally substituted with one of hydroxy or oxo. In some embodiments an X₁ group may be a C₃₋₄ alkyl group, that is optionally substituted with one hydroxy.

In some embodiments an X₁ group may comprise a carbon chain with 1-4 or 2-4 carbons (e.g. 1, 2, 3, or 4 carbons). In some embodiments an X_{I} group may comprise a carbon chain with 3-4 carbons (e.g. 3 or 4 carbons).

In some embodiments an X_{I} group may be substituted with 1-2 hydroxy groups (e.g. 1 or 2). In some embodiments an X_{I} group may be substituted with 1-2 hydroxy groups (e.g. 1 or 2).

A non-limiting example of a modified amino acid substance of [Formula 10-A] is methionine sulfoxide where X_{I1} is (CH₂)₂, R_{I1} is —(O═S)—CH₃ where R_{I1} is —(O_{I1}═S═O_{I2})—CH₃ and O_{I1} is oxygen and O_{I2} is absent, and Z_{I1}-Z_{I2} are H and Z_{I3} is absent.

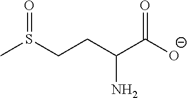

A non-limiting example of a modified amino acid substance of [Formula 10-A] is N-acetyl tyrosine (such as N-acetyl-L-tyrosine) where X_{I1} is CH₂, Rut is p-hydroxyphenyl, Z_{I1} is acetyl, and Z_{I2} is H and Z_{I3} is absent.

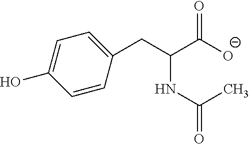 [SMILES: CC(=O)NC(Cc1ccc(O)cc1)C(=O)[O-]]

In some embodiments an RNA stabilizing substance may comprise a modified amino acid substance including, but not limited to: N-acetyl amino acids, quaternary amine amino acids, tertiary amine amino acids, tertiary sulfonium amino acids, phosphorylated amino acids, methionine sulfoxide, or methionine sulfone.

Modified amino acid substances may include the following non-limiting example N-acetyl amino acids: N-acetyl proline, N-acetyl tyrosine, N-acetyl methionine, N-acetyl cysteine, N-acetyl glutamate, N-acetyl aspartate, N-acetyl glutamine, N-acetyl asparagine, N-acetyl serine, N-acetyl threonine, N-acetyl valine, N-acetyl leucine, N-acetyl isoleucine, N-acetyl alanine, N-acetyl tryptophan, N-acetyl lysine (alpha or epsilon), N-acetyl histidine, N-acetyl arginine, N-acetyl phenylalanine, N-acetyl glycine, N-acetyl-S-methyl-methionine, N-acetyl methionine sulfoxide, and N-acetyl methionine sulfone.

N-acetyl amino acids may include one or more stereoisomers (e.g. L-isomers or D-isomers).

Other modified amino acid substances include S-methyl-methionine, methionine sulfoxide, methionine sulfone, N-acetyl-S-methyl-methionine, N-acetyl methionine sulfoxide, N-acetyl methionine sulfone, valine betaine, alanine betaine, epsilon-N-trimethyl lysine, or epsilon-N-dimethyl lysine, O-phosphotyrosine, O-phosphoserine, and O-phosphothreonine as non-limiting examples.

Modified amino acid substances may include one or more stereoisomers (e.g. L-isomers or D-isomers).

In some embodiments a modified amino acid substance may include: N-acetyl proline, N-acetyl tyrosine, N-acetyl methionine, N-acetyl cysteine, N-acetyl glutamate, N-acetyl aspartate, N-acetyl glutamine, N-acetyl asparagine, N-acetyl serine, N-acetyl threonine, N-acetyl valine, N-acetyl leucine, N-acetyl isoleucine, N-acetyl alanine, N-acetyl tryptophan, alpha-N-acetyl lysine, epsilon-N-acetyl lysine, N-acetyl histidine, N-acetyl arginine, N-acetyl phenylalanine, or N-acetyl glycine, O-acetyl carnitine, valine betaine, alanine betaine, epsilon-N-trimethyl lysine, epsilon-N-dimethyl lysine, S-methyl-methionine, methionine sulfoxide, methionine sulfone, N-acetyl-S-methyl-methionine, N-acetyl methionine sulfoxide, N-acetyl methionine sulfone, O-phosphotyrosine, O-phosphoserine, and O-phosphothreonine.

In some embodiments an RNA stabilizing substance comprising a modified amino acid substance may be used in one or more composition described herein. In some embodiments an RNA stabilizing substance comprising a modified amino acid substance may be used in one or more composition described herein, where the concentration of a modified amino acid substance may be between about 10 mM-2M, or between about 20 mM-1M, or between about 50 mM-1M (e.g. about 50 mM, 100 mM, 200 mM, 300 mM, 500 mM, 800 mM, or 1M as non-limiting examples).

### Non-Proteinogenic Amino Acids

As used herein a non-proteinogenic amino acid refers to an amino acid that is not naturally coded in the human genetic code.

In some embodiments an RNA stabilizing substance may comprise a non-proteinogenic amino acid substance. In some embodiments an RNA stabilizing substance may comprise a non-proteinogenic amino acid substance that has the formula [Formula 10-13]:

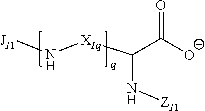

wherein:
q is an integer selected from 1-3 (e.g. 1, 2, or 3);
Z_{I1} is hydrogen (H) or acetyl (—(C═O)CH₃);
X_{Iq} is an independent X_{I} group and an X_{I} group is independently selected at each occurrence from a C₁₋₄ alkyl group, that is optionally substituted with one or two of hydroxy or oxo;
J_{I1} is selected from hydrogen (H) or amide (—(C═O)—NH₂).

In some embodiments an X_{I} group may be a C₁₋₄ alkyl group, that is optionally substituted with one of hydroxy or oxo. In some embodiments and X_{I} group may be a C₂₋₄ alkyl group, that is optionally substituted with one of hydroxy or oxo. In some embodiments an X_{I} group may be a C₃₋₄ alkyl group, that is optionally substituted with one hydroxy.

In some embodiments an X_{I} group may comprise a carbon chain with 1-4 or 2-4 carbons (e.g. 1, 2, 3, or 4 carbons). In some embodiments an X_{I} group may comprise a carbon chain with 3-4 carbons (e.g. 3 or 4 carbons).

In some embodiments an X_{I} group may be substituted with 1-2 hydroxy groups (e.g. 1 or 2). In some embodiments an X_{I} group may be substituted with 1-2 hydroxy groups (e.g. 1 or 2).

A non-limiting example of a non-proteinogenic amino acid substance of [Formula 10-B] is ornithine (such as L-ornithine as a non-limiting example) where q=1, X_{I1} is (CH₂)₃, J_{I1} and Z_{I1} are both H.

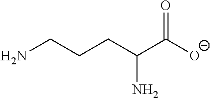

A non-limiting example of a non-proteinogenic amino acid substance of [Formula 10-B] is hypusine where q=2, X_{I1} is (CH₂)₄, X_{I2} is a C₄ alkyl substituted with one hydroxy group, and J_{I1} and Z_{I1} are both H.

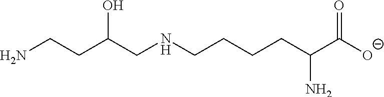 [SMILES: NCCC(O)CNCCCCC(N)C(=O)[O-]]

A non-limiting example of a non-proteinogenic amino acid substance of [Formula 10-B] is citrulline where q=1, X_{I1} is (CH₂)₃, J_{I1} is amide and Zit is H.

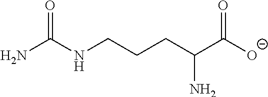 [SMILES: NC(=O)NCCCC(N)C(=O)[O-]]

Non-proteinogenic amino acid substances may include one or more stereoisomers (e.g. L-isomers or D-isomers).

In some embodiments an RNA stabilizing substance comprising a non-proteinogenic amino acid substance may be used in one or more composition described herein. In some embodiments an RNA stabilizing substance comprising a non-proteinogenic amino acid substance may be used in one or more composition described herein, where the concentration of a non-proteinogenic amino acid substance may be between about 10 mM-2M, or between about 20 mM-1M, or between about 20 mM-500 mM (e.g. about 20 mM, 50 mM, 100 mM, 200 mM, 300 mM, or 500 mM, as non-limiting examples).

### Sulfoxide Section

In some embodiments an RNA stabilizing substance may comprise a sulfoxide substance. In some embodiments an RNA stabilizing substance may comprise a sulfoxide substance that has the formula [Formula 11]:

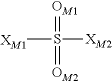

wherein
O_{M1} is oxygen (O);
O_{M2} is absent or oxygen (O);
X_{M1} and X_{M2} are independent X_{M} groups and an X_{M} group is independently selected from a C₁₋₄ alkyl group.

In some embodiments one or more X_{M} group may be a straight chain or branched. In some embodiments an X_{M} group is a methyl, ethyl, propyl, or butyl group. In some embodiments an X_{M} group may be carbon chain with 1-4, 1-3, 2-4, or 2-3 carbons (e.g. 1, 2, 3, or 4 carbons).

A non-limiting example of a sulfoxide substance of [Formula 11] is ethyl methyl sulfone where O_{M2} is oxygen and X_{M1} is an ethyl group and X_{M2} is a methyl group.

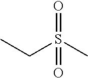

A non-limiting example of a sulfoxide substance of [Formula 11] is diethyl sulfoxide where O_{M2} is absent and X_{M1} and X_{M2} are both ethyl groups.

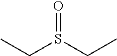

In some embodiments other sulfoxide substances may be selected from: dimethyl sulfoxide (DMSO), diethyl sulfoxide, dipropyl sulfoxide, ethyl methyl sulfoxide, methyl propyl sulfoxide, ethyl propyl sulfoxide, dimethyl sulfone, diethyl sulfone, ethyl methyl sulfone, dipropyl sulfone, methyl propyl sulfone, and ethyl propyl sulfone.

In some embodiments an RNA stabilizing substance comprising a sulfoxide substance may be used in one or more composition described herein. In some embodiments an RNA stabilizing substance comprising a sulfoxide substance may be used in one or more composition described herein, where the weight percent concentration of a sulfoxide substance may be at least 5%, or at least 10%, or at least 20%, or at least 30%, or at least 40%, or at least 50%, or at least 60%. In some embodiments an RNA stabilizing substance comprising a sulfoxide substance may be used in one or more composition described herein, where the concentration of a sulfoxide substance may be between about 50 mM-2M, or between about 50 mM-1M, or between about 100 mM-1M (e.g. 100 mM, 200 mM, 300 mM, 500 mM, 800 mM, or 1M, as non-limiting examples).

Polyphosphate Section

In some embodiments an RNA stabilizing substance may comprise a polyphosphate substance. In some embodiments an RNA stabilizing substance may comprise one or more polyphosphate substance that has the formula [Formula 12]:

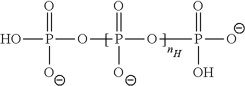

wherein
O is oxygen and P is phosphorus;
n_{H} is an integer selected from 1-10,000.

In some embodiments n H may be selected from between about 1-10,000, 1-1,000, 1-500, 1-100, 1-50, or 1-25. In some specific embodiments n H may be selected from between about 1-500, 1-100, 1-50, 1-40, 1-30, or 1-25. In some even more specific embodiments n H may be selected from between about 1-100, 1-50, 1-40, 1-30, or 1-25.

In some embodiments n H may be greater than 5, or greater than 10, or greater than 15, or greater 20, or greater than 30, or greater than 40, or greater than 50.

Embodiments comprising one or more polyphosphate substance may include one or more counter ions (e.g. NH₄, Li, Na, K, or Mg as non-limiting examples) or protonated or deprotonated forms (e.g. conjugate acids or conjugate bases).

In some embodiments one or more polyphosphate substance may include: triphosphate or polyphosphate.

Embodiments of the present disclosure may include one or more polyphosphate substance with a molecular weight between about 1 kDa-100 kDa, or between about 1 kDa-50 kDa, or between about 5 kDa-50 kDa, or between about 1 kDa-30 kDa, or between about 5 kDa-30 kDa (e.g about 1 kDa, 2.5 kDa, 5 kDa, 7.5 kDa, 10 kDa, 15 kDa, 20 kDa, 25 kDa, 30 kDa, 50 kDa, or 100 kDa).

In some embodiments an RNA stabilizing substance comprising a polyphosphate substance may be used in one or more composition described herein. In some embodiments an RNA stabilizing substance comprising a polyphosphate substance may be used in one or more composition described herein, where the concentration of a polyphosphate substance may be between about 1 mg/mL-300 mg/mL, or between about 5 mg/mL-200 mg/mL, or between about 5 mg/mL-100 mg/mL (e.g. about 5 mg/mL, 10 mg/mL, 20 mg/mL, 30 mg/mL, 40 mg/mL, 50 mg/mL, 60 mg/mL 80 mg/mL, or 100 mg/mL). In some embodiments an RNA stabilizing substance comprising a polyphosphate substance may be used in one or more composition described herein, where the concentration of a polyphosphate substance may be between about 5 mM-1M, or between about 10 mM-500 mM, or between about 10 mM-250 mM (e.g. about 10 mM, 20 mM, 30 mM, 40 mM, 50 mM, 100 mM, 200 mM, or 250 mM, as non-limiting examples).

Cyclic Phosphate Section

In some embodiments an RNA stabilizing substance may comprise a cyclic phosphate substance. In some embodiments an RNA stabilizing substance may comprise a cyclic phosphate substance that has the formula [Formula 13]:

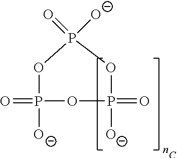

wherein
O is oxygen and P is phosphorus;
n_{C} is an integer selected from 1-100;

In some embodiments n_{C} may be selected from between 1-50, 1-40, 1-30, 1-20, 1-10, or 1-4. In some specific embodiments n_{C} may be selected from between 1-30, 1-20, 1-10, or 1-4. In some even more specific embodiments n_{C} may be selected from between 1-10, or 1-4

In some embodiments n_{C} may be less 50, or less than 40, or less than 30, or less 20, or less than 10. In some more specific embodiments n_{C} may be less than 30, or less 20, or less than 10. In some embodiments n_{C} may be selected from 1, 2, 3, 4, 5, 6, 7, 8, 9, or 10.

Embodiments comprising one or more cyclic phosphate substance may include one or more counter ions (e.g. NH₄, Li, Na, K, or Mg as non-limiting examples) or protonated or deprotonated forms (e.g. conjugate acids or conjugate bases).

In some embodiments one or more cyclic phosphate substance may be selected from: trimetaphosphate (TMP), tetrametaphosphate, pentametaphosphate, hexametaphosphate (HMP), heptametaphosphate, octametaphosphate, or decametaphosphate.

In some embodiments an RNA stabilizing substance comprising a cyclic phosphate substance may be used in one or more composition described herein. In some embodiments an RNA stabilizing substance comprising a cyclic phosphate substance may be used in one or more composition described herein, where the concentration of a cyclic phosphate substance may be between about 1 mg/mL-300 mg/mL, or between about 5 mg/mL-200 mg/mL, or between about 5 mg/mL-100 mg/mL (e.g. about 5 mg/mL, 10 mg/mL, 20 mg/mL, 30 mg/mL, 40 mg/mL, 50 mg/mL, 60 mg/mL 80 mg/mL, or 100 mg/mL). In some embodiments an RNA stabilizing substance comprising a cyclic phosphate substance may be used in one or more composition described herein, where the concentration of a cyclic phosphate substance may be between about 5 mM-1M, or between about 10 mM-500 mM, or between about 10 mM-250 mM (e.g. about 10 mM, 20 mM, 30 mM, 40 mM, 50 mM, 100 mM, 200 mM, or 250 mM, as non-limiting examples).

Modified Carbohydrates Section

The inventors have surprisingly discovered that modified carbohydrate substances may stabilize RNA substances. The inventors have discovered that an RNA stabilizing substance may comprise a modified carbohydrate substance.

A modified carbohydrate substance comprises at least one or more substituents selected from quaternary amine, tertiary amine, and phosphate groups, wherein at least one or more hydroxy group on the modified carbohydrate substance is substituted with a substituent.

The following examples describe carbohydrate modifications that may be suitable for use:

“N. Karić, M. Vukčević, M. Ristić, A. Perić-Grujić, A. Marinković, K. Trivunac, A green approach to starch modification by solvent-free method with betaine hydrochloride, Int J Biol Macromol. 193 (2021) 1962-1971.” Referred to as modified starch, betaine modified starch, or cationic starch.

“L. Passauer, F. Liebner, K. Fischer, Starch Phosphate Hydrogels. Part I: Synthesis by Mono-phosphorylation and Cross-linking of Starch, Starch-Stärke. 61 (2009) 621-627.” Referred to as modified starch, starch phosphates, monostarch monophosphates, cross-linked distarch phosphates, cross-linked monostarch monophosphates, cross-linked starch, or starch hydrogels.

“L. Passauer, F. Liebner, K. Fischer, Synthesis and Properties of Novel Hydrogels from Cross-linked Starch Phosphates, Macromolecular Symposia. 244 (2006) 180-193.” Referred to as modified starch, starch phosphates, monostarch monophosphates, cross-linked distarch phosphates, cross-linked mono starch monophosphates, cross-linked starch, or starch hydrogels.

“H. J. Prado, M. C. Matulewicz, Cationization of polysaccharides: A path to greener derivatives with many industrial applications, European Polymer Journal. 52 (2014) 53-75.” Referred to as cationized amylopectin, starch cationization, cationized chitosan, cationized hydroxyethyl cellulose, and cationic dextrans.

Other modified carbohydrates that may be suitable for use may include the following:

Cationic Dextran (Meito Industry Co., Ltd., Nishi Ward, Nagoya City, Aichi Prefecture, Japan Product CDC and CDC-L), (also known as dextran hydroxypropyltrimonium chloride)

Cationic dextran (CarboMer, San Diego California, SKU: 4-00658) (Also known as dextran hydroxypropyltrimonium chloride)

Polyquaternium-10 (Santa Cruz Biotechnology, Dallas, TX, Product #sc-495824) (also known as hydroxyethylcellulose ethoxylate, quaternized)

Polyquaternium-10 (The Biotek, Pasadena, CA, Cat #BT-1484482) (also known as hydroxyethylcellulose ethoxylate, quaternized)

Modified Polysaccharide Substance Section

Modified carbohydrate substances include modified polysaccharide substances.

The inventors have surprisingly discovered that modified polysaccharide substances may stabilize RNA substances. The inventors have discovered that an RNA stabilizing substance may comprise a modified polysaccharide substance.

A modified polysaccharide substance comprises at least one or more substituents selected from quaternary amine, tertiary amine, and phosphate groups, wherein at least one or more hydroxy group on the modified polysaccharide substance is substituted with a substituent.

In some embodiments a modified polysaccharide substance may comprise one or more modified species of polysaccharide, including modified species of amylose, amylopectin, dextran, dextrin, or cyclodextrin as non-limiting examples, wherein the modified species of polysaccharide comprises at least one or more substituents selected from a quaternary amine, tertiary amine, or phosphate group, such that at least one or more hydroxy group on the modified species of polysaccharide is substituted with a substituent.

A modified polysaccharide substance comprises at least one or more substituents selected from quaternary amine, tertiary amine, and phosphate groups, wherein at least one or more hydroxy group on the modified polysaccharide substance is substituted with a substituent. In some embodiments a modified polysaccharide substance may comprise at least one R_{V} group substituent, wherein at least one or more hydroxy group on the modified polysaccharide substance is substituted with an R_{V} group substituent as shown in the following non-limiting example [Polysaccharide Example—1]:

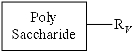

Where a [Poly Saccharide] is a modified polysaccharide substance (e.g. amylose, amylopectin, dextran, dextrin, or cyclodextrin as non-limiting examples) and an R_{V} group substituent is selected from the following groups:

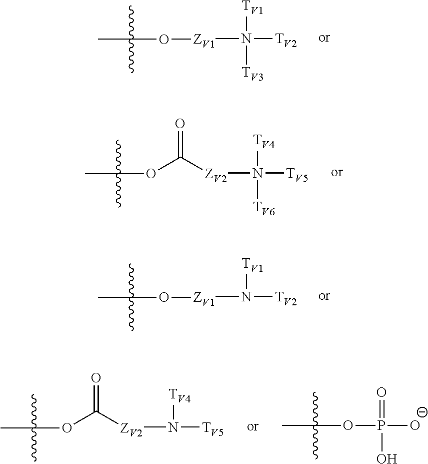

where Z_{V1} and Z_{V2} are independent Z_{V} groups and a Z_{V} group is independently selected at each occurrence from a C₁₋₆ alkyl or alkenyl group, that is optionally substituted with one or two of hydroxy or oxo or up to 2 heteroatoms;

T_{V1}-T_{V6} are independent T_{V} groups and a T_{V} group is independently selected at each occurrence from a C₁₋₄ alkyl group, that is optionally substituted with one hydroxy.

Therefore, a modified polysaccharide substance comprises at least one substituent selected from a quaternary amine, tertiary amine, or phosphate group, such that one or more hydroxy group on the modified polysaccharide is substituted with a substituent as shown in the following non-limiting example:

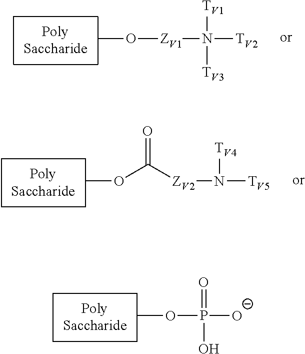

where [Poly Saccharide] is a modified polysaccharide substance;
Z_{V1}-Z_{V2} are independent Z_{V} groups as described in [Polysaccharide Example—1];
T_{V1}-T_{V5} are independent T_{V} groups as described in [Polysaccharide Example—1].

In some embodiments a modified polysaccharide substance may be substituted with an additional type of substituent to produce a modified polysaccharide substance with a combination of two or more types of substituents, such that at least two or more hydroxy groups on the modified polysaccharide substance are substituted with a different type of substituent.

In some embodiments a modified polysaccharide substance may be substituted with an additional type of substituent to produce a modified polysaccharide substance with a combination of two or more types of substituents, such that at least two or more hydroxy groups on the modified polysaccharide substance are substituted with a different type of substituent as shown in the following non-limiting example [Polysaccharide Example—2]:

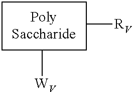

Where a [Poly Saccharide] is a modified polysaccharide substance (e.g. amylose, amylopectin, dextran, dextrin, or cyclodextrin as non-limiting examples);

An R_{V} group substituent is selected from the R_{V} groups described in [Polysaccharide Example—1];

A W_{V} group is selected from the R_{V} groups described in [Polysaccharide Example—1] and the following group:

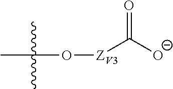

where Z_{V1} is independently selected at each occurrence from a C₁₋₆ alkyl or alkenyl group, that is optionally substituted with one or two of hydroxy or oxo or up to 2 heteroatoms;
and W_{V} is selected from a different type of substituent than R_{V}.

Therefore, in some embodiments a modified polysaccharide substance may be substituted with an additional type of substituent to produce a modified polysaccharide substance with a combination of two or more types of substituents, such that at least two or more hydroxy groups on the modified polysaccharide substance are substituted with a different type of substituent as shown in the following non-limiting examples:

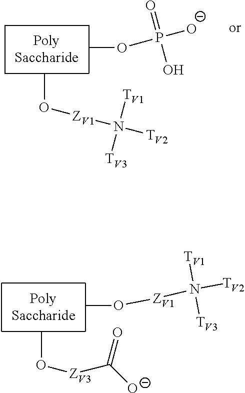

where [Poly Saccharide] is a modified polysaccharide substance;
Z_{V1} is an independent Z_{V} group as described in [Polysaccharide Example—1];
Z_{V1} is an independent Z_{V} group as described in [Polysaccharide Example—2];
T_{V1}-T_{V3} are independent T_{V} groups as described in [Polysaccharide Example—1].

In some embodiments a modified polysaccharide substance may have two or more, three or more, or four or more types of substituents (e.g. 1, 2, 3, or 4, or more types of substituents). In some embodiments a modified polysaccharide substance may be substituted with two or more of the following types of substituent groups: —O-Z_{V1}—N(T_{V1-3}), —O-(C═O)-Z_{V2}—N(T_{V4-6}), —O-Z_{V1}—N(T_{V1-2}), —O-(C═O)-Z_{V2}—N(T_{V4-5}), —(OPO₃⁻H), —O-Z_{V3}—(COO⁻). In some embodiments a modified polysaccharide substance may be substituted with a combination of two or more of the following types of substituents: a quaternary amine group, a tertiary amine group, a phosphate group, or a carboxylate group.

In some embodiments a Z_{V} group may be a C₁₋₆ alkyl group, that is optionally substituted with one or two of hydroxy or oxo. In some embodiments a Z_{V} group may be a C₁₋₄ alkyl group, that is optionally substituted with one or two of hydroxy or oxo. In some embodiments a Z_{V} group may be a C₁₋₄ alkyl group, that is optionally substituted with one of hydroxy or oxo. In some embodiments a heteroatom is selected from N or O. In some embodiments a heteroatom is O.

In some embodiments a Z_{V} group may comprise a carbon chain with 1-6, 1-4, or 1-3 carbons (e.g. 1, 2, 3, 4, 5, or 6 carbons).

In some embodiments a Z_{V} group may be saturated, monounsaturated, or polyunsaturated.

In some embodiments a Z_{V} group may be substituted with between 1-2 heteroatom substituents (e.g. 1 or 2 heteroatom substituents), wherein one or two heteroatoms may be substituted for one or two carbons. In some embodiments a Z_{V} group may be substituted with 1-2 hydroxy groups or oxo groups (e.g. 1 or 2 hydroxy groups or oxo groups).

In some embodiments a T_{V} group may be a C₁₋₄ alkyl group, that is optionally substituted with one hydroxy. In some embodiments a T_{V} group may be a C₁₋₃ alkyl group, that is optionally substituted with one hydroxy. In some embodiments a T_{V} group may be a C₁₋₂ alkyl group, that is optionally substituted with one hydroxy. In some embodiments a heteroatom is selected from N or O. In some embodiments a heteroatom is O. In some embodiments a T_{V} group may be selected from a methyl, ethyl, propyl, or butyl group. In some embodiments a T_{V} group may be an alcohol such as a methanol, ethanol, propanol, or butanol group. In some embodiments at least two T_{V} groups may be the same. In some embodiments three T_{V} groups may be the same.

In some embodiments a T_{V} group may comprise a carbon chain with 1-4, or 1-2 carbons (e.g. 1, 2, 3, or 4 carbons).

In some embodiments an R_{V} group may comprise one or more protonated or deprotonated forms (e.g. conjugate acid or conjugate base).

In some embodiments a modified polysaccharide substance may have different degrees of substitution. Degree of substitution (DS) is known in the art and is the average number of substituted hydroxy groups per polysaccharide monomer. As a non-limiting example, degree of substitution may be changed by changing the concentration of a desired substituent in relation to the polysaccharide during synthesis of a modified polysaccharide substance, such as by increasing or decreasing the concentration of the desired substituent.

In some embodiments a modified polysaccharide substance may have a DS between about 0.1-3, or between about 0.2-3, or between about 0.3-3, or between about 0.5-3, or between about 0.8-3, or between about 1-3.

In some embodiments a modified polysaccharide substance may have a DS greater than 0.1, or greater than 0.2, or greater than 0.3, or greater than 0.4, or greater than 0.5, or greater than 0.6, or greater than 0.7, or greater than 0.8, or greater than 0.9, or greater than 1.

In some embodiments when a modified polysaccharide substance is substituted with two or more types substituents to produce a modified polysaccharide substance with a combination of two or more types of substituents, the DS of each type of substituent may be different. As a non-limiting example, a modified polysaccharide substance substituted with both a quaternary amine substituent and a phosphate substituent may have a greater DS for the quaternary amine substituent than the phosphate substituent. In some embodiments the DS of two types of substituents may be a ratio of about 1:1, 1:1.2, 1:1.5, 1:2, 1:2.5, 1:3, 1:4, 1:5, or 1:10. In some embodiments the DS of two types of substituents may be a ratio between about 1:1-10, or about 1:1-5, or about 1:1-3, or about 1:1-2.

In some embodiments a modified polysaccharide substance may comprise one or more modified species of polysaccharide, wherein the modified species of polysaccharide comprises at least one or more substituents selected from a quaternary amine, tertiary amine, or phosphate group, such that at least one or more hydroxy group on the modified species of polysaccharide is substituted with a substituent. In some embodiments a modified polysaccharide substance may comprise one or more modified species of polysaccharide wherein the polysaccharide is selected from the following species: amylose, amylopectin, dextran, dextrin, cyclodextrin (e.g. alpha, beta, gamma, or greater), cellulose, beta-glucan, mixed beta-glucan, hyaluronic acid, xanthan gum, gellan gum, carboxymethyl cellulose, alginate, inulin, sinistrin, levan, chitosan, or chitin, or combinations thereof.

In some specific embodiments a modified polysaccharide substance may comprise one or more modified species of polysaccharide wherein the polysaccharide is selected from the following species: amylose, dextran, dextrin, cyclodextrin (e.g. alpha, beta, gamma, or greater), cellulose, beta-glucan, sinistrin, hyaluronic acid, or inulin, or combinations thereof.

In some even more specific a modified polysaccharide substance may comprise one or more modified species of polysaccharide wherein the polysaccharide is selected from the following species: amylose, dextran, dextrin, cyclodextrin (e.g. alpha, beta, gamma, or greater), or hyaluronic acid or combinations thereof.

In some embodiments of the present disclosure, one or more substituent is selected from the following: X-(trialkylamino)alkyl, X-(trialkylamino)-1-oxoalkyl, X-(dialkylamino)alkyl, and X-(dialkylamino)-1-oxoalkyl;
wherein
X is an integer selected from 1, 2, 3, and 4;
alkyl and 1-oxoalkyl comprise exactly X carbon atoms;
alkyl is selected from methyl, ethyl, propyl, and butyl;
1-oxoalkyl is selected from carbonyl, acetyl, propionyl, and 1-oxobutyl;
trialkyl is selected from trimethyl, triethyl, tripropyl, tributyl, and ethyl-dimethyl; and
dialkyl is selected from dimethyl and diethyl.

In some embodiments, one or more substituent is selected from: 2-(trimethylamino)ethyl; 2-(trimethylamino)-1-oxoethyl; and 4-(trimethylamino)-1-oxobutyl.

Non-limiting example tertiary and quaternary amine substituents may include:

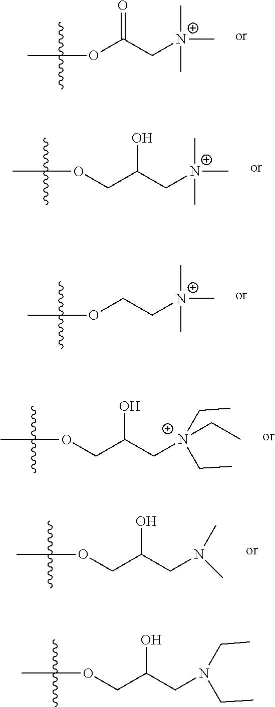

Embodiments of the present disclosure may include one or more modified polysaccharide substance with a molecular weight between about 1 kDa-1000 kDa, or between about 1 kDa-500 kDa, or between about 1 kDa-100 kDa, or between about 5 kDa-100 kDa, or between about kDa-100 kDa (e.g about 1 kDa, 2.5 kDa, 5 kDa, 7.5 kDa, 10 kDa, 15 kDa, 20 kDa, 25 kDa, 30 kDa, 50 kDa, 100 kDa, 500 kDa, or 1000 kDa).

Embodiments comprising one or more modified polysaccharide substance may include one or more protonated or deprotonated forms (e.g. conjugate acids or conjugate bases).

In some embodiments an RNA stabilizing substance comprising a modified polysaccharide substance may be used in one or more composition described herein. In some embodiments an RNA stabilizing substance comprising a modified polysaccharide substance may be used in one or more composition described herein, where the concentration of a modified polysaccharide substance may be between about 1 mg/mL-300 mg/mL, or between about 2 mg/mL-100 mg/mL, or between about 5 mg/L-50 mg/mL (e.g. 1 mg/mL, 2 mg/mL, 5 mg/mL, 10 mg/mL, 20 mg/mL, 30 mg/mL, 40 mg/mL, 50 mg/mL, 60 mg/mL, 80 mg/mL, 100 mg/mL, 200 mg/mL, or 300 mg/mL) as non-limiting examples.

Modified carbohydrate substances (including modified polysaccharide substances) may be modified by one or more means known in the art, which may include one or more of the following: 3-chloro-2-hydroxypropyltrimethylammonium chloride (CHPTAC), 2,3-epoxypropyltrimethylammonium chloride (glycidyltrimethylammonium chloride, GTMAC), 2-chlorotriethylamine, trimethyl glycine hydrochloride (also known as betaine hydrochloride), phosphate, polyphosphate, trimetaphosphate, carboxylic acids, dicarboxylic acids, tricarboxylic acids, or combinations thereof as non-limiting examples.

A modified carbohydrate may be a sugar alcohol, inositol, monosaccharide, or disaccharide, wherein a modified carbohydrate substance comprises at least one or more substituents selected from quaternary amine, tertiary amine, and phosphate groups, wherein at least one or more hydroxy group on the modified carbohydrate substance is substituted with a substituent.

One of ordinary skill in the art would appreciate that carbohydrates have different chiral forms and stereoisomers. For clarity the following descriptions are not presented as different isomers or stereoisomers, however embodiments of the present disclosure may include these different chiral forms and stereoisomers (such as D and L forms, as a non-limiting example).

Modified Sugar Alcohol Section

Modified carbohydrate substances include modified sugar alcohol substances.

The inventors have surprisingly discovered that modified sugar alcohol substances may stabilize RNA substances. The inventors have discovered that an RNA stabilizing substance may comprise a modified sugar alcohol substance.

A modified sugar alcohol substance comprises at least one or more substituents selected from quaternary amine, tertiary amine, and phosphate groups, wherein at least one or more hydroxy group on the modified sugar alcohol substance is substituted with a substituent.

A modified carbohydrate substance may be a modified sugar alcohol substance with the following formula [Formula 14-A]:

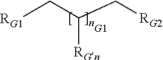

wherein
n_{G1} is an integer selected between 1-6;
R_{G1}, R_{G2} and R_{G′n} are independent R_{G} groups and an R_{G} group is independently selected at each occurrence from hydroxy (—OH) or the following groups:

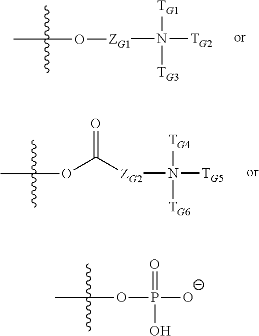

where Z_{G1}-Z_{G2} are independent Z_{G} groups and a Z_{G} group is independently selected at each occurrence from a C₁₋₆ alkyl or alkenyl group, that is optionally substituted with one or two of hydroxy or oxo;
T_{G1}, T_{G2}, T_{G4}, and T_{G5} are independent T_{G} groups and a T_{G} group is independently selected at each occurrence from a C₁₋₄ alkyl group, that is optionally substituted with one hydroxy;
T_{G3} and T_{G6} are absent or independent T_{G} groups and a T_{G} group is independently selected at each occurrence from a C₁₋₄ alkyl group, that is optionally substituted with one hydroxy;
and at least one R_{G} group is —O-Z_{G1}—N(T_{G1-3}), —O-(C═O)-Z_{G2}—N(T_{G4-6}), or —(OPO₃⁻H).

In some embodiments T_{G3} or T_{G6} may be absent.

In some embodiments an R_{G} group may be selected from hydroxy (—OH), —O-Z_{G1}—N(T_{G1-3}), —O-(C═O)-Z_{G2}—N(T_{G4-6}), or —(OPO₃⁻H).

In some embodiments n_{G1} may be selected from 1-6, 1-4, 1-3, or 1-2 (e.g. 1, 2, 3, 4, 5, or 6).

In some embodiments a Z_{G} group may be a C₁₋₆ alkyl group, that is optionally substituted with one or two of hydroxy or oxo. In some embodiments a Z_{G} group may be a C₁₋₄ alkyl group, that is optionally substituted with one or two of hydroxy or oxo. In some embodiments a Z_{G} group may be a C₁₋₃ alkyl group, that is optionally substituted with one or two of hydroxy or oxo.

In some embodiments a Z_{G} group may comprise a carbon chain with 1-6, 1-4, or 1-3 carbons (e.g. 1, 2, 3, 4, 5, or 6 carbons).

In some embodiments a Z_{G} group may be saturated, monounsaturated, or polyunsaturated.

In some embodiments a Z_{G} group may be substituted with 1-2 hydroxy groups or oxo groups (e.g. 1 or 2 hydroxy groups or oxo groups).

In some embodiments a T_{G} group may be a C₁₋₄ alkyl group, that is optionally substituted with one hydroxy. In some embodiments a T_{G} group may be a C₁₋₃ alkyl group, that is optionally substituted with one hydroxy. In some embodiments a T_{G} group may be a C₁₋₂ alkyl group, that is optionally substituted with one hydroxy. In some embodiments a T_{G} group may be selected from a methyl, ethyl, propyl, or butyl group. In some embodiments a T_{G} group may be an alcohol such as a methanol, ethanol, propanol, or butanol group. In some embodiments at least two T_{G} groups may be the same. In some embodiments three a T_{G} groups may be the same.

In some embodiments a T_{G} group may comprise a carbon chain with 1-4, or 1-2 carbons (e.g. 1, 2, 3, or 4 carbons).

In some embodiments an R_{G} group may comprise one or more protonated or deprotonated forms (e.g. conjugate acid or conjugate base).

A non-limiting example of a modified sugar alcohol substance of [Formula 14-A] is sorbitol 6-phosphate, where n_{G1}=4, R_{G′1}-R_{G′4} are OH, and R_{G2} is —(OPO₃⁻H).

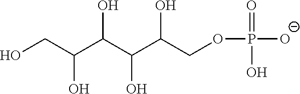 [SMILES: O=P([O-])(O)OCC(O)C(O)C(O)C(O)CO]

Embodiments comprising one or more modified sugar alcohol substance may include one or more protonated or deprotonated forms (e.g. conjugate acids or conjugate bases).

In some embodiments an RNA stabilizing substance comprising a modified sugar alcohol substance may be used in one or more composition described herein. In some embodiments an RNA stabilizing substance comprising a modified sugar alcohol substance may be used in one or more composition described herein, where the concentration of a modified sugar alcohol substance may be between about 10 mM-3M, or between about 20 mM-2M, or between about 20 mM-1M (e.g. 5 mM, 10 mM, 20 mM, 50 mM, 200 mM, 400 mM, 600 mM, 800 mM, 1M, 2M or 3M) as non-limiting examples.

Modified Inositol Section

Modified carbohydrate substances include modified inositol substances.

The inventors have surprisingly discovered that modified inositol substances may stabilize RNA substances. The inventors have discovered that an RNA stabilizing substance may comprise a modified inositol substance.

A modified inositol substance comprises at least one or more substituents selected from quaternary amine, tertiary amine, and phosphate groups, wherein at least one or more hydroxy group on the modified inositol substance is substituted with a substituent.

In some embodiments a modified carbohydrate may be a modified inositol with the following formula [Formula 14-B]:

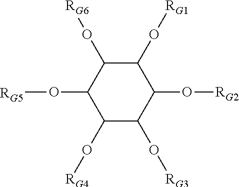

wherein:
O is oxygen;
R_{G1}-R_{G6} are independently selected R_{G} groups as described in [Formula 14-A];
and at least one R_{G} group is —O-Z_{G1}—N(T_{G1-3}), —O-(C═O)-Z_{G2}—N(T_{G4-6}), or —(OPO₃⁻H).

A non-limiting example of a modified inositol substance of [Formula 14-B] is phytate (also known as phytic acid), where R_{G1}-R_{G6} are —(PO₃⁻H).

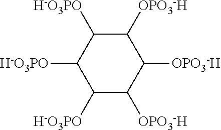

A modified inositol substance includes myo-inositol and may include one or more additional isomers such as: scyllo, muco, neo, allo, epi, cis, D-chiro, or L-chiro.

In some embodiments one or more modified inositol substance may include: myo-inositol monophosphate, myo-inositol bisphosphate, myo-inositol triphosphate, myo-inositol tetraphosphate, myo-inositol pentaphosphate, or phytate (also known as phytic acid). In some embodiments modified inositol substance may have 1, 2, 3, 4, 5, or 6 phosphate substituents, wherein the phosphate substituents may be one or more combinations at the 1, 2, 3, 4, 5, or 6 positions, such as myo-inositol 1,2,3,6 tetraphosphate or myo-inositol 1,3,4 triphosphate as non-limiting examples.

Embodiments comprising one or more modified inositol substance may include one or more protonated or deprotonated forms (e.g. conjugate acids or conjugate bases).

In some embodiments an RNA stabilizing substance comprising a modified inositol substance may be used in one or more composition described herein. In some embodiments an RNA stabilizing substance comprising a modified inositol substance may be used in one or more composition described herein, where the concentration of a modified inositol substance may be between about 5 mM-2M, or between about 10 mM-1M, or between about 10 mM-500 mM (e.g. 5 mM, 10 mM, 20 mM, 50 mM, 100 mM, 200 mM, 400 mM, or 500 mM, as non-limiting examples.)

Chemically Crosslinked Carbohydrate Section

In some embodiments a modified carbohydrate substance may be crosslinked using one or more crosslinkers, where a chemical crosslinker links two or more carbohydrate units together. Chemical crosslinking of carbohydrates is known art, where one or more carbohydrate is crosslinked by the substitution of a hydroxy group on two different carbohydrate monomers by the same substituent resulting in a crosslink across the substituent and the two monomers. The following examples describe crosslinking of carbohydrate that may be suitable for use:

“L. Passauer, F. Liebner, K. Fischer, Starch Phosphate Hydrogels. Part I: Synthesis by Mono-phosphorylation and Cross-linking of Starch, Starch-Stärke. 61 (2009) 621-627.” Referred to as cross-linked distarch phosphates, cross-linked mono starch monophosphates, cross-linked starch, or starch hydrogels

“L. Passauer, F. Liebner, K. Fischer, Synthesis and Properties of Novel Hydrogels from Cross-linked Starch Phosphates, Macromolecular Symposia. 244 (2006) 180-193.” Referred to as cross-linked distarch phosphates, cross-linked monostarch monophosphates, cross-linked starch, or starch hydrogels Non-limiting example crosslinkers that may be used to crosslink one or more modified carbohydrate substance include phosphates, cyclic phosphates, polyphosphates, aldehydes (e.g. glutaraldehyde or formaldehyde), dicarboxylic acids, tricarboxylic acids, or epoxides (e.g. epichlorohydrin).

In some embodiments a modified carbohydrate substance may be crosslinked with one or more carboxylic acids, including but not limited to one or more dicarboxylic acids, tricarboxylic acids, or higher order carboxylic acid comprising 1-5 carboxylic acid groups or greater (e.g. 1, 2, 3, 4, or 5, or more carboxylic acid groups), 1-10 carboxylic acid groups or greater, 1-20 carboxylic acid groups or greater, or 1-50 carboxylic acid groups or greater. Non-limiting example carboxylic acids that may be used to crosslink one or more modified carbohydrate substance include: maleic acid, citric acid, succinic acid, glutaric acid, pimelic acid, malonic acid, azelaic acid, tartaric acid, adipic acid, polyacrylic acid, or polymethacrylic acid, or combinations thereof.

In some embodiments a modified carbohydrate substance may be crosslinked with one or more phosphates, including but not limited to one or more diphosphates, triphosphates, polyphosphate, or cyclic phosphate comprising 1-5 phosphate groups or greater (e.g. 1, 2, 3, 4, or phosphate groups or more), 1-10 phosphate groups or greater, 1-20 phosphate groups or greater, or 1-50 phosphate groups or greater. Non-limiting example phosphates that may be used to crosslink one or more modified carbohydrate substance include: phosphate, orthophosphate, diphosphate, triphosphate, polyphosphate, pyrophosphate, trimetaphosphate, tetrametaphosphate, or hexametaphosphate, or combinations thereof.

In some embodiments a modified carbohydrate substance may be crosslinked with one or more additional types of crosslinkers, such as an epoxy, aldehyde, azide, acrylamide, carboxylic, phosphate, phosphorus oxychloride, or combinations thereof.

In some embodiments a modified carbohydrate substance may be crosslinked with one or more additional crosslinkers, including but not limited to one or more of the following: glutaraldehyde, phosphoryl chloride, phosphorus oxychloride, epichlorohydrin, formaldehyde, boric acid, N,N-methylenebis(acrylamide), diethylene glycol diglycidyl ether, or poly(ethylene glycol) diglycidyl ether, or combinations thereof.

Aldaric Acid Section

Aldaric acid substances as described herein may include one or more conjugate acid or conjugate base. The following descriptions of aldaric acid substances depict the conjugate base of an aldaric acid substance.

In some embodiments an RNA stabilizing substance may comprise an aldaric acid substance. In some embodiments an RNA stabilizing substance may comprise an aldaric acid substance with the following formula [Formula 15-A]:

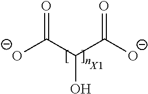

wherein:
O is oxygen;
n_{X1} is an integer selected between 1-6.

A non-limiting example of an aldaric acid of [Formula 15-A] is tartrate, where n_{X1}=2.

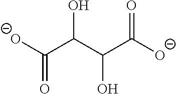

In some embodiments one or more aldaric acid substance may include: glucarate, tartrate, or mannarate.

Aldaric acid substances may include D or L isomers.

Embodiments comprising one or more aldaric acid substance may include one or more protonated or deprotonated forms of the substance (e.g. conjugate acids or conjugate bases).

In some embodiments an RNA stabilizing substance comprising an aldaric acid substance may be used in one or more composition described herein. In some embodiments an RNA stabilizing substance comprising an aldaric acid substance may be used in one or more composition described herein, where the concentration of an aldaric acid substance may be between about 5 mM-2M, or between about 20 mM-1M, or between about 50 mM-500 mM (e.g. 5 mM, 10 mM, 20 mM, 50 mM, 100 mM, 200 mM, 300 mM, 500 mM, 600 mM, 800 mM, 1M, or 2M) as non-limiting examples.

Aldonic Acid Section

Aldonic acid substances as described herein may include one or more conjugate acid or conjugate base. The following descriptions of aldonic acid substances depict the conjugate base of an aldonic acid substance.

In some embodiments an RNA stabilizing substance may comprise an aldonic acid substance. In some embodiments an RNA stabilizing substance may comprise an aldonic acid substance with the following formula [Formula 15-B]:

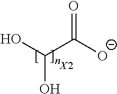

wherein:
O is oxygen;
n_{X2} is an integer selected between 1-6.

A non-limiting example of an aldonic acid of [Formula 15-B] is gluconic acid, where n_{X2}=4.

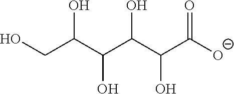 [SMILES: O=C([O-])C(O)C(O)C(O)C(O)CO]

In some embodiments one or more aldonic acid substance may include: L-threonate, D-gluconate, D-xylonate, L-erythronate, or glycerate.

Aldonic acid substances may include D or L isomers.

Embodiments comprising one or more aldonic acid substance may include one or more protonated or deprotonated forms of the substance (e.g. conjugate acids or conjugate bases).

In some embodiments an RNA stabilizing substance comprising an aldonic acid substance may be used in one or more composition described herein. In some embodiments an RNA stabilizing substance comprising an aldonic acid substance may be used in one or more composition described herein, where the concentration of an aldonic acid substance may be between about 5 mM-2M, or between about 20 mM-1M, or between about 50 mM-500 mM (e.g. 5 mM, 10 mM, 20 mM, 50 mM, 100 mM, 200 mM, 300 mM, 500 mM, 600 mM, 800 mM, 1M, or 2M) as non-limiting examples.

Stabilizing Polymer Section

In some embodiments an RNA stabilizing substance may comprise a stabilizing polymer substance. In some embodiments an RNA stabilizing substance may comprise a stabilizing polymer substance with one of the following formulas [Formula 16-A] or [Formula 16-13]:

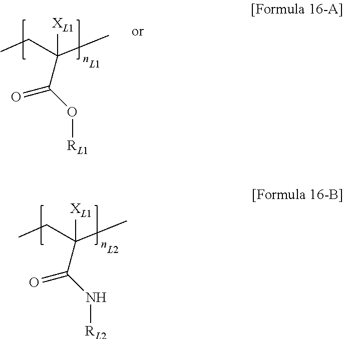

wherein
n_{L1} and n_{L2} are independent integers selected from 2-10,000;
X_{L1} is selected from hydrogen (H) or CH₃;
R_{L1} and R_{L2} are independent R_{L} groups and an R_{L} group is independently selected at each occurrence from the following groups:

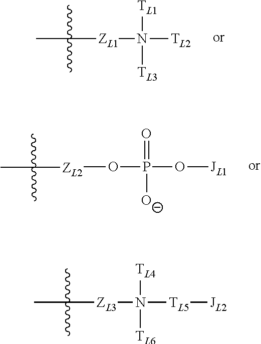

where Z_{L1}-Z_{L3} are independent Z_{L} groups and a Z_{L} group is independently selected at each occurrence from a C₁₋₈ alkyl or alkenyl group, that is optionally substituted with one or two of hydroxy or oxo or up to 2 heteroatoms;
T_{L1}-T_{L6} are independent T_{L} groups and a T_{L} group is independently selected at each occurrence from a C₁₋₄ alkyl group, that is optionally substituted with one hydroxy;
In some embodiments T_{L3} or T_{L6} may be absent;
J_{L1} and J_{L2} are independent J_{L} groups and a J_{L} group is independently selected at each occurrence from the following groups:

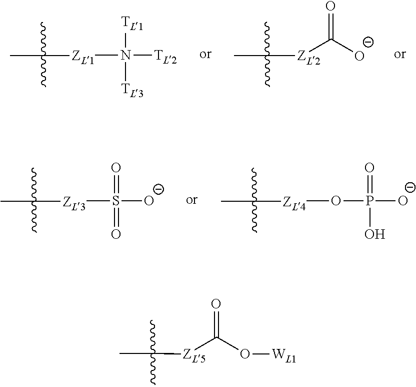

where Z_{L′1}-Z_{L′5} are independent Z_{L} groups and a Z_{L} group is independently selected at each occurrence from a C₁₋₈ alkyl or alkenyl group, that is optionally substituted with one or two of hydroxy or oxo or up to 2 heteroatoms;
T_{L′1}-T_{L′3} are independent T_{L} groups and a T_{L} group is independently selected at each occurrence from a C₁₋₄ alkyl group, that is optionally substituted with one hydroxy;
In some embodiments T_{L′3} may be absent;
W_{L1} is an independent W_{L} group and a W_{L} group is selected from the following group:

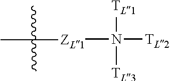

where Z_{L″1} is an independent Z_{L} group and a Z_{L} group is independently selected at each occurrence from a C₁₋₈ alkyl or alkenyl group, that is optionally substituted with one or two of hydroxy or oxo or up to 2 heteroatoms;
T_{L″1}-T_{L″3} are independent T_{L} groups and a T_{L} group is independently selected at each occurrence from a C₁₋₄ alkyl group, that is optionally substituted with one hydroxy;
In some embodiments T_{L″3} may be absent.

In some embodiments a J_{L} group may be selected from -Z_{L′2}—(COO⁻), -Z_{L′3}—(SO₃⁻), or —Z_{L′5}—(C═O)—O—W_{L1}. In some embodiments a J_{L} group may be selected from -Z_{L′2}—(COO⁻) or —Z_{L′3}—(SO₃⁻).

In some embodiments n_{L1} may be selected from between about 5-1000, 5-500, 5-250, 5-100, or 5-50. In some specific embodiments n_{L1} may be selected from between about 10-500, 10-250, 10-100, or 10-50. In some even more specific embodiments n_{L1} may be selected from between about 10-250, 10-100, or 10-50.

In some embodiments a Z_{L} group may be a C₁₋₆ alkyl group, that is optionally substituted with one or two of hydroxy or oxo or up to 2 heteroatoms. In some embodiments a Z_{L} group may be a C₁₋₄ alkyl group, that is optionally substituted with one hydroxy or 1 heteroatom. In some embodiments a heteroatom is selected from N or O. In some embodiments a heteroatom is O.

In some embodiments a T_{L} group may be a C₁₋₄ alkyl group, that is optionally substituted with one hydroxy. In some embodiments a T_{L} group may be a C₁₋₃ alkyl group, that is optionally substituted with one hydroxy. In some embodiments a T_{L} group may be a C₁₋₂ alkyl group, that is optionally substituted with one hydroxy. In some embodiments a T_{L} group may be selected from a methyl, ethyl, propyl, or butyl group. In some embodiments a T_{L} group may be an alcohol such as a methanol, ethanol, propanol, or butanol group. In some embodiments at least two a T_{L} groups may be the same. In some embodiments three a T_{L} groups may be the same.

In some embodiments a Z_{L} group may comprise a carbon chain with 1-8, 1-6, 1-4, or 1-3 carbons (e.g. 1, 2, 3, 4, 5, 6, 7, or 8 carbons). In some specific embodiments a Z_{L} group may comprise a carbon chain with 1-6, 1-4, or 1-3 carbons (e.g. 1, 2, 3, 4, 5, or 6 carbons). In some embodiments a Z_{L} group may be saturated, monounsaturated, or polyunsaturated. In some embodiments a Z_{L} group may be substituted with or 1-2 heteroatoms (e.g. 1 or heteroatoms), wherein one or more heteroatoms may be substituted for one or more carbons.

In some embodiments a T_{L} group may comprise a carbon chain with 1-4, or 1-2 carbons (e.g. 1, 2, 3, or 4 carbons).

In some embodiments an R L or J L group may comprise one or more protonated or deprotonated forms (e.g. a conjugate acid or conjugate base).

In some embodiments a stabilizing polymer substance may include one or more of the following polymers:

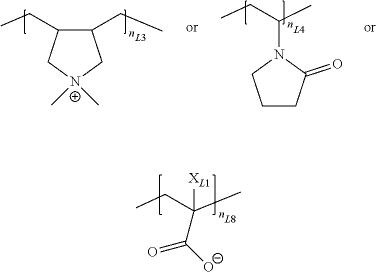

Wherein
X_{L1} is selected from H or CH₃;
n_{L3}, n_{L4}, and n_{L8} are independent integers selected from 2-10,000

In some embodiments n_{L3}, n_{L4}, and n_{L8} may be selected from between about 5-1000, 5-500, 5-250, 5-100, or 5-50. In some specific embodiments n_{L3}, n_{L4}, and n_{L8} may be selected from between about 10-500, 10-250, 10-100, or 10-50. In some even more specific embodiments n_{L3}, n_{L4}, and n_{L8} may be selected from between about 10-250, 10-100, or 10-50.

In some embodiments a stabilizing polymer substance may include: poly(2-(trimethylamino)ethyl methacrylate) (PTMAEMA), hexadimethrine, poly(diallyldimethylammonium) (PDADMAC), poly(2-methacryloyloxyethyl phosphorylcholine) (PMPC), poly(vinylpyrrolidone) (PVP), poly(acrylic acid) (PAA), poly(methacrylic acid), poly(2-(diethylamino)ethyl methacrylate) (PDEAEMA), poly((carboxybetaine methacrylate)ethyl ester)) (PCBMA-ethyl ester), poly (2-(N-3-sulfopropyl-N,N-dimethyl ammonium)ethyl methacrylate) (PSBMA) (also known as poly(sulfobetaine methacrylate), poly(carboxybetaine methacrylate), poly(ethylene glycol)-block-poly(sulfobetaine methacrylate) (PEG-PSBMA), poly(ethylene glycol)-block-poly(2-methacryloyloxyethyl phosphorylcholine) (PEG-PMPC), or poly [(2-ethyldimethylammonioethyl methacrylate ethyl sulfate)-co-(1-vinylpyrrolidone)] (Polyquat 11) (also known as polyquaternium 11).

Non-limiting examples of stabilizing polymers of the present disclosure that may be suitable for use, may include those described as poly(carboxybetaine)s, cationic polymers, or cationic polyelectrolytes in “D.-J. Liaw, C.-C. Huang, W.-F. Lee, J. Borbely, E.-T. Kang, Synthesis and characteristics of the poly(carboxybetaine)s and the corresponding cationic polymers, J. Polym. Sci. A Polym. Chem. 35 (1997) 3527-3536.”, incorporated herein by reference. The above reference also provides synthesis details for synthesizing non-limiting examples of one or more stabilizing polymer as described herein.

Non-limiting examples of stabilizing polymers of the present disclosure that may be suitable for use, may include those described as poly(sulfobetaine)s or cationic polymers in “W.-F. Lee, C.-C. Tsai, Synthesis and solubility of the poly(sulfobetaine)s and the corresponding cationic polymers: 1. Synthesis and characterization of sulfobetaines and the corresponding cationic monomers by nuclear magnetic resonance spectra, Polymer. 35 (1994) 2210-2217.”, incorporated herein by reference. The above reference also provides synthesis details for synthesizing non-limiting examples of one or more stabilizing polymer as described herein.

Non-limiting examples of stabilizing polymers of the present disclosure that may be suitable for use, may include those described as poly(sulfobetaine methacrylate) (PSBMA) in “R. Lalani, L. Liu, Synthesis, characterization, and electrospinning of zwitterionic poly(sulfobetaine methacrylate), Polymer. 52 (2011) 5344-5354.”, incorporated herein by reference. The above reference also provides synthesis details for synthesizing non-limiting examples of one or more stabilizing polymer as described herein.

Non-limiting examples of stabilizing polymers of the present disclosure that may be suitable for use, may include those described as zwitterionic polymers, polyzwitterions, polybetaines, polymeric zwitterions, polycarboxybetaines, poly sulfobetaines, polymeric phosphobetaines, or poly(phosphobetaine)s in “A. Laschewsky, Structures and Synthesis of Zwitterionic Polymers, Polymers. 6 (2014) 1544-1601.”, incorporated herein by reference. The above reference also provides synthesis details for synthesizing non-limiting examples of one or more stabilizing polymer as described herein.

Non-limiting examples of stabilizing polymers of the present disclosure that may be suitable for use, may include those described as polycarboxybetaine esters, cationic polycarboxybetaine esters, carboxybetaine ester polymers, polycarboxybetaines, or zwitterionic polycarboxybetaines in “Z. Zhang, G. Cheng, L. R. Carr, H. Vaisocherová, S. Chen, S. Jiang, The hydrolysis of cationic polycarboxybetaine esters to zwitterionic polycarboxybetaines with controlled properties, Biomaterials. 29 (2008) 4719-4725.”, incorporated herein by reference. The above reference also provides synthesis details for synthesizing non-limiting examples of one or more stabilizing polymer as described herein.

Non-limiting examples of stabilizing polymers of the present disclosure that may be suitable for use, may include those described as CBMA—ethyl ester polymers, pCBMA—ethyl ester, pCBMA—EE, or cationic CBMA ester polymer in “L. R. Carr, S. Jiang, Mediating high levels of gene transfer without cytotoxicity via hydrolytic cationic ester polymers, Biomaterials. 31 (2010) 4186-4193.”, incorporated herein by reference. The above reference also provides synthesis details for synthesizing non-limiting examples of one or more stabilizing polymer as described herein.

### Stabilizing Polymer Combinations and Blocks

Copolymers are known art, where monomeric units of two more polymers are combined to produce a hybrid polymer with the two different monomeric units. Copolymers may be assembled with alternating monomeric units or block copolymers with two different polymeric pieces combined. Copolymers may also be assembled in various combinations with different repetitions of certain monomeric units or a combination of blocks and alternating monomers.

A monomer or monomeric unit is a singular portion of a polymer that when bonded together creates the continuous chain of a polymer (not including end groups). A monomer or monomeric unit may be a part of a homopolymer or a part of a copolymer.

The inventors have discovered that RNA stabilizing substances may comprise a stabilizing polymer substance that is a copolymer, such as block copolymers or alternating copolymers. In some embodiments the monomeric units of two or more stabilizing polymers substances may be combined to create a copolymer. In some embodiments the monomeric units of two or more stabilizing polymer substances may be combined with one or more additional polymers to create a copolymer.

In some embodiments a stabilizing polymer substance may be a homopolymer. In some embodiments a stabilizing polymer substance may be copolymer, such as an alternating copolymer or block copolymer.

In some embodiments a stabilizing polymer substance may comprise one or more pendant groups wherein the pendant groups may be the same or different. In some embodiments a stabilizing polymer substance may comprise two or more pendant groups wherein the pendant groups may be the same or different.

In some embodiments a stabilizing polymer substance may comprise multiple types of pendant groups. In some embodiments a stabilizing polymer substance may comprise 2-5, 2-4, or 2-3 different types of pendant groups (e.g. 2, 3, 4, or 5 types of pendant groups). In some embodiments a stabilizing polymer substance may comprise multiple types of monomeric units. In some embodiments a stabilizing polymer substance may comprise 2-5, 2-4, or 2-3 different types of monomeric units (e.g. 2, 3, 4, or 5 types of monomeric units).

In some embodiments one or more following monomeric units may be combined to form one or more stabilizing polymer substances comprising a copolymer:

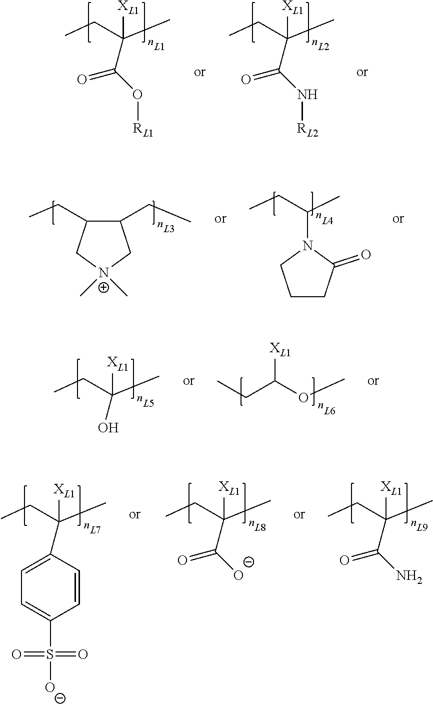

Wherein
R_{L1} and R_{L2} are independent R L groups independently selected at each occurrence as described herein this section;
X_{L1} is selected from H or CH₃;
n_{L1}-n_{L9} are independent integers selected from 1-10,000.

In some embodiments n_{L1}-n_{L9} may be selected from between about 1-500, 1-250, 1-100, or 1-50. In some specific embodiments n_{L1}-n_{L9} may be selected from between about 1-250, 1-100, or 1-50. In some even more specific embodiments n_{L1}-n_{L9} may be selected from between about 1-100, 1-50, or 1-20.

In some embodiments a stabilizing polymer substance may comprise a linear block copolymer. In some embodiments a stabilizing polymer substance may comprise a random block copolymer. In some embodiments a stabilizing polymer substance may comprise an alternating block copolymer.

In some embodiments a stabilizing polymer substance may comprise a block copolymer, wherein a stabilizing polymer substance may comprise a diblock polymer, triblock polymer, quaterblock polymer, or higher order multiblock polymer. In some embodiments a stabilizing polymer substance may comprise multiple types of blocks. In some embodiments a stabilizing polymer substance may comprise 2-5, 2-4, or 2-3 different types of blocks (e.g. 2, 3, 4, or 5 types of blocks).

In some embodiments a stabilizing polymer substance may comprise a copolymer, such as a bipolymer, terpolymer, or quaterpolymer, or higher order copolymer. In some embodiments a stabilizing polymer substance may comprise one or more the following types of copolymers, including, but not limited to: a linear copolymer, a random copolymer, an alternating copolymer, a statistical copolymer, a gradient copolymer, a periodic copolymer, a sequential copolymer, a block copolymer, a graft copolymer, a crosslinked copolymer, or a star copolymer, or combinations thereof.

In some embodiments a stabilizing polymer substance may comprise a block copolymer, wherein a stabilizing polymer substance may comprise a diblock polymer, triblock polymer, quaterblock polymer, or higher order multiblock polymer.

In some embodiments a stabilizing polymer substance may comprise a linear block copolymer. In some embodiments a stabilizing polymer substance may comprise a random block copolymer. In some embodiments a stabilizing polymer substance may comprise an alternating block copolymer.

In some embodiments an RNA stabilizing substance may comprise an anionic or polyanionic polymer. In some embodiments an RNA stabilizing substance may comprise a cationic or polycationic polymer. In some embodiments an RNA stabilizing substance may comprise a zwitterionic polymer.

In some embodiments a stabilizing polymer substance may be at least partially hydrolyzable. In some embodiments a stabilizing polymer substance may comprise one or more hydrolyzable bonds.

Embodiments of the present disclosure may include one or more stabilizing polymer substance with a molecular weight between about 1 kDa-100 kDa, or between about 1 kDa-50 kDa, or between about 5 kDa-50 kDa (e.g about 1 kDa, 2.5 kDa, 5 kDa, 7.5 kDa, 10 kDa, 15 kDa, kDa, 25 kDa, 30 kDa, 50 kDa, or 100 kDa).

Embodiments comprising one or more stabilizing polymer substance may include one or more protonated or deprotonated forms (e.g. conjugate acids or conjugate bases).

In some embodiments an RNA stabilizing substance comprising a stabilizing polymer substance may be used in one or more composition described herein. In some embodiments an RNA stabilizing substance comprising a stabilizing polymer substance may be used in one or more composition described herein, where the concentration of a stabilizing polymer substance may be between about 0.1 mg/mL-100 mg/mL, or between about 0.2 mg/mL-100 mg/mL, or between about 0.5 mg/L-50 mg/mL. In some embodiments an RNA stabilizing substance comprising a stabilizing polymer substance may be used in one or more composition described herein, where the concentration of a stabilizing polymer substance may be less than 10 mg/L, or less than 5 mg/mL, or less than 1 mg/mL. In some embodiments an RNA stabilizing substance comprising a stabilizing polymer substance may be used in one or more composition described herein, where the concentration of a stabilizing polymer substance may be greater than 1 mg/L, or greater than 5 mg/mL, or greater than 10 mg/mL, or greater than 20 mg/mL.

Physical Properties

In some embodiments a composition comprising one or more RNA substance and one or more RNA stabilizing substance may be a liquid. As non-limiting examples, one or more composition described herein, may be a solution, fluid, syrup, emulsion, or suspension, and may also include liquid or solid carriers. As non-limiting examples the liquid viscosity may be in the range between about 0.1 centipoise-100,000,000 centipoise at about 20-25° C. As non-limiting examples the liquid viscosity may be in the range between about 0.1 centipoise-1,000,000 centipoise at about 20-25° C. As non-limiting examples the liquid viscosity may be in the range between about 0.1 centipoise-100,000 centipoise at about 20-25° C. As non-limiting examples the liquid viscosity may be in the range between about 0.1 centipoise-10,000 centipoise at about 20-25° C. As non-limiting examples the liquid viscosity may be in the range between about 0.1 centipoise-1,000 centipoise at about 20-25° C.

In some embodiments a composition comprising one or more RNA substance and one or more RNA stabilizing substance may be a solid. As non-limiting examples, one or more composition described herein may be a pellet, powder, or tablet, and may also include solid carriers.

In some embodiments a composition comprising one or more RNA substance and one or more RNA stabilizing substance may be a gel. As non-limiting examples, one or more composition described herein, may be a hydrated solid, or porous solid filled with or retaining water or other liquid or solution, and may also include solid or liquid carriers.

In some embodiments a composition comprising one or more RNA substance and one or more RNA stabilizing substance may be a vapor or aerosol. As non-limiting examples, one or more composition described herein may be a gas, vapor, or aerosol, or suspension of particles or droplets suspended in one or more gases (such as, but not limited to, air, nitrogen, oxygen, carbon dioxide, or anesthetic gas) and may also include liquid or solid carriers.

Water Weight Percent

Compositions described herein comprising one or more RNA substance and one or more RNA stabilizing substance may comprise water.

Embodiments of the present disclosure may include one or more compositions described herein, wherein a composition may comprise a water weight percent between about X and Y, where X may be 10%, 20%, 30%, or 40%, and Y is greater than X, and Y may be 95%, 90%, 80%, 70%, 60%, or 50%, as non-limiting examples X may be 10% and Y may be 95% and a composition may comprise a water weight percent between about 10%-95%, or X may be 30% and Y may be 70% and a composition may comprise a water weight percent between about 30%-70%.

Embodiments of the present disclosure may include one or more compositions described herein, wherein a composition may comprise a water weight percent greater than about 10%, or greater than about 20%, or greater than about 30%, or greater than about 40%, or greater than about 50%, or greater than about 60%.

Embodiments of the present disclosure may include one or more compositions described herein, wherein a composition may comprise a water weight percent less than about 95%, less than about 90%, less than about 85%, less than about 80%, less than about 75%, less than about 70%, less than about 60%, or less than about 50%.

Embodiments of the present disclosure may include compositions comprising one or more RNA substance and one or more RNA stabilizing substance described herein, where the composition is a liquid at 20° C. In some embodiments a composition comprising one or more RNA substance and one or more RNA stabilizing substance may be a liquid at 0° C., 10° C., or 20° C.

RNA Stabilizing Substance Total Weight Percent

Compositions described herein comprising one or more RNA substance and one or more RNA stabilizing substance may include different total weight percentages of one or more RNA stabilizing substance. The following embodiments describing weight percentage of one or more RNA stabilizing substance, include the total weight percentage of all RNA stabilizing substances in a composition, as a non-limiting example if a composition comprises two RNA stabilizing substances and one substance is 15% by weight and the other is 50% by weight, then the total weight percentage of RNA stabilizing substances in a composition is 65%.

Embodiments of the present disclosure may include one or more compositions described herein, wherein a composition may comprise an RNA stabilizing substance total weight percent between about X and Y, where X may be 5%, 10%, 20%, 30%, or 40%, and Y is greater than X, and Y may be 95%, 90%, 80%, 70%, 60%, or 50%, as non-limiting examples X may be 10% and Y may be 70% and a composition may comprise an RNA stabilizing substance concentration between about 10%-70%, or X may be 30% and Y may be 60% and a composition may comprise an RNA stabilizing substance concentration between about 30%-60%.

Embodiments of the present disclosure may include one or more compositions described herein, wherein a composition may comprise an RNA stabilizing substance total weight percent greater than about 5%, or greater than about 10%, or greater than about 20%, or greater than about 30%, or greater than about 40%, or greater than about 50%, or greater than about 60%.

Embodiments of the present disclosure may include one or more compositions described herein, wherein a composition may comprise an RNA stabilizing substance total weight percent that is less than about 95%, or less than about 90%, or less than about 80%, or less than about 70%, or less than about 60%, or less than about 50%, or less than about 40%.

Additive Substances

The inventors have surprisingly discovered that additional substances may further improve RNA stability in one or more compositions comprising one or more RNA stabilizing substance and one or more RNA substance. These additional substances are herein referred to as additive substances. The inventors have surprisingly discovered that the addition of one or more additive substances to a composition comprising one or more RNA stabilizing substance and one or more RNA substance may further improve RNA stability.

Embodiments of the present disclosure comprising one or more RNA stabilizing substance and one or more RNA substance, may also comprise one or more additive substances. These embodiments comprising one or more additive substances may include one or more composition as described herein that may also comprise one or more additive substances.

Additive substances may include one or more buffer substance selected from the following (herein referred to as buffer additive substances): acetate, phosphate, citrate, or tris. Embodiments of the present disclosure comprising one or more RNA stabilizing substance and one or more RNA substance, may also comprise one or more buffer additive substance. In some embodiments one or more composition described herein may comprise one or more buffer additive substance, wherein the pH of the composition may be between about 5-9, or between about 5-8, or between about 6-9, or between about 6-8, or between about 7-8.

Embodiments of the present disclosure may include one or more composition described herein, wherein the concentration of a buffer additive substance may be greater than about 5 mM, or greater than about 10 mM, or greater than about 20 mM, or greater than about 50 mM, or greater than about 100 mM, or greater than about 150 mM, or greater than about 200 mM, or greater than about 250 mM, or greater than about 500 mM.

Additive substances may include one or more inorganic cation substances selected from the following (herein referred to as inorganic cation additive substances): Li, Na, or K. Embodiments of the present disclosure comprising one or more RNA stabilizing substance and one or more RNA substance, may also comprise one or more inorganic cation additive substances.

Embodiments of the present disclosure may include one or more composition described herein, wherein the concentration of an inorganic cation additive substance may be greater than about 5 mM, or greater than about 10 mM, or greater than about 20 mM, or greater than about 50 mM, or greater than about 100 mM, or greater than about 150 mM, or greater than about 200 mM, or greater than about 250 mM, or greater than about 500 mM.

Additive substances may include sugars, sugar alcohols, polyols, or cyclitols, herein referred to as carbohydrate additive substances. Non-limiting examples of carbohydrate additive substances that may be used, may include one or more of the following: sucrose, glucose, fructose, trehalose, sorbitol, glycerol, mannitol, xylitol, maltose, dextrose, xylose, mannitol, maltitol, isomalt, xylitol, lactitol, lactose, erythritol, threitol, arabitol, ribitol, galactitol, fucitol, iditol, inositol, myo-inositol, volemitol, maltotriitol, maltotetraitol, polyglycitol, hydrogenated starch hydrolysates, rhamnose, ribose, mannose, galactose, fucose, arabinose, dextrin, erythrose, altrose, allose, lyxose, or combinations thereof.

Embodiments of the present disclosure may include one or more composition described herein, wherein the concentration of a carbohydrate additive substance may be between about 10 mM-4M, such as between about 100 mM-4M, 250 mM-4M, 500 mM-4M, 100 mM-2M, 250 mM-2M, 500 mM-2M, 100 mM-1M, 250 mM-1M, or 500 mM-1M. Embodiments of the present disclosure may include one or more compositions described herein, wherein a composition may comprise one or more carbohydrate additive substance with a concentration greater than 10 mM, or greater than 50 mM, or greater than 100 mM, or greater than 200 mM, or greater than 500 mM, or greater than 1M.

Additive substances may also include one or more the following supplemental additive substances herein referred to as supplemental additive substances. Non-limiting examples of supplemental additive substances that may be used may include one or more of the following: acesulfame, saccharin, aspartame, hexylene glycol, creatine, creatine phosphate, thiamine, ectoine, pyridoxal 5′-phosphate, folic acid (e.g folate), biotin, (D or L) pantothenic acid (e.g. pantothenate), taurine, N,N-dimethylphenethylamine, and benzyltriethylammonium.

Embodiments of the present disclosure may include one or more composition described herein, wherein the concentration of a supplemental additive substance may be between about 5 mM-2M, such as between about 10 mM-2M, 20 mM-2M, 50 mM-2M, 10 mM-1M, 20 mM-1M, 50 mM-1M, 100 mM-1M. Embodiments of the present disclosure may include one or more compositions described herein, wherein a composition may comprise one or more supplemental additive substance with a concentration greater than 5 mM, or greater than 10 mM, or greater than 20 mM, or greater than 50 mM, or greater than 100 mM, or greater than 200 mM.

Embodiments of the present disclosure comprising one or more RNA stabilizing substance and one or more RNA substance, may comprise one or more excipient or diluent, such as one or more pharmaceutically acceptable excipient or diluent.

In some embodiments one or more composition described herein may optionally be lyophilized.

### Non-Limiting Example Multi-Component Compositions

The inventors have discovered that combinations of RNA stabilizing substances comprising compounds from more than one RNA stabilizing substance category may be synergistic and provide better RNA stability than either individual compound (e.g., trimethylglycine (TMG) with DMSO). The inventors have discovered that the stability of RNA substances may be enhanced in compositions comprising an RNA substance and multiple RNA stabilizing substances. Non-limiting embodiments of the present disclosure include RNA stabilizing substances that may comprise one or more compounds that are members of stabilizing substance categories as described herein. Embodiments of the present disclosure may include one or more composition comprising one or more RNA substance and one or more RNA stabilizing substance.

The following list includes non-limiting example multi-component compositions comprising at least one or more RNA substance and at least one or more RNA stabilizing substance.
1. Pyridine-2,6-dicarboxylate, L-carnitine, choline, sorbitol, and at least one RNA substance
2. Hexametaphosphate, N-acetyl proline, pyridine-2,6-dicarboxylate, trimethylglycine and at least one RNA substance.
3. Gamma-butyrobetaine, N-acetyl tyrosine, choline, mannitol, and at least one RNA substance.
4. Carnitine, citrulline, aspartame, NMN and at least one RNA substance.
5. HMP, N-acetyl proline, 3-glyceryl ascorbate, choline and at least one RNA substance.
6. Pyridine-3,5-dicarboxylate, alpha-GPC, citrulline, sorbitol and at least one RNA substance.
7. Choline, gamma-butyrobetaine, N-acetyl tyrosine, saccharin, and at least one RNA substance.
8. NMN, TMG, HMP, N-acetyl proline and at least one RNA substance.
9. Pyridine-2,6-dicarboxylate, TMG, choline, and at least and at least one RNA substance.
10. Citrulline, Pyridine-2,6-dicarboxylate, O-acetyl carnitine, and at least one RNA substance.
11. Proline betaine, 3-glyceryl ascorbate, pyridine-2,3-dicarboxylate, ectoine and at least one
RNA substance.
12. N-acetyl tyrosine, alpha-GPC, pyridine-3,5-dicarboxylate, choline, sorbitol and at least one
RNA substance.
13. Mepiquat, carnitine, N-acetyl proline, pyridine-2,6-dicarboxylate, sorbitol and at least one
RNA substance.
14. N-acetyl aspartate, TMG, mepiquat, ornithine, mannitol and at least one RNA substance.
15. NMN, pyridine-2,6-dicarboxylate, carnitine, choline, sorbitol and at least one RNA substance.

Embodiments of the present disclosure may include one or more compositions described herein, wherein the pH of a composition may be between about 5-9, or between about 5-8, or between about 6-9, or between about 6-8, or between about 7-8.

### Cellular Uptake Agents:

In some embodiments of the present disclosure a combination that comprises one or more RNA stabilizing substance and one or more RNA substance also comprises one or more substance to promote the RNA's ability to enter cells (herein referred to as cellular uptake agents), example substances being, including but not limited to, lipids, polymers, detergents, ionizable polymers, ionizable lipids, cationic polymers, cationic lipids, amino-lipids, cholesterols, cationic detergents, ionizable detergents, lipid nanoparticles, detergent micelles, micelles, liposomes, nanoliposomes, lipoparticles, nanolipoparticles, nanoparticles, lipid micelles, lipid bilayers, or membrane vesicles. Examples of cell entry may include, but are not limited to, fusion with the cellular membrane, endocytosis, pinocytosis, phagocytosis, passive diffusion, active diffusion, osmotic diffusion, facilitated diffusion, diffusion, hole formation, direct microinjection, electroporation, ultrasound, energy induced, electricity induced, electric field induced, or similar mechanisms to deliver the RNA substance to, including but not limited to, a cell, eukaryotic cell, prokaryotic cell, plant cell, fungal cell, plant, bacteria, fungus, insect, organ, tissue, animal, or vertebrate animal, including but not limited to a human, by entering cells.

As used herein, cellular uptake agents means substances that promote RNA's ability to enter cells, such as eukaryotic cells, prokaryotic cells, fungal cells, mammalian cells, animal cells, human cells, plant cells, bacterial cells, mycoplasma, or insect cells as non-limiting examples.

Cellular uptake agents are known art when used with RNA and may also be referred to as gene delivery agents, transfection agents, cellular delivery agents, intracellular delivery agents, or complexation agents. The present disclosure uses cellular uptake agents in the novel configuration of one or more cellular uptake agent with one or more RNA stabilizing substance and one or more RNA substance. At least one or more cellular uptake agent may be combined with at least one or more RNA stabilizing substance and at least one or more RNA substance either in advance and stored together or stored separately, such as in a two-compartment container, and combined close to the time of administration.

In some embodiments of the present disclosure may include a composition comprising one or more RNA substance, one or more RNA stabilizing substance, and one or more cellular uptake agent.

In some embodiments of the present disclosure may include a combination or mixture comprising one or more RNA substance, one or more RNA stabilizing substance, and one or more cellular uptake agent.

Embodiments of the present disclosure that comprise one or more RNA substance, one or more RNA stabilizing substance, and one or more cellular uptake agent may include combining, such as by mixing, one or more RNA substance with one or more RNA stabilizing substance and one or more cellular uptake agent.

In some embodiments of the present disclosure a composition comprising one or more RNA substance, one or more RNA stabilizing substance, and one or more cellular uptake agent produces a mixture with at least one or more RNA substance, at least one or more RNA stabilizing substance, and at least one or more cellular uptake agent.

In some embodiments of the present disclosure a combination comprising one or more RNA substance, one or more RNA stabilizing substance, and one or more cellular uptake agent produces a mixture with at least one or more RNA substance, at least one or more RNA stabilizing substance, and at least one or more cellular uptake agent.

Environment

As described in this disclosure, the stability of stored RNA substances may be improved by RNA substances being in storage environments comprising an RNA stabilizing substance. As non-limiting examples, the improved stability may occur when the storage environment has temperatures higher than an ultracold (as a non-limiting example, about −80° C.), cold (as a non-limiting example, about −20° C.), refrigerated (as a non-limiting example, about 4° C.) temperature, or warmer temperature. As a non-limiting example, the stability of an RNA substance may be determined by comparing the starting average molecular weight of a sample of the RNA substance to the average molecular weight of a sample of the RNA substance that has been stored for at least one predetermined time and temperature. The stability of an RNA substance may be determined by exposing the RNA substance to a specified temperature for a specified time duration and comparing the ending average molecular weight to the starting molecular weight to determine to amount of degradation. The amount of degradation of an RNA substance between the beginning and the end of a time interval is calculated using the formula D=[1−(AMWe/AMWs)]*100 where D is the amount of degradation, AMWe is the average molecular weight of the RNA substance at the end of time interval, AMWs is the average molecular weight of the RNA substance at the start of the time interval with AMWe divided by AMWs with the quotient subtracted from 1 and the result multiplied by 100 for the result to be expressed as a percentage. For example, if the average molecular weight at the end of the time interval is the same as the average molecular weight at the start of the time interval the amount of degradation is zero (0) percent and if the average molecular weight at the end is one-half of the starting molecular weight then the amount of degradation is 50 percent and if the ending average molecular weight is 25 percent of the starting average molecular weight then the degradation is 75 percent. The rate of degradation for an RNA substance is the percent degradation divided by the duration of the time interval, for example 50 percent over 7 days or 50 percent per 7 days. As a non-limiting example, the time and temperature selected may be a predetermined duration at a defined temperature such as 48 hours at 40° C. The average molecular weight of an RNA substance may be determined by means known in the art, including but not limited to, liquid chromatography (e.g. HPLC or FPLC), gel electrophoresis, mass spectrometry, size exclusion chromatography, or other means known in the art. The degradation of RNA substances may be determined using parameters other than direct molecular weight measurements by using parameters known to those skilled in the art that directly relate to molecular weight such as using apparent molecular weight determined using gel electrophoresis with reference markers indicating the number of bases in references' and samples' lanes.

As non-limiting example embodiments of the present disclosure, a composition comprising at least one RNA substance and at least one RNA stabilizing substance may stabilize the RNA substance to degrade no more than about X % of RNA molecules in an environment with temperatures exceeding a defined temperature of about T° C. where X may be 50, 40, 30, 20, 10 and T may be −80, −60, −40, −30, −20, −10, 0, 2, 4, 6, 8, 10, 20, 30, 40 for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent. As a non-limiting example, a composition comprising at least one RNA substance and at least one RNA stabilizing substance may stabilize the RNA substance to degrade no more than about 50% of RNA molecules in an environment with temperatures exceeding a defined temperature of about 20° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent. As a non-limiting example, a composition comprising at least one RNA substance and at least one RNA stabilizing substance may stabilize the RNA substance to degrade no more than about 50% of RNA molecules in an environment with temperatures exceeding about 40° C. continuously for about 48 hours or about 72 hours.

In some embodiments RNA degradation may be a reduction in molecular weight, wherein the molecular weight of the RNA molecule may be less than 95%, or less than 90%, or less than 85%, or less than 80%, or less than 75%, or less than 60%, or less than 50% of the molecular weight compared to that of a control RNA that has not been stored for the predetermined amount of time exceeding the defined temperature. In some specific embodiments RNA degradation may be a reduction in molecular weight, wherein the molecular weight of the RNA molecule may be less than 90%, or less than 85%, or less than 80%, or less than 75%, or less than 60% of the molecular weight compared to that of a control RNA that has not been stored for the predetermined amount of time exceeding the defined temperature. In some even more specific embodiments RNA degradation may be a reduction in molecular weight, wherein the molecular weight of the RNA molecule may be less than 90%, or less than 80%, or less than 60% of the molecular weight compared to that of a control RNA that has not been stored for the predetermined amount of time exceeding the defined temperature.

As used herein, chamber means an enclosed volume capable of containing at least one of a solid, powder, liquid, aerosol, vapor, or gas that may be sealed and later allow at least some of its contents to be at least one of partially delivered, removed, emptied, dispensed, opened, accessed, or penetrated. As a non-limiting example, a chamber may be at least one of, including but not limited to, bottles, containers, vials, tubes, jars, syringes (including prefilled syringes), blisters, capsules, tablets, cartridges, inhalers, packets, pods, bags, boxes, or other packages that may hold a solid, powder, liquid, aerosol, or gas. As non-limiting examples, chambers may be single-piece gel soft capsules or may be two-piece gel hard capsules. As non-limiting examples, chambers may be capsules containing at least one RNA stabilizing substance and at least one RNA substance that may be used in an inhaler.

Chambers contain substantially more than one nucleic acid molecule (e.g., may contain more than 100 molecules) as opposed to particles with a wall surrounding small amounts, (e.g, low count, approximately single, number of molecules) of nucleic acid. Chambers have at least one outer dimension (e.g., diameter, length, width, depth, height, and so on) greater than the size of nanoparticles (nanoparticles typically have dimensions less than about 0.0005 mm) by having at least one outer dimension being at least about 0.01 mm. As non-limiting examples, chambers may have at least one outer dimension (e.g., diameter, length, width, depth, height, and so on) of at least 0.002 mm or of at least 0.005 mm of at least 0.01 mm or of at least 0.1 mm or of at least 1 mm. As a non-limiting example, chambers containing at least one RNA stabilizing substance and at least one RNA substance for nasal spray-type administration may have dimensions in the approximate range of 0.02 mm to 0.12 mm. As a non-limiting example, for chambers containing at least one RNA stabilizing substance and at least one RNA substance for oral administration the length may be about 5 mm or larger or the sum of length+width+depth may be between about 15 mm and about 50 mm or the sum of length+width+depth may be less than about 25 mm or the sum of length+width+depth may be between about 15 mm and 25 mm.

As non-limiting examples, chambers may contain more than 1,000 kg of stabilized RNA composition, or may contain up to 1,000 kg of stabilized RNA composition, or may contain up to 100 kg of stabilized RNA composition or may contain up to 10 kg of stabilized RNA composition. As non-limiting examples, chambers may contain between about 1 kg and 10 kg of stabilized RNA composition, or may contain between about 100 g and 1 kg of stabilized RNA composition, or may contain between about 10 g and about 100 g of stabilized RNA composition, or may contain between about 1 g and 10 g of stabilized RNA composition, or may contain between about 100 mg and 1 g of stabilized RNA composition, or may contain between about 10 mg and 100 mg of stabilized RNA composition, or may contain between about 1 mg and 10 mg of stabilized RNA composition, or may contain between about 100 μg and 1 mg of stabilized RNA composition, or may contain between about 1 μg and 1 mg of stabilized RNA composition, or may contain more than about 0.2 ng of stabilized RNA composition. As a non-limiting example, a chamber may contain more than about 4 ng and less than about 0.7 g of stabilized RNA composition.

In some embodiments of the present disclosure a chamber may comprise one or more RNA substance with one or more RNA stabilizing substance. In some embodiments of the present disclosure a chamber may comprise one or more RNA substance with two or more RNA stabilizing substances. In some embodiments the chamber may be a vial. In some embodiments the chamber may be a prefilled syringe. In some embodiments the chamber may be a capsule or tablet.

In some embodiments of the present disclosure a chamber may comprise one or more RNA substance with one or more RNA stabilizing substance and one or more cellular uptake agent. In some embodiments the chamber may be a vial. In some embodiments the chamber may be a prefilled syringe. In some embodiments the chamber may be a capsule or tablet.

In some embodiments of the present disclosure a chamber may comprise one or more RNA substance with one or more RNA stabilizing substance, and one or more cellular uptake agent. In some embodiments of the present disclosure a chamber may comprise one or more RNA substance with two or more RNA stabilizing substance, and one or more cellular uptake agent. In some embodiments the chamber may be a vial. In some embodiments the chamber may be a prefilled syringe. In some embodiments the chamber may be a capsule or tablet.

In some embodiments of the present disclosure a chamber may comprise one or more RNA substance with one or more RNA stabilizing substance, and one or more cellular uptake agent. In some embodiments of the present disclosure a chamber may comprise one or more RNA substance with two or more RNA stabilizing substances, and one or more cellular uptake agent. In some embodiments the chamber may be a vial. In some embodiments the chamber may be a prefilled syringe. In some embodiments the chamber may be a capsule or tablet.

In some embodiments of the present disclosure, each component included in a composition comprising one or more RNA substance and one or more RNA stabilizing substance and optionally comprising one or more cellular uptake agent, may be stored separately, such as in a kit, or such as individually or as mixtures of one or more substance, and then combined later to produce a composition comprising one or more RNA substance, one or more RNA stabilizing substance, and one or more cellular uptake agent.

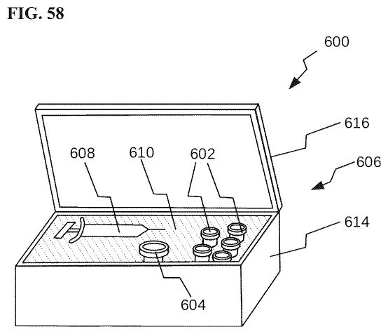

An example of such a kit 600 is shown in FIG. 58. As shown, the kit 600 includes one or more component vials 602 and one or more mixing/dispensing vials 604 contained in a package 606, such as a hinged box. Each of the component vials 602 may contain one or more components of the composition as described herein. For example, each component may be provided in a separate vial 602 or certain compatible components may be combined in a single vial 602 with other components provided individually or in combination in other vials 602.

The components can then be mixed in mixing/dispensing vial 604, for example, immediately prior to use, in order to minimize RNA degradation. In this regard, the components from the vials 602 may be transferred into the vial 604, e.g., poured or injected into the vial 604 using a syringe 608, and then shaken or otherwise mixed. The components in the vials 602 may be premeasured and may provide a single unit or dose of the composition or multiple units/doses. Alternatively, the components from the vials 602 may be measured and combined by skilled workers. Optionally, e.g., in the case of vaccines, one or more syringes 608 may be provided in the kit 600 for drawing the composition from the vial 604 and administering to subjects. The vials 602 and 604 and syringes 608 may be secured by packing material 610 such as a foam material.

As described herein, many compositions, components, or combinations of components in accordance with the present disclosure can be stored without requiring extremely low temperatures. In cases where cold storage is required, the kit 600 can be transported in a cold storage unit or cold storage vehicles. The packaging 606 may be formed from materials suitable to withstand such cold storage such as various plastics or metals. In such cases, the vials 602, 604 and syringes 608 (if provided in the kit 600) may be formed from materials selected to withstand cold storage.

Although the kit is shown as including vials 602, 604 and syringes 608 for purposes of illustration, it will be appreciated that the components may be provided in other forms, e.g., non-liquid forms, and the composition may be provided for purposes other than vaccination. Accordingly, while a kit including some or all of the components of a composition in accordance with the present disclosure is useful and convenient, the nature of the kit can vary from the kit shown.

As a non-limiting example, vials 602 may comprise a concentrated composition comprising at least one RNA substance and at least one RNA stabilizing substance that after being mixed with at least one diluent is suitable for use, such as suitable for injection. In this embodiment the diluent may or may not be part of kit 600.

In another embodiment packaging 606 may comprise vials 602 that may be ready for use and packaging 606 provides a package for uses comprising at least one of as storage and transport and maintaining a desired environment for vials 602. Packaging 606 may comprise a cooling pack (not shown) that at least partly offsets thermal energy transferred from the storage and transport environment to the inside of package 606 where at least some of vials 602 are desired to experience a maximum target temperature.

In some embodiments kit 600 may comprise a cooling substance (not shown) that maintains the temperature of at least one of vials 602 at no more than a maximum target temperature. As a non-limiting example, the maximum target temperature may be about 4° C. or less. As a non-limiting example, the maximum target temperature may be about 20° C. or less. As a non-limiting example, a cooling substance that maintains the maximum target temperature or less may undergo a phase change to maintain the maximum target temperature. Non-limiting examples of cooling substances that may be used to maintain the maximum target temperature are solid phase water that may change to liquid phase water, solid phase carbon dioxide that may change phase to vapor phase carbon dioxide, or liquid phase nitrogen that may change from liquid phase to vapor phase nitrogen.

A non-limiting example embodiment of kit 600 comprises component vials 602 that may contain one or more components of the composition as described herein in which at least one component is an RNA substance and at least one component is an RNA stabilizing substance and a substance that maintains the maximum target temperature of at least one of the vials 602 to about 4° C. or less for at least one hour. In a non-limiting embodiment the kit may maintain the temperature of at least one vial at about 20° C. or less for at least 24 hours. As another non-limiting embodiment, kit 600 may maintain the temperature of at least one vial at about 20° C. or less for at least 60 hours.

In another embodiment at least one or more cellular uptake agent may be combined with at least one or more RNA stabilizing substance, as described herein, and at least one or more RNA substance either in advance and stored together or stored separately, such as in a two-compartment chamber, and combined close to the time of administration.

### Examples of Cellular Uptake Agents:

In some embodiments a cellular uptake agent may comprise, but is not limited to, at least one or more of the following: a lipid, polymer, zwitterionic polymer, zwitterionic lipid, ionizable polymer, ionizable lipid, cationic polymer, cationic lipid, amino-lipid, cholesterol, cationic detergent, zwitterionic detergent, ionizable detergent, non-ionic detergent, detergent, polyethylenimine (PEI), polyplexes, polyamines, lipid nanoparticles, detergent micelles, micelles, liposomes, nanoliposomes, lipoparticles, nanolipoparticles, dendrimers, nanoparticles, lipid membrane, lipid micelle, or membrane vesicles, or combinations thereof.

In some embodiments a cellular uptake agent may comprise, but is not limited to, at least one or more of the following surrounding an aqueous core or a hydrophobic core: a lipid, polymer, zwitterionic polymer, zwitterionic lipid, ionizable polymer, ionizable lipid, cationic polymer, cationic lipid, amino-lipid, cholesterol, cationic detergent, zwitterionic detergent, ionizable detergent, non-ionic detergent, detergent, polyethylenimine (PEI), polyplexes, polyamines, lipid nanoparticles, detergent micelles, micelles, liposomes, nanoliposomes, lipoparticles, nanolipoparticles, dendrimers, nanoparticles, lipid membrane, lipid micelle, or membrane vesicles, or combinations thereof.

As used herein, PEG is polyethylene glycol.

As used herein, a PEG lipid is a lipid modified with or conjugated to polyethylene glycol.

In some embodiments a cellular uptake agent may include, but is not limited: phospholipids, sterols, cholesterol, phospholipid-free lipid particle, non-cationic lipid, cholesterol-free lipid particle, noncyclic phosphate containing lipids, lipid conjugates, PEG-conjugated lipids, PEG-lipid conjugates, cationic-polymer-lipid conjugates, PEG coupled to dialkyloxypropyls, PEG coupled to diacylglycerol, PEG coupled to phospholipids such as phosphatidylethanolamine, PEG conjugated to ceramides, or PEG conjugated to cholesterol, or combinations thereof.

In some embodiments, a cellular uptake agent may include a polyethylene glycol-lipid, PEG or PEG-modified lipids (also known as PEGylated lipids), including but not limited to, at least one or more of the following: PEG-modified phosphatidylethanolamines, PEG-modified phosphatidic acids, PEG-modified ceramides, PEG-modified dialkylamines, PEG-modified diacylglycerols, or PEG-modified dialkylglycerols, or combinations thereof.

In some embodiments a cellular uptake agent may include, but not limited to hydrophilic polymers substituted for or used in combination with PEG as described herein: polyvinylpyrrolidone, polymethyloxazoline, polyethyloxazoline, polyhydroxypropyl methacrylamide, polymethacrylamide or polydimethylacrylamide, polylactic acid, polyglycolic acid, or derivatized celluloses such as hydroxymethylcellulose or hydroxyethylcellulose, or combinations thereof.

In another embodiment a cellular uptake agent may include, but not limited to: cholesterol, fecosterol, sitosterol, ergosterol, campesterol, stigmasterol, brassicasterol, tomatidine, tomatine, ursolic acid, alpha-tocopherol, corticosteroids, prednisolone, dexamethasone, prednisone, or hydrocortisone, or combinations thereof.

In some embodiments, a cellular uptake agent may include a polymer, cationic polymer, cationic or polycationic compounds, or cationic polysaccharides, for example chitosan, polybrene, or polyethyleneimine (PEI), or derivatives, combinations, or mixtures thereof.

In some embodiments, a cellular uptake agent may include, but not limited to, cationic peptides or proteins, cell penetrating peptides, basic polypeptides, basic amino acids or their derivatives, cationic dendrimers, polyamines, polyamine sugars, amino polysaccharides, oligofectamine, modified polyaminoacids, β-aminoacid-polymers, reversed polyamides, modified polyethylenes, modified acrylates, modified amidoamines, modified polybetaaminoester, dendrimers, polypropylamine dendrimers, poly(amidoamine) PAMAM based dendrimers, polyimines, poly(ethyleneimine), poly(propyleneimine), polyallylamine, polylysine, polyornithine, poly/lysine/ornithine, poly(propylene imine), poly(vinyl amine), poly(2-aminoethyl methacrylate), sugar backbone based polymers, cyclodextrin based polymers, dextran based polymers, chitosan, or silane backbone based polymers, or derivatives, combinations, or mixtures thereof.

Non-limiting examples of cellular uptake agents that may be suitable for use with the RNA stabilizing substances of the present disclosure are described in US Patent Application Pub. No. US 2020/0383922 A1, incorporated herein by reference, as cationic or polycationic compounds that may be used as transfection or complexation agents.

Non-limiting examples of cellular uptake agents that may be suitable for use with the RNA stabilizing substances of the present disclosure are described in U.S. Pat. Nos. 10,702,600 and 10,933,127, incorporated herein by reference, as nanoparticle formulations, cationic lipid nanoparticles, nanoparticles, liposomes, lipoplexes, lipid nanoparticles (LNPs), lipids, cationic lipids, ionizable lipids, PEG lipids, structural lipids, neutral lipids, non-cationic lipids, therapeutic nanoparticles, polymeric material, polymer-vitamin conjugate, block co-polymer or tri-block co-polymer, surface altering agents, or cationic or polycationic compounds.

Non-limiting examples of cellular uptake agents that may be suitable for use with the RNA stabilizing substances of the present disclosure are described in U.S. Pat. Nos. 8,058,069 and 9,364,435, incorporated herein by reference, as lipid particles, stable nucleic acid-lipid particle (SNALP), lipids, lipid conjugates, amphipathic lipids, neutral lipids, non-cationic lipids, anionic lipids, cationic lipids, hydrophobic lipids, or sterols.

Non-limiting examples of cellular uptake agents that may be suitable for use with the RNA stabilizing substances of the present disclosure are described in WO Patent Application Pub. No. WO 2021/156267 A1, incorporated herein by reference, as polymeric carriers, lipidoids or cationic lipidoids, lipid nanoparticles (LNPs), liposomes, lipoplexes, nanoliposomes, lipids, cationic or polycationic lipids, neutral lipids, ionizable lipids, polymer conjugated lipids, cationic or polycationic compounds, cationic or polycationic polymers, cationic or polycationic polysaccharides, cationic or polycationic proteins, or cationic or polycationic peptides.

Non-limiting examples of cellular uptake agents that may be suitable for use with the RNA stabilizing substances of the present disclosure are described in U.S. Pat. No. 8,367,628, incorporated herein by reference, as lipids, amphoteric lipids, amphoteric liposomes, amphoteric liposomal mixtures, liposomal mixtures, sterols, cationic lipids, chargeable cationic lipids, chargeable anionic lipids, stable anionic lipids, neutral lipids, or mixtures of lipid components with amphoteric properties.

Non-limiting examples of cellular uptake agents that may be suitable for use with the RNA stabilizing substances of the present disclosure are described in US Patent Application Pub. No. US 2021/0260097 A1, incorporated herein by reference, as nanoparticles, lipid nanoparticles (LNPs), lipids, cationic or ionizable lipids, anionic lipids, neutral lipids, amphipathic lipids, PEGylated lipids, or structural lipids.

Non-limiting examples of cellular uptake agents that may be suitable for use with the RNA stabilizing substances of the present disclosure are described in US Patent Application Pub. No. US 2021/0261627 A1, incorporated herein by reference, as cationic or polycationic compounds, polymeric carriers, cationic polysaccharides, cationic lipids, polymers, cationic or polycationic polymers, copolymers, blockpolymers, or cationic or polycationic proteins or peptides, which may be used as transfection or complexation agents.

### Containers and Mixing

One or more RNA stabilizing substance and one or more RNA substance may be in a chamber, as illustrated in the non-limiting example shown in FIG. 65. Chambers may include containers, such as syringes or vials, which may hold a combination of at least one or more RNA stabilizing substance and at least one or more RNA substance and the containers may also contain one or more additional substance, such as at least one or more cellular uptake agents, or additive substances, or one or more additional RNA stabilizing substances as non-limiting examples.

Furthermore, chambers may include containers, such as syringes or vials, which may hold a combination of at least one or more RNA stabilizing substance and at least one or more RNA substance and the containers may also contain one or more additional substance, such as at least one or more cellular uptake agents or other substances, that are kept separate from the RNA stabilizing substance and the RNA substance until close to the time of use.

As a non-limiting example, a container may hold a combination of materials comprising at least one or more RNA substance and at least one or more RNA stabilizing substance that is ready for injection after removal from the container such as by withdrawal using a syringe and needle. As a non-limiting example, a container, which may be a vial as a non-limiting example, may hold a combination of materials that is a concentrate for injection comprising at least one or more RNA substance and at least one or more RNA stabilizing substance that after dilution is ready for injection upon removal from the container such as by withdrawal using a syringe and needle. As a non-limiting example, the dilution may occur by adding a solution comprising water to a container, which may be a vial, that is partially filled to leave volume for adding and mixing diluting solution. As a non-limiting example, the diluting solution may be 0.9% sodium chloride (normal saline, preservative-free). As a non-liming example, the container may be a multi-dose container, which may be a multi-dose vial, containing at least two doses or containing at least 5 doses or containing at least 10 doses or containing at least 15 doses. As a non-liming example, the container may be a multi-dose vial that is at least 2 ml size or that is at least 5 ml size or that is at least 10 ml size or that is at least 20 ml size or that is at least 30 ml size.

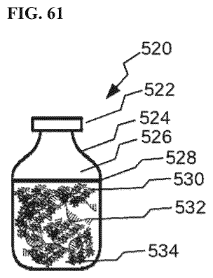

As a non-limiting example, a vial 520, as shown in FIG. 61, may have a seal 522 affixed to a container 524 forming an enclosed volume 526 that is at least partially filled to a predetermined quantity or level 528 with liquid material 534 comprising at least one RNA stabilizing substance 530 (depicted as dots in FIG. 61) at least one RNA substance 532 (depicted as the wave lines and straight lines in FIG. 61). As a non-limiting example, seal 522 may comprise an elastomeric reseal that may be penetrated by a needle (not shown) affixed to syringe (not shown), enabling liquid material 534 to be at least partially withdrawn from vial 520 and transferred to the syringe using techniques known to those skilled in the art. A non-limiting alternative example use of vial 520 is to have the syringe at least partially contain a material, such as normal saline as a non-limiting example, suitable for diluting liquid material 534 comprising at least RNA stabilizing substance 530 and RNA substance 532 by injecting the material from the syringe (not shown) into vial 520 prior to withdrawing at least part of the combined, and as a non-limiting example, mixed, materials from vial 520 into the syringe. One or more syringes may be used to withdraw materials from vial 520. Other devices besides syringes may be used to withdraw materials, non-limiting examples include pumping devices and devices that pressurize enclosed volume 526 of vial 520.

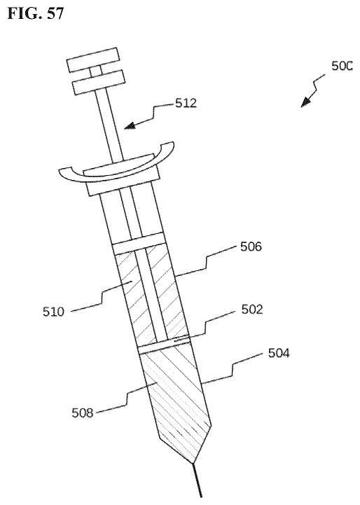

As a non-limiting example, a multi-compartment syringe 500, as shown in FIG. 57, with a breakable seal 502 between compartments may have a first mixture 508 including at least one or more RNA stabilizing substance, at least one or more RNA substance, and at least one or more cellular uptake agents, such as lipids, in one compartment 504 and an aqueous solution 510, such as water, in a second compartment 506. At time of use the seal 502 between the compartments 504 and 506 is broken and the contents of both compartments 504 and 506 are mixed to induce at least one or more cellular uptake agent, such as lipids, to combine with one or more RNA substance prior to use. For example, the seal 502 may be broken by advancing or turning a plunger assembly 512 or portion thereof to puncture a membrane or otherwise enable mixing. In another non-limiting example, at least one or more RNA substance and at least one or more RNA stabilizing substance are in one compartment and both an aqueous solution, such as water, and at least one or more cellular uptake agent, such as a polymer, are in a second compartment with a breakable seal between the compartments. At time of use the seal is broken and the contents of the two compartments are mixed to induce at least one or more cellular uptake agent, such as a polymer, to combine with one or more RNA substance prior to use.

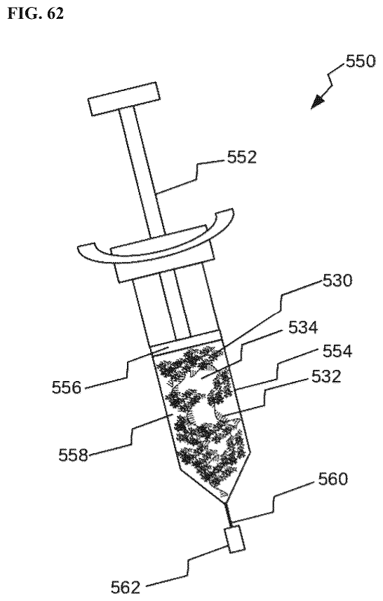

As a non-limiting example, single compartment syringe 550, is shown in FIG. 62, As a non-limiting example, a syringe 550, may have plunger assembly 552 slidably moving relative to syringe container 554 with sliding seal element 556 as part of plunger assembly 552 forming an enclosed volume 558 that is at least partially filled with liquid material 534 comprising at one least RNA stabilizing substance 530 (depicted as dots in FIG. 62) and at least one RNA substance 532 (depicted as the wave lines and straight lines in FIG. 62). Delivery port 560 fluidically communicates with enclosed volume 558 and may, as non-limiting examples, be a needle or an opening (not shown) syringe 550 to which a needle or other delivery component attaches. Port sealing element 562 substantially retains liquid material 534 inside enclosed container space 558 after liquid material 534 is loaded into enclosed container space such as during prefilling syringe 550 during manufacturing or other preparation. Port sealing element 562 may, as non-limiting examples, seal port 560 by surrounding the outside of the port or by plugging the inside of one or more fluid channels in port 560. Liquid material 534 is transferred from the syringe using techniques known to those skilled in the art by operating plunger assembly 552.

In other embodiments of the present disclosure, containers may be embedded complexes comprising at least one RNA stabilizing substance and at least one RNA substance that remain substantially non-communicative across at least one boundary until interaction with a biologic material, as a non-limiting example the biologic material may be fluid or tissue in a living organism, alters the embedded complex so that at least one RNA substance moves from the embedded complex. As a non-limiting example, the embedded complex may be implanted in a living organism and upon being altered by biologic material of the living organism at least one RNA substance at least partially moves from the embedded complex into at least one tissue of the living organism. As a non-limiting example, one or more other substances besides RNA substance may at least partially move from the embedded complex into at least one tissue. As non-limiting examples, the substances besides RNA substances may comprise at least one of RNA stabilizing substances, cellular uptake agents, materials antagonistic to cellular activity either alone or in combination with metabolic processes influenced by one or more RNA substance, and substances benefiting cellular activity either alone or in combination with metabolic process influenced by one or more RNA substance. As a non-limiting example, the embedded complex may deliver one or more RNA substances that produce a response in neoplasms that alter, inhibit, or terminate metabolic activity of at least one neoplastic cell. As a non-limiting example the embedded complex may be implanted in at least the vicinity or at least partially in a neoplasm or where communication between at least one RNA substance and at least one neoplasm may occur through biologic transport processes such through fluid transfer, which may include transfer through the blood system or through the lymph system. As a non-limiting example, the embedded complex may comprise at least one RNA substance that affects the survival or growth of at least one non-malignant neoplasm. As a non-limiting example, the embedded complex may comprise at least one RNA substance that affects the survival or growth of at least one malignant neoplasm.

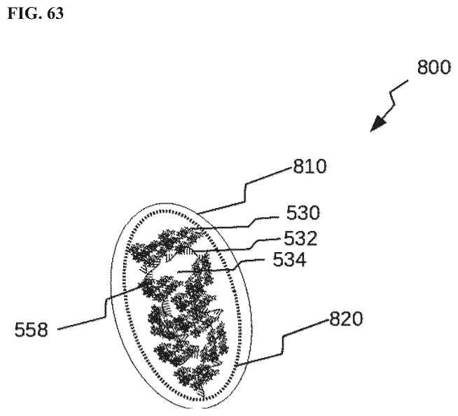

As a non-limiting example, embedded complex 800, is shown in FIG. 63. As a non-limiting example, embedded complex 800, may have a packaging layer 810 that that protects the implantable material during shipping, storage, and provides a sterile barrier. Polymeric material 820 form an enclosed space 558 that at least partially contains internal material 534 comprising at least one RNA substance 532 (depicted as the wave lines and straight lines in FIG. 63). Polymeric material 820 may comprise an RNA stabilizing substance, as a non-limiting example polymeric material 820 may comprise one or more RNA stabilizing polymer substance. Polymeric material 820 may degrade when exposed to biologic materials, such biologic fluids and such degradation may allow internal material 534 to enter one or more tissues in which embedded complex 800 is embedded, such by implanted in one or more tissues. The tissues in which embedded complex 800 is embedded may be from implanting into animal tissue and as non-limiting examples, the animal tissues may be one or more mammalian tissues. As a non-limiting example the mammalian tissues and may be one or more human tissues.

Embedded complex 800 may have diffusion controlling barrier in addition to or instead of polymeric material that degrades when exposed to biologic materials.

As another non-limiting example, embedded complex 800 may be a container such as a tablet, lozenge, capsule, gel capsule, or other container that contacts biologic fluids without being implanted. As non-limiting examples, embedded complex 800 may be ingested orally, be a suppository, or be at least part of an inhalable material such as a mist or droplets produced by a nebulizer.

The containers that may hold a combination comprising at least one or more RNA stabilizing substance and at least one or more RNA substance may be made of any materials suitable for storing and shipping at least one RNA stabilizing substance and at least one RNA substance including, but not limited to, glass, metal, ceramic, plastic or other polymeric material that does not degrade or modify the container's contents or be degraded or modified by the container's contents. The container may have an access port that is penetrated or removed to access the interior of the container including accessing at least part of the contents of the container. The containers may have an access port, such as a screw lid, removable tab, resealable plug or cap or closure that may be penetrated, such as by a hollow tube, such as a hollow needle, to access the interior of the container either to add one or more materials to the contents of the container or to remove at least part of the contents from the interior of the container.

The containers may be used to add materials to the at least one or more RNA stabilizing substance and at least one or more RNA substance. As a non-limiting example, a syringe containing a substance comprising at least one or more lipid may have at least part of its contents transferred to the interior of the container through the access port such as by using a hollow needle penetrating a resealing closure. As another non-limiting example, the syringe may contain an aqueous solution, such as water, that is at least partially transferred to a container having contents comprising at least one RNA stabilizing substance and at least one RNA substance.

The containers, described above, may also hold a combination comprising at least one or more RNA stabilizing substance, at least one or more RNA substance, and at least one or more cellular uptake agent. The containers, described above, may be made of any materials suitable for storing and shipping at least one RNA stabilizing substance, at least one RNA substance and at least one cellular uptake agent including, but not limited to, glass, metal, ceramic, plastic or other polymeric material that does not degrade or modify the container's contents or be degraded or modified by the container's contents.

The containers, described above, may be used to add materials to the at least one or more RNA stabilizing substance, at least one or more RNA substance, and at least one or more cellular uptake agent. As another non-limiting example, the syringe may contain an aqueous solution, such as water, that is at least partially transferred to a container having contents comprising at least one RNA stabilizing substance and at least one RNA substance and at least one cellular uptake agent.

### Uses and Applications:

In other methods of use a combination comprising at least one or more RNA stabilizing substance and at least one or more RNA substance is a combination stored in a chamber at a temperature greater than or equal to about −20° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In other methods of use a combination comprising at least one or more RNA stabilizing substance and at least one or more RNA substance is a combination stored in a chamber at a temperature greater than or equal to about 0° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In other methods of use a combination comprising at least one or more RNA stabilizing substance and at least one or more RNA substance is a combination stored in a chamber at a temperature greater than or equal to about 4° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In other methods of use a combination comprising at least one or more RNA stabilizing substance and at least one or more RNA substance is a combination stored in a chamber at a temperature greater than or equal to about 10° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In other methods of use a combination comprising at least one or more RNA stabilizing substance and at least one or more RNA substance is a combination stored in a chamber at a temperature greater than or equal to about 20° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In other methods of use a combination comprising at least one or more RNA stabilizing substance and at least one or more RNA substance is a combination stored in a chamber at a temperature greater than or equal to about 30° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In other methods of use a combination comprising at least one or more RNA stabilizing substance and at least one or more RNA substance is a combination stored in a chamber at a temperature greater than or equal to about 40° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In other methods of use a combination comprising at least one or more RNA stabilizing substance and at least one or more RNA substance is a combination stored in a chamber at a temperature greater than or equal to about 50° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

Embodiments of the foregoing methods of use include using one or more RNA stabilizing substance mixed with one or more RNA substance.

In some methods of use a combination comprising at least one or more RNA stabilizing substance and at least one or more RNA substance is a combination stored at a temperature less than the melting point of the combination of substances.

In some methods of use a combination comprising at least one or more RNA stabilizing substance and at least one or more RNA substance is a combination stored at a temperature less than the melting point of the combination of substances.

In some methods of use a combination comprising at least one or more RNA stabilizing substance and at least one or more RNA substance and at least one or more cellular uptake agent is a combination stored at a temperature less than the melting point of the combination of substances.

In some methods of use a combination comprising at least one or more RNA stabilizing substance and at least one or more RNA substance and at least one or more cellular uptake agent is a combination stored at a temperature less than the melting point of the combination of substances.

### Applications and Methods of Use

Some embodiments of the present disclosure are the methods whereby one or more RNA stabilizing substance may be combined, such as by mixing, with at least one or more RNA substance to produce a mixture comprising at least one or more RNA stabilizing substance and at least one or more RNA substance. As a non-limiting example one or more RNA substance may be mixed with one or more RNA stabilizing substance.

Some embodiments of the present disclosure are the methods whereby one or more RNA stabilizing substance may be combined with one or more RNA substance such as by, including but not limited to, mixing, pipetting, blending, stiffing, inverting, submerging, vortexing, shaking, lyophilizing, vaporizing, or sublimating such that at least one or more RNA stabilizing substance is at least intimately associated with or at least partially contacting or at least partially encapsulating at least one or more RNA substance.

Some embodiments of the present disclosure are the methods whereby one or more RNA stabilizing substance may be combined with one or more RNA substance by mixing the substances using known methods. These methods include, but are not limited to, stiffing, fluid flow agitation, vortexing, inverting, pipetting, blending, multiple channel fluidics, low shear blending, microfluidic mixing, or using static mixers. These same methods may be used to combine other substances, including, but not limited to cellular uptake agents, water, additive substances, or additional RNA stabilizing substances, with one or more RNA substances and one or more RNA stabilizing substances.

Embodiments of methods described herein may be independent of the order in which each substance may be combined or mixed together. As a non-limiting example one or more RNA stabilizing substance may be combined with one or more RNA substance or one or more RNA substance may be combined with one or more RNA stabilizing substance by the same method.

Some embodiments of the present disclosure are the methods whereby one or more RNA stabilizing substances may be combined with at least one or more RNA substance in a container comprising glass, plastic, ceramic, an elastomer, a polymer, or metal.

Some embodiments of the present disclosure are the methods whereby one or more RNA stabilizing substances may be combined with at least one or more RNA substance and introduced into a container comprising glass, plastic, ceramic, an elastomer, a polymer, or metal.

In some embodiments, single doses of a stabilized RNA composition may be packaged and sealed. In some embodiments, multiple doses of a stabilized RNA composition may be packaged and sealed in one packaging unit. As a non-limiting example, single doses or multiple doses may be packaged in chambers.

In some embodiments of the present disclosure one or more RNA stabilizing substance may also be used in conjunction with lyophilization of at least one or more RNA substance. In some embodiments of the present disclosure one or more RNA stabilizing substance may not be used in conjunction with lyophilization of at least one or more RNA substance.

As used herein, stabilized RNA compositions or RNA stabilizing compositions, may be one or more composition, combination, or mixture comprising at least one or more RNA stabilizing substance and at least one or more RNA substance as described herein. Stabilized RNA compositions or RNA stabilizing compositions may also comprise one or more or more additional substances, including but not limited to, cellular uptake agents, water, additive substances or additional RNA stabilizing substances as non-limiting examples.

In a further aspect, the present disclosure further provides the use of the inventive method in the manufacture of a pharmaceutical composition.

In some embodiments of the present disclosure, one or more RNA stabilizing compositions may be used in the manufacture of a pharmaceutical composition.

According to yet another aspect of the present disclosure, a pharmaceutical composition may be provided, wherein a pharmaceutical composition may comprise one or more RNA stabilizing compositions as described herein.

In some embodiments a pharmaceutical composition may be used to treat, prevent, cure, or diagnose one or more disease or improve or prolong the health of humans, plants, or animals, including non-human primates, vertebrate animals, and non-vertebrate animals.

In some embodiments a pharmaceutical composition may comprise one or more composition, combination, or mixture comprising at least one or more RNA stabilizing substance and at least one or more RNA substance as described herein. In some embodiments a pharmaceutical composition may comprise one or more composition, combination, or mixture comprising at least one or more RNA stabilizing substance, at least one or more RNA substance, and at least one or more cellular uptake agent as described herein.

In some embodiments, a pharmaceutical composition may comprise one or more additional pharmaceutically acceptable ingredient, such as a pharmaceutically acceptable carrier or vehicle.

In some embodiments one or more RNA substance within a pharmaceutical composition may comprise at least one or more pharmaceutically active ingredients.

In some embodiments a pharmaceutical composition may comprise at least one or more pharmaceutically active RNA component. In some embodiments a pharmaceutical composition may comprise at least one or more biologically active RNA component.

In some embodiments a pharmaceutical composition may comprise one or more non-RNA pharmaceutically active component. Wherein a non-RNA pharmaceutically active component may be a compound that has a therapeutic effect against a particular medical indication, such as, but not limited to, cancer diseases, autoimmune disease, allergies, or infectious diseases as non-limiting examples. Non-limiting examples of such compounds may include, but are not limited to: peptides or proteins, (therapeutically active) low molecular weight organic or inorganic compounds (molecular weight less than 5,000), sugars, antigens or antibodies, therapeutic agents already known in the art, antigenic cells, antigenic cellular fragments, cellular fractions, modified, attenuated or de-activated pathogens (e.g. virus, bacteria, fungus, protozoa, plasmodium, or mycobacterium), wherein a pathogen may be attenuated or deactivated chemically, by irradiation, mutation, serial passage, or other known method.

In some embodiments one or more pharmaceutical compositions may be administered orally, sublingually, transdermally, ophthalmically, parenterally, subcutaneous, intravenous, intramuscular, by inhalation, topically, rectally, nasally, buccally, vaginally, or via an implant. The term parenteral or parenterally as used herein includes, but is not limited to, subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, or sublingual injection or infusion techniques.

In some methods of use one or more pharmaceutical compositions may be administered orally, sublingually, transdermally, ophthalmically, parenterally, subcutaneous, intravenous, intramuscular, by inhalation, topically, rectally, nasally, buccally, vaginally, or via an implant. The term parenteral or parenterally as used herein includes, but is not limited to, subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, or sublingual injection or infusion techniques.

In some methods of use one or more RNA stabilizing composition as described herein may be used to produce a pharmaceutical composition comprising at least one or more RNA substance and at least one or more RNA stabilizing substance.

In some methods of use one or more RNA stabilizing composition as described herein may be used to produce a pharmaceutical composition comprising at least one or more RNA substance, at least one or more RNA stabilizing substance, and at least one or more cellular uptake agent.

In some methods of use one or more RNA stabilizing composition as described herein may be combined with one or more pharmaceutical composition comprising at least one or more RNA substance and at least one or more RNA stabilizing substance.

In some methods of use one or more RNA stabilizing composition as described herein may be combined with one or more pharmaceutical composition comprising at least one or more RNA substance, at least one or more RNA stabilizing substance, and at least one or more cellular uptake agent.

In some embodiments one or more RNA stabilizing composition as described herein may be combined with one or more pharmaceutical composition comprising at least one or more RNA substance and at least one or more RNA stabilizing substance.

In some embodiments one or more RNA stabilizing composition as described herein may be combined with one or more pharmaceutical composition comprising at least one or more RNA substance, at least one or more RNA stabilizing substance, and at least one or more cellular uptake agent.

In some embodiments one or more pharmaceutical composition may comprise one or more medicament, vaccine, therapeutic agent, or biostimulant.

In a further aspect, the present disclosure further provides the use of the inventive method in the manufacture of a medicament, vaccine, or therapeutic agent.

In some embodiments of the present disclosure, one or more RNA stabilizing compositions may be used in the manufacture of a medicament, vaccine, or therapeutic agent.

According to yet another aspect of the present disclosure, a medicament, vaccine, or therapeutic agent may be provided, wherein a medicament, vaccine, or therapeutic agent may comprise one or more RNA stabilizing compositions as described herein.

In some embodiments a medicament, vaccine, or therapeutic agent may comprise one or more composition, combination, or mixture comprising at least one or more RNA stabilizing substance and at least one or more RNA substance as described herein. In some embodiments a medicament, vaccine, or therapeutic agent may comprise one or more composition, combination, or mixture comprising at least one or more RNA stabilizing substance, at least one or more RNA substance, and at least one or more cellular uptake agent as described herein.

In some embodiments, a medicament, vaccine, or therapeutic agent may comprise one or more additional pharmaceutically acceptable ingredient, such as a pharmaceutically acceptable carrier or vehicle.

In some embodiments one or more RNA substance within a medicament, vaccine, or therapeutic agent may comprise at least one or more pharmaceutically active ingredients.

In some embodiments a medicament, vaccine, or therapeutic agent may comprise one or more pharmaceutically active RNA component. In some embodiments a medicament, vaccine, or therapeutic agent may comprise one or more biologically active RNA component.

In some embodiments a medicament, vaccine, or therapeutic agent may comprise one or more non-RNA pharmaceutically active component. Wherein a non-RNA pharmaceutically active component may be a compound that has a therapeutic effect against a particular medical indication, such as, but not limited to, cancer diseases, autoimmune disease, allergies, infectious diseases or a further disease, as non-limiting examples. Non-limiting examples of such compounds may include, but are not limited to: peptides or proteins, (therapeutically active) low molecular weight organic or inorganic compounds (molecular weight less than 5,000), sugars, antigens or antibodies, therapeutic agents already known in the art, antigenic cells, antigenic cellular fragments, cellular fractions, modified, attenuated or de-activated pathogens (e.g. virus, bacteria, fungus, protozoa, plasmodium, or mycobacterium), wherein a pathogen may be attenuated or deactivated chemically, by irradiation, mutation, serial passage, or other known method.

In some embodiments one or more medicament, vaccine, or therapeutic agent, may be administered orally, sublingually, transdermally, ophthalmically, parenterally, subcutaneous, intravenous, intramuscular, by inhalation, topically, rectally, nasally, buccally, vaginally, or via an implant. The term parenteral or parenterally as used herein includes, but is not limited to, subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, or sublingual injection or infusion techniques.

In some methods of use one or more medicament, vaccine, or therapeutic agent, may be administered orally, sublingually, transdermally, ophthalmically, parenterally, subcutaneous, intravenous, intramuscular, by inhalation, topically, rectally, nasally, buccally, vaginally, or via an implant. The term parenteral or parenterally as used herein includes, but is not limited to, subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, or sublingual injection or infusion techniques.

In some methods of use one or more RNA stabilizing composition as described herein may be used to produce a medicament, vaccine, or therapeutic agent comprising at least one or more RNA substance and at least one or more RNA stabilizing substance.

In some methods of use one or more RNA stabilizing composition as described herein may be used to produce a medicament, vaccine, or therapeutic agent comprising at least one or more RNA substance, at least one or more RNA stabilizing substance, and at least one or more cellular uptake agent.

In some methods of use one or more RNA stabilizing composition as described herein may be combined with a medicament, vaccine, or therapeutic agent comprising at least one or more RNA substance and at least one or more RNA stabilizing substance.

In some methods of use one or more RNA stabilizing composition as described herein may be combined with a medicament, vaccine, or therapeutic agent comprising at least one or more RNA substance, at least one or more RNA stabilizing substance, and at least one or more cellular uptake agent.

In some embodiments one or more RNA stabilizing composition as described herein may be combined with one or more medicament, vaccine, or therapeutic agent comprising at least one or more RNA substance and at least one or more RNA stabilizing substance.

In some embodiments one or more RNA stabilizing composition as described herein may be combined with one or more medicament, vaccine, or therapeutic agent comprising at least one or more RNA substance, at least one or more RNA stabilizing substance, and at least one or more cellular uptake agent.

In a further aspect, the present disclosure further provides the use of the inventive method in the manufacture of one or more biostimulant composition, including, but not limited to one or more of the following: plant biostimulant, crop biostimulant, agricultural biostimulant, agricultural treatment, soil treatment, horticultural biostimulant, horticultural treatment, microbial biostimulant, bacterial biostimulant, fungal biostimulant, antifungal, antibiotic, antiviral, viricide, microbial treatment, microbial disinfectant, biocide, microbicide, pesticide, insecticide, or insect biostimulant.

As used herein a biostimulant is one or more substance delivered to an organism (such as plants, algae, fungi, or bacteria as non-limiting examples) with the aim to alter or enhance the characteristics, nutrition efficiency, stress tolerance (including biotic or abiotic stress tolerance), or organism quality traits.

As used herein a biostimulant composition is a composition, combination, or mixture comprising at least one or more biostimulants.

In some embodiments of the present disclosure, one or more RNA stabilizing compositions may be used in the manufacture of one or more biostimulant composition, including, but not limited to one or more of the following: plant biostimulant, crop biostimulant, agricultural biostimulant, agricultural treatment, soil treatment, horticultural biostimulant, horticultural treatment, microbial biostimulant, bacterial biostimulant, fungal biostimulant, antifungal, antibiotic, antiviral, viricide, microbial treatment, microbial disinfectant, biocide, microbicide, pesticide, insecticide, or insect biostimulant.

According to yet another aspect of the present disclosure, a biostimulant composition may be provided, wherein a biostimulant composition may comprise one or more RNA stabilizing compositions as described herein.

In some embodiments a biostimulant composition may comprise one or more composition, combination, or mixture comprising at least one or more RNA stabilizing substance and at least one or more RNA substance as described herein. In some embodiments a biostimulant composition may comprise one or more composition, combination, or mixture comprising at least one or more RNA stabilizing substance, at least one or more RNA substance, and at least one or more cellular uptake agent as described herein.

In some embodiments, a biostimulant composition may comprise one or more additional biologically, environmentally, or agriculturally acceptable ingredient, such as a biologically, environmentally, or agriculturally acceptable carrier or vehicle.

In some embodiments one or more RNA substance within a biostimulant composition may comprise at least one or more biologically active ingredients.

In some embodiments a biostimulant composition may comprise one or more biologically active RNA component.

In some embodiments a biostimulant composition may comprise one or more non-RNA biologically active component.

In some embodiments one or more biostimulant compositions may be administered via spray, powder, aerosol, mist, solution, water additive, soil additive, fertilizer additive, liquid, tablet, or other known methods.

In some methods of use one or more biostimulant compositions may be administered via spray, powder, aerosol, mist, solution, water additive, soil additive, fertilizer additive, liquid, tablet, or other known methods.

In some methods of use one or more RNA stabilizing composition as described herein may be used to produce a biostimulant composition comprising at least one or more RNA substance and at least one or more RNA stabilizing substance.

In some methods of use one or more RNA stabilizing composition as described herein may be used to produce a biostimulant composition comprising at least one or more RNA substance, at least one or more RNA stabilizing substance, and at least one or more cellular uptake agent.

In some methods of use one or more RNA stabilizing composition as described herein may be combined with a biostimulant composition comprising at least one or more RNA substance and at least one or more RNA stabilizing substance.

In some methods of use one or more RNA stabilizing composition as described herein may be combined with a biostimulant composition comprising at least one or more RNA substance, at least one or more RNA stabilizing substance, and at least one or more cellular uptake agent.

In some embodiments one or more RNA stabilizing composition as described herein may be combined with one or more biostimulant composition comprising at least one or more RNA substance and at least one or more RNA stabilizing substance.

In some embodiments one or more RNA stabilizing composition as described herein may be combined with one or more biostimulant composition comprising at least one or more RNA substance, at least one or more RNA stabilizing substance, and at least one or more cellular uptake agent.

In a further aspect, the present disclosure further provides the use of the inventive method in the manufacture of one or more implants, wherein an implant may be implanted, attached or otherwise contacting tissues, skin, blood, fluid, or cells of a living organism, such as humans, plants, or animals as non-limiting examples.

In some embodiments of the present disclosure, one or more RNA stabilizing compositions may be used in the manufacture of one or more implant, wherein an implant may be implanted, attached or otherwise contacting tissues, skin, blood, fluid, or cells of a living organism, such as humans, plants, or animals as non-limiting examples.

According to yet another aspect of the present disclosure, an implant may be provided, wherein an implant may comprise one or more RNA stabilizing compositions as described herein.

In some embodiments an implant may be used to treat, prevent, cure, or diagnose one or more disease or improve or prolong the health of humans, plants, or animals, including non-human primates, vertebrate animals, and non-vertebrate animals.

In some embodiments an implant may comprise one or more composition, combination, or mixture comprising at least one or more RNA stabilizing substance and at least one or more RNA substance as described herein. In some embodiments an implant may comprise one or more composition, combination, or mixture comprising at least one or more RNA stabilizing substance, at least one or more RNA substance, and at least one or more cellular uptake agent as described herein.

In some embodiments, an implant may comprise one or more additional pharmaceutically, biologically, environmentally, or agriculturally acceptable ingredient, such as a pharmaceutically, biologically, environmentally, or agriculturally acceptable carrier or vehicle.

In some embodiments one or more RNA substance within an implant may comprise at least one or more pharmaceutically active ingredients. In some embodiments one or more RNA substance within an implant may comprise at least one or more biologically active ingredients.

In some embodiments an implant may comprise one or more pharmaceutically active RNA component. In some embodiments an implant may comprise one or more biologically active RNA component.

In some embodiments an implant may comprise one or more non-RNA pharmaceutically or biologically active component. Wherein a non-RNA pharmaceutically active component may be a compound that has a therapeutic effect against a particular medical indication, such as, but not limited to, cancer diseases, autoimmune disease, allergies, infectious diseases or a further disease, as non-limiting examples. Non-limiting examples of such compounds may include, but are not limited to: peptides or proteins, (therapeutically active) low molecular weight organic or inorganic compounds (molecular weight less than 5,000), sugars, antigens or antibodies, therapeutic agents already known in the art, antigenic cells, antigenic cellular fragments, cellular fractions, modified, attenuated or de-activated pathogens (e.g. virus, bacteria, fungus, protozoa, plasmodium, or mycobacterium), wherein a pathogen may be attenuated or deactivated chemically, by irradiation, mutation, serial passage, or other known method.

In some embodiments one or more implants may be implanted or administered orally, sublingually, transdermally, ophthalmically, parenterally, subcutaneous, intravenous, intramuscular, by inhalation, topically, rectally, nasally, buccally, vaginally, or via implant. The term parenteral or parenterally as used herein includes, but is not limited to, subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, or sublingual injection or infusion techniques.

In some methods of use one or more implants may be implanted or administered orally, sublingually, transdermally, ophthalmically, parenterally, subcutaneous, intravenous, intramuscular, by inhalation, topically, rectally, nasally, buccally, vaginally, or via implant. The term parenteral or parenterally as used herein includes, but is not limited to, subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, or sublingual injection or infusion techniques.

In some embodiments an implant may be one or more device (such as a pump, implanted pump, reservoir, or port) or one or more material or matrix (such as a dissolvable material or matrix, biodegradable material or matrix, gel, or other material or matrix capable of mass transport such as by diffusion) located internally or externally comprising one or more substance (such as a solid, liquid, fluid, biostimulant, medicament, vaccine, or therapeutic agent) that enters into an organism, and is thereby implanted, attached or otherwise contacting tissues, skin, blood, fluid, or cells of a living organism, such as humans, plants, or animals as non-limiting examples.

In some embodiments one or more implant, may be implanted or administered via patch, transdermal patch, suppository, orally, swallowed, injected, sprayed, inhaled, aerosol, powder, mist, water additive, soil additive, fertilizer additive, liquid, tablet, or other known methods.

In some methods of use one or more implant may be implanted or administered via patch, transdermal patch, suppository, orally, swallowed, injected, sprayed, inhaled, aerosol, powder, mist, water additive, soil additive, fertilizer additive, liquid, tablet, or other known methods.

In some methods of use one or more RNA stabilizing composition as described herein may be used to produce an implant comprising at least one or more RNA substance and at least one or more RNA stabilizing substance.

In some methods of use one or more RNA stabilizing composition as described herein may be used to produce an implant comprising at least one or more RNA substance, at least one or more RNA stabilizing substance, and at least one or more cellular uptake agent.

In some methods of use one or more RNA stabilizing composition as described herein may be combined with one or more implant comprising at least one or more RNA substance and at least one or more RNA stabilizing substance.

In some methods of use one or more RNA stabilizing composition as described herein may be combined with one or more implant comprising at least one or more RNA substance, at least one or more RNA stabilizing substance, and at least one or more cellular uptake agent.

In some embodiments one or more RNA stabilizing composition as described herein may be combined with one or more implant comprising at least one or more RNA substance and at least one or more RNA stabilizing substance.

In some embodiments one or more RNA stabilizing composition as described herein may be combined with one or more implant comprising at least one or more RNA substance, at least one or more RNA stabilizing substance, and at least one or more cellular uptake agent.

In some embodiments one or more implant may comprise one or more pharmaceutical composition or may be used in conjunction with one or more pharmaceutical composition. In some embodiments one or more implant may comprise one or more medicament, vaccine, or therapeutic agent or may be used in conjunction with one or more medicament, vaccine, or therapeutic agent. In some embodiments one or more implant may comprise one or more biostimulant or biostimulant composition or may be used in conjunction with one or more bio stimulant or bio stimulant composition.

In some embodiments an embedded complex may be an implant.

In other methods of use one or more RNA stabilizing composition as described herein may be used for one or more of the following applications, including, but not limited to, treating a disease, preventing a disease, or producing a cellular response in one or more of the following organisms or cells, which may include but are not limited to: humans, primates, animals, vertebrate animals, eukaryotic cells, eukaryotes, protozoa, prokaryotic cells, plant cells, plants, fungal cells, fungi, insect cells, insects, bacterial cells, bacteria, mycoplasma, protozoa, plasmodium, or mammalian cells, including but not limited to the cells of primates, animals, vertebrate animals, and the cells of humans.

In some methods of use one or more RNA stabilizing composition as described herein may be used for in vivo, in vitro, in situ, or ex vivo applications. Such applications may include, but are not limited to, one or more of the following: agricultural applications, agricultural treatment, fertilizer, biostimulant, veterinary applications, animal treatment, pharmaceutical applications, human treatment, therapeutic applications, soil treatment, pesticide, herbicide, bacterial treatment, fungal treatment, antiviral, antibiotic, antifungal, antimicrobial, plant applications, plant treatment, antibody production, protein expression applications, insect treatment, insecticide, vaccine production, therapeutic agent production, implant production, embedded complex production, drug production, or medicament production, or combinations thereof.

In other methods of use one or more RNA stabilizing composition as described herein may be used for one or more of the following applications, including but not limited: syringe, prefilled syringe, injection, nasal spray, transdermal patch, eye drop, oral spray, aerosol, inhaler, nebulizer, oral tablet, pill, sublingual tablet, sublingual drop, suppository, mucosal spray, cream, lozenge, lotion, balm, syrup, ointment, fertilizer, biostimulant, implant, solution, powder, mist, spray, powder, or tablet.

In other methods of use one or more RNA stabilizing composition as described herein may be used in composition that also comprises a cellular uptake agent and used as a mucosal spray. Wherein, a mucosal spray may be any container that can be squeezed, pressurized, or applied in such a manner to aerosolize, spray, mist, aspirate, drop, squirt, apply, administer, or direct a combination comprising at least one RNA stabilizing substance and at least one or more RNA substance combined in a mixture comprising at least one cellular uptake agent into or onto a mucosal surface such as a nasal passage, airway, throat, lung, eye, or other mucosal surface within a human, primate, animal, or vertebrate animal.

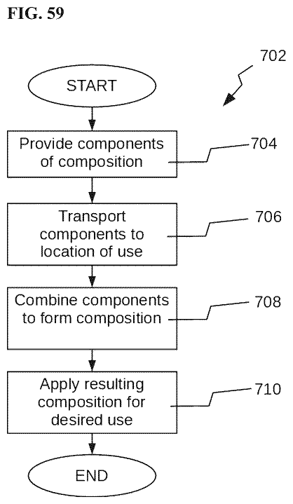

FIG. 59 is a flowchart that summarizes a process 702 for producing and using a stabilized RNA product in accordance with the present disclosure. The process 702 is initiated by providing (704) components of the RNA product or composition. The components may be obtained separately or may be provided as part of a kit or in a preloaded, multi-chamber syringe as described above, among other possibilities. Moreover, the composition, components, or combinations of components, may be provided in bottles, containers, vials, tubes, syringes, blisters, capsules, cartridges, or other packaging methods. The components may then be stored at the location of use or transported (706) to the location of use. Depending on the specific implementation, the components or some of the components may be refrigerated, frozen, or otherwise maintained in a temperature-controlled environment during transportation and storage.

When ready for use, the components can be combined (708) to yield the desired composition. For example, the individual components may be combined in a bottle, vial, or other container by adding the individual components to the container, e.g., by pouring, using a pipette, syringe, or the like, by adding lyophilized pellets, by measuring powders, or by any other suitable method. In certain implementations, the components may be mixed by breaking a breakable seal of a multi-compartment container such as a multi-compartment syringe. Finally, the resulting composition may be applied (710) for the desired use. As otherwise noted herein, the stabilized RNA products of the present disclosure may be utilized in a variety of fields such as therapeutics, diagnostics, or agriculture. In addition, the product may be packaged and distributed as a medicament, a therapeutic, vaccine, biostimulant, embedded complex, or implant.

Moreover, in the case of pharmaceutical compositions, the product may be packaged and distributed for administration orally, sublingually, transdermally, ophthalmically, parenterally, subcutaneously, intravenously, intramuscularly, by inhalation, topically, rectally, nasally, buccally, vaginally, or via an implant as otherwise described herein. The RNA product may be used for a variety of applications including treating a disease, preventing a disease, or producing a cellular response as otherwise described herein. The product may be used in in vivo, in vitro, in situ, or ex vivo applications. Such applications may include agricultural applications, agricultural treatment, fertilizer, biostimulant, veterinary applications, animal treatment, pharmaceutical applications, human treatment, therapeutic applications, soil treatment, pesticide, herbicide, bacterial treatment, fungal treatment, antiviral, antibiotic, antifungal, antimicrobial, plant applications, plant treatment, antibody production, protein expression applications, insect treatment, insecticide, vaccine production, therapeutic agent production, implant production, embedded complex production, drug production, medicament production, or combinations thereof. Accordingly, applying the resulting composition for the desired use will vary depending on the nature of the composition and the intended use among other things.

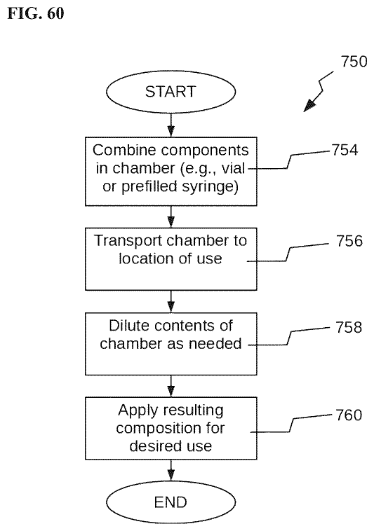

FIG. 60 is a flowchart that summarizes a process 750 for producing and using an RNA product in accordance with the present disclosure. The process 750 is initiated by providing (754) components of the RNA product or composition and combining them in a chamber or combining them and adding the combination to a chamber. The chamber may be any suitable chamber and may be, as non-limiting examples, a single use or multiuse vial. Moreover, the chamber may be, as non-limiting examples, bottles, containers, vials, tubes, syringes (including prefilled or single use syringes), blisters, capsules, cartridges, or other packaging. The chamber with components may then be stored at the location of use or transported (756) to the location of use. Depending on the specific implementation, the chambers, holding components, may be refrigerated, frozen, or otherwise maintained in a temperature-controlled environment during transportation and storage.

When ready for use, the components in the chamber may be combined with one or more diluents (758) to yield the desired concentration for final use. For example, the chambers produced at step (754) may, for example, contain concentrated mixture needing dilution or solids needing to be dissolved, for example in a bottle, vial, syringe, or other container. Alternatively, when ready for use, if the components introduced into the chamber are such that no dilution is needed then the contents are not diluted. Alternatively, part or all of the contents of the chamber may be withdrawn and added to another container, a non-limiting example being a bag containing an IV solution. At this stage other materials may be added.

Finally, the resulting composition may be applied (760) for the desired use. As otherwise noted herein, the stabilized RNA products of the present disclosure may be utilized in a variety of fields such as therapeutics, diagnostics, or agriculture. In addition, the product may be packaged and distributed as a medicament, a therapeutic, vaccine, biostimulant, embedded complex, or implant.

Moreover, in the case of pharmaceutical compositions, the product may be packaged and distributed for administration orally, sublingually, transdermally, ophthalmically, parenterally, subcutaneously, intravenously, intramuscularly, by inhalation, topically, rectally, nasally, buccally, vaginally, or via an implant as otherwise described herein. The RNA product may be used for a variety of applications including treating a disease, preventing a disease, or producing a cellular response as otherwise described herein. The product may be used in in vivo, in vitro, in situ, or ex vivo applications. Such applications may include agricultural applications, agricultural treatment, fertilizer, biostimulant, veterinary applications, animal treatment, pharmaceutical applications, human treatment, therapeutic applications, soil treatment, pesticide, herbicide, bacterial treatment, fungal treatment, antiviral, antibiotic, antifungal, antimicrobial, plant applications, plant treatment, antibody production, protein expression applications, insect treatment, insecticide, vaccine production, therapeutic agent production, implant production, embedded complex production, drug production, medicament production, or combinations thereof. Accordingly, applying the resulting composition for the desired use will vary depending on the nature of the composition and the intended use among other things.

As a non-limiting example, a chamber may be at least partially filled with components comprising one or more RNA substance and one or more RNA stabilizing substance followed by the chamber being prepared for shipping and storage (as a non-limiting example by undergoing steps comprising being packaged or placed in a shipping and storage container) followed by the chamber being transported to the location use, then removed from packaging and prepared for use by adding one or more diluents to the chamber and mixing, and with the appropriate amount of diluted mixture withdrawn from the chamber and administered to a patient as a therapeutic agent.

### EXAMPLES

The following non-limiting examples describe examples of the invention in more detail and in no way are to be construed as limiting the scope thereof.

The following examples of modified carbohydrates may include one more type of carbohydrate. The following examples also describe using one or more types of carbohydrates to create a modified carbohydrate. One of the modified carbohydrates discovered to stabilize RNA is modified starch. As known to those skilled in the art, starch is a polysaccharide made of amylose (a linear polymer of glucose), amylopectin (a branched polymer), or a combination of both amylose and amylopectin. Those skilled in the art will recognize that, as a non-limiting example, potato starch is a commonly used example starch. As non-limiting examples of modified carbohydrate starches, potato starch from Sigma-Aldrich was used as described herein to synthesize modified starches used in compositions with RNA to produce stabilized RNA compositions. Another non-limiting example starch known to those skilled in the art is potato starch treated according to Zulkowsky (treated with glycerol at 190° C.) (herein referred to as Zulkowsky starch), obtained from Sigma-Aldrich for modification to synthesize non-limiting examples of modified starches used in compositions with RNA to produce stabilized RNA compositions.

Among the materials used in the following examples include the following materials: Potato starch (Sigma-Aldrich Product #S9765); Inositol (myo-inositol) (Sigma-Aldrich Product #57570); Malic acid (Sigma-Aldrich Product #02288); Sucrose (Sigma-Aldrich Product #84097); D-Glucose (Sigma-Aldrich Product #G5767); D-Sorbitol (Sigma-Aldrich Product #S1876); Glycerol (Sigma-Aldrich Product #G5516); Na-Carboxymethylcellulose (CMC) (Sigma-Aldrich Product #419273); Citric acid (Sigma-Aldrich Product #251275); Trimethyl glycine hydrochloride (TMG-HCl) (also known as betaine-HCl) (Sigma-Aldrich Product #B3501); Trehalose (Cayman Chemical Product #20517); Fucoidan (Cayman Chemical Product #20357); Hyaluronic Acid (Pure Health Botanicals, LLC)

### Example 1

### Synthesis of Phosphate Modified Carbohydrates—Herein Referred to as Protocol 1

Synthesis of phosphate modified starch was performed according to the following reference: L. Passauer, F. Liebner, K. Fischer, Synthesis and Properties of Novel Hydrogels from Cross-linked Starch Phosphates, Macromolecular Symposia. 244 (2006) 180-193.

Briefly:

Phosphate modified starch was synthesized by mixing 0.5 g of potato starch (Sigma-Aldrich, St. Louis, MO; Product #S9765) with 3 mL of a sodium phosphate solution (pH 5.5-6) containing about 1 g of sodium phosphate (sodium phosphate monobasic (Sigma-Aldrich, Product #74092) and sodium phosphate dibasic solution (Sigma-Aldrich, Product #94046). The starch and phosphate mixture was then gently mixed for 30 min at room temperature to create a uniform slurry. The slurry was then dried at 120° F.-140° F. Following drying, the mixture was heated to about 300° F. for 3 hrs. Following heating, the dried cake was washed 3 times with 2 mL of ethanol and allowed to dry overnight. The following day, the cake was resuspended in molecular biology grade water at the desired concentration (typically 500-250 mg/mL) and the pH of the suspension was adjusted to 7 using NaOH. This solution was used in future RNA stability tests and referred to as Na—PO4 Starch 300F.

Protocol 1 was used to produce several variations of phosphate modified carbohydrates using sodium phosphate as listed in Table 1. Depending on the type of carbohydrate used, the time and temperature of heating was selected as shown in Table 1.

TABLE 1
Sodium Phosphate Modified Carbohydrates
Produced Using Protocol 1

| | Heating | Heating | |
|---|---|---|---|
| Carbohydrate | Temperature | Time | Reference Name |
| Inositol | 300 F. | 2 hrs | Na—PO4 Inositol 300 F. |
| Inositol | 280 F. | 2 hrs | Na—PO4 Inositol 280 F. |
| Sorbitol | 280 F. | 2 hrs | Na—PO4 Sorbitol 280 F. |
| Starch | 280 F. | 2 hrs | Na—PO4 Starch 280 F. |
| Starch | 250 F. | 2 hrs | Na—PO4 Starch 250 F. |
| Sucrose | 200 F. | 2 hrs | Na—PO4 Sucrose 200 F. |
| Glucose | 200 F. | 2 hrs | Na—PO4 Glucose 200 F. |
| Zulkowsky | 250 F. | 2 hrs | Na—PO4 Zulkowsky Starch |
| | | | 250 F. |
| Zulkowsky | 220 F. | 2 hrs | Na—PO4 Zulkowsky Starch |
| | | | 220 F. |

Protocol 1 was also used to produce several variations of phosphate modified carbohydrates using potassium phosphate as listed in Table 2. Depending on the type of carbohydrate used, the time and temperature of heating was selected as shown in Table 2.

TABLE 2
Potassium Phosphate Modified Carbohydrates
Produced Using Protocol 1

| | Heating | Heating | |
|---|---|---|---|
| Carbohydrate | Temperature | Time | Reference Name |
| Inositol | 300 F. | 2 hrs | K—PO4 Inositol 300 F. |
| Sorbitol | 300 F. | 2 hrs | K—PO4 Sorbitol 300 F. |
| Starch | 300 F. | 2 hrs | K—PO4 Starch 300 F. |
| Starch | 250 F. | 2 hrs | K—PO4 Starch 250 F. |
| Sucrose | 200 F. | 2 hrs | K—PO4 Sucrose 200 F. |
| Glucose | 200 F. | 2 hrs | K—PO4 Glucose 200 F. |
| Zulkowsky | 200 F. | 2 hrs | K—PO4 Zulkowsky Starch |
| | | | 200 F. |

### Example 2

Production of Cationic Quaternary Ammonium Modified Starch—Herein referred to as Protocol 2

### Synthesis of Cationic Quaternary Ammonium Modified Starch—Using Trimethylglycine-HCl

Synthesis of cationic quaternary ammonium modified carbohydrates was performed according to the following reference: “N. Karić, M. Vukčević, M. Ristić, A. Perić-Grujić, A. Marinković, K. Trivunac, A green approach to starch modification by solvent-free method with betaine hydrochloride, Int J Biol Macromol. 193 (2021) 1962-1971.”

Briefly:

Cationic quaternary ammonium modified starch was synthesized by mixing 0.5 g of soluble potato starch with 1 g trimethyl glycine hydrochloride (TMG-HCl) (also known as betaine-HCl) (Sigma-Aldrich, St. Louis, MO; Product #B3501). Then 1-2 mL of water, supplemented with 0.025 mL lactic acid solution (≥85%) (Sigma-Aldrich, St. Louis, MO; Product #252476), was added to the starch TMG-HCl mixture, to produce a semi wet paste. The resulting paste was then heated to 200° F. for 1 hr and the water was allowed to evaporate. Following heating, the dried paste was washed 3 times with 2 mL of ethanol and allowed to dry overnight. The following day, the resulting dried paste was resuspended in molecular biology grade water at the desired concentration (typically 500-250 mg/mL) and the pH of the suspension was adjusted to 7 using NaOH. This solution was used in future RNA stability tests and referred to as Cationic-Starch 200F.

Protocol 2 was used to produce several variations of cationic quaternary ammonium modified carbohydrates using TMG-HCl as listed in Table 3. Depending on the type of carbohydrate used, the time and temperature of heating was selected as shown in Table 3.

TABLE 3
Cationic Quaternary Ammonium Modified
Carbohydrates Produced Using Protocol 2

| | Heating | Heating | |
|---|---|---|---|
| Carbohydrate | Temperature | Time | Reference Name |
| Inositol | 300 F. | 1 hr | Cationic-Inositol 300 F. |
| Inositol | 280 F. | 1 hr | Cationic-Inositol 280 F. |
| Sorbitol | 300 F. | 1 hr | Cationic-Sorbitol 300 F. |
| Sorbitol | 250 F. | 1 hr | Cationic-Sorbitol 250 F. |
| Starch | 220 F. | 1 hr | Cationic-Starch 220 F. |
| Starch | 200 F. | 1 hr | Cationic-Starch 200 F. |
| Sucrose | 200 F. | 1 hr | Cationic-Sucrose 200 F. |
| Glucose | 200 F. | 1 hr | Cationic-Glucose 200 F. |
| Zulkowsky | 200 F. | 1 hr | Cationic-Zulkowsky Starch |
| | | | 200 F. |

### Example 3

Production of Dual Phosphate and Cationic Quaternary Ammonium Modified Carbohydrates—Herein referred to as Protocol 3.

Protocol 1 and was used to further modify a cationic quaternary ammonium modified starch with phosphate in addition to the previously added cationic quaternary ammonium modification.

Briefly:

Dual phosphate and cationic quaternary ammonium modified starch was synthesized by mixing 0.1 g of Cationic-Starch 200F with 0.5 mL of a sodium phosphate solution (pH 5.5-6) containing about 0.2 g of sodium phosphate. The starch and phosphate mixture was then gently mixed for 30 min at room temperature to create a uniform slurry. The slurry was then dried at 120° F.-140° F. Following drying, the mixture was heated to about 240° F. for 2 hrs. Following heating, the dried cake was washed 3 times with 2 mL of ethanol and allowed to dry overnight. The following day, the cake was resuspended in molecular biology grade water at the desired concentration (typically 500-250 mg/mL) and the pH of the suspension was adjusted to 7 using NaOH. This solution was used in future RNA stability tests and referred to as Dual PO4-Cationic Starch.

Protocol 3 was also used to modify cationic-Inositol by mixing 0.5 g of Cationic-Inositol 300F with 1 mL of a sodium phosphate solution (pH 5.5-6) containing about 0.25 g of sodium phosphate and heating to 300° F. for 2 hrs. This solution was used in future RNA stability tests and referred to as Dual PO4-Cationic Inositol.

### Example 4

Production of Dual Cationic Quaternary Ammonium and Phosphate Modified Starch—Herein referred to as Protocol 4.

Protocol 2 and was used to further modify a phosphate modified starch with a cationic quaternary ammonium in addition to the previously added phosphate modification.

Briefly:

Dual cationic quaternary ammonium modified starch and phosphate modified starch was synthesized by mixing 0.1 g of Na—PO4 Starch 300F with 0.2 g trimethyl glycine hydrochloride (TMG-HCl) (also known as betaine-HCl). Then 0.2-0.4 mL of water, supplemented with 0.05 mL lactic acid solution, was added to the starch TMG-HCl mixture, to produce a semi wet paste. The resulting paste was then heated to 220° F. for 1 hr and the water was allowed to evaporate. Following heating, the dried paste was washed 3 times with 2 mL of ethanol and allowed to dry overnight. The following day, the resulting dried paste was resuspended in molecular biology grade water at the desired concentration (typically 500-250 mg/mL) and the pH of the suspension was adjusted to 7 using NaOH. This solution was used in future RNA stability tests and referred to as Dual Cationic-PO4 Starch.

Protocol 4 was also used to modify phosphate-Inositol by mixing 0.5-g of PO4-Inositol 300F with 0.2 g trimethyl glycine hydrochloride (TMG-HCl) and then adding 0.2-0.4 mL of water, supplemented with 0.05 mL lactic acid solution and heating to 220° F. This solution was used in future RNA stability tests and referred to as Dual Cationic-PO4 Inositol.

### Example 5-A

Production of Additional Modified Polysaccharides

Additional phosphate modified polysaccharides were synthesized using Protocol 1. The type of polysaccharide used and time and temperature variations are listed in Table 4

TABLE 4
Phosphate Modified Carbohydrates Produced Using Protocol 1

| | Heating | Heating | |
|---|---|---|---|
| Carbohydrate | Temperature | Time | Reference Name |
| Carboxymethylcellulose | 250 F. | 1 hr | Na—PO4-CMC 250 F. |
| (CMC) | | | |
| Fucoidan | 250 F. | 1 hr | Na—PO4-Fucoidan |
| | | | 250 F. |
| Polyquaternium-10 | 250 F. | 1 hr | Na—PO4-Polyquat-10 |
| | | | 250 F. |
| Lambda Carrageenan | 250 F. | 1 hr | Na—PO4-Lambda |
| | | | Carrageenan 250 F. |
| Kappa Carrageenan | 250 F. | 1 hr | Na—PO4-Kappa |
| | | | Carrageenan 250 F. |
| Alginate | 250 F. | 1 hr | Na—PO4—Na- |
| | | | Alginate 250 F. |
| Gellan Gum | 250 F. | 1 hr | Na—PO4-Gellan |
| | | | Gum 250 F. |
| Xanthan Gum | 250 F. | 1 hr | Na—PO4-Xanthan |
| | | | Gum 250 F. |
| Hyaluronic Acid | 200 F. | 2 hrs | Na—PO4-Hyaluronic |
| | | | Acid 200 F. |

Additional cationic quaternary ammonium modified polysaccharides were synthesized using Protocol 2. The type of polysaccharide used and time and temperature variations are listed in Table 5

TABLE 5
Cationic Quaternary Ammonium Modified
Carbohydrates Produced Using Protocol 2

| | Heating | Heating | |
|---|---|---|---|
| Carbohydrate | Temperature | Time | Reference Name |
| CMC | 250 F. | 1 hr | Cationic-CMC 200 F. |
| Fucoidan | 250 F. | 1 hr | Cationic-Fucoidan 200 F. |
| Polyquaternium- | 250 F. | 1 hr | Cationic-Polyquat-10 200 F. |
| 10 | | | |
| Lambda | 250 F. | 1 hr | Cationic-Lambda Carrageenan |
| Carrageenan | | | 200 F. |
| Kappa | 250 F. | 1 hr | Cationic-Kappa Carrageenan |
| Carrageenan | | | 200 F. |
| Alginate | 250 F. | 1 hr | Cationic-Alginate 200 F. |
| Gellan Gum | 250 F. | 1 hr | Cationic-Gellan Gum 200 F. |
| Xanthan Gum | 250 F. | 1 hr | Cationic-Xanthan Gum 200 F. |
| Hyaluronic Acid | 200 F. | 2 hrs | Cationic-Hyaluronic Acid |
| | | | 200 F. |

### Example 5-B

Production of Crosslinked Modified Starch/Carbohydrates—Herein referred to as Protocol 5

Crosslinking of modified starch/carbohydrates was performed according to the following reference: L. Passauer, F. Liebner, K. Fischer, Synthesis and Properties of Novel Hydrogels from Cross-linked Starch Phosphates, Macromolecular Symposia. 244 (2006) 180-193.

Briefly:

Crosslinked modified starch was synthesized by mixing 0.125 g of Na-PO4-Starch 250F in 500 μL water supplemented with 1 mM citric acid (Sigma-Aldrich, Product #251275). The modified starch citric acid was gently mixed to create a uniform slurry. The slurry was then dried at 120° F.-140° F. Following drying, the mixture was heated to about 300° F. for 1 hr. Following heating, the dried cake was washed 3 times with 2 mL of ethanol and allowed to dry overnight. The following day, the cake was resuspended in molecular biology grade water at the desired concentration (typically 500-250 mg/mL) and the pH of the suspension was adjusted to 7 using NaOH. This solution was used in future RNA stability tests and referred to as PO4-Starch-X-Link-Citric Acid.

Protocol 5 was used to produce several variations of crosslinked modified carbohydrates using different concentrations of citric acid as listed in Table 6. Depending on the type of carbohydrate used, the time and temperature of heating was selected as shown in Table 6. In some cases both a phosphate modified carbohydrate and a cationic quaternary ammonium modified carbohydrate were crosslinked together.

TABLE 6
Crosslinked Modified Carbohydrates Produced Using Protocol 5

| | Cross- | Heat- | | |
|---|---|---|---|---|
| | linker | ing | Heat- | |
| | Concen- | Temper- | ing | |
| Carbohydrate | tration | ature | Time | Reference Name |
| Na—PO4 | 100 mM | 300° F. | 2 hr | PO4-Sorbitol-X- |
| Sorbitol 280 F. | Citric | | | Link-Citric Acid |
| | Acid | | | |
| Na—PO4 Starch | 1 mM Citric | 300° F. | 1 hr | PO4-Starch-X- |
| 250 F. | Acid | | | Link-Citric Acid |
| Na—PO4 | 10 mM | 250° F. | 1 hr | PO4-Zulkowsky |
| Zulkowsky Starch | Citric | | | Starch-X-Link- |
| 220 F. | Acid | | | Citric Acid |
| Cationic-Sorbitol | 100 mM | 300° F. | 2 hr | Cationic-Sorbitol- |
| 300 F. | Citric | | | X-Link-Citric |
| | Acid | | | Acid |
| Cationic-Starch | 1 mM Citric | 300° F. | 1 hr | Cationic-Starch- |
| 200 F. | Acid | | | X-Link-Citric |
| | | | | Acid |
| Cationic- | 10 mM | 250° F. | 1 hr | Cationic- |
| Zulkowsky Starch | Citric | | | Zulkowsky |
| 200 F. | Acid | | | Starch -X-Link- |
| | | | | Citric Acid |
| Na—PO4 | 100 mM | 300° F. | 2 hr | PO4-Cationic- |
| Sorbitol 280 F. + | Citric | | | Sorbitol-X-Link- |
| Cationic-Sorbitol | Acid | | | Citric Acid |
| 300 F. | | | | |
| Na—PO4 Starch | 1 mM Citric | 300° F. | 1 hr | PO4-Cationic- |
| 250 F. + Cationic- | Acid | | | Starch-X-Link- |
| Sorbitol 300 F. | | | | Citric Acid |
| Na—PO4 | 10 mM | 250° F. | 1 hr | PO4-Cationic- |
| Zulkowsky Starch | Citric | | | Zulkowsky |
| 220 F. + Cationic- | Acid | | | Starch-X-Link- |
| Starch 200 F. | | | | Citric Acid |

Protocol 5 was used to produce several variations of crosslinked modified carbohydrates using different concentrations of malic acid using the same conditions as listed in Table 7.

TABLE 7
Crosslinked Modified Carbohydrates Produced Using Protocol 5

| | Cross- | Heat- | | |
|---|---|---|---|---|
| | linker | ing | Heat- | |
| | Concen- | Temper- | ing | |
| Carbohydrate | tration | ature | Time | Reference Name |
| Na—PO4 | 100 mM | 300° F. | 2 hr | PO4-Sorbitol-X- |
| Sorbitol 280 F. | Malic | | | Link-Malic Acid |
| | Acid | | | |
| Na—PO4 | 1 mM Malic | 300° F. | 1 hr | PO4-Starch-X- |
| Starch 250 F. | Acid | | | Link-Malic Acid |
| Na—PO4 | 10 mM | 250° F. | 1 hr | PO4-Zulkowsky |
| Zulkowsky Starch | Malic | | | Starch-X-Link- |
| 220 F. | Acid | | | Malic Acid |
| Cationic-Sorbitol | 100 mM | 300° F. | 2 hr | Cationic-Sorbitol- |
| 300 F. | Malic | | | X-Link-Malic |
| | Acid | | | Acid |
| Cationic-Starch | 1 mM Malic | 300° F. | 1 hr | Cationic-Starch- |
| 200 F. | Acid | | | X-Link-Malic |
| | | | | Acid |
| Cationic- | 10 mM | 250° F. | 1 hr | Cationic- |
| Zulkowsky Starch | Malic | | | Zulkowsky |
| 200 F. | Acid | | | Starch -X-Link- |
| | | | | Malic Acid |
| Na—PO4 | 100 mM | 300° F. | 2 hr | PO4-Cationic- |
| Sorbitol 280 F. + | Malic | | | Sorbitol-X-Link- |
| Cationic-Sorbitol | Acid | | | Malic Acid |
| 300 F. | | | | |
| Na—PO4 Starch | 1 mM Malic | 300° F. | 1 hr | PO4-Cationic- |
| 250 F. + Cationic- | Acid | | | Starch-X-Link- |
| Sorbitol 300 F. | | | | Malic Acid |
| Na—PO4 | 10 mM | 250° F. | 1 hr | PO4-Cationic- |
| Zulkowsky Starch | Malic | | | Zulkowsky |
| 220 F. + Cationic- | Acid | | | Starch-X-Link- |
| Starch 200 F. | | | | Malic Acid |

### Example 6

In Vitro Transcription and Denaturing Agarose Gel Electrophoresis

The RNA was synthesized by in vitro transcription from a linear DNA construct with an upstream T7 RNA Polymerase promoter followed by the coding sequence for gene of interest. In vitro transcription was performed using the HiScribe T7 Quick High Yield RNA Synthesis Kit (New England Biolabs, Ipswich, MA; Product #E2050) according to the manufacturer's directions. Briefly, 2.5 μg template DNA was mixed with 25 μL NTP buffer mix and Silt T7 RNA polymerase mix. The entire reaction volume was brought to 50 μL with molecular biology grade H₂O and incubated in a thermal cycler at 37° C. for 2 hrs.

Following in vitro transcription, the RNA was purified using a Monarch RNA Cleanup Kit (New England Biolabs, Ipswich, MA; Product #T2050) according to the manufacturer's directions. Briefly, 1 spin column was used for each 50 μL reaction. Following binding of the RNA to the spin column, 2 washes of 500 μL were performed and the RNA was eluted with 100-150 μL of molecular biology grade H₂O. The purified RNA was then stored at −80° C.

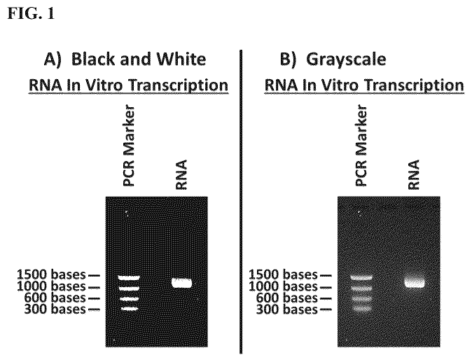

The in vitro transcribed and purified RNA was analyzed by denaturing agarose gel electrophoresis. Briefly, about 5 μg RNA was diluted 1:2 with 2× RNA loading dye (New England Biolabs, Ipswich, MA; Product #B0363) and heated to about 70° C. for about 2 minutes to denature the RNA. The final concentration of RNA and loading dye was about: 5 μg RNA, 47.5% formamide, 0.01% SDS, 0.01% bromophenol blue, 0.005% xylene cyanol and 0.5 mM EDTA. The RNA was run on a 1.5% agarose gel in 1× Tris Acetate EDTA (TAE) (Tris 40 mM, Acetic acid 20 mM, EDTA 1 mM, pH 8.0) supplemented with 0.06% sodium hypochlorite (NaClO) to prevent renaturing and degradation of the RNA during electrophoresis. The running buffer also contained 1× TAE supplemented with 0.06% sodium hypochlorite. RNA was visualized using SmartGlow fluorescent nucleic acid prestain (Accuris Instruments, Edison, NJ; Product #E4500-PS) according to the manufacturer's directions. A double stranded DNA PCR Marker (New England Biolabs, Ipswich, MA; Product #N3234) was used to estimate the apparent molecular weight of the RNA during electrophoresis. Denaturing agarose gel electrophoresis was carried out for about 1 hr at 80 v. The in vitro transcribed and purified RNA analyzed by denaturing agarose gel electrophoresis is shown in FIG. 1.

RNA concentration was measured by absorbance of the purified RNA at 260 nm using a Nanodrop ND-1000 (Thermo Fisher Scientific, Waltham, MA). Typical RNA concentration following in vitro transcription and purification was about 2-5 mg/mL.

### Example 7

Stability of RNA at Various Temperatures

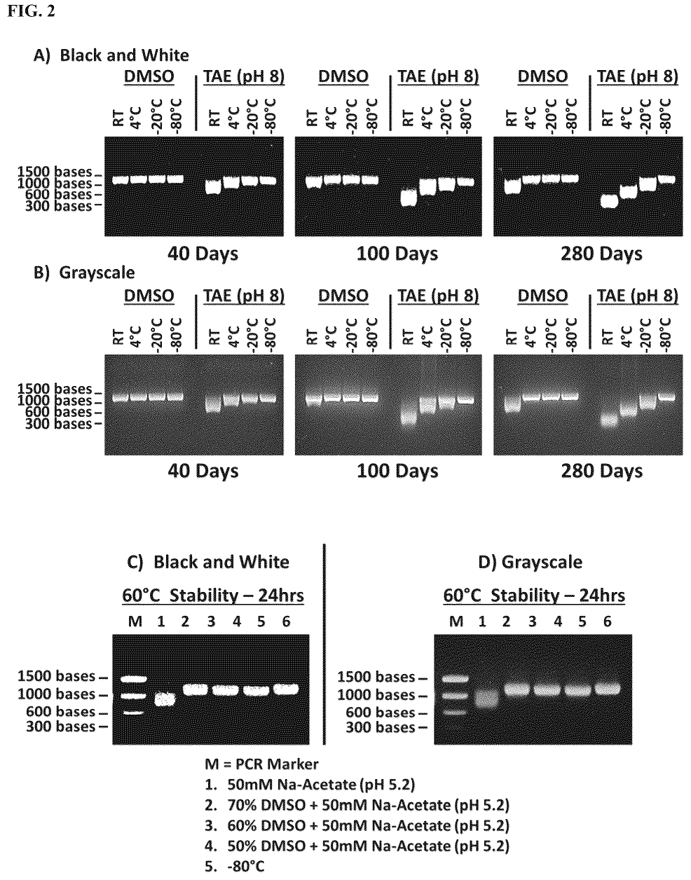

In vitro transcribed RNA was diluted at a ratio of about 1:10 (about 400-500n/mL) in either DMSO (Sigma-Aldrich, St. Louis, MO; Product #D8418) or 1× Tris Acetate EDTA (TAE) (pH 8) (Bioland Scientific LLC, Paramount, CA; Product #TAE01). The final concentration of DMSO was about 90% DMSO. The final concentration of TAE was about Tris 40 mM, Acetic acid 20 mM, EDTA 1 mM, pH 8.0. Following dilution of the RNA in either DMSO or TAE, the samples were then stored at 4 different temperatures: room temperature (RT) (about 20-25° C.), about 4° C., about −20° C., and about −80° C. Samples were then analyzed by denaturing agarose gel electrophoresis as described above at selected timepoints to measure RNA degradation and the stability of the RNA samples stored in either DMSO or TAE. During storage, 10 μL of each sample was analyzed at selected timepoints by agarose gel electrophoresis to measure RNA degradation and the stability of the RNA samples stored at each temperature in either DMSO or TAE. FIG. 2 A and B shows the agarose gel electrophoresis of each RNA sample following storage of the RNA for about 40 days to about 280 days at different temperatures.

The RNA sample stored in DMSO displays increased stability and reduced rate of degradation of the RNA sample as shown by agarose gel electrophoresis. The RNA sample stored in TAE begins to show notable degradation at room temperature after about 40 days as indicated by the smearing of the RNA band, decreased fluorescence intensity, and lower apparent molecular weight compared to the −80° C. RNA sample. While the RNA sample stored in DMSO does not show notable signs of degradation until about 100 days at room temperature. Furthermore, the RNA sample stored in TAE begins to show notable signs of degradation following about 40 days at 4° C. While the RNA sample stored in DMSO does not show notable signs of degradation up to about 280 days at 4° C. In addition, the RNA sample stored in TAE begins to show notable signs of degradation following about 100 days at −20° C. While the RNA sample stored in DMSO does not show notable signs of degradation up to about 280 days at −20° C. Following 100 days, it becomes apparent that the RNA sample stored in DMSO shows comparable and/or better stability at room temperature when compared to the RNA sample stored in TAE at 4° C. or −20° C. Furthermore, RNA stored in DMSO at 4° C. still displays a measurable band of comparable size and fluorescence intensity compared to the −80° C. RNA sample following 280 days, while the RNA stored in TAE shows little to no band of comparable size and fluorescence intensity compared to the −80° C. RNA sample following 280 days at 4° C. or −20° C.

The following examples compare RNA degradation in different storage environments containing different substances or combinations of substances stored at elevated temperatures and sampled at selected timepoints.

The evaluation of RNA stability in the following examples was carried out as follows:

Accelerated Stability Testing

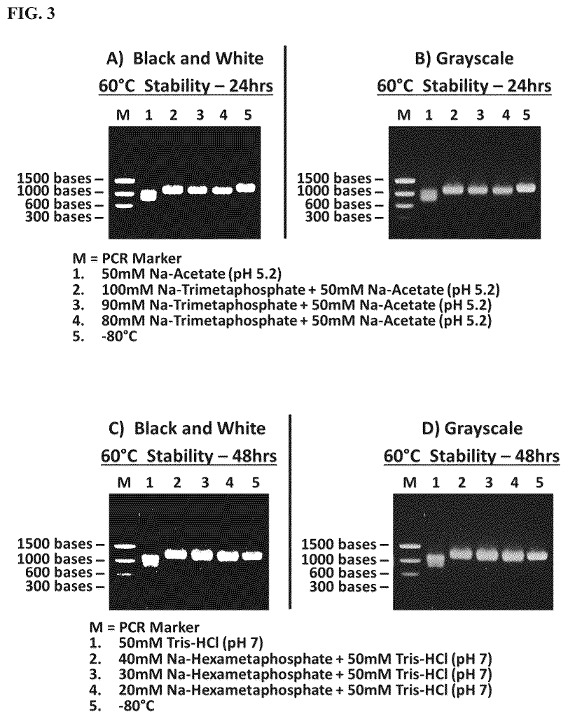

Accelerated stability testing is known in the art, where a sample is incubated at elevated temperature to increase the rate of degradation. FIG. 3-FIG. 56 show the results of agarose gel electrophoresis comparing accelerated RNA stability in different RNA storage environments comprising different substances, either alone or in combination.

Accelerated stability testing was performed as follows:

In vitro transcribed RNA was diluted at a ratio of about 1:10 (about 100-300 ug/mL) in different compositions. A control sample was diluted in a composition containing either a buffer only control sample or a control without one or more substance tested. The RNA stability of these control samples were then compared to samples diluted in compositions containing different substances or combinations of multiple substances.

Following dilution of each sample in each respective RNA storage environment, samples were stored in a thermal cycler at elevated temperatures (e.g about 60° C.-70° C.) for about 24-96 hours. During storage at elevated temperature samples were analyzed at selected timepoints by denaturing agarose gel electrophoresis to measure RNA degradation and the stability of the RNA samples in each RNA storage environment. FIG. 3-FIG. 56 shows the results of denaturing agarose gel electrophoresis following storage of RNA for about 24-96 hours at elevated temperatures (e.g. 60° C.-70° C.). A DNA PCR marker was used to estimate the apparent molecular weight. A control sample (typically a buffer only control) was compared to the subsequent samples with at least one or more additional substance not present in the control sample. An RNA sample stored at −80° C. was run in the final lane to compare the RNA stored at elevated temperatures to a full-length RNA sample stored at −80° C.

Following analysis by agarose gel electrophoresis, RNA degradation was measured by a combination of decreasing apparent molecular weight, broadening of an RNA band, and decreasing fluorescence intensity as compared to the −80° C. full length sample.

Samples comprising substances with high background fluorescence or that may have distorted analysis (e.g. salicylic acid, N-acetyl tyrosine, biotin and others) were buffer exchanged using spin columns, precipitation or dialysis or other suitable means. Samples that had high salt concentrations, cationic groups, or cationic polymers (e.g. PTMAEMA, PDADMAC, PDEAEMA) were incubated with 1-2.5 mM-8 kDa polyacrylic acid-sodium salt (PAA) at room temp or 70° C. to help facilitate analysis by agarose electrophoresis.

Common abbreviations in following examples: hexametaphosphate (HMP) and trimetaphosphate (TMP)

### Example 8

Accelerated RNA Stability Testing at 60° C.

In vitro transcribed RNA was diluted at a ratio of about 1:10 (about 100-300 ug/mL) in different compositions containing either 50 mM sodium acetate (pH 5.2) (Sigma-Aldrich, St. Louis, MO; Product #S7899), or a mixture of DMSO and 50 mM sodium acetate (pH 5.2) as follows:
1. 50 mM Na-Acetate (pH 5.2)
2. 70% DMSO+50 mM Na-Acetate (pH 5.2)
3. 60% DMSO+50 mM Na-Acetate (pH 5.2)
4. 50% DMSO+50 mM Na-Acetate (pH 5.2)
5. −80° C.

Following dilution of each sample in each respective RNA storage environment, samples were stored in a thermal cycler at 60° C. for about 24 hours and then analyzed by denaturing agarose gel electrophoresis as shown in FIG. 2 C and D.

### Example 9

Accelerated RNA Stability Testing at 60° C.

In vitro transcribed RNA was diluted at a ratio of about 1:10 (about 100-300 ug/mL) in different compositions containing either 50 mM sodium acetate (pH 5.2), or a mixture of sodium trimetaphosphate (TMP) (Sigma-Aldrich, St. Louis, MO; Product #PHR2204) and 50 mM sodium acetate (pH 5.2) as follows:
1. 50 mM Na-Acetate (pH 5.2)
2. 100 mM Na-Trimetaphosphate+50 mM Na-Acetate (pH 5.2)
3. 90 mM Na-Trimetaphosphate+50 mM Na-Acetate (pH 5.2)
4. 80 mM Na-Trimetaphosphate+50 mM Na-Acetate (pH 5.2)
5. −80° C.

Following dilution of each sample in each respective RNA storage environment, samples were stored in a thermal cycler at 60° C. for about 24 hours and then analyzed by denaturing agarose gel electrophoresis as shown in FIG. 3A and B.

### Example 10

Accelerated RNA Stability Testing at 60° C.

In vitro transcribed RNA was diluted at a ratio of about 1:10 (about 100-300n/mL) in different compositions containing either 50 mM Tris-HCl (pH 7) (Bioland Scientific LLC, Paramount, CA; Product #Tris70), or a mixture of sodium hexametaphosphate (HMP) and 50 mM Tris-HCl (pH 7) as follows:
1. 50 mM Tris-HCl (pH 7)
2. 40 mM Na-Hexametaphosphate+50 mM Tris-HCl (pH 7)
3. 30 mM Na-Hexametaphosphate+50 mM Tris-HCl (pH 7)
4. 20 mM Na-Hexametaphosphate+50 mM Tris-HCl (pH 7)
5. −80° C.

Following dilution of each sample in each respective RNA storage environment, samples were stored in a thermal cycler at 60° C. for about 48 hours and then analyzed by denaturing agarose gel electrophoresis as shown in FIG. 3 C and D.

### Example 11

Accelerated RNA Stability Testing at 60° C.

In vitro transcribed RNA was diluted at a ratio of about 1:10 (about 100-300n/mL) in different compositions containing either 50 mM sodium acetate (pH 5.2), or a mixture of hexylene glycol (Sigma-Aldrich, St. Louis, MO; Product #M9671) and 50 mM sodium acetate (pH 5.2) as follows:
1. 50 mM Na-Acetate (pH 5.2)
2. 30% Hexylene Glycol+50 mM Na-Acetate (pH 5.2)
3. 27.5% Hexylene Glycol+50 mM Na-Acetate (pH 5.2)
4. 25% Hexylene Glycol+50 mM Na-Acetate (pH 5.2)
5. 22.5% Hexylene Glycol+50 mM Na-Acetate (pH 5.2)
6. −80° C.

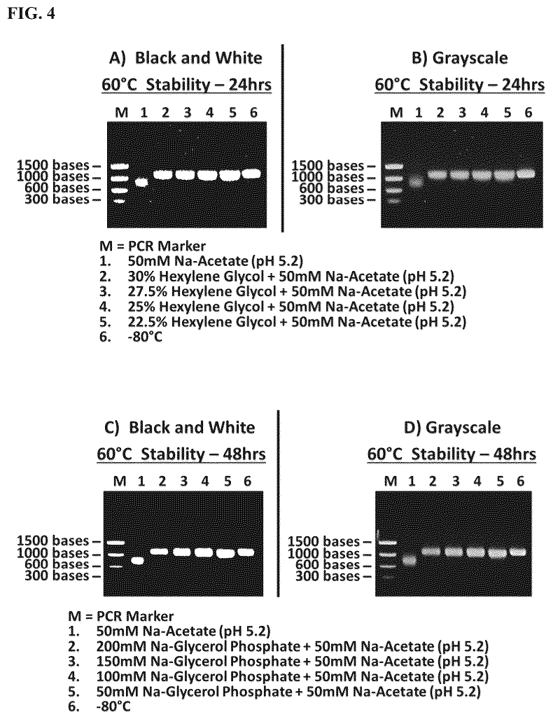

Following dilution of each sample in each respective RNA storage environment, samples were stored in a thermal cycler at 60° C. for about 24 hours and then analyzed by denaturing agarose gel electrophoresis as shown in FIG. 4A and B.

### Example 12

Accelerated RNA Stability Testing at 60° C.

In vitro transcribed RNA was diluted at a ratio of about 1:10 (about 100-300n/mL) in different compositions containing either 50 mM sodium acetate (pH 5.2), or a mixture of glycerol phosphate disodium salt (Sigma-Aldrich, St. Louis, MO; Product #G6501) and 50 mM sodium acetate (pH 5.2) as follows:
1. 50 mM Na-Acetate (pH 5.2)
2. 200 mM Na-Glycerol Phosphate+50 mM Na-Acetate (pH 5.2)
3. 150 mM Na-Glycerol Phosphate+50 mM Na-Acetate (pH 5.2)
4. 100 mM Na-Glycerol Phosphate+50 mM Na-Acetate (pH 5.2)
5. 50 mM Na-Glycerol Phosphate+50 mM Na-Acetate (pH 5.2)
6. −80° C.

Following dilution of each sample in each respective RNA storage environment, samples were stored in a thermal cycler at 60° C. for about 48 hours and then analyzed by denaturing agarose gel electrophoresis as shown in FIG. 4 C and D.

### Example 13

Accelerated RNA Stability Testing at 60° C.

In vitro transcribed RNA was diluted at a ratio of about 1:10 (about 100-300n/mL) in different compositions containing either 50 mM sodium acetate (pH 5.2), or a mixture of 1-butyl-1-methylpyrrolidinium bromide (Sigma-Aldrich, St. Louis, MO; Product #04275), benzyltriethylammonium chloride (Sigma-Aldrich, St. Louis, MO; Product #146552), or N,N-dimethylphenethylamine (Sigma-Aldrich, St. Louis, MO; Product #523801) and 50 mM sodium acetate (pH 5.2) as follows:
1. 50 mM Na-Acetate (pH 5.2)
2. 750 mM 1-Buty-1-Methylpyrrolidinium-Br+50 mM Na-Acetate (pH 5.2)
3. 500 mM Benzyltriethylammonium-Cl+50 mM Na-Acetate (pH 5.2)
4. 100 mM N,N-Dimethylphenethylamine+50 mM Na-Acetate (pH 5.2)
5. −80° C.

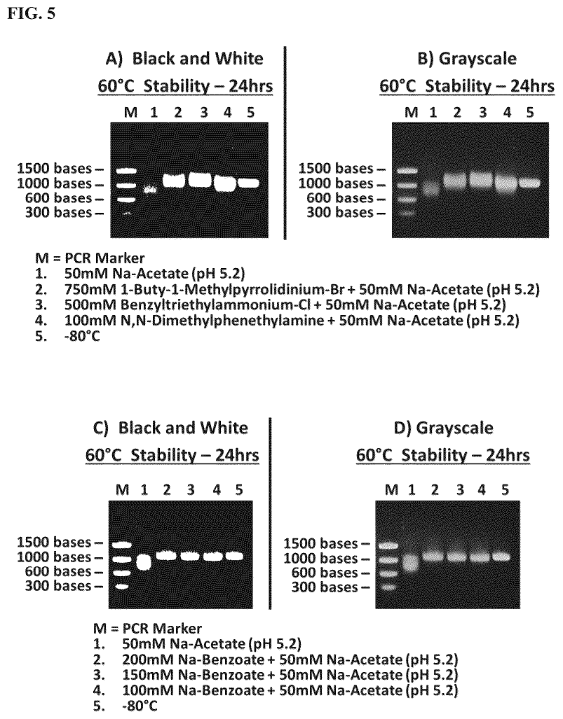

The N,N-dimethylphenethylamine sample dilution was made fresh from the stock solution immediately prior to mixing with RNA. Following dilution of each sample in each respective RNA storage environment, samples were stored in a thermal cycler at 60° C. for about 24 hours and then analyzed by denaturing agarose gel electrophoresis as shown in FIG. 5A and B. Prior to performing gel electrophoresis, the 1-butyl-1-methylpyrrolidinium and benzyltriethylammonium samples were incubated with 2.5 mM-8 kDa PAA sodium salt for about 1 hour at room temperature (about 20-25° C.) to improve analysis by gel electrophoresis.

### Example 14

Accelerated RNA Stability Testing at 60° C.

In vitro transcribed RNA was diluted at a ratio of about 1:10 (about 100-300n/mL) in different compositions containing either 50 mM sodium acetate (pH 5.2), or a mixture of sodium benzoate (Sigma-Aldrich, St. Louis, MO; Product #109169) and 50 mM sodium acetate (pH 5.2) as follows:
1. 50 mM Na-Acetate (pH 5.2)
2. 200 mM Na-Benzoate+50 mM Na-Acetate (pH 5.2)
3. 150 mM Na-Benzoate+50 mM Na-Acetate (pH 5.2)
4. 100 mM Na-Benzoate+50 mM Na-Acetate (pH 5.2)
5. −80° C.

Following dilution of each sample in each respective RNA storage environment, samples were stored in a thermal cycler at 60° C. for about 24 hours and then analyzed by denaturing agarose gel electrophoresis as shown in FIG. 5 C and D.

### Example 15

Accelerated RNA Stability Testing at 60° C.

In vitro transcribed RNA was diluted at a ratio of about 1:10 (about 100-300n/mL) in different compositions containing either 50 mM Tris-HCl (pH 7), or a mixture of sodium benzoate with 1M trimethylglycine (TMG) (Sigma-Aldrich, St. Louis, MO; Product #B0300) and 50 mM Tris-HCl (pH 7) as follows:
1. 50 mM Tris-HCl (pH 7)
2. 300 mM Na-Benzoate+1M TMG+50 mM Tris-HCl (pH 7)
3. 250 mM Na-Benzoate+1M TMG+50 mM Tris-HCl (pH 7)
4. 200 mM Na-Benzoate+1M TMG+50 mM Tris-HCl (pH 7)
5. −80° C.

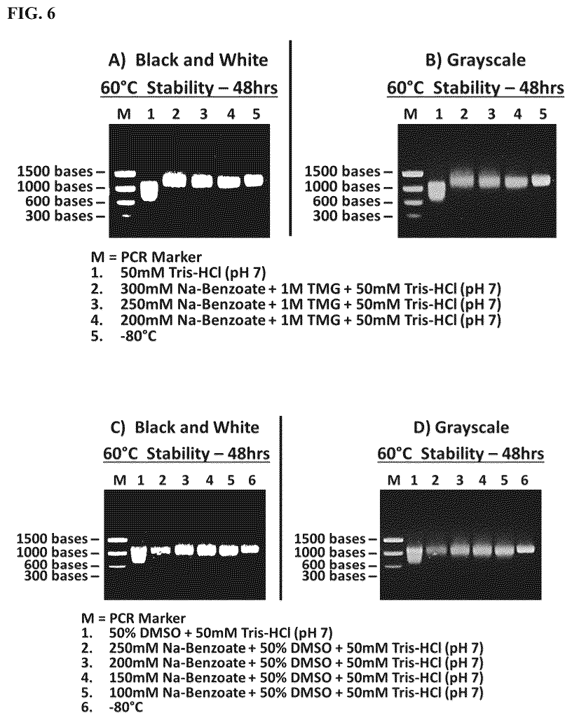

Following dilution of each sample in each respective RNA storage environment, samples were stored in a thermal cycler at 60° C. for about 48 hours and then analyzed by denaturing agarose gel electrophoresis as shown in FIG. 6A and B.

### Example 16

Accelerated RNA Stability Testing at 60° C.

In vitro transcribed RNA was diluted at a ratio of about 1:10 (about 100-300 μg/mL) in different compositions containing either 50% DMSO with 50 mM Tris-HCl (pH 7), or a mixture of sodium benzoate with 50% DMSO and 50 mM Tris-HCl (pH 7) as follows:
1. 50% DMSO+50 mM Tris-HCl (pH 7)
2. 250 mM Na-Benzoate+50% DMSO+50 mM Tris-HCl (pH 7)
3. 200 mM Na-Benzoate+50% DMSO+50 mM Tris-HCl (pH 7)
4. 150 mM Na-Benzoate+50% DMSO+50 mM Tris-HCl (pH 7)
5. 100 mM Na-Benzoate+50% DMSO+50 mM Tris-HCl (pH 7)
6. −80° C.

Following dilution of each sample in each respective RNA storage environment, samples were stored in a thermal cycler at 60° C. for about 48 hours and then analyzed by denaturing agarose gel electrophoresis as shown in FIG. 6 C and D.

### Example 17

Accelerated RNA Stability Testing at 60° C.

In vitro transcribed RNA was diluted at a ratio of about 1:10 (about 100-300 μg/mL) in different compositions containing either 50 mM sodium acetate (pH 5.2), or varying concentrations of trimethyloctylammonium bromide (Sigma-Aldrich, St. Louis, MO; Product #75091) and 50 mM sodium acetate (pH 5.2) as follows:
1. 50 mM Na-Acetate (pH 5.2)
2. 70 mM Trimethyloctylammonium-Br+50 mM Na-Acetate (pH 5.2)
3. 60 mM Trimethyloctylammonium-Br+50 mM Na-Acetate (pH 5.2)
4. 50 mM Trimethyloctylammonium-Br+50 mM Na-Acetate (pH 5.2)
5. 40 mM Trimethyloctylammonium-Br+50 mM Na-Acetate (pH 5.2)
6. −80° C.

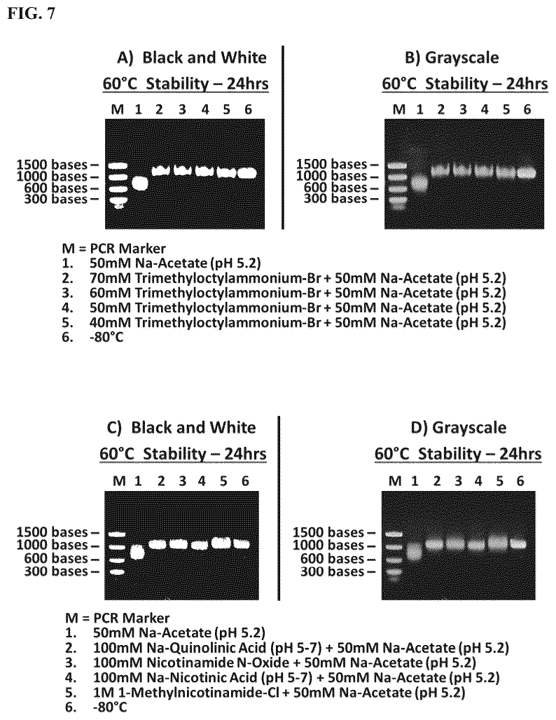

Following dilution of each sample in each respective RNA storage environment, samples were stored in a thermal cycler at 60° C. for about 24 hours and then analyzed by denaturing agarose gel electrophoresis as shown in FIG. 7A and B. Prior to performing gel electrophoresis, the trimethyloctylammonium samples were diluted 1.5× with molecular biology H₂O (10 μL H₂O added to a 20 μL sample) and then incubated with 2.5 mM-8 kDa PAA sodium salt for about 1 hour at room temperature (about 20-25° C.) to improve analysis by gel electrophoresis.

### Example 18

Accelerated RNA Stability Testing at 60° C.

In vitro transcribed RNA was diluted at a ratio of about 1:10 (about 100-300n/mL) in different compositions containing either 50 mM sodium acetate (pH 5.2), or a mixture of quinolinic acid (Cayman Chemical, Ann Arbor, MI; Product #14941), nicotinamide N-oxide (Cayman Chemical, Ann Arbor, MI; Product #28441), nicotinic acid (Sigma-Aldrich, St. Louis, MO; Product #N4126), or 1-methylnicotinamide chloride (Cayman Chemical, Ann Arbor, MI; Product #16604) and 50 mM sodium acetate (pH 5.2) as follows:
1. 50 mM Na-Acetate (pH 5.2)
2. 100 mM Na-Quinolinic Acid (pH 5-7)+50 mM Na-Acetate (pH 5.2)
3. 100 mM Nicotinamide N-Oxide+50 mM Na-Acetate (pH 5.2)
4. 100 mM Na-Nicotinic Acid (pH 5-7)+50 mM Na-Acetate (pH 5.2)
5. 1M 1-Methylnicotinamide-Cl+50 mM Na-Acetate (pH 5.2)
6. −80° C.

The quinolinic acid and nicotinic acid stocks were adjusted to a pH of about 5-7 using NaOH. The nicotinamide N-oxide stock pH was adjusted using NaOH to facilitate dissolving in H₂O and then subsequently adjusted to a pH of about 5-7 using HCl once dissolved. Following dilution of each sample in each respective RNA storage environment, samples were stored in a thermal cycler at 60° C. for about 24 hours and then analyzed by denaturing agarose gel electrophoresis as shown in FIG. 7 C and D. Prior to performing gel electrophoresis, the nicotinamide N-oxide and 1-methylnicotinamide samples were incubated with 1 mM-8 kDa PAA sodium salt about 1 hour at room temperature (about 20-25° C.) to improve analysis by gel electrophoresis.

### Example 19

Accelerated RNA Stability Testing at 60° C.

In vitro transcribed RNA was diluted at a ratio of about 1:10 (about 100-300n/mL) in different compositions containing either 5 mM sodium phosphate (pH 8) (Sigma-Aldrich, St. Louis, MO; Product #94046 & 74092) and 10 mM sodium acetate (pH 7) (Sigma-Aldrich, St. Louis, MO; Product #S2404), or a mixture of ectoine (Sigma-Aldrich, St. Louis, MO; Product #81619), L-proline (Sigma-Aldrich, St. Louis, MO; Product #81709), glycine (Sigma-Aldrich, St. Louis, MO; Product #50046), or taurine (Sigma-Aldrich, St. Louis, MO; Product #T0625), and 5 mM sodium phosphate (pH 8) and 10 mM sodium acetate (pH 7) as follows:
1. 5 mM Na-Phosphate (pH 8)+10 mM Na-Acetate (pH 7)
2. 2M Ectoine+5 mM Na-Phosphate (pH 8)+10 mM Na-Acetate (pH 7)
3. 500 mM Proline+5 mM Na-Phosphate (pH 8)+10 mM Na-Acetate (pH 7)
4. 500 mM Glycine+5 mM Na-Phosphate (pH 8)+10 mM Na-Acetate (pH 7)
5. 250 mM Taurine+5 mM Na-Phosphate (pH 8)+10 mM Na-Acetate (pH 7)
6. −80° C.

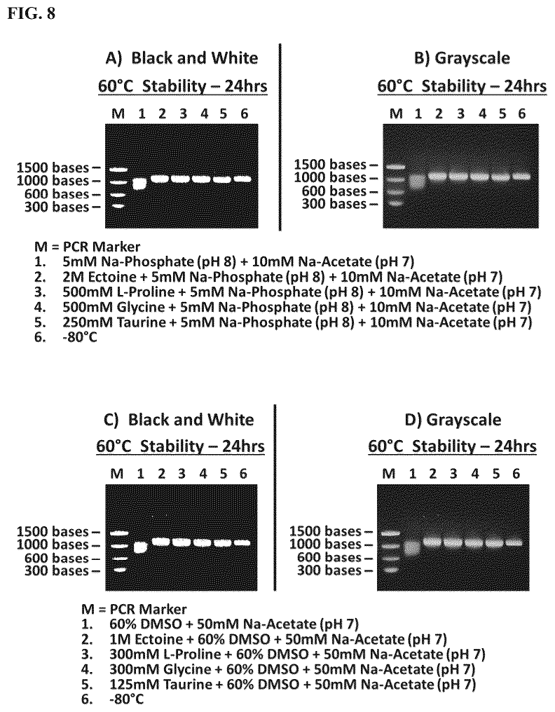

Following dilution of each sample in each respective RNA storage environment, samples were stored in a thermal cycler at 60° C. for about 24 hours and then analyzed by denaturing agarose gel electrophoresis as shown in FIG. 8A and B.

### Example 20

Accelerated RNA Stability Testing at 60° C.

In vitro transcribed RNA was diluted at a ratio of about 1:10 (about 100-300n/mL) in different compositions containing either 60% DMSO with 50 mM sodium acetate (pH 7), or a mixture of ectoine, L-proline, glycine, or taurine, and 60% DMSO with 50 mM sodium acetate (pH 7) as follows:
1. 60% DMSO+50 mM Na-Acetate (pH 7)
2. 1M Ectoine+60% DMSO+50 mM Na-Acetate (pH 7)
3. 300 mM Proline+60% DMSO+50 mM Na-Acetate (pH 7)
4. 300 mM Glycine+60% DMSO+50 mM Na-Acetate (pH 7)
5. 125 mM Taurine+60% DMSO+50 mM Na-Acetate (pH 7)
6. −80° C.

Following dilution of each sample in each respective RNA storage environment, samples were stored in a thermal cycler at 60° C. for about 24 hours and then analyzed by denaturing agarose gel electrophoresis as shown in FIG. 8 C and D.

### Example 21

Accelerated RNA Stability Testing at 60° C.

In vitro transcribed RNA was diluted at a ratio of about 1:10 (about 100-300 μg/mL) in different compositions containing either TAE (Tris 40 mM, Acetic acid 20 mM, EDTA 1 mM) (pH 8), or a mixture of dimethylsulfoniopropionate (DMSP) (Sigma-Aldrich, St. Louis, MO; Product #80828) with TAE (pH 8) as follows:
1. TAE (pH 8)
2. 700 mM DMSP+TAE (pH 8)
3. 600 mM DMSP+TAE (pH 8)
4. 500 mM DMSP+TAE (pH 8)
5. 400 mM DMSP+TAE (pH 8)
6. −80° C.

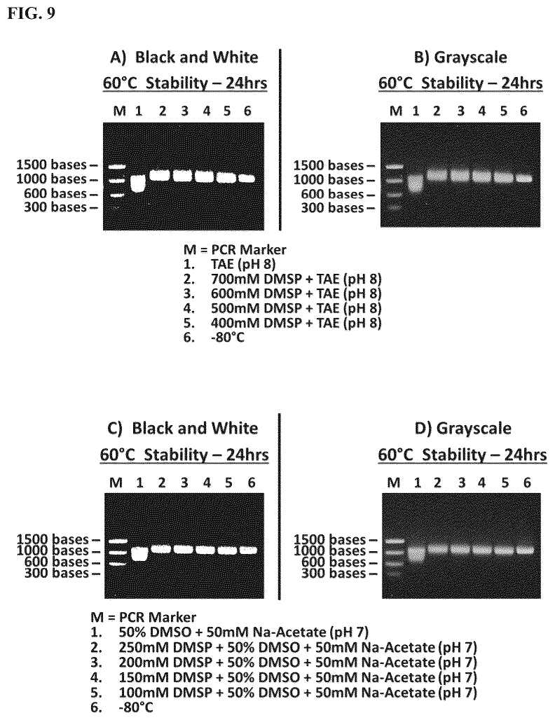

The DMSP stock was adjusted to a pH of about 7 using NaOH. Following dilution of each sample in each respective RNA storage environment, samples were stored in a thermal cycler at 60° C. for about 24 hours and then analyzed by denaturing agarose gel electrophoresis as shown in FIG. 9A and B. Prior to performing gel electrophoresis, the DMSP samples were incubated with 1 mM-8 kDa PAA sodium salt about 1 hour at room temperature (about 20-25° C.) to improve analysis by gel electrophoresis.

### Example 22

Accelerated RNA Stability Testing at 60° C.

In vitro transcribed RNA was diluted at a ratio of about 1:10 (about 100-300 μg/mL) in different compositions containing either 50% DMSO with 50 mM sodium acetate (pH 7), or a mixture of DMSP and 50% DMSO with 50 mM sodium acetate (pH 7) as follows:
1. 50% DMSO+50 mM Na-Acetate (pH 7)
2. 250 mM DMSP+50% DMSO+50 mM Na-Acetate (pH 7)
3. 200 mM DMSP+50% DMSO+50 mM Na-Acetate (pH 7)
4. 150 mM DMSP+50% DMSO+50 mM Na-Acetate (pH 7)
5. 100 mM DMSP+50% DMSO+50 mM Na-Acetate (pH 7)
6. −80° C.

The DMSP stock was adjusted to a pH of about 7 using NaOH. Following dilution of each sample in each respective RNA storage environment, samples were stored in a thermal cycler at 60° C. for about 24 hours and then analyzed by denaturing agarose gel electrophoresis as shown in FIG. 9 C and D. Prior to performing gel electrophoresis, the DMSP samples were incubated with 1 mM-8 kDa PAA sodium salt for about 1 hour at room temperature (about 20-25° C.) to improve analysis by gel electrophoresis.

### Example 23

Accelerated RNA Stability Testing at 70° C.

In vitro transcribed RNA was diluted at a ratio of about 1:10 (about 100-300 μg/mL) in different compositions containing either 50 mM Tris-HCl (pH 7), or a mixture of choline chloride and 50 mM Tris-HCl (pH 7) as follows:
1. 50 mM Tris-HCl (pH 7)
2. 1M Choline-Cl+50 mM Tris-HCl (pH 7)
3. 900 mM Choline-Cl+50 mM Tris-HCl (pH 7)
4. 800 mM Choline-Cl+50 mM Tris-HCl (pH 7)
5. 700 mM Choline-Cl+50 mM Tris-HCl (pH 7)
6. −80° C.

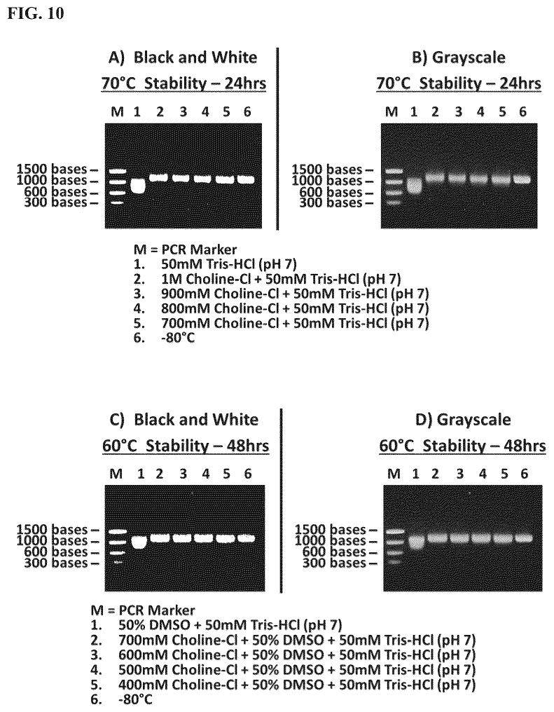

Following dilution of each sample in each respective RNA storage environment, samples were stored in a thermal cycler at 70° C. for about 24 hours and then analyzed by denaturing agarose gel electrophoresis as shown in FIG. 10A and B.

### Example 24

Accelerated RNA Stability Testing at 60° C.

In vitro transcribed RNA was diluted at a ratio of about 1:10 (about 100-300 μg/mL) in different compositions containing either 50% DMSO with 50 mM Tris-HCl (pH 7), or a mixture of choline chloride and 50% DMSO with 50 mM Tris-HCl (pH 7) as follows:
1. 50% DMSO+50 mM Tris-HCl (pH 7)
2. 700 mM Choline-Cl+50% DMSO+50 mM Tris-HCl (pH 7)
3. 600 mM Choline-Cl+50% DMSO+50 mM Tris-HCl (pH 7)
4. 500 mM Choline-Cl+50% DMSO+50 mM Tris-HCl (pH 7)
5. 400 mM Choline-Cl+50% DMSO+50 mM Tris-HCl (pH 7)
6. −80° C.

Following dilution of each sample in each respective RNA storage environment, samples were stored in a thermal cycler at 60° C. for about 48 hours and then analyzed by denaturing agarose gel electrophoresis as shown in FIG. 10 C and D.

### Example 25

Accelerated RNA Stability Testing at 60° C.

In vitro transcribed RNA was diluted at a ratio of about 1:10 (about 100-300 μg/mL) in different compositions containing either 50 mM sodium acetate (pH 5.2), or a mixture of acetylcholine chloride (Sigma-Aldrich, St. Louis, MO; Product #A2661) and 50 mM sodium acetate (pH 5.2) as follows:
1. 50 mM Na-Acetate (pH 5.2)
2. 1M Acetylcholine-Cl+50 mM Na-Acetate (pH 5.2)
3. 900 mM Acetylcholine-Cl+50 mM Na-Acetate (pH 5.2)
4. 800 mM Acetylcholine-Cl+50 mM Na-Acetate (pH 5.2)
5. 700 mM Acetylcholine-Cl+50 mM Na-Acetate (pH 5.2)
6. −80° C.

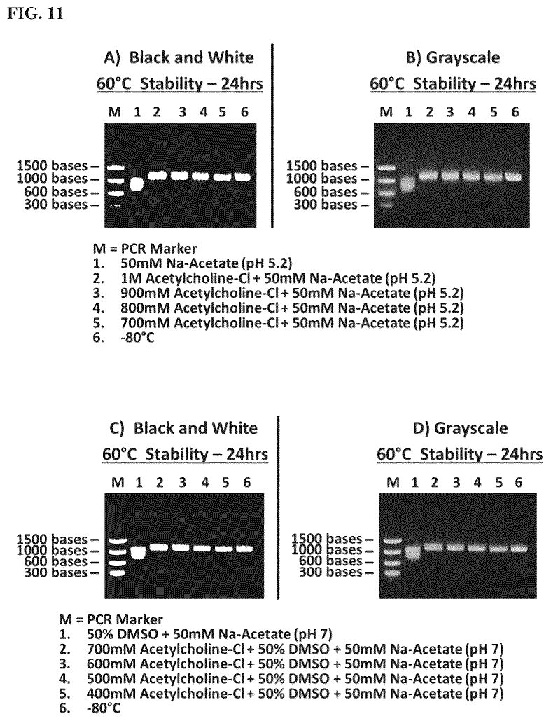

Following dilution of each sample in each respective RNA storage environment, samples were stored in a thermal cycler at 60° C. for about 24 hours and then analyzed by denaturing agarose gel electrophoresis as shown in FIG. 11A and B.

### Example 26

Accelerated RNA Stability Testing at 60° C.

In vitro transcribed RNA was diluted at a ratio of about 1:10 (about 100-300 μg/mL) in different compositions containing either 50% DMSO with 50 mM sodium acetate (pH 7), or a mixture of acetylcholine chloride and 50% DMSO with 50 mM sodium acetate (pH 7) as follows:
1. 50% DMSO+50 mM Na-Acetate (pH 7)
2. 700 mM Acetylcholine-Cl+50% DMSO+50 mM Na-Acetate (pH 7)
3. 600 mM Acetylcholine-Cl+50% DMSO+50 mM Na-Acetate (pH 7)
4. 500 mM Acetylcholine-Cl+50% DMSO+50 mM Na-Acetate (pH 7)
5. 400 mM Acetylcholine-Cl+50% DMSO+50 mM Na-Acetate (pH 7)
6. −80° C.

Following dilution of each sample in each respective RNA storage environment, samples were stored in a thermal cycler at 60° C. for about 24 hours and then analyzed by denaturing agarose gel electrophoresis as shown in FIG. 11 C and D.

### Example 27

Accelerated RNA Stability Testing at 60° C.

In vitro transcribed RNA was diluted at a ratio of about 1:10 (about 100-300 μg/mL) in different compositions containing either 5 mM sodium phosphate (pH 8) and 10 mM sodium acetate (pH 7), or a mixture of TMG (Sigma-Aldrich, St. Louis, MO; Product #B0300), NDSB-195 (Sigma-Aldrich, St. Louis, MO; Product #D0195), NDSB-221 (Hampton Research, Aliso Viejo, CA; Product #HR2-791), or L-carnitine (Cayman Chemical, Ann Arbor, MI; Product #21489), and 5 mM sodium phosphate (pH 8) and 10 mM sodium acetate (pH 7) as follows:
1. 5 mM Na-Phosphate (pH 8)+10 mM Na-Acetate (pH 7)
2. 3M TMG+5 mM Na-Phosphate (pH 8)+10 mM Na-Acetate (pH 7)
3. 1.5M NDSB-195+5 mM Na-Phosphate (pH 8)+10 mM Na-Acetate (pH 7)
4. 2M NDSB-221+5 mM Na-Phosphate (pH 8)+10 mM Na-Acetate (pH 7)
5. 2M Carnitine+5 mM Na-Phosphate (pH 8)+10 mM Na-Acetate (pH 7)
6. −80° C.

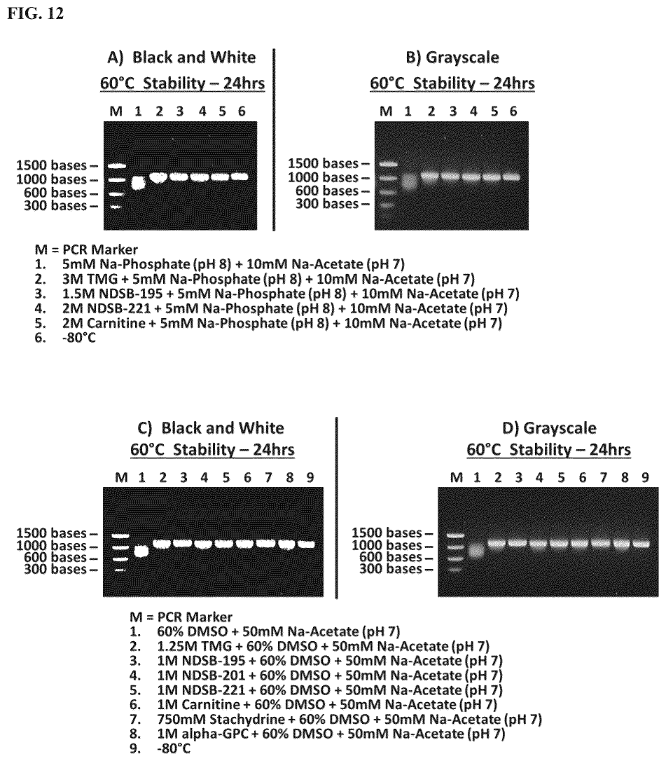

Following dilution of each sample in each respective RNA storage environment, samples were stored in a thermal cycler at 60° C. for about 24 hours and then analyzed by denaturing agarose gel electrophoresis as shown in FIG. 12A and B.

### Example 28

Accelerated RNA Stability Testing at 60° C.

In vitro transcribed RNA was diluted at a ratio of about 1:10 (about 100-300 μg/mL) in different compositions containing either 60% DMSO with 50 mM sodium acetate (pH 7), or a mixture of TMG, NDSB-195, NDSB-201 (Sigma-Aldrich, St. Louis, MO; Product #82804), NDSB-221, L-carnitine, stachydrine (Cayman Chemical, Ann Arbor, MI; Product #20506), or L-alpha-glycerylphosphorylcholine (alpha-GPC) (Botany Bio, San Luis Obispo, CA; Product #alphagpc-99-powder) and 60% DMSO with 50 mM sodium acetate (pH 7) as follows:
1. 60% DMSO+50 mM Na-Acetate (pH 7)
2. 1.25M TMG+60% DMSO+50 mM Na-Acetate (pH 7)
3. 1M NDSB-195+60% DMSO+50 mM Na-Acetate (pH 7)
4. 1M NDSB-201+60% DMSO+50 mM Na-Acetate (pH 7)
5. 1M NDSB-221+60% DMSO+50 mM Na-Acetate (pH 7)
6. 1M Carnitine+60% DMSO+50 mM Na-Acetate (pH 7)
7. 750 mM Stachydrine+60% DMSO+50 mM Na-Acetate (pH 7)
8. 1M alpha-GPC+60% DMSO+50 mM Na-Acetate (pH 7)
9. −80° C.

The stachydrine stock was adjusted to a pH of about 7 using NaOH. Following dilution of each sample in each respective RNA storage environment, samples were stored in a thermal cycler at 60° C. for about 24 hours and then analyzed by denaturing agarose gel electrophoresis as shown in FIG. 12 C and D.

### Example 29

Accelerated RNA Stability Testing at 70° C.

In vitro transcribed RNA was diluted at a ratio of about 1:10 (about 100-300 μg/mL) in different compositions containing either 50 mM Tris-HCl (pH 7), or a mixture of −8.5 kDa poly(2-(trimethylamino)ethyl methacrylate) chloride (PTMAEMA) (Sigma-Aldrich, St. Louis, MO; Product #657670) and 50 mM Tris-HCl (pH 7) as follows:
1. 50 mM Tris-HCl (pH 7)
2. 50 μM PTMAEMA+50 mM Tris-HCl (pH 7)
3. 40 μM PTMAEMA+50 mM Tris-HCl (pH 7)
4. 30 μM PTMAEMA+50 mM Tris-HCl (pH 7)
5. 20 μM PTMAEMA+50 mM Tris-HCl (pH 7)
6. −80° C.

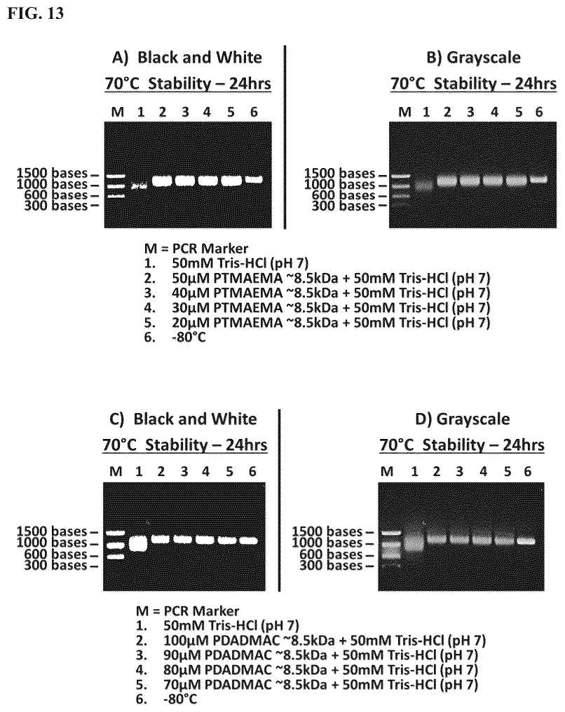

Following dilution of each sample in each respective RNA storage environment, samples were stored in a thermal cycler at 70° C. for about 24 hours and then analyzed by denaturing agarose gel electrophoresis as shown in FIG. 13A and B. Prior to performing gel electrophoresis, the PTMAEMA samples were incubated with 1 mM-8 kDa PAA sodium salt for 30 minutes at 70° C. and then incubated overnight at room temperature (about 20-25° C.) to improve analysis by gel electrophoresis.

### Example 30

Accelerated RNA Stability Testing at 70° C.

In vitro transcribed RNA was diluted at a ratio of about 1:10 (about 100-300n/mL) in different compositions containing either 50 mM Tris-HCl (pH 7), or a mixture of −8.5 kDa poly(diallyldimethylammonium chloride) (PDADMAC) (Polysciences Inc., Warrington, PA; Product #24828-100) and 50 mM Tris-HCl (pH 7) as follows:
1. 50 mM Tris-HCl (pH 7)
2. 100 μM PDADMAC+50 mM Tris-HCl (pH 7)
3. 90 μM PDADMAC+50 mM Tris-HCl (pH 7)
4. 80 μM PDADMAC+50 mM Tris-HCl (pH 7)
5. 70 μM PDADMAC+50 mM Tris-HCl (pH 7)
6. −80° C.

Following dilution of each sample in each respective RNA storage environment, samples were stored in a thermal cycler at 70° C. for about 24 hours and then analyzed by denaturing agarose gel electrophoresis as shown in FIG. 13 C and D. Prior to performing gel electrophoresis, the PDADMAC samples were incubated with 1 mM-8 kDa PAA sodium salt overnight at room temperature (about 20-25° C.) to improve analysis by gel electrophoresis.

### Example 31

Accelerated RNA Stability Testing at 60° C.

In vitro transcribed RNA was diluted at a ratio of about 1:10 (about 100-300n/mL) in different compositions containing either 50 mM sodium acetate (pH 5.2), or a mixture of −10 kDa poly(2-(diethylamino)ethyl methacrylate) (PDEAEMA) (Sigma-Aldrich, St. Louis, MO; Product #910104) and 50 mM sodium acetate (pH 5.2) as follows:
1. 50 mM Na-Acetate (pH 5.2)
2. 35 μM PDEAEMA+50 mM Na-Acetate (pH 5.2)
3. 30 μM PDEAEMA+50 mM Na-Acetate (pH 5.2)
4. 25 μM PDEAEMA+50 mM Na-Acetate (pH 5.2)
5. 20 μM PDEAEMA+50 mM Na-Acetate (pH 5.2)
6. −80° C.

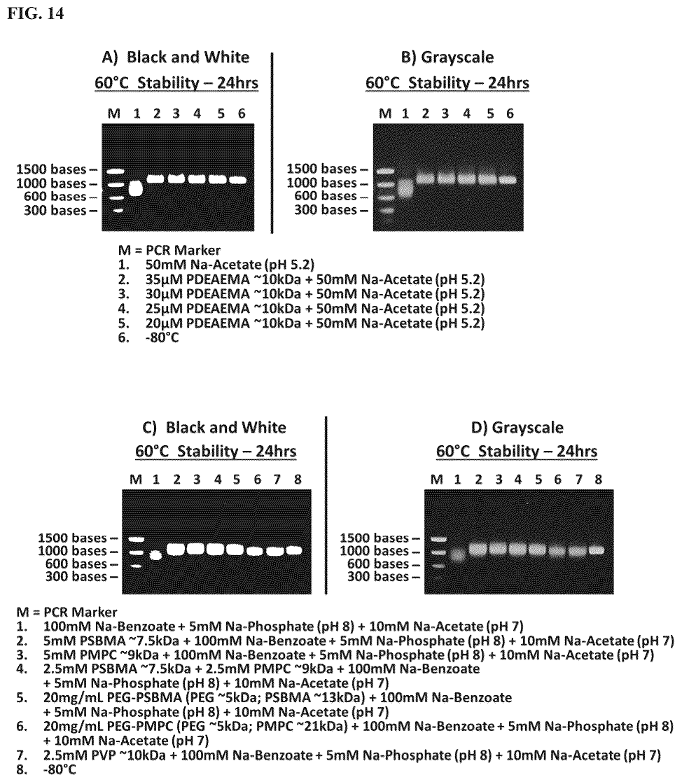

The PDEAEMA stock solution was made in 50 mM sodium acetate (pH 5.2). Following dilution of each sample in each respective RNA storage environment, samples were stored in a thermal cycler at 60° C. for about 24 hours and then analyzed by denaturing agarose gel electrophoresis as shown in FIG. 14A and B. Prior to performing gel electrophoresis, the PDEAEMA samples were incubated with 2.5 mM ˜8 kDa PAA sodium salt for about 1 hour at room temperature (about 20-25° C.) to improve analysis by gel electrophoresis.

### Example 32

Accelerated RNA Stability Testing at 60° C.

In vitro transcribed RNA was diluted at a ratio of about 1:10 (about 100-300n/mL) in different compositions containing either 100 mM sodium benzoate with 5 mM sodium phosphate (pH 8) and 10 mM sodium acetate (pH 7), or ˜7.5 kDa poly(2-(N-3-sulfopropyl-N,N-dimethyl ammonium)ethyl methacrylate) (PSBMA) (Sigma-Aldrich, St. Louis, MO; Product #922390), ˜9 kDa poly(2-methacryloyloxyethyl phosphorylcholine) (PMPC) (Sigma-Aldrich, St. Louis, MO; Product #922749), a combination of both ˜7.5 kDa PSBMA and ˜9 kDa PMPC, PEG-block-PSBMA block copolymer (PEG-PSBMA) (PEG Mₙ 5,000; PSBMA Mₙ 13,000) (Sigma-Aldrich, St. Louis, MO; Product #925640), PEG-block-PMPC block copolymer (PEG-PMPC) (PEG Mₙ 5,000; PMPC Mₙ 21,000) (Sigma-Aldrich, St. Louis, MO; Product #925632), or −10 kDa polyvinylpyrrolidone (PVP) (Sigma-Aldrich, St. Louis, MO; Product #P2307) and 100 mM sodium benzoate with 5 mM sodium phosphate (pH 8) and 10 mM sodium acetate (pH 7) as follows:
1. 100 mM Na-Benzoate+5 mM Na-Phosphate (pH 8)+10 mM Na-Acetate (pH 7)
2. 5 mM PSBMA+100 mM Na-Benzoate+5 mM Na-Phosphate (pH 8)+10 mM Na-Acetate (pH 7)
3. 5 mM PMPC+100 mM Na-Benzoate+5 mM Na-Phosphate (pH 8)+10 mM Na-Acetate (pH 7)
4. 2.5 mM PSBMA+2.5 mM PMPC+100 mM Na-Benzoate+5 mM Na-Phosphate (pH 8)+10 mM Na-Acetate (pH 7)
5. 20 mg/mL PEG-PSBMA+100 mM Na-Benzoate+5 mM Na-Phosphate (pH 8)+10 mM Na-Acetate (pH 7)
6. 20 mg/mL PEG-PMPC+100 mM Na-Benzoate+5 mM Na-Phosphate (pH 8)+10 mM Na-Acetate (pH 7)
7. 2.5 mM PVP+100 mM Na-Benzoate+5 mM Na-Phosphate (pH 8)+10 mM Na-Acetate (pH 7)
8. −80° C.

Following dilution of each sample in each respective RNA storage environment, samples were stored in a thermal cycler at 60° C. for about 24 hours and then analyzed by denaturing agarose gel electrophoresis as shown in FIG. 14 C and D. Prior to performing gel electrophoresis, the samples were incubated with 1 mM-8 kDa PAA sodium salt for about 1 hour at room temperature (about 20-25° C.) to improve analysis by gel electrophoresis.

### Example 33

Accelerated RNA Stability Testing at 60° C.

In vitro transcribed RNA was diluted at a ratio of about 1:10 (about 100-300n/mL) in different compositions containing either 50 mM sodium acetate (pH 5.2), or a mixture of ˜8 kDa poly(acrylic acid, sodium salt) (PAA) (Sigma-Aldrich, St. Louis, MO; Product #416029) and 50 mM sodium acetate (pH 5.2) as follows:
1. 50 mM Na-Acetate (pH 5.2)
2. 5 mM PAA sodium salt+50 mM Na-Acetate (pH 5.2)
3. 2.5 mM PAA sodium salt+50 mM Na-Acetate (pH 5.2)
4. 1 mM PAA sodium salt+50 mM Na-Acetate (pH 5.2)
5. 500 μM PAA sodium salt+50 mM Na-Acetate (pH 5.2)
6. −80° C.

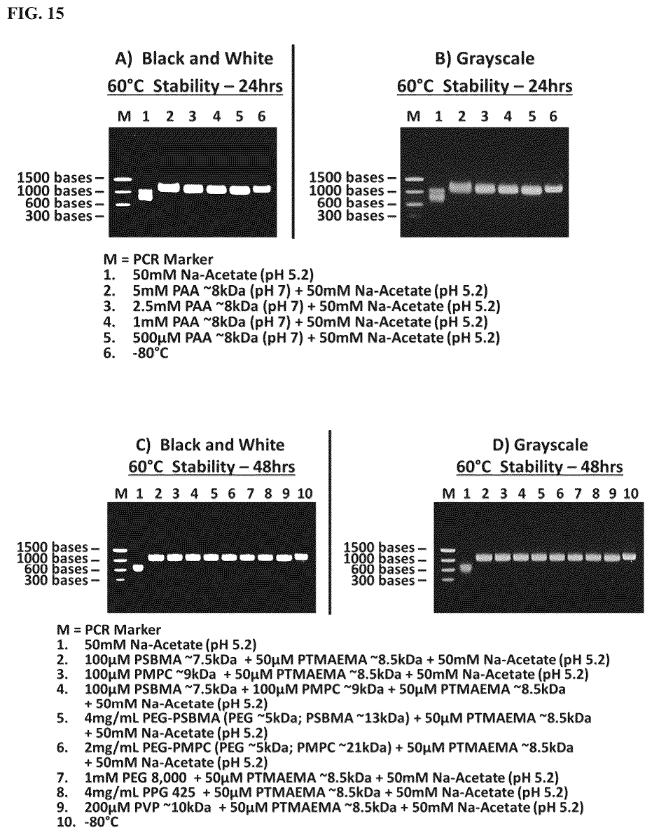

Following dilution of each sample in each respective RNA storage environment, samples were stored in a thermal cycler at 60° C. for about 24 hours and then analyzed by denaturing agarose gel electrophoresis as shown in FIG. 15A and B.

### Example 34

Accelerated RNA Stability Testing at 60° C.

In vitro transcribed RNA was diluted at a ratio of about 1:10 (about 100-300n/mL) in different compositions containing either 50 mM sodium acetate (pH 5.2), or 50 μM ˜8.5 kDa PTMAEMA in combination with ˜7.5 kDa PSBMA, ˜9 kDa PMPC, a combination of both ˜7.5 kDa PSBMA and ˜9 kDa PMPC, PEG-PSBMA (PEG Mₙ 5,000; PSBMA Mₙ 13,000), PEG-PMPC (PEG Mₙ 5,000; PMPC Mₙ 21,000), poly(ethylene glycol) 8,000 (PEG) (Sigma-Aldrich, St. Louis, MO; Product #89510), poly(propylene glycol) 425 (PPG) (Mₙ ˜425) (Sigma-Aldrich, St. Louis, MO; Product #202304), or ˜10 kDa PVP, and 50 mM sodium acetate (pH 5.2) as follows:
1. 50 mM Na-Acetate (pH 5.2)
2. 100 μM PSBMA+50 μM PTMAEMA+50 mM Na-Acetate (pH 5.2)
3. 100 μM PMPC+50 μM PTMAEMA+50 mM Na-Acetate (pH 5.2)
4. 100 μM PSBMA+100 μM PMPC+50 μM PTMAEMA+50 mM Na-Acetate (pH 5.2)
5. 4 mg/mL PEG-PSBMA+50 μM PTMAEMA+50 mM Na-Acetate (pH 5.2)
6. 2 mg/mL PEG-PMPC+50 μM PTMAEMA+50 mM Na-Acetate (pH 5.2)
7. 1 mM PEG 8,000+50 μM PTMAEMA+50 mM Na-Acetate (pH 5.2)
8. 4 mg/mL PPG 425+50 μM PTMAEMA+50 mM Na-Acetate (pH 5.2)
9. 200 μM PVP+50 μM PTMAEMA+50 mM Na-Acetate (pH 5.2)
10. −80° C.

Following dilution of each sample in each respective RNA storage environment, samples were stored in a thermal cycler at 60° C. for about 48 hours and then analyzed by denaturing agarose gel electrophoresis as shown in FIG. 15 C and D. Prior to performing gel electrophoresis, the samples were incubated with 2.5 mM ˜8 kDa PAA sodium salt for 30 minutes at 70° C. to improve analysis by gel electrophoresis.

### Example 35

Accelerated RNA Stability Testing at 60° C.

In vitro transcribed RNA was diluted at a ratio of about 1:10 (about 100-300 μg/mL) in different compositions containing either 50 mM sodium acetate (pH 5.2), or 100 μM ˜8.5 kDa PDADMAC in combination with ˜7.5 kDa PSBMA, ˜9 kDa PMPC, a combination of both ˜7.5 kDa PSBMA and ˜9 kDa PMPC, PEG-PSBMA (PEG Mₙ 5,000; PSBMA Mₙ 13,000), PEG-PMPC (PEG Mₙ 5,000; PMPC Mₙ 21,000), PEG 8,000, PPG 425, or ˜10 kDa PVP, and 50 mM sodium acetate (pH 5.2) as follows:
1. 50 mM Na-Acetate (pH 5.2)
2. 100 μM PSBMA+100 μM PDADMAC+50 mM Na-Acetate (pH 5.2)
3. 100 μM PMPC+100 μM PDADMAC+50 mM Na-Acetate (pH 5.2)
4. 100 μM PSBMA+100 μM PMPC+100 μM PDADMAC+50 mM Na-Acetate (pH 5.2)
5. 4 mg/mL PEG-PSBMA+100 μM PDADMAC+50 mM Na-Acetate (pH 5.2)
6. 2 mg/mL PEG-PMPC+100 μM PDADMAC+50 mM Na-Acetate (pH 5.2)
7. 1 mM PEG 8,000+100 μM PDADMAC+50 mM Na-Acetate (pH 5.2)
8. 4 mg/mL PPG 425+100 μM PDADMAC+50 mM Na-Acetate (pH 5.2)
9. 200 μM PVP+100 μM PDADMAC+50 mM Na-Acetate (pH 5.2)
10. −80° C.

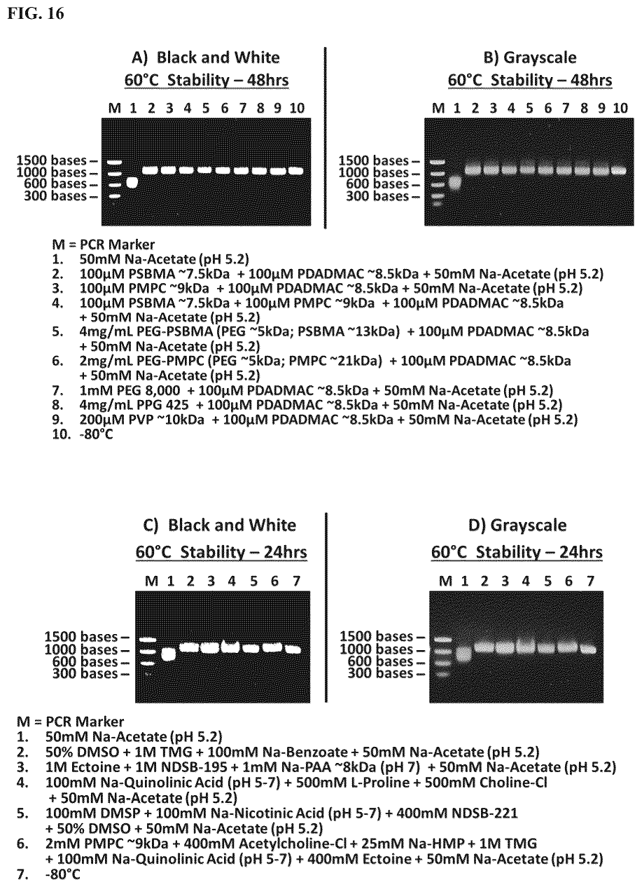

Following dilution of each sample in each respective RNA storage environment, samples were stored in a thermal cycler at 60° C. for about 48 hours and then analyzed by denaturing agarose gel electrophoresis as shown in FIG. 16A and B. Prior to performing gel electrophoresis, the samples were incubated with 2.5 mM-8 kDa PAA sodium salt for about 1 hour at room temperature (about 20-25° C.) to improve analysis by gel electrophoresis.

### Example 36

Accelerated RNA Stability Testing at 60° C.

In vitro transcribed RNA was diluted at a ratio of about 1:10 (about 100-300 μg/mL) in different compositions containing either 50 mM sodium acetate (pH 5.2), or one or more compounds in compositions with 50 mM sodium acetate (pH 5.2) as follows:
1. 50 mM Na-Acetate (pH 5.2)
2. 50% DMSO+1M TMG+100 mM Na-Benzoate+50 mM Na-Acetate (pH 5.2)
3. 1M Ectoine+1M NDSB-195+1 mM PAA ˜8 kDa sodium salt+50 mM Na-Acetate (pH 5.2)
4. 100 mM Na-Quinolinic Acid (pH 7)+500 mM L-Proline+500 mM Choline-Cl+50 mM Na-Acetate (pH 5.2)
5. 100 mM DMSP+100 mM Na-Nicotinic Acid (pH 7)+400 mM NDSB-221+50% DMSO+50 mM Na-Acetate (pH 5.2)
6. 2 mM PMPC ˜9 kDa+400 mM Acetylcholine-Cl+25 mM Na-HMP+1M TMG+100 mM Na-Quinolinic Acid (pH 7)+400 mM Ectoine+50 mM Na-Acetate (pH 5.2)
7. −80° C.

The quinolinic acid, nicotinic acid, and DMSP stocks were adjusted to a pH of about 5-7 using NaOH. Following dilution of each sample in each respective RNA storage environment, samples were stored in a thermal cycler at 60° C. for about 24 hours and then analyzed by denaturing agarose gel electrophoresis as shown in FIG. 16 C and D. Prior to performing gel electrophoresis, sample 5 comprising DMSP was incubated with 1 mM-8 kDa PAA sodium salt for about 1 hour at room temperature (about 20-25° C.) to improve analysis by gel electrophoresis.

### Example 37

Accelerated RNA Stability Testing at 60° C.

In vitro transcribed RNA was diluted at a ratio of about 1:10 (about 100-300n/mL) in different compositions containing either 50 mM sodium acetate (pH 5.2), or one or more compounds in compositions with 50 mM sodium acetate (pH 5.2) as follows:
1. 50 mM Na-Acetate (pH 5.2)
2. 50% DMSO+1M TMG+100 mM Na-Benzoate+50 mM Na-Acetate (pH 5.2)
3. 100 mM Na-TMP+500 mM Choline-Cl+1 mM PAA ˜8 kDa sodium salt+50 mM Na-Acetate (pH 5.2)
4. 100 mM DMSP+100 mM Na-Quinolinic Acid (pH 7)+600 mM NDSB-195+50% DMSO+50 mM Na-Acetate (pH 5.2)
5. 1 mM PSBMA ˜7.5 kDa+100 mM Acetylcholine-Cl+40 mM Na-HMP+1M TMG+100 mM Na-Benzoate+400 mM Ectoine+50 mM Na-Acetate (pH 5.2)
6. −80° C.

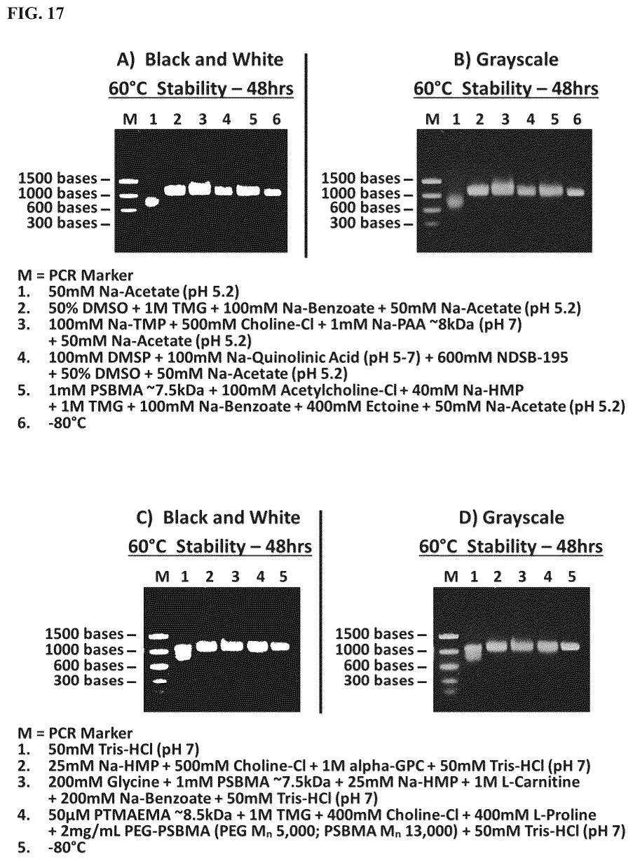

The quinolinic acid and DMSP stocks were adjusted to a pH of about 5-7 using NaOH. Following dilution of each sample in each respective RNA storage environment, samples were stored in a thermal cycler at 60° C. for about 48 hours and then analyzed by denaturing agarose gel electrophoresis as shown in FIG. 17A and B. Prior to performing gel electrophoresis, sample 4 comprising DMSP was incubated with 1 mM-8 kDa PAA sodium salt for about 1 hour at room temperature (about 20-25° C.) to improve analysis by gel electrophoresis.

### Example 38

Accelerated RNA Stability Testing at 60° C.

In vitro transcribed RNA was diluted at a ratio of about 1:10 (about 100-300n/mL) in different compositions containing either 50 mM Tris-HCl (pH 7), or one or more compounds in compositions with 50 mM Tris-HCl (pH 7) as follows:
1. 50 mM Tris-HCl (pH 7)
2. 25 mM Na-HMP+500 mM Choline-Cl+1M alpha-GPC+50 mM Tris-HCl (pH 7)
3. 200 mM Glycine+1 mM PSBMA ˜7.5 kDa+25 mM Na-HMP+1M L-Carnitine+200 mM Na-Benzoate+50 mM Tris-HCl (pH 7)
4. 50 μM PTMAEMA ˜8.5 kDa+1M TMG+400 mM Choline-Cl+400 mM L-Proline+2 mg/mL PEG-PSBMA (PEG Mₙ 5,000; PSBMA Mₙ 13,000)+50 mM Tris-HCl (pH 7)
5. −80° C.

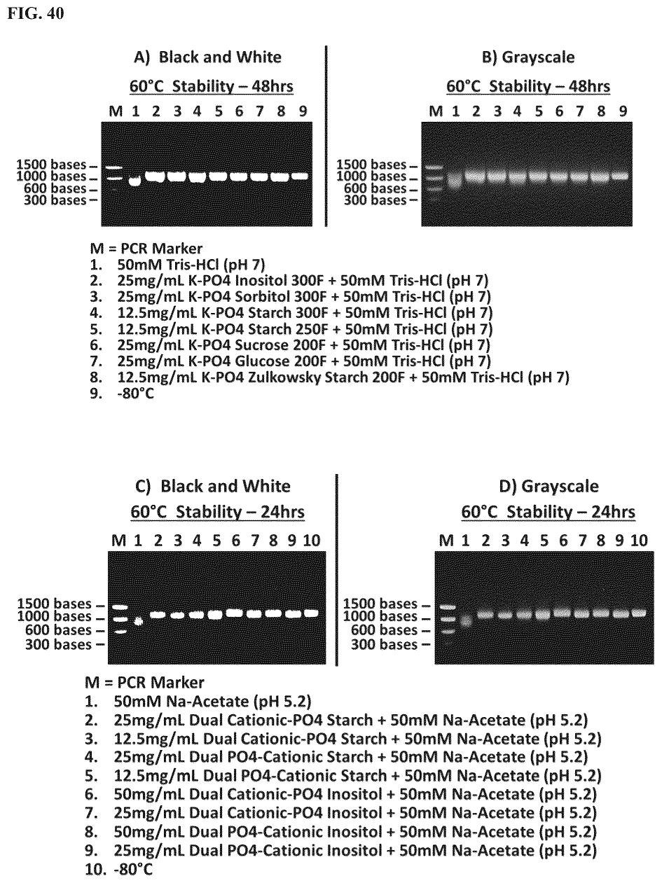

Following dilution of each sample in each respective RNA storage environment, samples were stored in a thermal cycler at 60° C. for about 48 hours and then analyzed by denaturing agarose gel electrophoresis as shown in FIG. 17 C and D. Prior to performing gel electrophoresis, sample 4 comprising PTMAEMA was incubated with 2.5 mM-8 kDa PAA sodium salt for 30 minutes at 70° C. to improve analysis by gel electrophoresis. FIG. 40 shows the agarose gel following storage of the RNA for about 48 hours at 60° C.

### Example 39

Accelerated RNA Stability Testing at 60° C.

In vitro transcribed RNA was diluted at a ratio of about 1:10 (about 100-300 μg/mL) in different compositions containing either 50 mM Tris-HCl (pH 7), or one or more compounds in compositions with 50 mM Tris-HCl (pH 7) as follows:
1. 50 mM Tris-HCl (pH 7)
2. 40 mM Na-HMP+500 mM Choline-Cl+1M alpha-GPC+50 mM Tris-HCl (pH 7)
3. 500 mM Choline-Cl+50% DMSO+1 mM PMPC ˜9 kDa+500 mM NDSB-195+50 mM Tris-HCl (pH 7)
4. 50 μM PTMAEMA ˜8.5 kDa+100 mM TMG+100 mM Ectoine+400 mM alpha-GPC+100 μM PSBMA ˜7.5 kDa+50 mM Tris-HCl (pH 7)
5. −80° C.

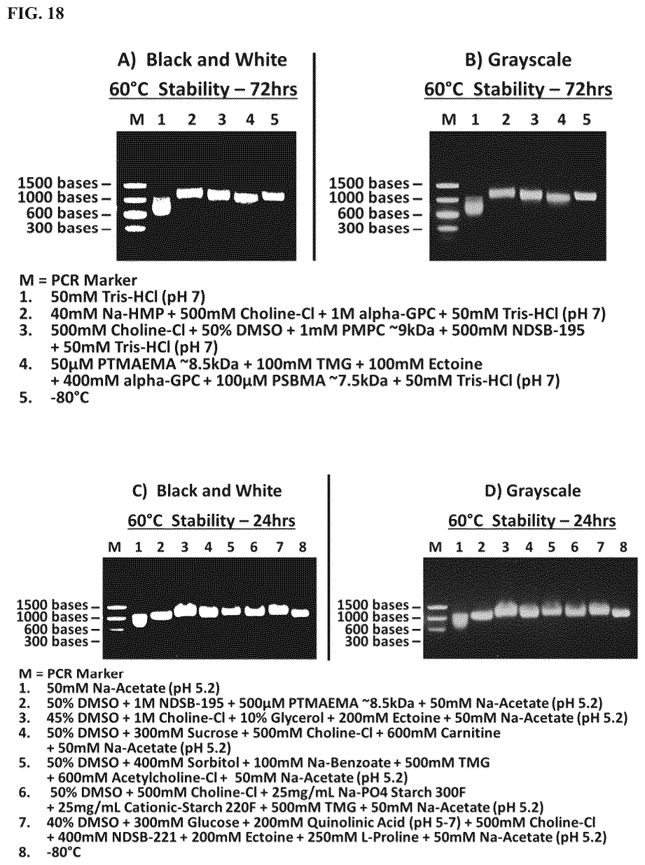

Following dilution of each sample in each respective RNA storage environment, samples were stored in a thermal cycler at 60° C. for about 72 hours and then analyzed by denaturing agarose gel electrophoresis as shown in FIG. 18A and B. Prior to performing gel electrophoresis, sample 4 comprising PTMAEMA was incubated with 2.5 mM-8 kDa PAA sodium salt for 30 minutes at 70° C. to improve analysis by gel electrophoresis.

### Example 40

Accelerated RNA Stability Testing at 60° C.

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 18 C and D.

Following dilution of each sample in each respective RNA storage environment, samples were stored in a thermal cycler at 60° C. for about 24 hours and then analyzed by denaturing agarose gel electrophoresis as shown in FIG. 18 C and D. Prior to performing gel electrophoresis, sample 1 comprising PTMAEMA was incubated with 2.5 mM-8 kDa PAA sodium salt for 30 minutes at 70° C. to improve analysis by gel electrophoresis.

The following substances in one or more compositions were purchased as follows: Sucrose (Sigma-Aldrich, St. Louis, MO; Product #84097); D-Glucose (Sigma-Aldrich, Product #G5767) Trehalose (Cayman Chemical, Ann Arbor, MI, Product #20517), D-Sorbitol (Sigma-Aldrich, Product #S1876) and Glycerol (Sigma-Aldrich, Product #G5516).

### Example 41

Accelerated RNA Stability Testing at 60° C.

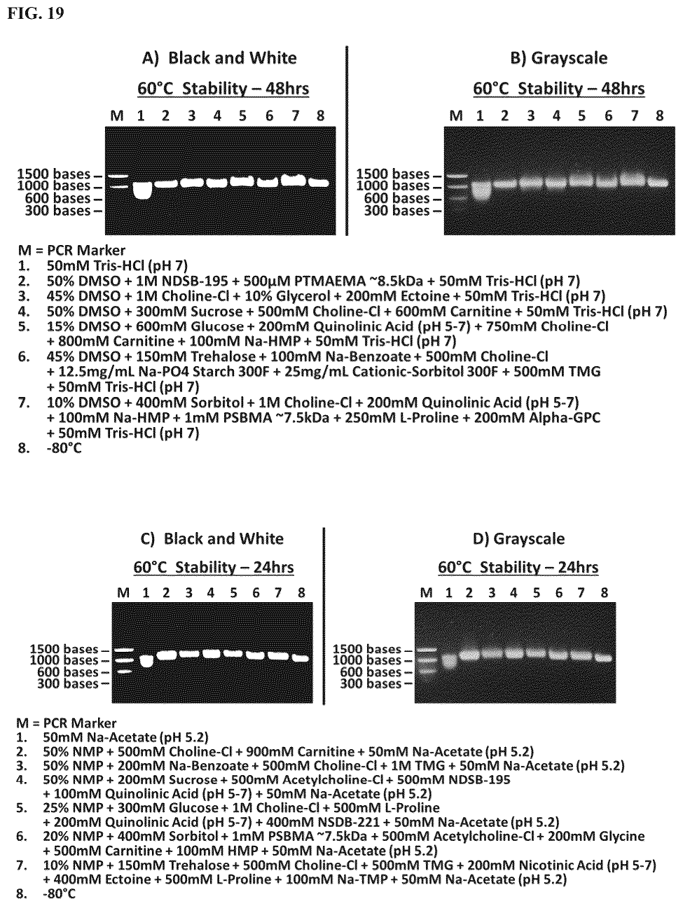

In vitro transcribed RNA was diluted at a ratio of about 1:10 (about 100-300n/mL) in different compositions containing either 50 mM Tris-HCl (pH 7), or one or more compounds in compositions with 50 mM Tris-HCl (pH 7) as shown in FIG. 19A and B:

Following dilution of each sample in each respective RNA storage environment, samples were stored in a thermal cycler at 60° C. for about 48 hours and then analyzed by denaturing agarose gel electrophoresis as shown in FIG. 19A and B. Prior to performing gel electrophoresis, sample 1 comprising PTMAEMA was incubated with 2.5 mM-8 kDa PAA sodium salt for 30 minutes at 70° C. to improve analysis by gel electrophoresis.

### Example 42

Accelerated RNA Stability Testing at 60° C.

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 19 C and D:

Following dilution of each sample in each respective RNA storage environment, samples were stored in a thermal cycler at 60° C. for about 24 hours and then analyzed by denaturing agarose gel electrophoresis as shown in FIG. 19 C and D.

### Example 43

Accelerated RNA Stability Testing at 60° C.

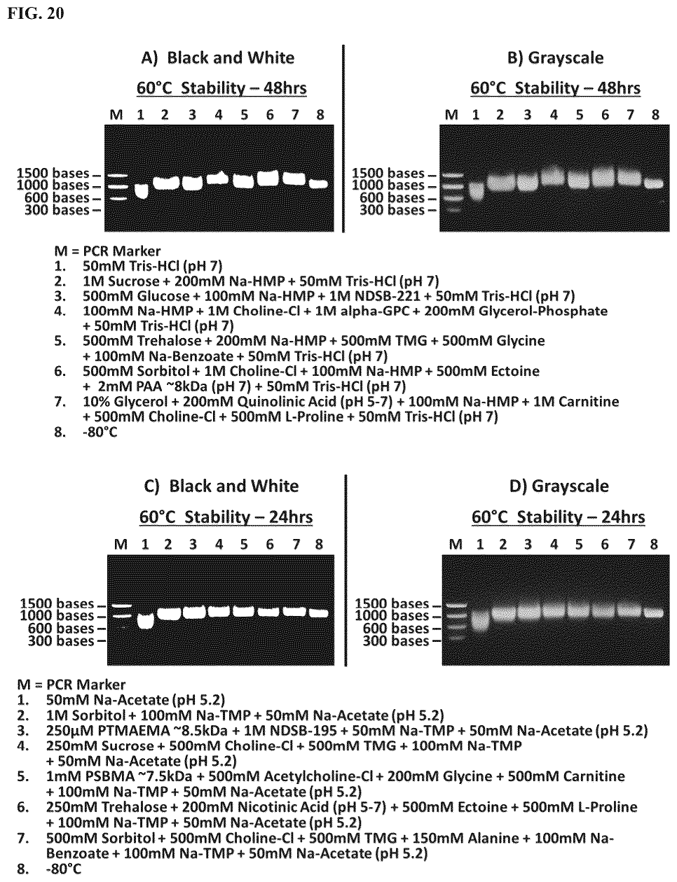

In vitro transcribed RNA was diluted at a ratio of about 1:10 (about 100-300n/mL) in different compositions containing either 50 mM Tris-HCl (pH 7), or one or more compounds in compositions with 50 mM Tris-HCl (pH 7) as shown in FIG. 20A and B:

Following dilution of each sample in each respective RNA storage environment, samples were stored in a thermal cycler at 60° C. for about 48 hours and then analyzed by denaturing agarose gel electrophoresis as shown in FIG. 20A and B.

### Example 44

Accelerated RNA Stability Testing at 60° C.

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 20 C and D:

Following dilution of each sample in each respective RNA storage environment, samples were stored in a thermal cycler at 60° C. for about 24 hours and then analyzed by denaturing agarose gel electrophoresis as shown in FIG. 20 C and D.

### Example 45

Accelerated RNA Stability Testing at 60° C.

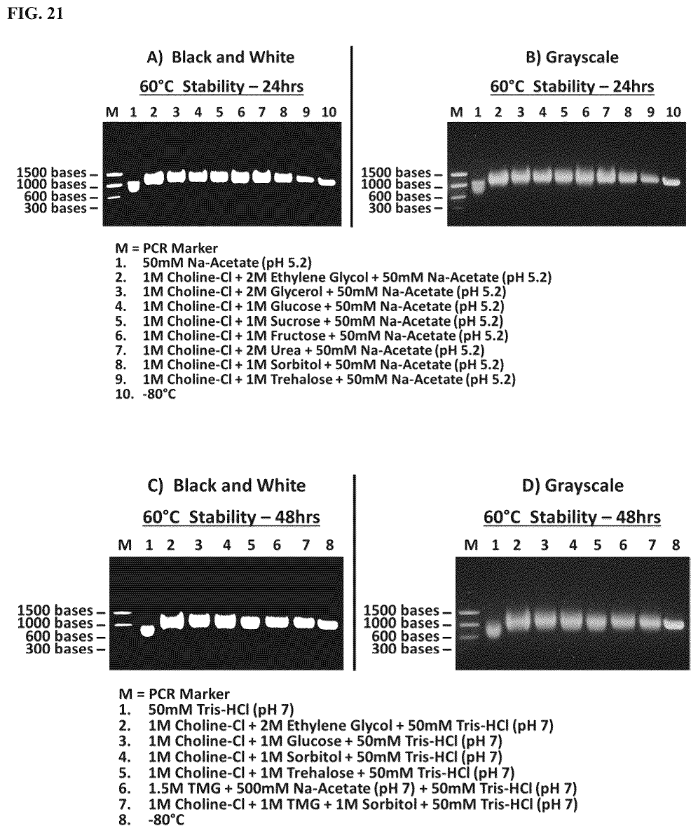

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 21A and B:

Following dilution of each sample in each respective RNA storage environment, samples were stored in a thermal cycler at 60° C. for about 24 hours and then analyzed by denaturing agarose gel electrophoresis as shown in FIG. 21A and B.

The following substances in one or more compositions were purchased as follows: Urea (Sigma-Aldrich, Product #U5378); Ethylene Glycol (Sigma-Aldrich, Product #102466).

### Example 46

Accelerated RNA Stability Testing at 60° C.

In vitro transcribed RNA was diluted at a ratio of about 1:10 (about 100-300n/mL) in different compositions containing either 50 mM Tris-HCl (pH 7), or one or more compounds in compositions with 50 mM Tris-HCl (pH 7) as shown in FIG. 21 C and D:

Following dilution of each sample in each respective RNA storage environment, samples were stored in a thermal cycler at 60° C. for about 48 hours and then analyzed by denaturing agarose gel electrophoresis as shown in FIG. 21 C and D.

### Example 47

Accelerated RNA Stability Testing at 60° C.

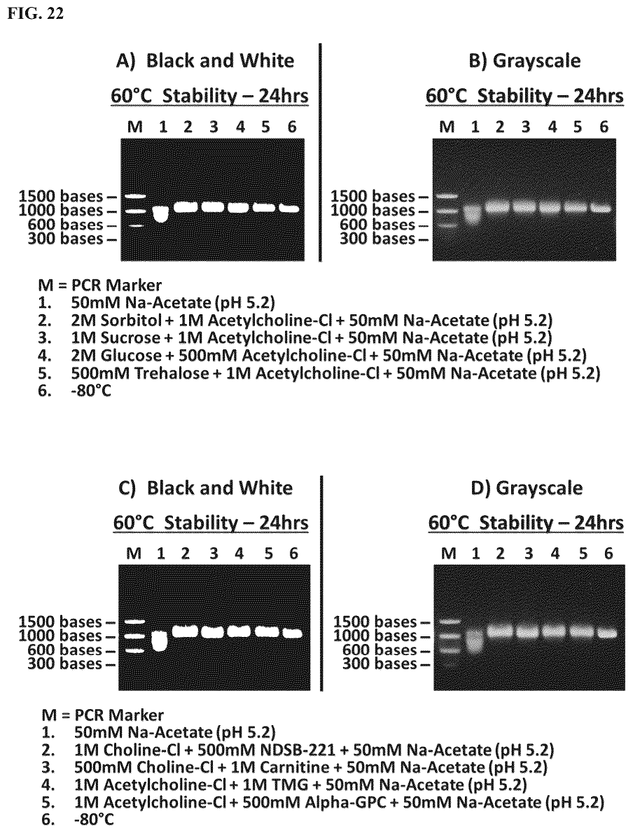

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 22A and B:

Following dilution of each sample in each respective RNA storage environment, samples were stored in a thermal cycler at 60° C. for about 24 hours and then analyzed by denaturing agarose gel electrophoresis as shown in FIG. 22A and B.

### Example 48

Accelerated RNA Stability Testing at 60° C.

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 22 C and D:

Following dilution of each sample in each respective RNA storage environment, samples were stored in a thermal cycler at 60° C. for about 24 hours and then analyzed by denaturing agarose gel electrophoresis as shown in FIG. 22 C and D.

### Example 49

Accelerated RNA Stability Testing at 60° C.

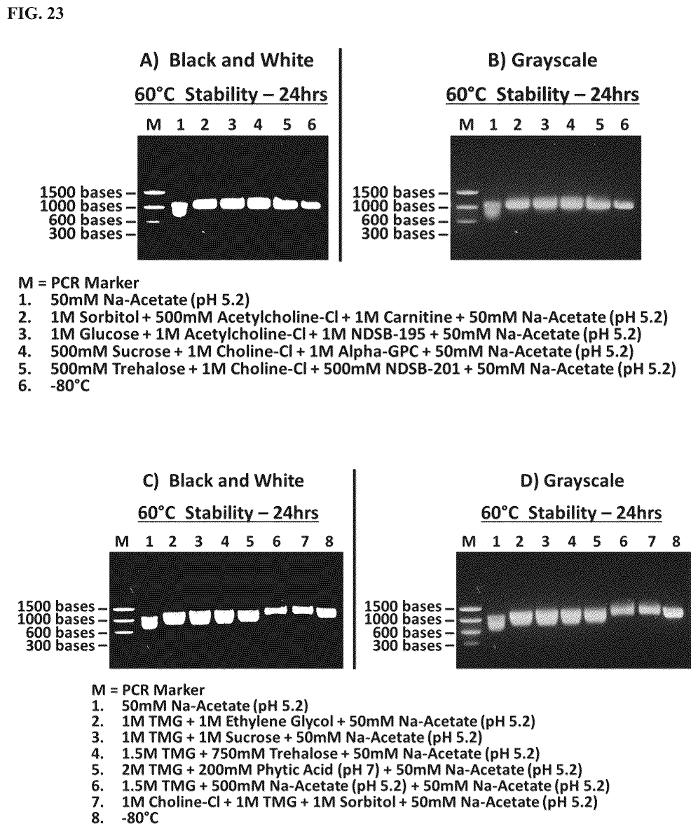

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 23A and B:

Following dilution of each sample in each respective RNA storage environment, samples were stored in a thermal cycler at 60° C. for about 24 hours and then analyzed by denaturing agarose gel electrophoresis as shown in FIG. 23A and B.

### Example 50

Accelerated RNA Stability Testing at 60° C.

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 23 C and D.

Following dilution of each sample in each respective RNA storage environment, samples were stored in a thermal cycler at 60° C. for about 24 hours and then analyzed by denaturing agarose gel electrophoresis as shown in FIG. 23 C and D. The pH of phytic acid was adjusted to about 7 using NaOH.

The following substances in one or more compositions were purchased as follows: Phytic Acid (Sigma-Aldrich, Product #593648).

### Example 51

Accelerated RNA Stability Testing at 60° C.

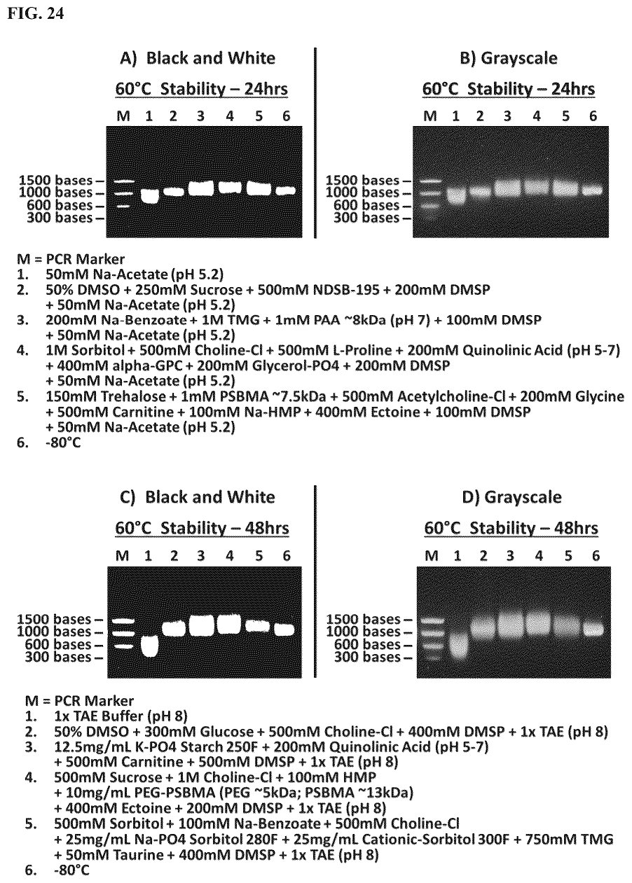

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 24A and B.

Following dilution of each sample in each respective RNA storage environment, samples were stored in a thermal cycler at 60° C. for about 24 hours and then analyzed by denaturing agarose gel electrophoresis as shown in FIG. 24A and B.

PSMBA was ˜7.5 kDa

### Example 52

Accelerated RNA Stability Testing at 60° C.

In vitro transcribed RNA was diluted at a ratio of about 1:10 (about 100-300n/mL) in different compositions containing either TAE buffer (pH 8), or one or more compounds in compositions with TAE buffer (pH 8) as shown in FIG. 24 C and D:

Following dilution of each sample in each respective RNA storage environment, samples were stored in a thermal cycler at 60° C. for about 48 hours and then analyzed by denaturing agarose gel electrophoresis as shown in FIG. 24 C and D.

PEG-PSBMA was (PEG Mn 5,000; PSBMA Mn 13,000)

### Example 53

Accelerated RNA Stability Testing at 60° C.

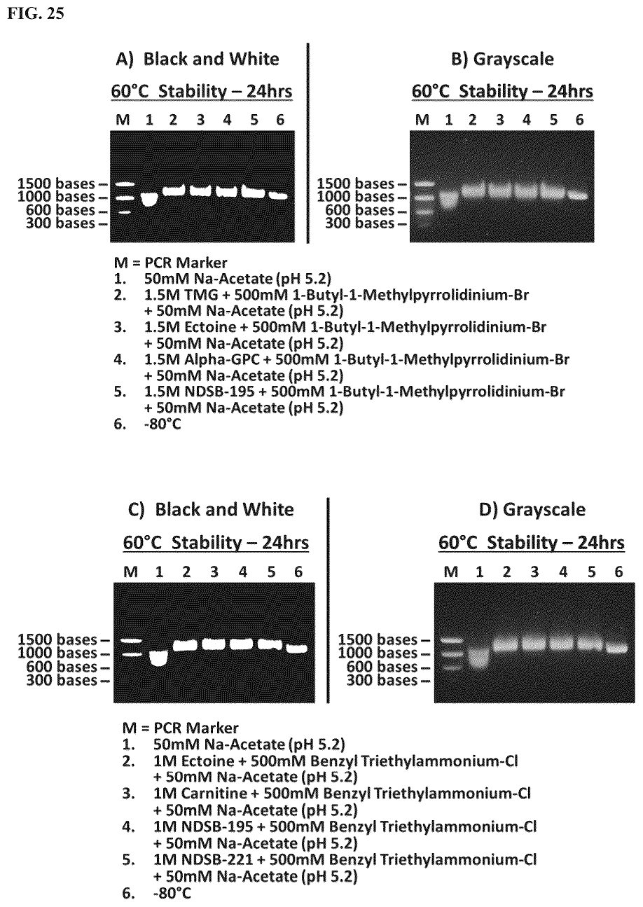

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 25A and B.

Following dilution of each sample in each respective RNA storage environment, samples were stored in a thermal cycler at 60° C. for about 24 hours and then analyzed by denaturing agarose gel electrophoresis as shown in FIG. 25A and B.

### Example 54

Accelerated RNA Stability Testing at 60° C.

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 25 C and D.

Following dilution of each sample in each respective RNA storage environment, samples were stored in a thermal cycler at 60° C. for about 24 hours and then analyzed by denaturing agarose gel electrophoresis as shown in FIG. 25 C and D.

### Example 55

Accelerated RNA Stability Testing at 60° C.

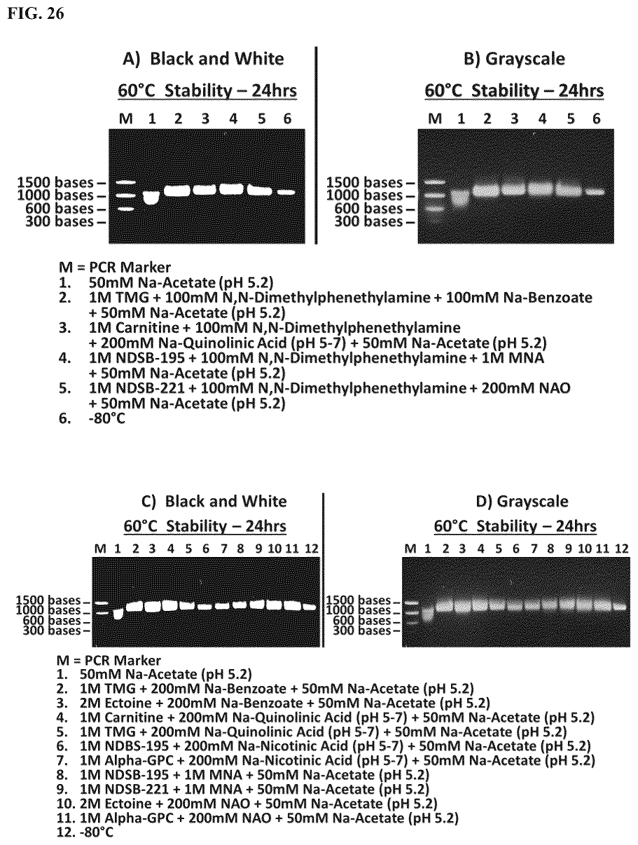

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 26A and B.

Following dilution of each sample in each respective RNA storage environment, samples were stored in a thermal cycler at 60° C. for about 24 hours and then analyzed by denaturing agarose gel electrophoresis as shown in FIG. 26A and B.

Abbreviations: 1-Methylnicotinamide-Cl (MNA) and Nicotinamide N-oxide (NAO)

### Example 56

Accelerated RNA Stability Testing at 60° C.

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 26 C and D.

Following dilution of each sample in each respective RNA storage environment, samples were stored in a thermal cycler at 60° C. for about 24 hours and then analyzed by denaturing agarose gel electrophoresis as shown in FIG. 26 C and D.

Abbreviations: 1-Methylnicotinamide-C₁ (MNA) and Nicotinamide N-oxide (NAO)

### Example 57

Accelerated RNA Stability Testing at 60° C.

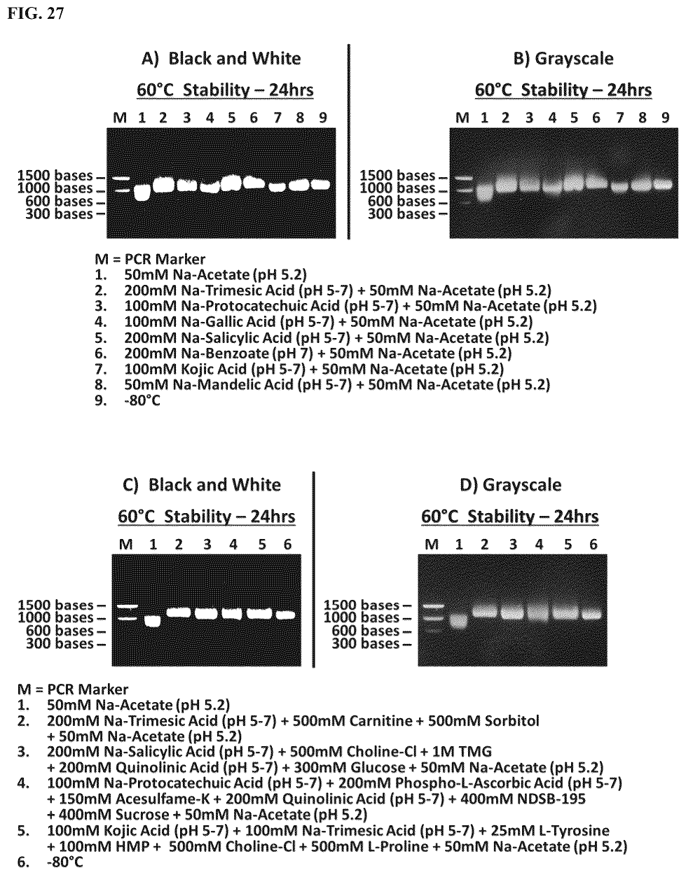

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 27A and B.

Following dilution of each sample in each respective RNA storage environment, samples were stored in a thermal cycler at 60° C. for about 24 hours and then analyzed by denaturing agarose gel electrophoresis as shown in FIG. 27A and B.

The following were purchased from: Trimesic Acid (Cayman Chemical, Product #19198), Protocatechuic Acid (Cayman Chemical, Product #14916), Gallic Acid (Cayman Chemical, Product #11846), Salicylic Acid (Talsen Chemicals, Richmond Hill, NY), Kojic Acid (Talsen Chemicals), Mandelic Acid (Talsen Chemicals).

### Example 58

Accelerated RNA Stability Testing at 60° C.

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 27 C and D.

Following dilution of each sample in each respective RNA storage environment, samples were stored in a thermal cycler at 60° C. for about 24 hours and then analyzed by denaturing agarose gel electrophoresis as shown in FIG. 27 C and D.

The following were purchased from: L-Tyrosine (PureBulk, Roseburg, OR), Acesulfame-K (Cayman Chemical, Product #33356), 2-Phospho-L-ascorbic acid (Sigma-Aldrich, Product #49752).

### Example 59

Accelerated RNA Stability Testing at 60° C.

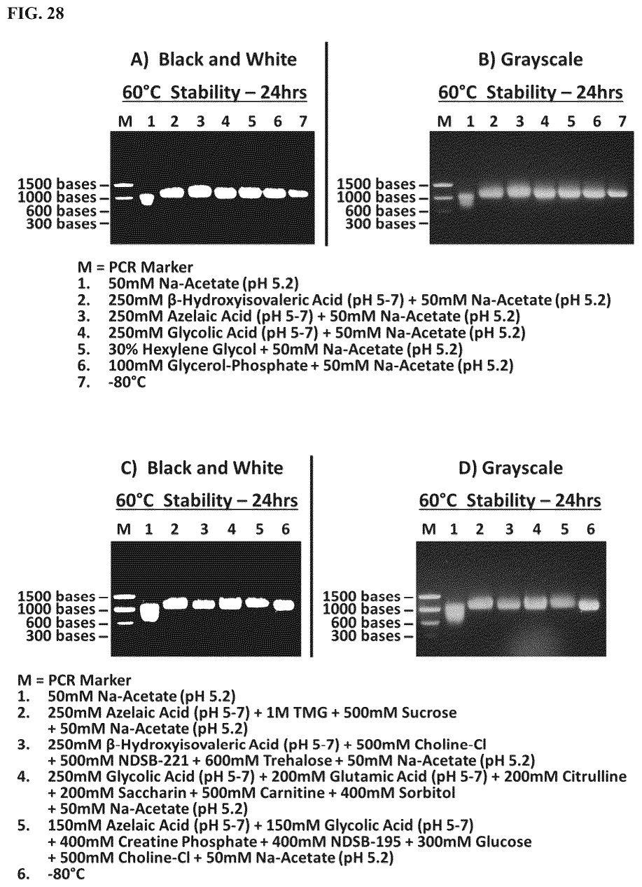

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 28A and B.

Following dilution of each sample in each respective RNA storage environment, samples were stored in a thermal cycler at 60° C. for about 24 hours and then analyzed by denaturing agarose gel electrophoresis as shown in FIG. 28A and B.

The following were purchased from: beta-Hydroxyisovaleric Acid (Cayman Chemical, Product #34030), Azelaic Acid (Talsen Chemicals), Glycolic Acid (Talsen Chemicals).

### Example 60

Accelerated RNA Stability Testing at 60° C.

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 28 C and D.

Following dilution of each sample in each respective RNA storage environment, samples were stored in a thermal cycler at 60° C. for about 24 hours and then analyzed by denaturing agarose gel electrophoresis as shown in FIG. 28 C and D.

The following were purchased from: Glutamic Acid (Modernist Pantry, Eliot, ME, Product #1594-50), Citrulline (Cayman Chemical, Product #35648), Na-Saccharin (Eisen-Golden, Dublin, CA), Creatine Phosphate (Cayman Chemical, Product #37803).

### Example 61

Accelerated RNA Stability Testing at 60° C.

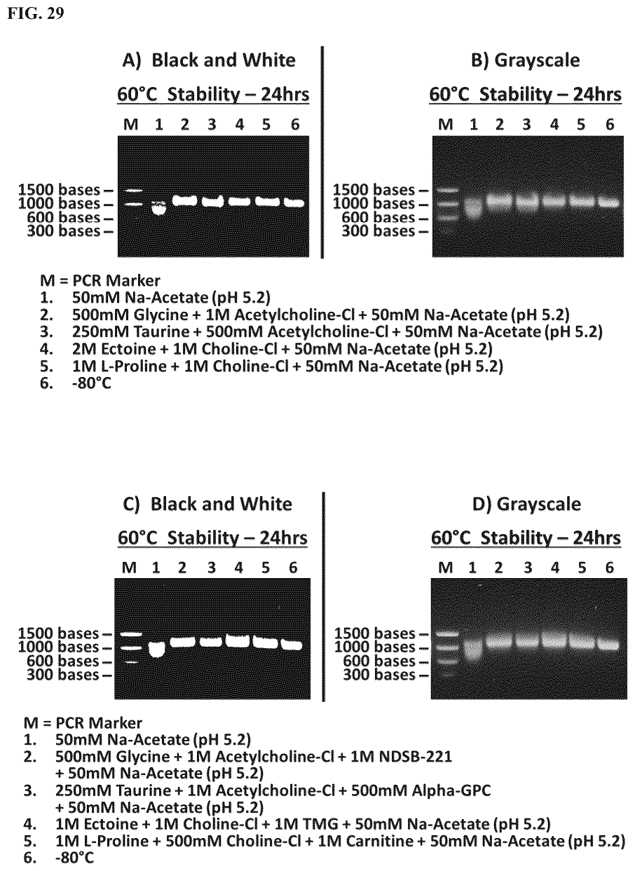

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 29A and B.

Following dilution of each sample in each respective RNA storage environment, samples were stored in a thermal cycler at 60° C. for about 24 hours and then analyzed by denaturing agarose gel electrophoresis as shown in FIG. 29A and B.

### Example 62

Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIG. 29 C and D.

Following dilution of each sample in each respective RNA storage environment, samples were stored in a thermal cycler at 60° C. for about 24 hours and then analyzed by denaturing agarose gel electrophoresis as shown in FIG. 29 C and D.

### Example 63

Accelerated RNA Stability Testing at 60° C.

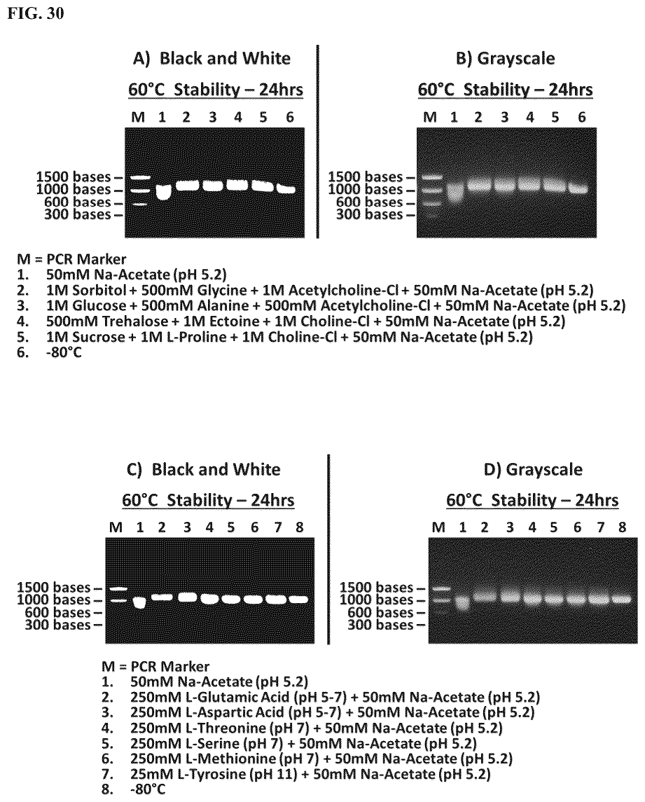

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIG. 30A and B.

Following dilution of each sample in each respective RNA storage environment, samples were stored in a thermal cycler at 60° C. for about 24 hours and then analyzed by denaturing agarose gel electrophoresis as shown in FIG. 30A and B.

### Example 64

Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIG. 30 C and D.

Following dilution of each sample in each respective RNA storage environment, samples were stored in a thermal cycler at 60° C. for about 24 hours and then analyzed by denaturing agarose gel electrophoresis as shown in FIG. 30 C and D.

L-Tyrosine pH was adjusted to 11 to increase solubility and then added to sample. Some precipitation occurred following addition to 50 mM Acetate (pH 5.2)

The following were purchased from: L-Aspartic Acid (PureBulk), L-Threonine (PureBulk), L-Serine (PureBulk, L-Methionine (PureBulk), L-Tyrosine (PureBulk).

### Example 65

Accelerated RNA Stability Testing at 60° C.

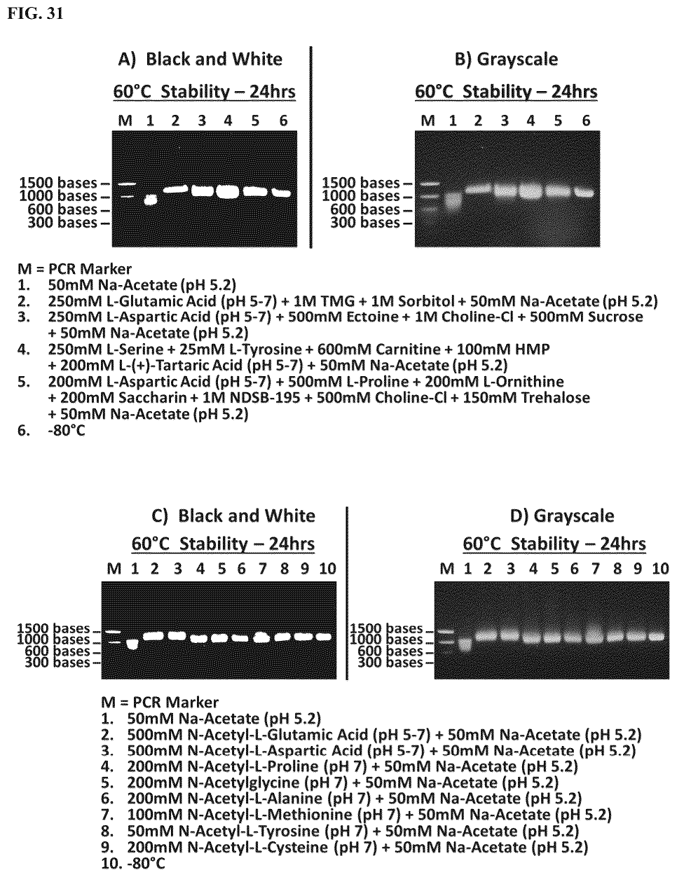

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 31A and B.

Following dilution of each sample in each respective RNA storage environment, samples were stored in a thermal cycler at 60° C. for about 24 hours and then analyzed by denaturing agarose gel electrophoresis as shown in FIG. 31A and B.

The following were purchased from: L-Ornithine (Sigma-Aldrich, Product #02375)

### Example 66

Accelerated RNA Stability Testing at 60° C.

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 31 C and D.

Following dilution of each sample in each respective RNA storage environment, samples were stored in a thermal cycler at 60° C. for about 24 hours and then analyzed by denaturing agarose gel electrophoresis as shown in FIG. 31 C and D.

The following were purchased from: N-Acetyl-L-Glutamic Acid (Sigma-Aldrich, Product #855642), N-Acetyl-L-Aspartic Acid (Cayman Chemical, Product #34635), N-Acetyl-L-Proline (Sigma-Aldrich, Product #A0783), N-Acetylglycine (Sigma-Aldrich, Product #A16300), N-Acetyl-L-Alanine (Sigma-Aldrich, Product #A4625), N-Acetyl-L-Methionine (Sigma-Aldrich, Product #01310), N-Acetyl-L-Tyrosine (PureBulk), N-Acetyl-L-Cysteine (Sigma Aldrich, Product #A9165).

### Example 67

Accelerated RNA Stability Testing at 60° C.

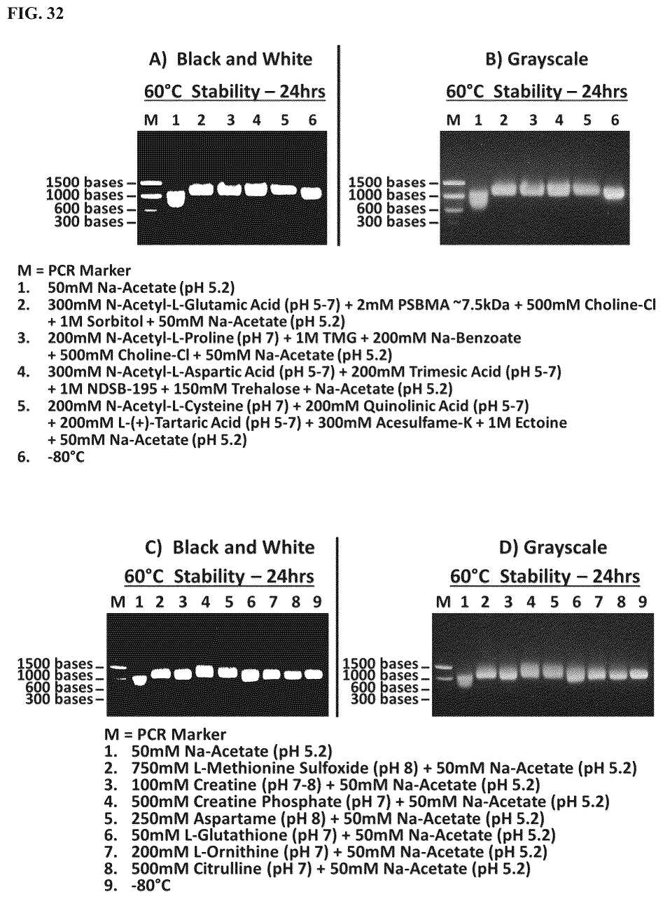

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIG. 32A and B.

Following dilution of each sample in each respective RNA storage environment, samples were stored in a thermal cycler at 60° C. for about 24 hours and then analyzed by denaturing agarose gel electrophoresis as shown in FIG. 32A and B.

### Example 68

Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIG. 32 C and D.

Following dilution of each sample in each respective RNA storage environment, samples were stored in a thermal cycler at 60° C. for about 24 hours and then analyzed by denaturing agarose gel electrophoresis as shown in FIG. 32 C and D.

Aspartame was adjusted to pH 8 to improve solubility prior to adding to sample.

The following were purchased from: L-Methionine Sulfoxide (Cayman Chemical, Product #36255), L-Glutathione (Cayman Chemical, Product #35825), Aspartame (Cayman Chemical, Product #26089), Creatine (Myogenix, Maria, CA).

### Example 69

Accelerated RNA Stability Testing at 60° C.

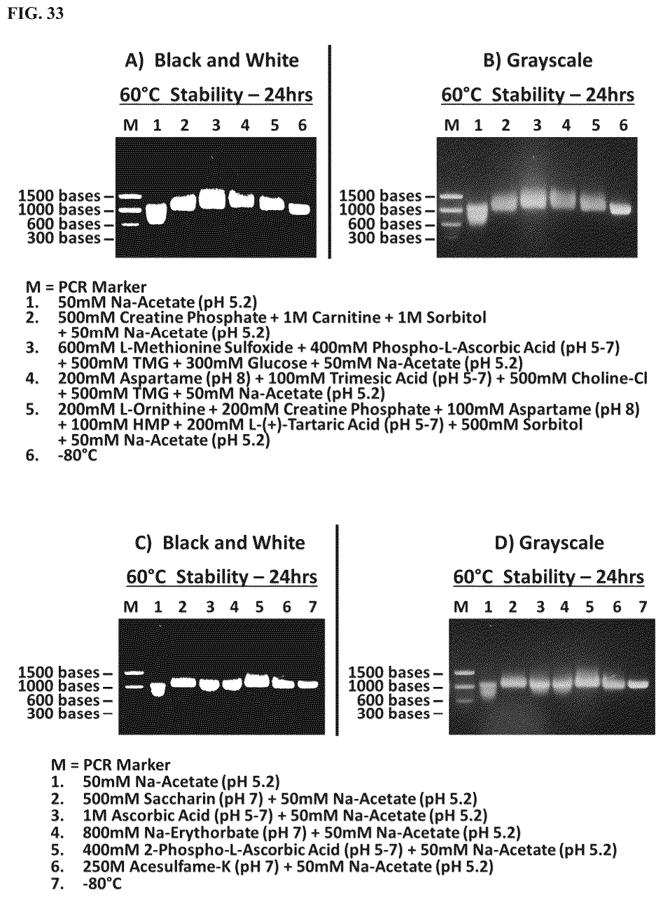

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIG. 33A and B.

Following dilution of each sample in each respective RNA storage environment, samples were stored in a thermal cycler at 60° C. for about 24 hours and then analyzed by denaturing agarose gel electrophoresis as shown in FIG. 33A and B.

### Example 70

Accelerated RNA Stability Testing at 60° C.

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 33 C and D.

Following dilution of each sample in each respective RNA storage environment, samples were stored in a thermal cycler at 60° C. for about 24 hours and then analyzed by denaturing agarose gel electrophoresis as shown in FIG. 33 C and D.

Ascorbic Acid and Na-Erythorbate were made fresh prior to adding to sample.

The following were purchased from: Ascorbic Acid (Eisen-Golden), Na-Erythorbate (Eisen-Golden).

### Example 71

Accelerated RNA Stability Testing at 60° C.

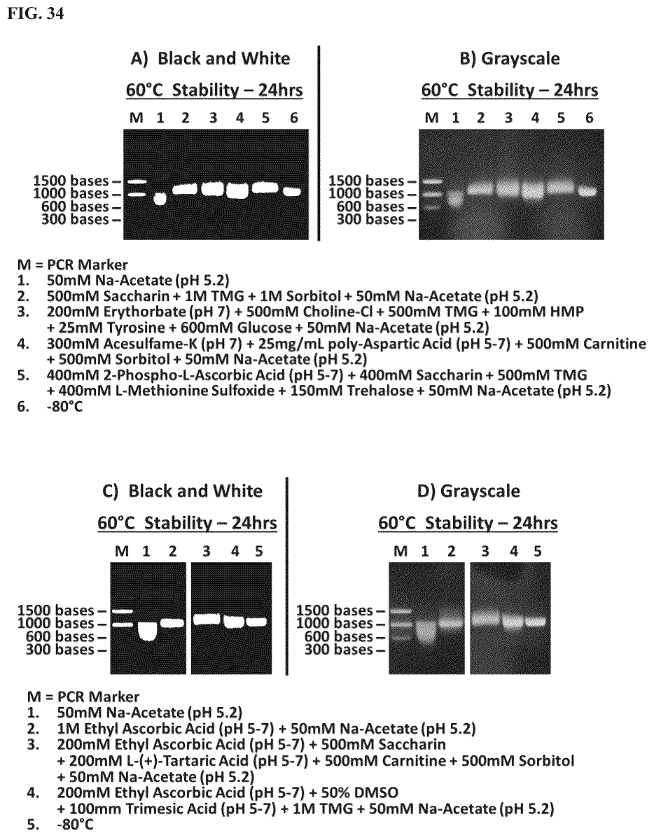

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 34A and B.

Following dilution of each sample in each respective RNA storage environment, samples were stored in a thermal cycler at 60° C. for about 24 hours and then analyzed by denaturing agarose gel electrophoresis as shown in FIG. 34A and B.

Ascorbic Acid and Na-Erythorbate were made fresh prior to adding to sample.

The following were purchased from: poly-Aspartic Acid (Mark Nature, Fullerton, CA).

### Example 72

Accelerated RNA Stability Testing at 60° C.

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 34 C and D.

Following dilution of each sample in each respective RNA storage environment, samples were stored in a thermal cycler at 60° C. for about 24 hours and then analyzed by denaturing agarose gel electrophoresis as shown in FIG. 34 C and D.

Ethyl Ascorbic Acid was made fresh prior to adding to sample.

The following were purchased from: Ethyl Ascorbic Acid (Pure Health Botanicals, LLC, Saint Charles, IL, dba as ebay purehealthsolutions), L-(+)-Tartaric Acid (Sigma Aldrich, Product #251380).

### Example 73

Accelerated RNA Stability Testing at 60° C.

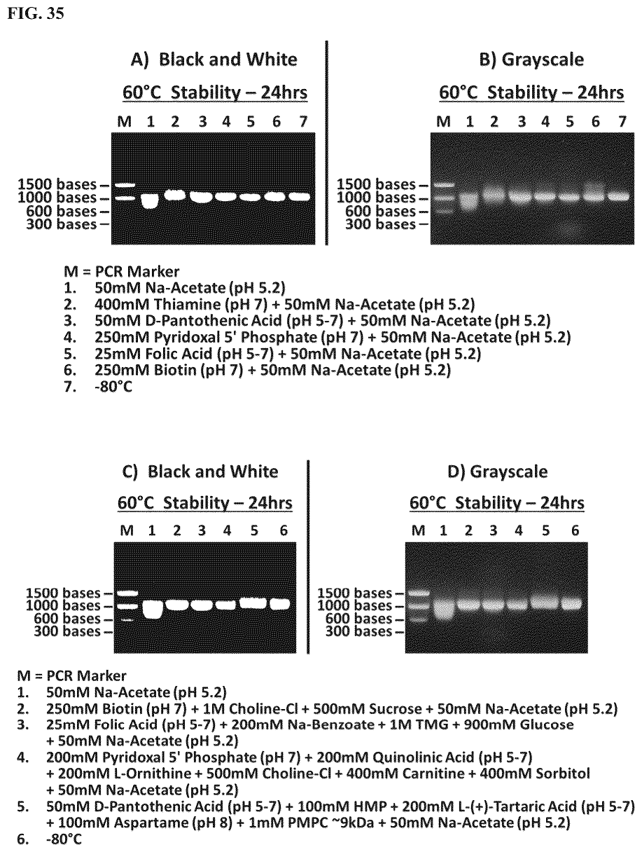

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIG. 35A and B.

Following dilution of each sample in each respective RNA storage environment, samples were stored in a thermal cycler at 60° C. for about 24 hours and then analyzed by denaturing agarose gel electrophoresis as shown in FIG. 35A and B.

Samples 5 and 6 were buffer exchanged to remove folic acid and biotin prior to running samples to improve analysis by gel electrophoresis.

The following were purchased from: Thiamine (Cayman Chemical, Product #25656), D-Pantothenic Acid (Cayman Chemical, Product #17288), Pyridoxal 5′ Phosphate (Cayman Chemical, Product #20352), Folic Acid (Cayman Chemical, Product #20515), Biotin (Cayman Chemical, Product #22582).

### Example 74

Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIG. 35 C and D.

Following dilution of each sample in each respective RNA storage environment, samples were stored in a thermal cycler at 60° C. for about 24 hours and then analyzed by denaturing agarose gel electrophoresis as shown in FIG. 35 C and D.

Samples 1 and 2 were buffer exchanged to remove folic acid and biotin prior to running sample to improve analysis by gel electrophoresis.

### Example 75

Accelerated RNA Stability Testing at 60° C.

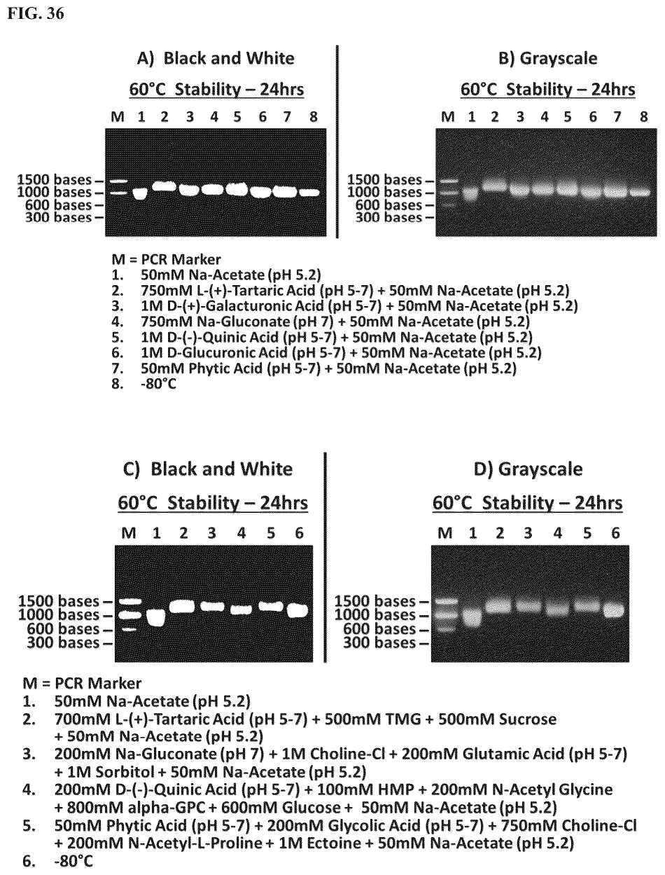

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIG. 36A and B.

Following dilution of each sample in each respective RNA storage environment, samples were stored in a thermal cycler at 60° C. for about 24 hours and then analyzed by denaturing agarose gel electrophoresis as shown in FIG. 36A and B.

Samples 3, 5, and 6 were buffer exchanged to remove galacturonic acid, quinic acid, and glucuronic acid prior to running samples to improve analysis by gel electrophoresis.

The following were purchased from: D-(+)-Galacturonic acid (Sigma-Aldrich, Product #48280), Na-Gluconate (Sigma Aldrich, Product #S2054), D-(−)-Quinic Acid (Sigma-Aldrich, Product #138622) D-Glucuronic Acid (Sigma-Aldrich, Product #31531).

### Example 76

Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIG. 36 C and D.

Following dilution of each sample in each respective RNA storage environment, samples were stored in a thermal cycler at 60° C. for about 24 hours and then analyzed by denaturing agarose gel electrophoresis as shown in FIG. 36 C and D.

### Example 77

Accelerated RNA Stability Testing at 60° C.

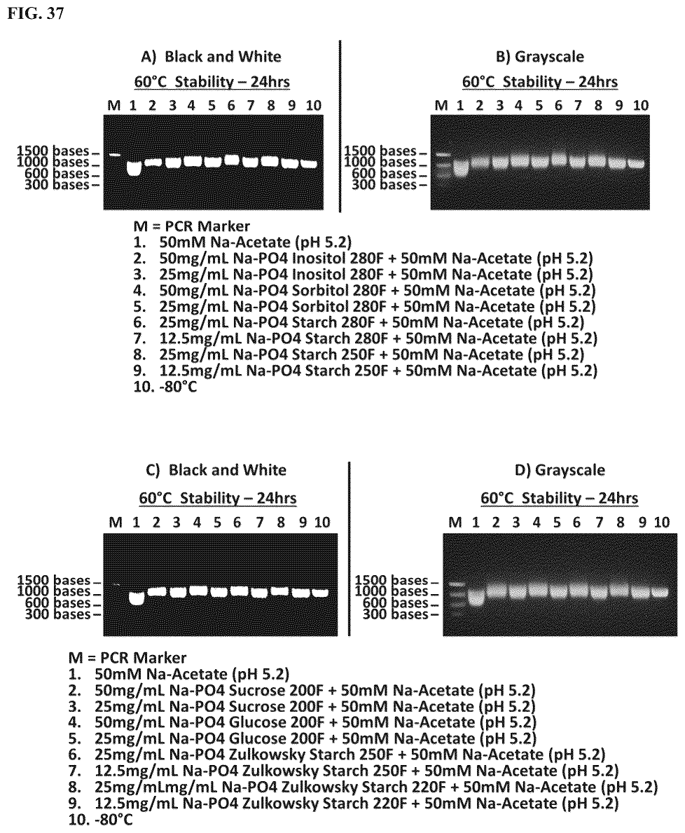

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIG. 37A and B.

Following dilution of each sample in each respective RNA storage environment, samples were stored in a thermal cycler at 60° C. for about 24 hours and then analyzed by denaturing agarose gel electrophoresis as shown in FIG. 37A and B.

### Example 78

Accelerated RNA Stability Testing at 60° C.

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 37 C and D.

Following dilution of each sample in each respective RNA storage environment, samples were stored in a thermal cycler at 60° C. for about 24 hours and then analyzed by denaturing agarose gel electrophoresis as shown in FIG. 37 C and D.

### Example 79

Accelerated RNA Stability Testing at 60° C.

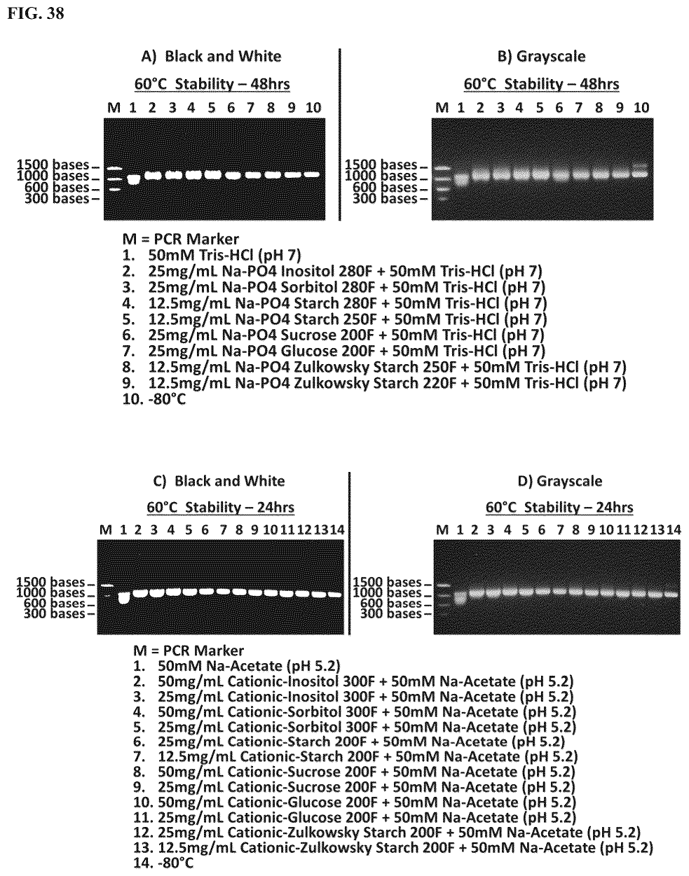

In vitro transcribed RNA was diluted at a ratio of about 1:10 (about 100-300n/mL) in different compositions containing either 50 mM Tris-HCl (pH 7), or one or more compounds in compositions with 50 mM Tris-HCl (pH 7) as shown in FIG. 38A and B.

Following dilution of each sample in each respective RNA storage environment, samples were stored in a thermal cycler at 60° C. for about 48 hours and then analyzed by denaturing agarose gel electrophoresis as shown in FIG. 38A and B.

### Example 80

Accelerated RNA Stability Testing at 60° C.

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 38 C and D.

Following dilution of each sample in each respective RNA storage environment, samples were stored in a thermal cycler at 60° C. for about 24 hours and then analyzed by denaturing agarose gel electrophoresis as shown in FIG. 38 C and D.

### Example 81

Accelerated RNA Stability Testing at 60° C.

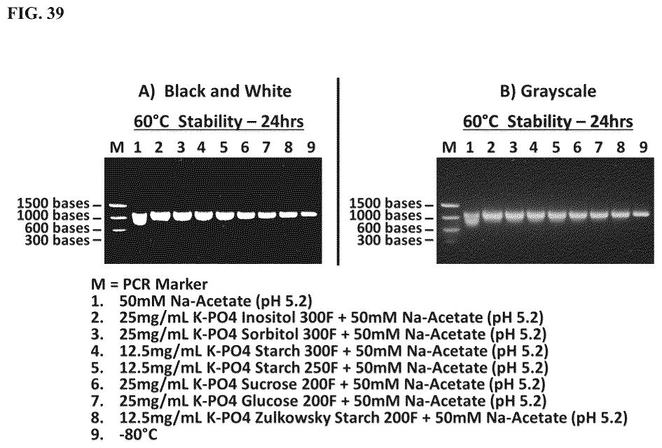

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 39A and B.

Following dilution of each sample in each respective RNA storage environment, samples were stored in a thermal cycler at 60° C. for about 24 hours and then analyzed by denaturing agarose gel electrophoresis as shown in FIG. 39A and B.

### Example 82

Accelerated RNA Stability Testing at 60° C.

In vitro transcribed RNA was diluted at a ratio of about 1:10 (about 100-300n/mL) in different compositions containing either 50 mM Tris-HCl (pH 7), or one or more compounds in compositions with 50 mM Tris-HCl (pH 7) as shown in FIG. 40A and B.

Following dilution of each sample in each respective RNA storage environment, samples were stored in a thermal cycler at 60° C. for about 48 hours and then analyzed by denaturing agarose gel electrophoresis as shown in FIG. 40A and B.

### Example 83

Accelerated RNA Stability Testing at 60° C.

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 40 C and D.

Following dilution of each sample in each respective RNA storage environment, samples were stored in a thermal cycler at 60° C. for about 24 hours and then analyzed by denaturing agarose gel electrophoresis as shown in FIG. 40 C and D.

### Example 84

Accelerated RNA Stability Testing at 60° C.

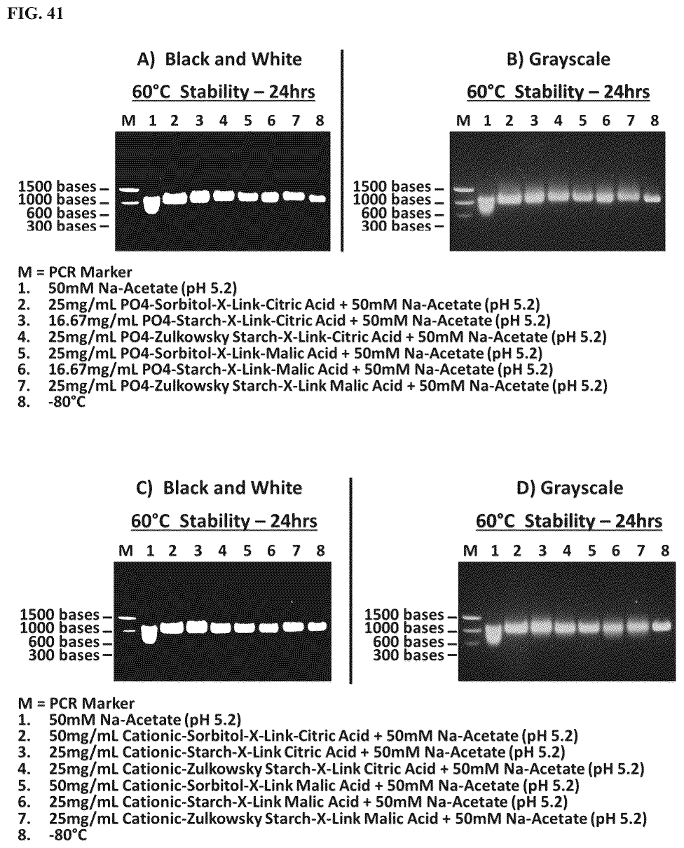

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 41A and B.

Following dilution of each sample in each respective RNA storage environment, samples were stored in a thermal cycler at 60° C. for about 24 hours and then analyzed by denaturing agarose gel electrophoresis as shown in FIG. 41A and B.

### Example 85

Accelerated RNA Stability Testing at 60° C.

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 41 C and D.

Following dilution of each sample in each respective RNA storage environment, samples were stored in a thermal cycler at 60° C. for about 24 hours and then analyzed by denaturing agarose gel electrophoresis as shown in FIG. 41 C and D.

### Example 86

Accelerated RNA Stability Testing at 60° C.

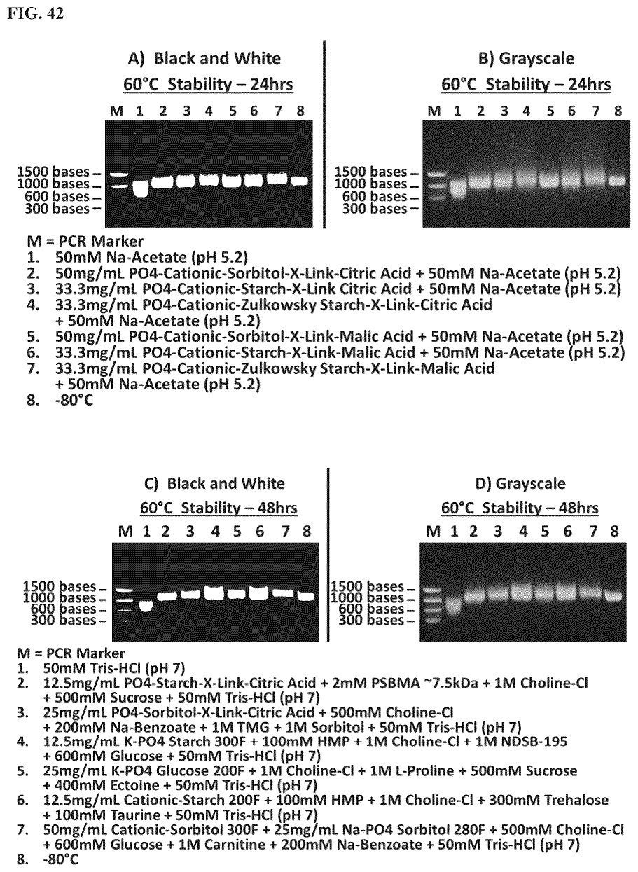

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 42A and B.

Following dilution of each sample in each respective RNA storage environment, samples were stored in a thermal cycler at 60° C. for about 24 hours and then analyzed by denaturing agarose gel electrophoresis as shown in FIG. 42A and B.

### Example 87

Accelerated RNA Stability Testing at 60° C.

In vitro transcribed RNA was diluted at a ratio of about 1:10 (about 100-300n/mL) in different compositions containing either 50 mM Tris-HCl (pH 7), or one or more compounds in compositions with 50 mM Tris-HCl (pH 7) as shown in FIG. 42 C and D.

Following dilution of each sample in each respective RNA storage environment, samples were stored in a thermal cycler at 60° C. for about 48 hours and then analyzed by denaturing agarose gel electrophoresis as shown in FIG. 42 C and D.

### Example 88

Accelerated RNA Stability Testing at 60° C.

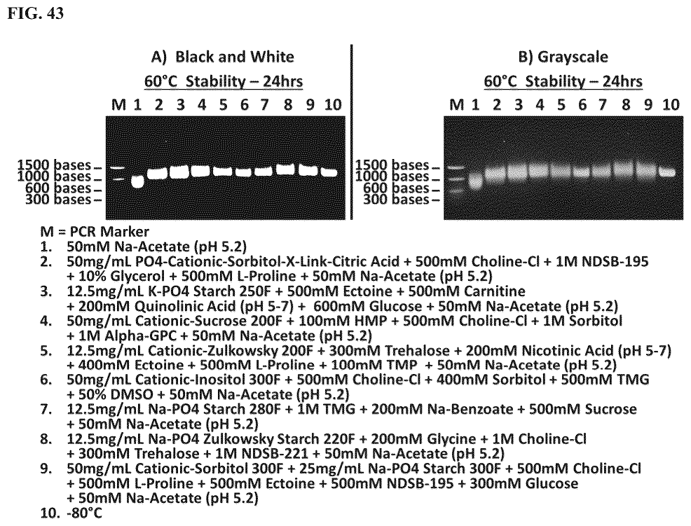

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 43A and B.

Following dilution of each sample in each respective RNA storage environment, samples were stored in a thermal cycler at 60° C. for about 24 hours and then analyzed by denaturing agarose gel electrophoresis as shown in FIG. 43A and B.

### Example 89

Accelerated RNA Stability Testing at 60° C.

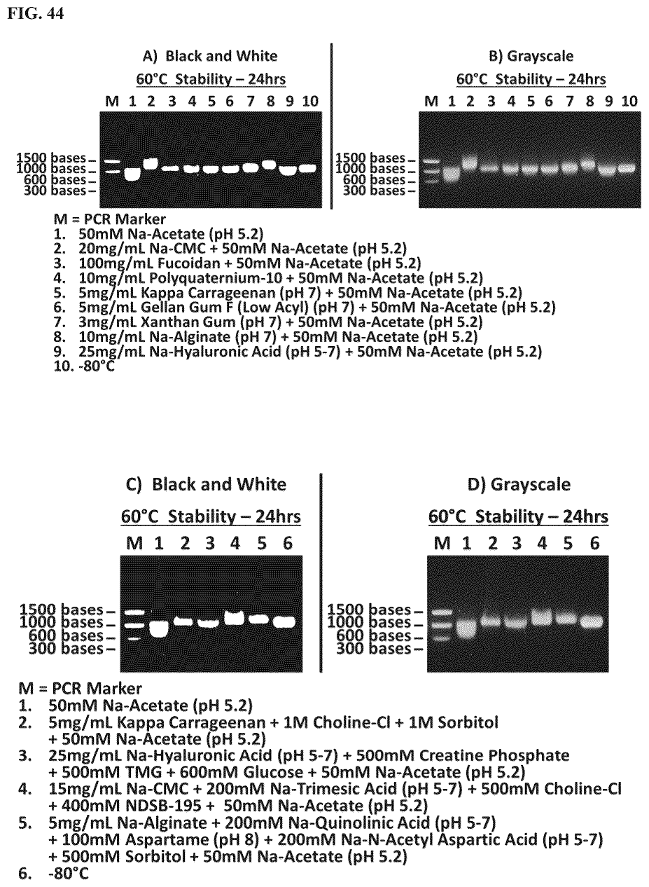

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 44A and B.

Following dilution of each sample in each respective RNA storage environment, samples were stored in a thermal cycler at 60° C. for about 24 hours and then analyzed by denaturing agarose gel electrophoresis as shown in FIG. 44A and B.

The sample with Polyquaternium-10 was incubated with 2 mM PAA for 1 hr at room temperature prior to running to improve analysis by agarose gel electrophoresis. All samples were diluted between 2-3× with water prior to running to reduce viscosity and improve analysis. The following were purchased from: Na-Carboxymethylcellulose (CMC) ˜90 kDa (Sigma-Aldrich, Product #419273), Fucoidan (Cayman Chemical, Product #20357), Polyquaternium-10 (Divinity Cosmetic Supply, Port St Lucie, FL), Kappa Carrageenan (Modernist Pantry, Product #1011-50), Gellan Gum F—Low Acyl (Modernist Pantry, Product #1028), Xanthan Gum (Modernist Pantry, Product #1019-50), Na-Alginate (Modernist Pantry, Product #1007-50), Hyaluronic Acid ˜10 kDa (Pure Health Botanicals, LLC, Saint Charles, IL, dba as ebay purehealthsolutions)

### Example 90

Accelerated RNA Stability Testing at 60° C.

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 44 C and D.

Following dilution of each sample in each respective RNA storage environment, samples were stored in a thermal cycler at 60° C. for about 24 hours and then analyzed by denaturing agarose gel electrophoresis as shown in FIG. 44 C and D. All samples were diluted between 2-3× with water prior to running to reduce viscosity and improve analysis.

### Example 91

Accelerated RNA Stability Testing at 60° C.

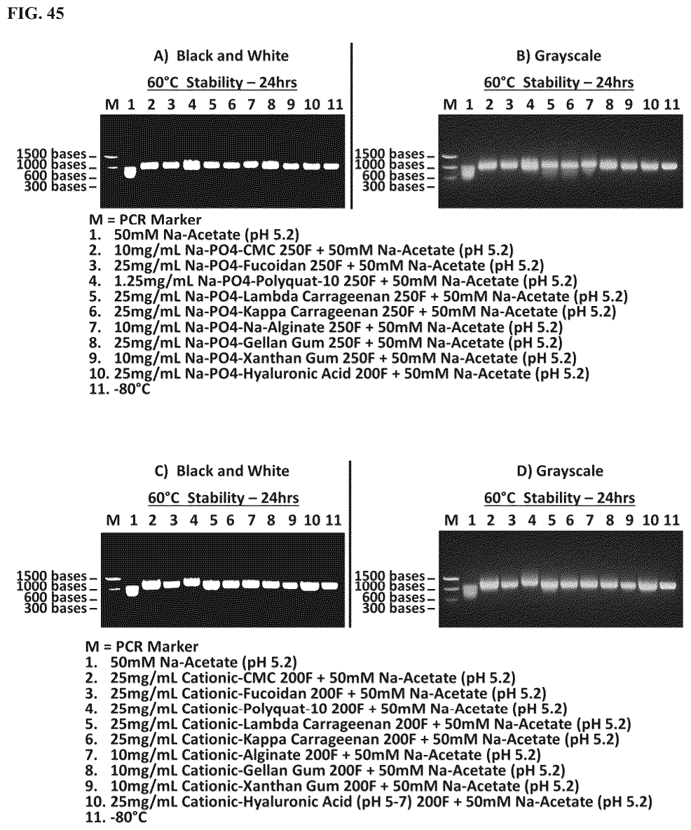

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 45A and B.

Following dilution of each sample in each respective RNA storage environment, samples were stored in a thermal cycler at 60° C. for about 24 hours and then analyzed by denaturing agarose gel electrophoresis as shown in FIG. 45A and B.

The sample with Na-PO4-Polyquat-10 250F was incubated with 2 mM PAA ˜8 kDa for 1 hr at room temperature prior to running to improve analysis by agarose gel electrophoresis.

### Example 92

Accelerated RNA Stability Testing at 60° C.

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 45 C and D.

Following dilution of each sample in each respective RNA storage environment, samples were stored in a thermal cycler at 60° C. for about 24 hours and then analyzed by denaturing agarose gel electrophoresis as shown in FIG. 45 C and D.

The sample with Cationic-Polyquat-10 200F was incubated with 1 mM PAA ˜8 kDa for 1 hr at room temperature prior to running to improve analysis by agarose gel electrophoresis.

### Example 93

Accelerated RNA Stability Testing at 60° C.

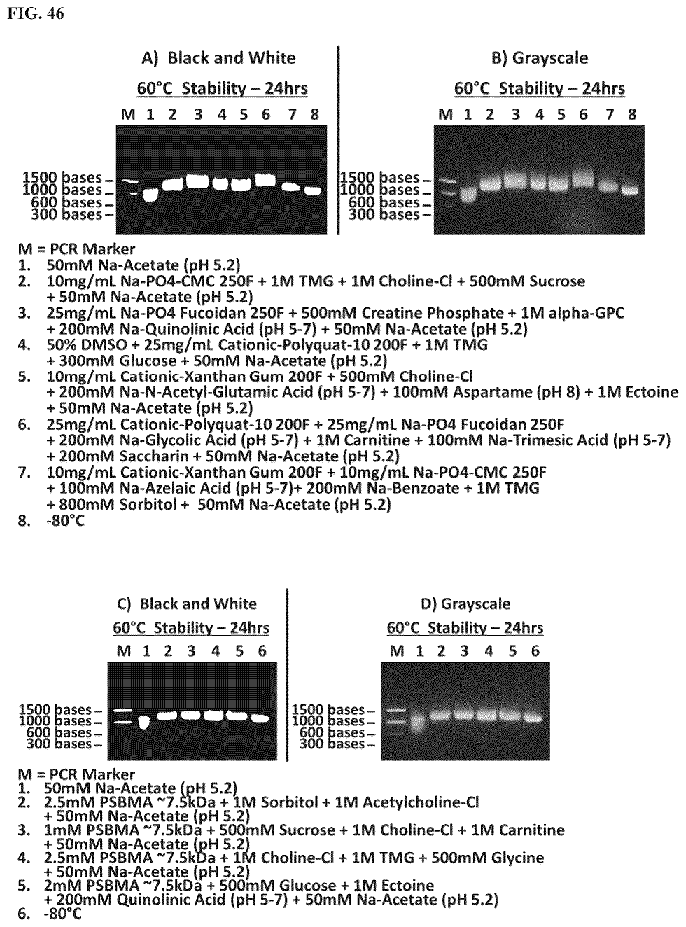

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 46A and B.

Following dilution of each sample in each respective RNA storage environment, samples were stored in a thermal cycler at 60° C. for about 24 hours and then analyzed by denaturing agarose gel electrophoresis as shown in FIG. 46A and B.

The sample with Cationic-Polyquat-10 200F was incubated with 1 mM PAA ˜8 kDa for 1 hr at room temperature prior to running to improve analysis by agarose gel electrophoresis.

### Example 94

Accelerated RNA Stability Testing at 60° C.

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 46 C and D.

Following dilution of each sample in each respective RNA storage environment, samples were stored in a thermal cycler at 60° C. for about 24 hours and then analyzed by denaturing agarose gel electrophoresis as shown in FIG. 46 C and D.

### Example 95

Accelerated RNA Stability Testing at 60° C.

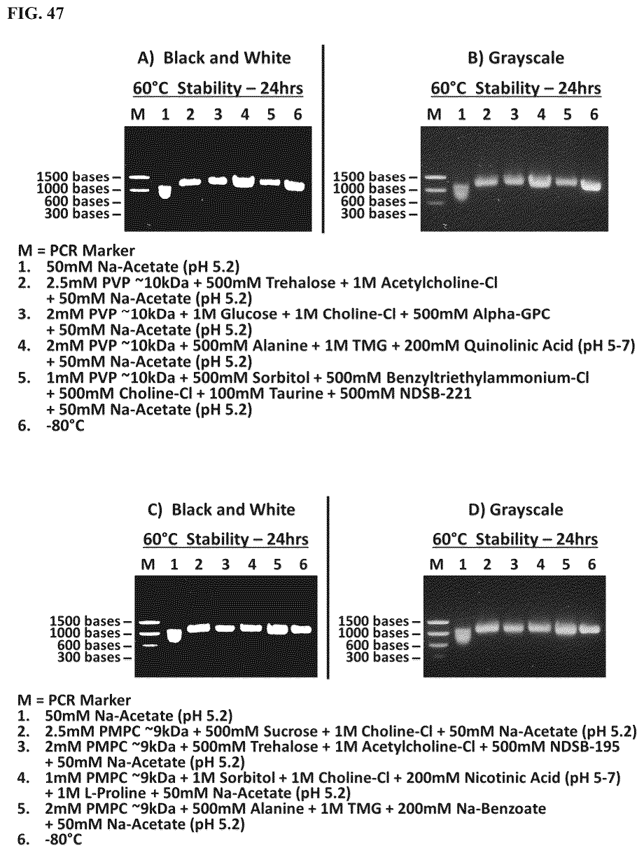

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 47A and B.

Following dilution of each sample in each respective RNA storage environment, samples were stored in a thermal cycler at 60° C. for about 24 hours and then analyzed by denaturing agarose gel electrophoresis as shown in FIG. 47A and B.

The sample with benzyltriethylammonium was incubated with 1 mM PAA ˜8 kDa for 1 hr at room temperature prior to analysis.

### Example 96

Accelerated RNA Stability Testing at 60° C.

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 47 C and D.

Following dilution of each sample in each respective RNA storage environment, samples were stored in a thermal cycler at 60° C. for about 24 hours and then analyzed by denaturing agarose gel electrophoresis as shown in FIG. 47 C and D.

### Example 97

Accelerated RNA Stability Testing at 60° C.

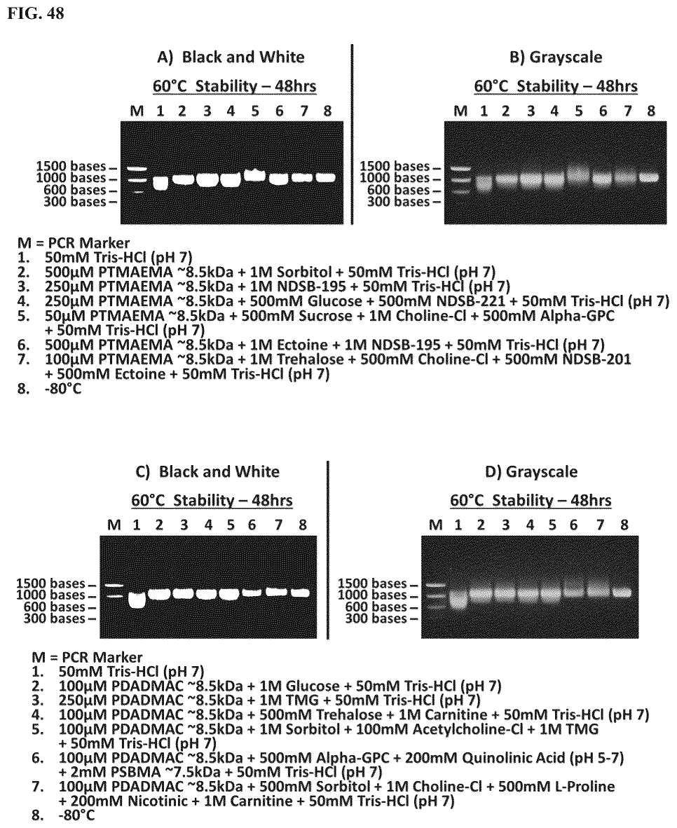

In vitro transcribed RNA was diluted at a ratio of about 1:10 (about 100-300n/mL) in different compositions containing either 50 mM Tris-HCl (pH 7), or one or more compounds in compositions with 50 mM Tris-HCl (pH 7) as shown in FIG. 48A and B.

Following dilution of each sample in each respective RNA storage environment, samples were stored in a thermal cycler at 60° C. for about 48 hours and then analyzed by denaturing agarose gel electrophoresis as shown in FIG. 48A and B.

All PTMAEMA samples were incubated with 2 mM PAA ˜8 kDa for 30 min at 70° C. prior to analysis.

### Example 98

Accelerated RNA Stability Testing at 60° C.

In vitro transcribed RNA was diluted at a ratio of about 1:10 (about 100-300n/mL) in different compositions containing either 50 mM Tris-HCl (pH 7), or one or more compounds in compositions with 50 mM Tris-HCl (pH 7) as shown in FIG. 48 C and D.

Following dilution of each sample in each respective RNA storage environment, samples were stored in a thermal cycler at 60° C. for about 48 hours and then analyzed by denaturing agarose gel electrophoresis as shown in FIG. 48 C and D.

All PDADMAC samples were incubated with 2 mM PAA ˜8 kDa for 1 hr at room temperature prior to analysis.

### Example 99

Accelerated RNA Stability Testing at 60° C.

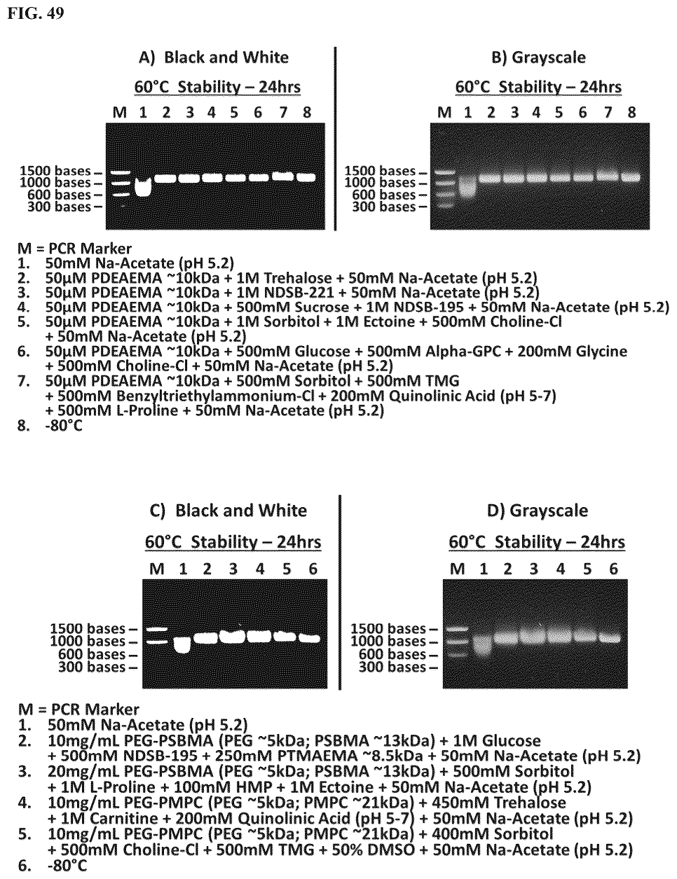

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 49A and B.

Following dilution of each sample in each respective RNA storage environment, samples were stored in a thermal cycler at 60° C. for about 24 hours and then analyzed by denaturing agarose gel electrophoresis as shown in FIG. 49A and B.

All PDEAEMA samples were incubated with 1 mM PAA ˜8 kDa for 1 hr at room temperature prior to analysis.

### Example 100

Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIG. 49 C and D.

Following dilution of each sample in each respective RNA storage environment, samples were stored in a thermal cycler at 60° C. for about 24 hours and then analyzed by denaturing agarose gel electrophoresis as shown in FIG. 49 C and D.

The sample with PTMAEMA was incubated with 1 mM PAA ˜8 kDa for 30 min at 70° C. prior to analysis.

### Example 101

Accelerated RNA Stability Testing at 60° C.

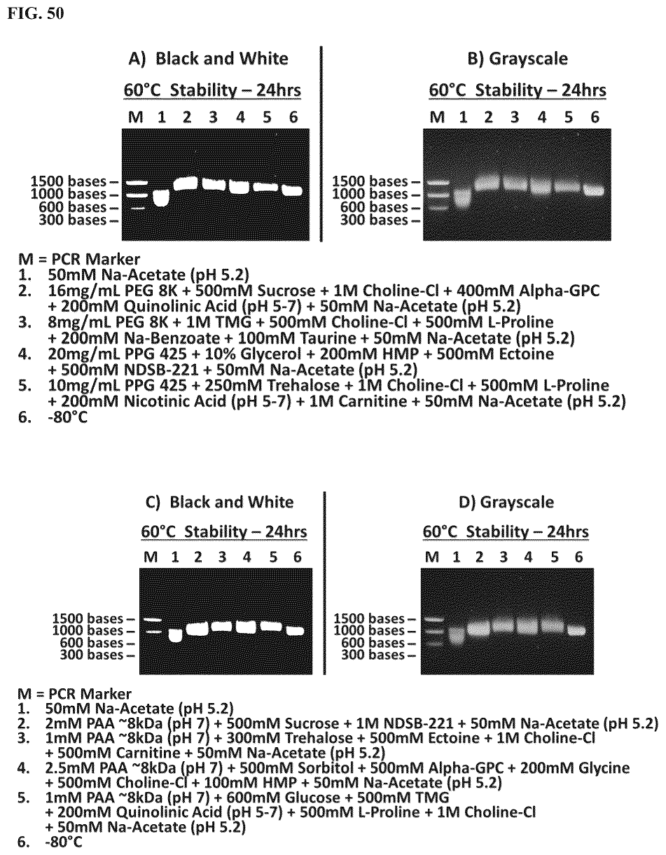

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIG. 50A and B.

Following dilution of each sample in each respective RNA storage environment, samples were stored in a thermal cycler at 60° C. for about 24 hours and then analyzed by denaturing agarose gel electrophoresis as shown in FIG. 50A and B.

The following were purchased from: polyethylene glycol (˜8 kDa) (PEG 8K) (Sigma-Aldrich, Product #89510), polypropylene glycol (Mn 425) (PPG 425) (Sigma-Aldrich, Product #202304)

### Example 102

Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIG. 50 C and D.

Following dilution of each sample in each respective RNA storage environment, samples were stored in a thermal cycler at 60° C. for about 24 hours and then analyzed by denaturing agarose gel electrophoresis as shown in FIG. 50 C and D.

### Example 103

Accelerated RNA Stability Testing at 60° C.

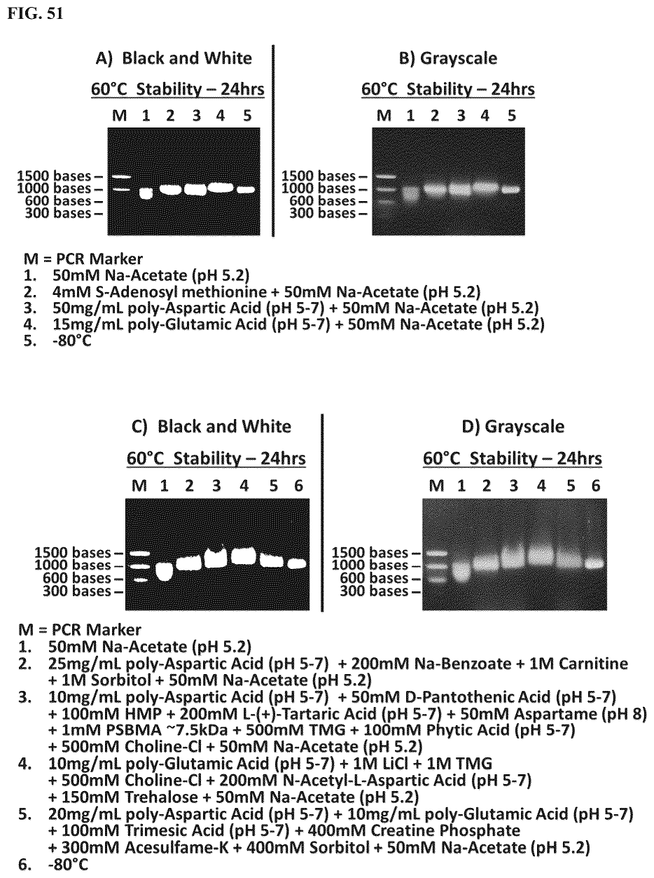

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 51A and B.

Following dilution of each sample in each respective RNA storage environment, samples were stored in a thermal cycler at 60° C. for about 24 hours and then analyzed by denaturing agarose gel electrophoresis as shown in FIG. 51A and B.

The following were purchased from: poly-Glutamic Acid (Pure Health Botanicals, LLC, Saint Charles, IL, dba as ebay purehealthsolutions), S-Adenosyl Methionine (Pure Bulk).

### Example 104

Accelerated RNA Stability Testing at 60° C.

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 51 C and D.

Following dilution of each sample in each respective RNA storage environment, samples were stored in a thermal cycler at 60° C. for about 24 hours and then analyzed by denaturing agarose gel electrophoresis as shown in FIG. 51 C and D.

### Example 105

Accelerated RNA Stability Testing at 60° C.

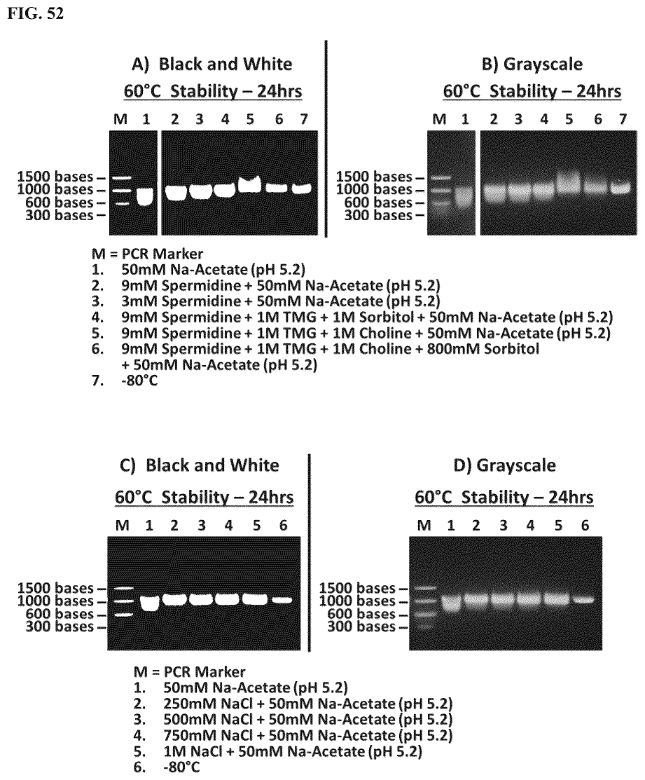

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 52A and B.

Following dilution of each sample in each respective RNA storage environment, samples were stored in a thermal cycler at 60° C. for about 24 hours and then analyzed by denaturing agarose gel electrophoresis as shown in FIG. 52A and B. Samples were incubated with 1 mM PAA ˜8 kDa for 1 hr at room temperature prior to analysis to improve analysis by agarose gel electrophoresis.

The following were purchased from: spermidine (Cayman Chemical, Product #14918).

### Example 106

Accelerated RNA Stability Testing at 60° C.

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 52 C and D.

Following dilution of each sample in each respective RNA storage environment, samples were stored in a thermal cycler at 60° C. for about 24 hours and then analyzed by denaturing agarose gel electrophoresis as shown in FIG. 52 C and D. Samples were incubated with 1 mM PAA ˜8 kDa for 1 hr at room temperature prior to analysis to improve analysis by agarose gel electrophoresis.

Sodium Chloride (NaCl) (Sigma-Aldrich, Product #S3014)

### Example 107

Accelerated RNA Stability Testing at 60° C.

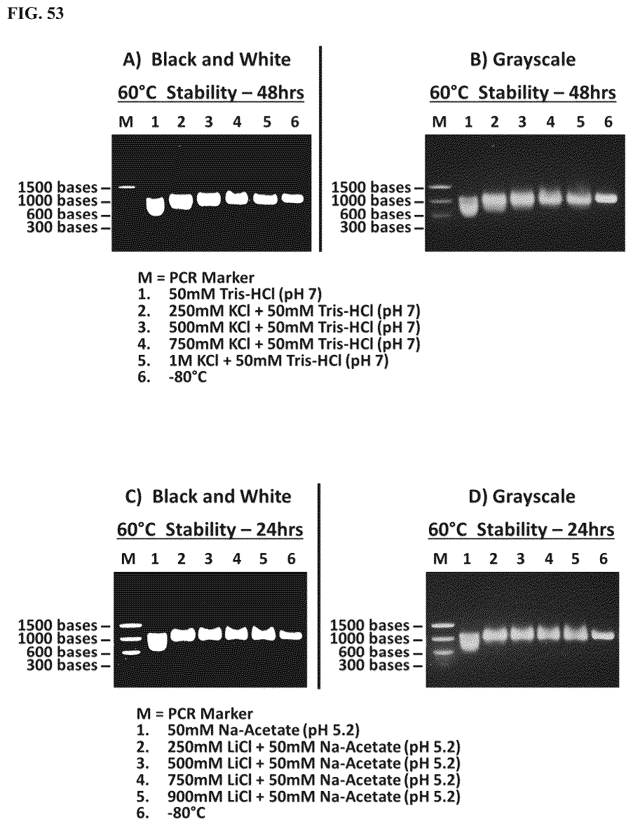

In vitro transcribed RNA was diluted at a ratio of about 1:10 (about 100-300n/mL) in different compositions containing either 50 mM Tris-HCl (pH 7), or one or more compounds in compositions with 50 mM Tris-HCl (pH 7) as shown in FIG. 53A and B.

Following dilution of each sample in each respective RNA storage environment, samples were stored in a thermal cycler at 60° C. for about 48 hours and then analyzed by denaturing agarose gel electrophoresis as shown in FIG. 53A and B. Samples were incubated with 1 mM PAA ˜8 kDa for 1 hr at room temperature prior to analysis to improve analysis by agarose gel electrophoresis.

Potassium Chloride (KCl) (Sigma-Aldrich, Product #P9541)

### Example 108

Accelerated RNA Stability Testing at 60° C.

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 53 C and D.

Following dilution of each sample in each respective RNA storage environment, samples were stored in a thermal cycler at 60° C. for about 24 hours and then analyzed by denaturing agarose gel electrophoresis as shown in FIG. 53 C and D.

Lithium Chloride (LiCl), Lithium Chloride Solution (New England Biolabs, as component B2051AVIAL in Product #E2050S).

Samples were incubated with 1 mM PAA ˜8 kDa for 1 hr at room temperature prior to analysis to improve analysis by agarose gel electrophoresis.

### Example 109

Accelerated RNA Stability Testing at 60° C.

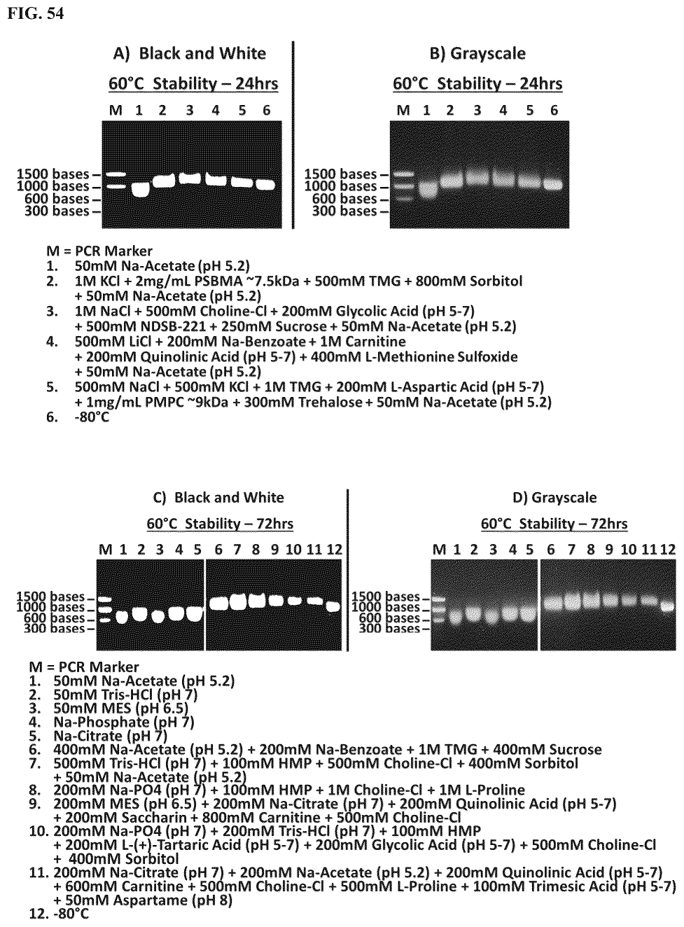

In vitro transcribed RNA was diluted at a ratio of about 1:10 (about 100-300n/mL) in different compositions containing either 50 mM Na-Acetate (pH 5.2), or one or more compounds in compositions with 50 mM Na-Acetate (pH 5.2) as shown in FIG. 54A and B.

Following dilution of each sample in each respective RNA storage environment, samples were stored in a thermal cycler at 60° C. for about 24 hours and then analyzed by denaturing agarose gel electrophoresis as shown in FIG. 54A and B.

Samples were incubated with 1 mM PAA ˜8 kDa for 1 hr at room temperature prior to analysis to improve analysis by agarose gel electrophoresis.

### Example 110

Accelerated RNA Stability Testing at 60° C.

In vitro transcribed RNA was diluted at a ratio of about 1:10 (about 100-300n/mL) in different compositions containing either 50 mM Na-Acetate (pH 5.2), 50 mM Tris-HCl (pH 7), 50 mM MES buffer (pH 6.5), Na-Phosphate (pH 7), Na-Citrate (pH 7) or various combinations of more than one RNA stabilizing substance with selected buffers as shown in FIG. 54 C and D.

Following dilution of each sample in each respective RNA storage environment, samples were stored in a thermal cycler at 60° C. for about 72 hours and then analyzed by denaturing agarose gel electrophoresis as shown in FIG. 54 C and D.

### Example 111

Accelerated RNA Stability Testing at 60° C.

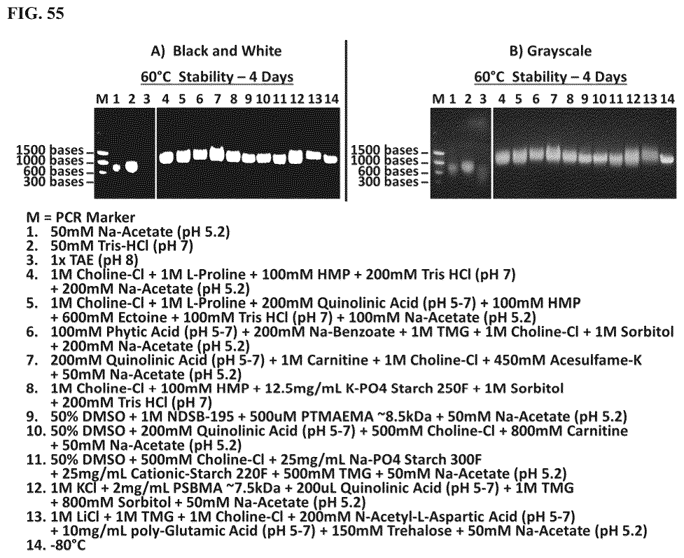

In vitro transcribed RNA was diluted at a ratio of about 1:10 (about 100-300n/mL) in different compositions containing either 50 mM Na-Acetate (pH 5.2), 50 mM Tris-HCl (pH 7), TAE buffer (pH 8) or various combinations of more than one RNA stabilizing substance with selected buffers as shown in FIG. 55A and B.

Following dilution of each sample in each respective RNA storage environment, samples were stored in a thermal cycler at 60° C. for about 4 days and then analyzed by denaturing agarose gel electrophoresis as shown in FIG. 55A and B.

### Example 112

Accelerated RNA Stability Testing at 60° C.

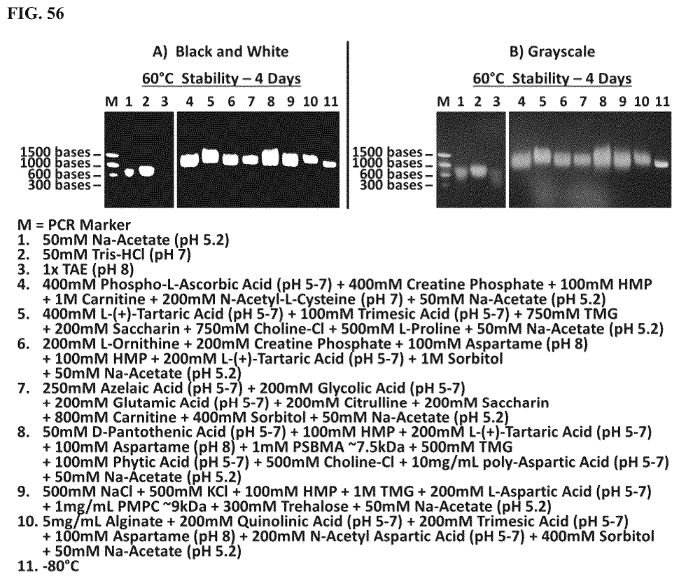

In vitro transcribed RNA was diluted at a ratio of about 1:10 (about 100-300n/mL) in different compositions containing either 50 mM Na-Acetate (pH 5.2), 50 mM Tris-HCl (pH 7), TAE buffer (pH 8) or various combinations of more than one RNA stabilizing substance with selected buffers as shown in FIG. 56A and B.

Following dilution of each sample in each respective RNA storage environment, samples were stored in a thermal cycler at 60° C. for about 4 days and then analyzed by denaturing agarose gel electrophoresis as shown in FIG. 56A and B.

1. A composition, comprising at least one RNA substance and at least one RNA stabilizing substance, wherein:
the at least one RNA stabilizing substance comprises a modified polysaccharide substance that comprises one or more substituents selected from quaternary amines, tertiary amines, and phosphate groups;
the modified polysaccharide substance is a modified species of polysaccharide;
when the modified polysaccharide substance comprises one or more substituents selected from quaternary amines, then the one or more substituents are selected from formulas A and B: 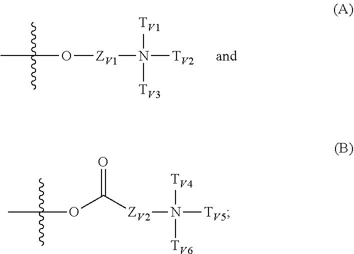
Z_{V1} and Z_{V2} are independently selected from a C₁₋₆ alkyl or alkenyl that is optionally substituted with up to 2 heteroatoms and that is optionally substituted with one or both of hydroxy and oxo;
T_{V1}, T_{V2}, T_{V3}, T_{V4}, T_{V5}, and T_{V6} are independently selected from a C₁₋₄ alkyl that is optionally substituted with one hydroxy;
when the modified polysaccharide substance comprises one or more substituents selected from tertiary amines, then the substituents are selected from formulas C and D: 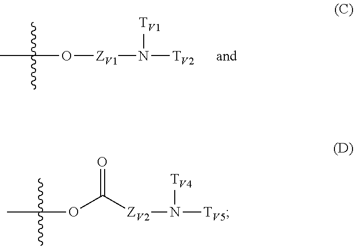
Z_{V1} and Z_{V2} are independently selected from a C₁₋₆ alkyl or alkenyl that is optionally substituted with up to 2 heteroatoms and that is optionally substituted with one or both of hydroxy and oxo;
T_{V1}, T_{V2}, T_{V4}, and T_{V5} are independently selected from a C₁₋₄ alkyl that is optionally substituted with one hydroxy; and
each heteroatom is independently selected from oxygen, sulfur, and nitrogen.

2. The composition of claim 1, further comprising an acyclic quaternary ammonium substance selected from trimethylglycine, choline, carnitine, O-acetyl-carnitine, alpha-glycerophosphorylcholine, gamma-butyrobetaine, 3-[dimethyl-(2-hydroxyethyl)ammonio]-1-propanesulfonate, and 3-[ethyl(dimethyl)ammonio]-1-propanesulfonate.

3. The composition of claim 1 or 2, further comprising a substituted pyridine substance comprising a pyridine dicarboxylate selected from pyridine-2,3-dicarboxylate, pyridine-2,4-dicarboxylate, pyridine-2,5-dicarboxylate, pyridine-2,6-dicarboxylate, pyridine-3,4-dicarboxylate, pyridine-3,5-dicarboxylate, and 4-hydroxy-pyridine-2,6-dicarboxylate.

4. The composition of claim 1, wherein the modified polysaccharide substance is a modified species of cyclodextrin.

5. The composition of any one of claims 1-3, further comprising a cellular uptake agent selected from cationic lipids, ionizable lipids, and zwitterionic lipids.

6. The composition of any one of claims 1-3, wherein the RNA substance comprises one or more of mRNA and self-amplifying RNA.

7. The composition of any one of claims 1-3 wherein the composition is a pharmaceutical composition.

8. The pharmaceutical composition of claim 7, wherein the pharmaceutical composition is a medicament, a therapeutic, or a vaccine.

9. The composition of claim 7, wherein the composition comprises a biostimulant.

10. A chamber that contains the composition of any one of claims 1-3, wherein the chamber comprises a container; the container contains the composition; and either the container is a syringe or the chamber is hermetically sealed.

11. The composition of claim 2 or 3, wherein the RNA substance has a rate of degradation at 20° C. for 72 hours that is less than 50 percent per 72 hours.

12. The composition of claim 2 or 3, wherein the RNA substance has a rate of degradation at 40° C. for 72 hours that is less than 50 percent per 72 hours.

13. The composition of claim 1, wherein the modified polysaccharide substance is a modified species of polysaccharide selected from amylose, amylopectin, dextran, dextrin, cellulose, beta-glucan, mixed beta-glucan, hyaluronic acid, xanthan gum, gellan gum, carboxymethyl cellulose, alginate, inulin, sinistrin, levan, chitosan, and chitin.

14. The composition of claim 1, wherein the modified polysaccharide substance is a modified species of polysaccharide selected from amylose, dextran, dextrin, and sinistrin.

15. The composition of claim 1, wherein:
the substituent is selected from X-(trialkylamino)alkyloxy, X-(trialkylamino)-1-oxoalkyloxy, X-(dialkylamino)alkyloxy, and X-(dialkylamino)-1-oxoalkyloxy;
X is an integer selected from 1, 2, 3, and 4;
alkyloxy and 1-oxoalkyloxy comprise exactly X carbon atoms;
alkyloxy is selected from methyloxy, ethyloxy, propyloxy, and butyloxy;
1-oxoalkyloxy is selected from carbonyloxy, acetyloxy, propionyloxy, and 1-oxobutyloxy;
trialkyl is selected from trimethyl, triethyl, tripropyl, tributyl, and ethyl-dimethyl; and
dialkyl is selected from dimethyl and diethyl.

16. The composition of claim 1, wherein the substituent is selected from 2-(trimethylamino)ethyloxy; 2-(trimethylamino)-1-oxoethyloxy; and 3-(trimethylamino)-2-hydroxypropyloxy.

17. A composition, comprising at least one RNA substance and at least two RNA stabilizing substances, wherein:
the at least two RNA stabilizing substances comprise at least one acyclic quaternary ammonium substance and at least one substituted pyridine substance;
the at least one acyclic quaternary ammonium substance is selected from trimethylglycine, choline, carnitine, 0-acetyl-carnitine, alpha-glycerophosphorylcholine, gamma-butyrobetaine, 3-[dimethyl-(2-hydroxyethyl)ammonio]-1-propanesulfonate, and 3-[ethyl(dimethyl)ammonio]-1-propanesulfonate; and
the at least one substituted pyridine substance is selected from pyridine-2,3-dicarboxylate, pyridine-2,4-dicarboxylate, pyridine-2,5-dicarboxylate, pyridine-2,6-dicarboxylate, pyridine-3,4-dicarboxylate, pyridine-3,5-dicarboxylate, and 4-hydroxy-pyridine-2,6-dicarboxylate.

18. The composition of claim 17, further comprising an ascorbic acid derivative substance selected from 3-O-methyl-ascorbic acid, 3-O-ethyl-ascorbic acid, 3-O-propyl-ascorbic acid, 3-O-butyl-ascorbic acid, 2-O-methyl-ascorbic acid, 2-O-ethyl-ascorbic acid, 2-O-propyl-ascorbic acid, 2-O-butyl-ascorbic acid, 2-O-(2,3-dihydroxypropyl) ascorbic acid, 3-O-(2,3-dihydroxypropyl) ascorbic acid, 2-O-alpha-D-glucopyranosyl-ascorbic acid, 2-phospho-ascorbic acid, and 3-phospho-ascorbic acid.

19. The composition of claim 17, further comprising a polyphosphate substance of the formula (F) 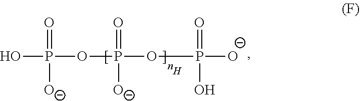
wherein n_{H} is an integer selected from 1-10,000.

20. The composition of claim 17 further comprising a cellular uptake agent selected from cationic lipids, ionizable lipids, and zwitterionic lipids.

21. The composition of claim 17, wherein the RNA substance further comprises one of mRNA and self-amplifying RNA.

22. The composition of claim 17, wherein the composition is a pharmaceutical composition.

23. The pharmaceutical composition of claim 22, wherein the pharmaceutical composition is selected from a medicament, a therapeutic, and a vaccine.

24. The composition of claim 17, wherein said composition comprises a chamber.

25. The composition of claim 17, comprising at least one additive substance selected from: sucrose, glucose, glycerol, sorbitol, trehalose, saccharin saccharine, acesulfame, aspartame, and maltitol.

26. The composition of claim 17, wherein the RNA substance has a rate of degradation at 20° C. for 72 hours that is less than 50 percent per 72 hours.

27. The composition of claim 17, wherein the RNA substance has a rate of degradation at 40° C. for 72 hours that is less than 50 percent per 72 hours.

28-37. (canceled)
